(19) United States
(12) Patent Application Publication
(10) Pub. No.: US 2023/0043046 A1
(43) Pub. Date: Feb. 9, 2023

(54) Recombinant CDKL5 Proteins, Gene Therapy and Production Methods

(71) Applicant: Amicus Therapeutics, Inc., Philadelphia, PA (US)

(72) Inventors: Sean Clark, Montgomery Township, NJ (US); Sean Sullivan, Milltown, NJ (US); Hilary Gray, Philadelphia, PA (US)

(73) Assignee: Amicus Therapeutics, Inc., Philadelphia, PA (US)

(21) Appl. No.: 17/773,416

(22) PCT Filed: Oct. 30, 2020
(86) PCT No.: PCT/US20/58247

§ 371 Date: Apr. 29, 2022

Related U.S. Application Data
(60) Provisional application No. 62/928,134, filed on Oct. 30, 2019.
(60) Provisional application No. 62/928,140, filed on Oct. 30, 2019.
(60) Provisional application No. 63/090,520, filed on Oct. 12, 2020.

(51) Int. Cl. A61K 48/00; A61P 25/28; C12N 9/12; C12N 15/86

(52) U.S. Cl. CPC A61K 48/0025; A61P 25/28; C12N 9/12; C12N 15/86; C12Y 207/11; A61K 38/00

(57) ABSTRACT

Compositions for CDKL5 gene therapy are provided, as well as recombinant CDKL5 proteins. Such CDKL5 gene therapy compositions and/or recombinant CDKL5 proteins may incorporate cell-penetrating polypeptides and/or leader signal polypeptides. Also provided are methods of producing such gene therapy compositions and recombinant CDKL5 proteins, as well as pharmaceutical compositions, methods of treatment, and uses of the gene therapy compositions and recombinant CDKL5 proteins.

### TECHNICAL FIELD

The present invention generally relates to the treatment of kinase deficiency disorders, particularly novel recombinant proteins and gene therapy for the treatment of disorders involving deficiency of CDKL5.

### BACKGROUND

CDKL5 is a serine/threonine kinase and was previously known as STK9. Mutations in this gene have recently been associated with a number of neurological disorders such as mental retardation, loss of communication and motor skills, infantile spasms and seizures, atypical Rett Syndrome, and X-linked West Syndromes. Mutations or deletions of the X-linked gene cyclin-dependent kinase-like 5 (CDKL5) have been shown to cause an epileptic encephalopathy with early-onset severe neurological impairment and intractable seizures.

Currently, the oldest known people described in medical literature with CDKL5 deficiency have reached an age of 41 years old. Many others are in their twenties and teens, but because the disease has only been identified in the last 15 years, the majority of newly diagnosed are toddlers or infants. Individuals diagnosed with CDKL5 deficiency disorder generally suffer delays in neurological development and are at a high risk for seizures, with a median onset age of 6 weeks. One study of 111 participants found that 85.6% of individuals had epilepsy with a daily occurrence of seizures, and a mean of 6 seizures per day.

Current treatments range from seizure medications, ketogenic diets, vagal nerve stimulation, and surgery. Commonly administered anti-epileptic medications include clobazam, valproic acid, and topiramate, and in many cases two or more medication regiments are used at the same time. Individuals seemed to have a “honeymoon period” in which they are seizure free for a period of time after starting a new type of medication, but ultimately there is a recurrence of seizures. The duration of observed honeymoon ranges from 2 months to 7 years, with a median of 6 months. For example, the study found that 16 of the 111 participants were currently seizure free, and one individual had never developed seizures.

The exact mechanisms for pathogenic manifestations remain unclear. Some experimental data suggest that certain non-sense mutations in the C-terminus cause the protein to be constitutively localized to the nucleus, while other missense mutations are highly represented in the cytoplasm. Nuclear localization signals and nuclear export signals have both been identified in the C-terminus of the protein.

Some mutant enzyme variants result in partial or total loss of phosphorylation function, while other mutations and truncations result in an increase in phosphorylation capacity, suggesting that both loss and gain of function may be pathogenic. Interactions and pathogenic effects arising from enzymatic activity loss/gain of function and enzyme nuclear localization versus residence in the cytoplasm remain unclear. An analysis of patients with a wide range of CDKL5 mutations and presenting clinical symptoms suggests that mutations causing clinical symptoms are more likely to be found either in the C-terminus or the kinase activity domain, suggesting that both the kinase activity and protein translocation capacity of CDKL5 could affect the clinical manifestation of symptoms.

### SUMMARY

Accordingly, various aspects of the invention pertain to new recombinant CDKL5 proteins and gene therapy compositions, which can be used to treat CDKL5-mediated neurological disorders such as a CDKL5 deficiency or an atypical Rett syndrome caused by a CDKL5 mutation or deficiency. Other aspects of the invention pertain to methods of producing such recombinant CDKL5 proteins and gene therapy compositions, as well as pharmaceutical compositions, methods of treatment, and uses of such recombinant proteins and gene therapy compositions.

One aspect of the present invention relates to a composition comprising a gene therapy delivery system and a CDKL5 polynucleotide encoding a CDKL5 polypeptide. In various embodiments, the CDKL5 polypeptide has at least 98% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26.

In one or more embodiments, the CDKL5 polypeptide has at least 98% sequence identity to SEQ ID NO: 1 or SEQ ID NO: 26. In one or more embodiments, the CDKL5 polynucleotide has at least 90% sequence identity to SEQ ID NO: 123.

In one or more embodiments, the CDKL5 polypeptide has at least 98% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

In one or more embodiments, the CDKL5 polypeptide has at least 98% sequence identity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25. In one or more embodiments, the CDKL5 polynucleotide has at least 90% sequence identity to SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147 or 1 SEQ ID NO: 149.

In one or more embodiments, the gene therapy delivery system comprises one or more of a viral vector, a liposome, a lipid-nucleic acid nanoparticle, an exosome and a gene editing system. In one or more embodiments, the gene editing system comprises one or more of Clustered Regularly Interspaced Short Palindromic Repeat (CRISPR) associated protein 9 (CRISPR-Cas-9), Transcription activator-like effector nuclease (TALEN) or ZNF (Zinc finger protein).

In one or more embodiments, the gene therapy delivery system comprises a viral vector. In one or more embodiments, the viral vector comprises one or more of an adenoviral vector, an adeno-associated viral vector, a lentiviral vector, a retroviral vector, a poxviral vector or a herpes simplex viral vector. In one or more embodiments, the viral vector comprises a viral polynucleotide operably linked to the CDKL5 polynucleotide. In one or more embodiments, the viral vector comprises at least one inverted terminal repeat (ITR).

In one or more embodiments, the composition further comprises one or more of an SV40 intron, a polyadenylation signal or a stabilizing element.

In one or more embodiments, the composition further comprises a promoter. In one or more embodiments, the promoter has at least 90% sequence identity to SEQ ID NO: 29 or SEQ ID NO: 30.

In one or more embodiments, the composition further comprises a polynucleotide encoding a cell-penetrating polypeptide. In one or more embodiments, the cell-penetrating polypeptide has at least 90% sequence identity to SEQ ID NO: 32, SEQ ID NO: 34, SEQ ID NO: 35, SEQ ID NO: 36, SEQ ID NO: 37 or SEQ ID NO: 167. In one or more embodiments, the polynucleotide encoding the cell-penetrating peptide has at least 90% sequence identity to SEQ ID NO: 150, SEQ ID NO: 151, SEQ ID NO: 152, SEQ ID NO: 153, SEQ ID NO: 154, SEQ ID NO: 170, SEQ ID NO: 171, SEQ ID NO: 172 or SEQ ID NO: 173.

In one or more embodiments, the composition further comprises a polynucleotide encoding a leader signal polypeptide. In one or more embodiments, the leader signal polypeptide has at least 90% sequence identity to SEQ ID NO: 38, SEQ ID NO: 39, SEQ ID NO: 40, SEQ ID NO: 41, SEQ ID NO: 42, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166 or SEQ ID NO: 168. In one or more embodiments, the polynucleotide encoding the leader signal polypeptide has at least 90% sequence identity to SEQ ID NO: 155. In one or more embodiments, the polynucleotide encoding the leader signal polypeptide has at least 90% sequence identity to SEQ ID NO: 169.

Another aspect of the present invention relates to a pharmaceutical formulation comprising a composition as described herein and a pharmaceutically acceptable carrier.

Another aspect of the present invention relates to a method of treating a CDKL5-mediated neurological disorder, the method comprising administering a composition or formulation as described herein to a patient in need thereof. In one or more embodiments, the composition or the formulation is administered intrathecally, intravenously, intracistnerally, intracerebroventrically or intraparenchymally. In one or more embodiments, the CDKL5-mediated neurological disorder is one or more of a CDKL5 deficiency or an atypical Rett syndrome caused by a CDKL5 mutation or deficiency.

Another aspect of the present invention relates to a method of treating a CDKL5-mediated neurological disorder, the method comprising administering a composition or formulation as described herein to an ex vivo cell and administering the ex vivo cell to a patient in need thereof. In one or more embodiments, ex vivo cell is administered intrathecally, intravenously, intracistnerally, intracerebroventrically or intraparenchymally. In one or more embodiments, the CDKL5-mediated neurological disorder is one or more of a CDKL5 deficiency or an atypical Rett syndrome caused by a CDKL5 mutation or deficiency.

Another aspect of the present invention relates to a novel CDKL5 polypeptide. In various embodiments, the CDKL5 polypeptide comprises a sequence having at least 99% sequence identity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25. In one or more embodiments, the CDKL5 polypeptide comprises the sequence of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25. In one or more embodiments, the CDKL5 polypeptide comprises the sequence of SEQ ID NO: 13. In one or more embodiments, the CDKL5 polypeptide comprises the sequence of SEQ ID NO: 14. In one or more embodiments, the CDKL5 polypeptide comprises the sequence of SEQ ID NO: 15. In one or more embodiments, the CDKL5 polypeptide comprises the sequence of SEQ ID NO: 16. In one or more embodiments, the CDKL5 polypeptide comprises the sequence of SEQ ID NO: 17. In one or more embodiments, the CDKL5 polypeptide comprises the sequence of SEQ ID NO: 18. In one or more embodiments, the CDKL5 polypeptide comprises the sequence of SEQ ID NO: 19. In one or more embodiments, the CDKL5 polypeptide comprises the sequence of SEQ ID NO: 20. In one or more embodiments, the CDKL5 polypeptide comprises the sequence of SEQ ID NO: 21. In one or more embodiments, the CDKL5 polypeptide comprises the sequence of SEQ ID NO: 22. In one or more embodiments, the CDKL5 polypeptide comprises the sequence of SEQ ID NO: 23. In one or more embodiments, the CDKL5 polypeptide comprises the sequence of SEQ ID NO: 24. In one or more embodiments, the CDKL5 polypeptide comprises the sequence of SEQ ID NO: 25.

Another aspect of the present invention relates to a fusion protein comprising a CDKL5 polypeptide as described herein and a leader signal polypeptide operatively coupled to the CDKL5 polypeptide. In one or more embodiments, the leader signal polypeptide has at least 90% sequence identity to SEQ ID NO: 38, SEQ ID NO: 39, SEQ ID NO: 40, SEQ ID NO: 41, SEQ ID NO: 42 or SEQ ID NO: 168. In one or more embodiments, the leader signal polypeptide comprises the sequence of SEQ ID NO: 38, SEQ ID NO: 39, SEQ ID NO: 40, SEQ ID NO: 41, SEQ ID NO: 42 or SEQ ID NO: 168.

Another aspect of the present invention relates to a fusion protein comprising a CDKL5 polypeptide as described herein and a cell-penetrating polypeptide operatively coupled to the CDKL5 polypeptide. In one or more embodiments, the cell-penetrating polypeptide has at least 90% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37 or SEQ ID NO: 167. In one or more embodiments, the cell-penetrating polypeptide comprises the sequence of SEQ ID NO: 31, SEQ ID NO: 32, SEQ ID NO: 33, SEQ ID NO: 34, SEQ ID NO: 35, SEQ ID NO: 36, SEQ ID NO: 37 or SEQ ID NO: 167. In one or more embodiments, the fusion protein further comprises a leader signal polypeptide. In one or more embodiments, the leader signal polypeptide has at least 90% sequence identity to SEQ ID NO: 38, SEQ ID NO: 39, SEQ ID NO: 40, SEQ ID NO: 41, SEQ ID NO: 42 or SEQ ID NO: 168. In one or more embodiments, the leader signal polypeptide comprises the sequence of SEQ ID NO: 38, SEQ ID NO: 39, SEQ ID NO: 40, SEQ ID NO: 41, SEQ ID NO: 42 or SEQ ID NO: 168.

In one or more embodiments, the fusion protein further comprises one or more affinity-tags, one or more protease cleavage sites, or combinations thereof. In some embodiments, the affinity-tag comprises one or more of MYC, HA, V5, NE, StrepII, Twin-Strep-tag®, glutathione S-transferase (GST), maltose-binding protein (MBP), calmodulin-binding peptide (CBP), FLAG®, 3×FLAG®, polyhistidine (His), HPC4, or combinations thereof. In some embodiments, the protease cleavage site is sensitive to one or more of thrombin, furin, factor Xa, metalloproteases, enterokinases, cathepsin, HRV3C, TEV, or combinations thereof.

Another aspect of the present invention relates to a pharmaceutical formulation comprising a CDKL5 polypeptide or fusion protein as described herein and a pharmaceutically acceptable carrier.

Another aspect of the present invention relates to a method of treating a CDKL5-mediated neurological disorder, the method comprising administering a CDKL5 polypeptide or fusion protein or formulation as described herein to a patient in need thereof. In one or more embodiments, the polypeptide, fusion protein or formulation is administered intrathecally, intravenously, intracisternally, intracerebroventrically or intraparenchymally. In one or more embodiments, the CDKL5-mediated neurological disorder is one or more of a CDKL5 deficiency or an atypical Rett syndrome caused by a CDKL5 mutation or deficiency.

Another aspect of the present invention relates to a method of producing a CDKL5 polypeptide or fusion protein as described herein. In various embodiments, the method comprises expressing the CDKL5 polypeptide or the fusion protein and purifying the CDKL5 polypeptide or the fusion protein. In one or more embodiments, the CDKL5 polypeptide or the fusion protein is expressed in Chinese hamster ovary (CHO) cells, HeLa cells, human embryonic kidney (HEK) cells or Escherichia coli cells.

Another aspect of the present invention relates to a method of producing a protein comprising a CDKL5 polypeptide, the method comprising expressing the protein in insect cells and purifying the protein from the insect cells. In one or more embodiments, the insect cells are Sf9 cells or BTI-Tn-5B1-4 cells.

In one or more embodiments, the protein comprises a fusion protein comprising the CDKL5 polypeptide and a cell-penetrating polypeptide operatively coupled to the CDKL5 polypeptide. In one or more embodiments, the cell-penetrating polypeptide is operatively coupled to the N-terminus of the CDKL5 polypeptide. In one or more embodiments, the cell-penetrating polypeptide is operatively coupled to the C-terminus of the CDKL5 polypeptide. In one or more embodiments, the fusion protein further comprises a leader signal polypeptide.

In one or more embodiments, the fusion protein further comprises one or more affinity-tags, one or more protease cleavage sites, or combinations thereof. In some embodiments, the affinity-tag comprises MYC, HA, V5, NE, StrepII, Twin-Strep-tag®, glutathione S-transferase (GST), maltose-binding protein (MBP), calmodulin-binding peptide (CBP), FLAG®, 3×FLAG®, polyhistidine (His), HPC4, or combinations thereof. In some embodiments, the protease cleavage site is sensitive to one or more of thrombin, furin, factor Xa, metalloproteases, enterokinases, cathepsin, HRV3C, TEV, or combinations thereof.

In one or more embodiments, the CDKL5 polypeptide has at least 98% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26. In one or more embodiments, the CDKL5 polypeptide has at least 98% sequence identity to SEQ ID NO: 1 or SEQ ID NO: 26. In one or more embodiments, the CDKL5 polypeptide has at least 98% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a polypeptide map of CDKL5₁₀₇. The map identifies important features of the polypeptide, including the ATP binding site, kinase domain and kinase active site, two nuclear localization signals, and a nuclear export signal.

FIGS. 1B and 1C show a graphic depicting the synthesized CDKL5 construct variants (1B) and a legend describes the length of the polypeptides, along with the relevant amino acid deletion information to describe how the constructs were synthesized (1C).

FIGS. 2A-2BK show exemplary plasmids for expressing various CDKL5 polypeptides and fusion proteins in cells such as CHO cells, HEK cells, Sf9 or E. Coli cells.

FIGS. 3A and 3B show Western blots of various CDKL5 fusion proteins expressed in E. coli cells. FIGS. 4A and 4B show Western blots of various CDKL5 fusion proteins expressed in CHO and HEK cells, respectively.

FIG. 4A shows expression of CDKL5 variants in CHO cells. FIG. 4B shows expression of CDKL5 variants in HEK293F cells.

FIG. 5 shows a Western blot demonstrating methotrexate amplification of various CDKL5 fusion proteins in CHO Cells.

FIGS. 6A and 6B show Western blots demonstrating expression and secretion of various CDKL5 fusion proteins in culture medium and cell lysates, respectively.

FIG. 7 shows a Western blot of a CDKL5 fusion protein that was co-expressed in the cytoplasm of HEK293F with several potential substrates.

FIG. 8 shows a Western blot of various CDKL5 fusion proteins expressed in a HeLa-based in vitro transcription/translation system.

FIGS. 9A and 9B show Western blots demonstrating glycosylation of various CDKL5 fusion proteins expressed in CHO and HEK cells, respectively.

FIG. 10 shows a quantitative analysis of relative expression and yield of CDKL5 protein in bacterial, mammalian and insect cell expression system.

FIGS. 11A and 11B show Sypro Ruby Red stained gels of various CDKL5 fusion proteins expressed in Sf9 insect cells.

FIG. 12A shows a Sypro Ruby Red stained gel of a CDKL5 fusion protein in cell lysate and the purified fusion protein.

FIG. 12B shows a Sypro Ruby Red stained gel demonstrating HRV3C protease cleavage of the CDKL5 fusion protein of FIG. 11A.

FIG. 13 shows Coomassie stained gels demonstrating solubility of CDKL5 fusion proteins in various salt and excipient systems.

FIG. 14A shows a schematic of TwinStrep-HRV3C-TATκ28-CDKL5-HRV3C-FLAG-His-HPC4 protein.

FIG. 14B shows purification and cleavage of TwinStrep-HRV3C-TATκ28-CDKL5-HRV3C-FLAG-His-HPC4 protein.

FIG. 15 shows a Western blot analysis of TwinStrep-HRV3C-TATκ28-CDKL5-HRV3C-FLAG-His-HPC4 protein purification and cleavage. FIG. 15A shows a Western-blot analysis using anti-strepII antibody. FIG. 15B shows a Western blot analysis using anti-HPC4 antibody.

FIG. 16 shows IMAC purification of TwinStrep-HRV3C-TATκ28-CDKL5-HRV3C-FLAG-His-HPC4 protein.

FIG. 17 shows a schematic of TwinStrep-HRV3C-TATκ28-CDKL5-HRV3C-FLAG-His-TwinStrep protein.

FIG. 18A shows purification and cleavage of TwinStrep-HRV3C-TATκ28-CDKL5-HRV3C-FLAG-His-TwinStrep protein. FIG. 18B shows a Western blot analysis of TwinStrep-HRV3C-TATκ28-CDKL5-HRV3C-FLAG-His-TwinStrep protein purification and cleavage.

FIG. 19 shows cation exchange chromatographic purification of TwinStrep-HRV3C-TATκ28-CDKL5-HRV3C-FLAG-His-TwinStrep protein.

FIG. 20 shows uptake of TATκ28-CDKL5 protein in rat DIV14 embryonic primary cortical neurons.

FIG. 21 shows uptake of TATκ28-CDKL5 protein in rat DIV7 embryonic primary cortical neurons.

FIG. 22 shows uptake of TATκ28-CDKL5 protein in rat DIV14 embryonic primary cortical neurons.

FIG. 23 shows time dependent uptake of TATκ28-CDKL5 protein in DIV14 embryonic primary cortical neurons.

FIG. 24 shows statistical analysis of TATκ28-CDKL5 protein uptake in DIV14 embryonic primary cortical neurons over time.

FIG. 25A shows co-localization of TATκ28-CDKL5 protein with PSD95.

FIG. 25B shows co-localization of TATκ28-CDKL5 protein with Synapsin 1.

FIGS. 26A-26E show rat neurons treated by lentiviral delivery of various CDKL5 fusion proteins.

FIG. 27A-271 shows BIP-TATκ28-CDKL5 induced cross-correction in striatum.

FIG. 28A-271 shows BIP-TATκ28-CDKL5 induced cross-correction in thalamus.

FIG. 29A-291 shows BIP-TATκ28-CDKL5 induced cross-correction in hippocampal formation.

FIG. 30A-30D shows raw-image and overlap image of DAPI stained cells, neurons, neurons having BIP-TATκ28-CDKL5 mRNA and BIP-TATκ28-CDKL5 protein, neurons having BIP-TATκ28-CDKL5 mRNA only, cross-corrected neurons and cross-corrected non-neurons.

FIG. 31A-31B shows quantifying cross-corrected cells using visiopharm.

FIG. 32A shows a statistical analysis of cross-corrected neurons in sagittal section. FIG. 32B shows a statistical analysis of cross-corrected neurons in particular brain regions including isocortex, striatum, thalamus and hippocampal formation.

FIG. 33 shows an exemplary plasmid for transfecting fusion proteins as described herein.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

It has surprisingly been discovered that proteins comprising the wild-type CDKL5 sequence have significant N-linked glycosylation when expressed and secreted in various host cell systems. Such N-linked glycosylation may have a negative impact on enzyme function due to changes in folding and/or interactions with binding partners. Accordingly, various aspects of the present invention relate to recombinant proteins comprising CDKL5 polypeptides that have one or more mutations to remove N-linked glycosylation sites.

Moreover, without wishing to be bound by any particular theory, it is believed that shorter CDKL5 variants that retain functional activity can provide benefits over the full-length, wild-type CDKL5 polypeptide, particularly when incorporated into a fusion protein comprising the CDKL5 polypeptide. In one or more embodiments, such benefits can include improved secretion from host cells during protein production, improved solubility, enhanced ability to cross the blood-brain barrier (BBB), and/or enhanced ability to penetrate target cells.

Other aspects of the present invention relate to novel cell systems for expressing and secreting recombinant proteins comprising CDKL5 polypeptides (e.g., wild-type CDKL5 polypeptides, CDKL5 variants with one or more N-linked glycosylation sites removed and/or shorter CDKL5 variants).

Other aspects of the present invention relate to gene therapy compositions and methods that utilize a CDKL5 polynucleotide encoding a CDKL5 polypeptide as described herein and a gene therapy delivery system.

### Definitions

As used herein, the term “CDKL5-mediated neurological disorder” refers to any disease or disorder that can be treated by expression or overexpression of the CDKL5 protein.

As used herein, the term “CDKL5 deficiency” refers to any deficiency in the biological function of the protein. The deficiency can result from any DNA mutation in the DNA coding for the protein or a DNA related regulatory region or any change in the function of the protein due to any changes in epigenetic DNA modification, including but not limited to DNA methylation or histone modification, any change in the secondary, tertiary, or quaternary structure of the CDKL5 protein, or any change in the ability of the CDKL5 protein to carry out its biological function as compared to a wild-type or normal subject. The deficiency can also include a lack of CDKL5 protein, such as a null mutation or underexpression of a fully functioning protein.

As used herein, the term “atypical Rett syndrome caused by a CDKL5 mutation or deficiency” refers to an atypical form of Rett syndrome with similar clinical signs to Rett syndrome but is caused by a CDKL5 mutation or deficiency.

Symptoms or markers of a CDKL5 deficiency, Rett syndrome, or an atypical Rett syndrome include but are not limited to seizures, cognitive disability, hypotonia, as well as autonomic, sleep, and gastrointestinal disturbances.

As used herein, the term “gene therapy delivery system” refers to any system that can be used to deliver an exogenous gene of interest to a target cell so that the gene of interest will be expressed or overexpressed in the target cell. In one or more embodiments, the target cell is an in vivo patient cell. In one or more embodiments, the target cell is an ex vivo cell and the cell is then administered to the patient.

As used herein, the term “carrier” is intended to refer to a diluent, adjuvant, excipient, or vehicle with which a compound is administered. Suitable pharmaceutical carriers are known in the art and, in at least one embodiment, are described in “Remington's Pharmaceutical Sciences” by E. W. Martin, 18th Edition, or other editions.

As used herein, the term “enzyme replacement therapy” or “ERT” is intended to refer to the introduction of an exogenous, purified enzyme into an individual having a deficiency in such enzyme. The administered protein can be obtained from natural sources or by recombinant expression. The term also refers to the introduction of a purified enzyme in an individual otherwise requiring or benefiting from administration of a purified enzyme. In at least one embodiment, such an individual suffers from enzyme insufficiency. The introduced enzyme may be a purified, recombinant enzyme produced in vitro, or a protein purified from isolated tissue or fluid, such as, for example, placenta or animal milk, or from plants.

As used herein, the terms “subject” or “patient” are intended to refer to a human or non-human animal. In at least one embodiment, the subject is a mammal. In at least one embodiment, the subject is a human.

As used herein, the “therapeutically effective dose” and “effective amount” are intended to refer to an amount of gene therapy composition (e.g. comprising CDKL5 polynucleotides) or recombinant protein (e.g. CDKL5 variants or fusion proteins) which is sufficient to result in a therapeutic response in a subject. A therapeutic response may be any response that a user (for example, a clinician) will recognize as an effective response to the therapy, including any surrogate clinical markers or symptoms described herein and known in the art. Thus, in at least one embodiment, a therapeutic response can be an amelioration or inhibition of one or more symptoms or markers of a CDKL5 deficiency, Rett syndrome, or an atypical Rett syndrome such as those known in the art.

### Function of CDKL5 Proteins

The human CDKL5 gene is composed of 24 exons, of which the first three (exons 1, 1a and 1b) are untranslated.

The originally discovered human CDKL5 variant was 1030 amino acids with a molecular mass of 115 kDa (CDKL5₁₁₅). Another prominent variant, CDKL5₁₀₇, contains an altered C-terminal region because alternative splicing combines different exons than in the CDKL5₁₁₅ variant. CDKL5₁₀₇ (107 kDa) is shorter because it harbors an alternate version of exon 19 and does not contain exons 20-21 that are present in the CDKL5₁₁₅ variant. The hCDKL5₁₀₇ mRNA has been found to be 37-fold more abundant in human brain than the hCDKL5₁₁₅ transcript, and murine CDKL5₁₀₇ has been found to be 160-fold more abundant than the murine CDKL5₁₀₅ variant in murine brain. Both the human and murine CDKL5₁₀₇ isoforms have demonstrated a longer half-life and resistance to degradation as compared to the human CDKL5₁₁₅ variant.

CDKL5 knockout mouse models have been generated using the Lox-Cre recombination system and these mice present symptoms of autistic-like deficits in social interactions, impairment of motor control, and loss of fear memory (Wang et al., Proc Natl Acad Sci U.S.A, 109(52), 21516-21521). For example, knockout CDKL5 mice have symptoms of reduced motor coordination and demonstrate impaired memory and fear responses when repeatedly exposed to stimuli. These changes have led scientists to hypothesize that loss of CDKL5 kinase activity leads to impaired neuronal network development. Previous data have suggested that CDKL5 phosphorylates methyl-CpG binding protein 2 (MeCP2), and independent loss-of-function mutations in MeCP2 lead to the Rett syndrome phenotype. Other substrates of CDKL5 include Netrin G1 ligand (NGL-1), Shootin1 (SHTN1), Mindbomb 1 (MIB1), DNA (cytosine-5)-methyltransferase 1 (DNMT1), Amphiphysin 1 (AMPH1), end-binding protein EB2, microtubule associated protein 1S (MAP1S) and histone deacetylase 4 (HDAC4). Although the exact role of CDKL5 has yet to be identified, these data suggest that CDKL5 plays a role in phosphorylation of downstream targets that are critical for correct neuronal development, including MeCP2. In humans, mutations in CDKL5 are associated with a phenotype that overlaps with Rett syndrome, and additionally presents with early-onset seizures. While CDKL5 KO mice did not exhibit any early-onset seizure symptoms, they did exhibit motor defects, decreased sociability, and impaired learning and memory (Chen et al. CDKL5, a protein associated with Rett Syndrome, regulates neuronal morphogenesis via Rac1 signaling, J Neurosci 30: 12777-12786).

Two CDKL5 isoforms are found in rat, one labeled CDKL5a and the other CDKL5b. (Chen et al.). In general, there is a high level of sequence conservation in CDKL5 genes across human, rat, and mouse species except for the last 100-150 amino acids near the C-terminus. Western blot data show that both variants are present during rat development yet adults appear to predominately express a single variant. Furthermore, CDKL5 is present in identifiable quantities in brain, liver, and lung.

CDKL5 functions in the nucleus but it is also found in the dendrites of cultured neurons, suggesting a possible alternate cytoplasmic role. Down regulation of CDKL5 expression by RNAi (RNA Interference) in cultured cortical neurons inhibited neurite growth and dendritic arborization (branching), where over expression of CDKL5 had opposite effects (Chen et al.). In order to characterize both the nuclear and cytoplasmic effect of CDKL5, a variant of CDKL5a with a nuclear export sequence (NES) was expressed in the cultured cortical neuron RNAi model. This NES-CDKL5a variant was resistant to the RNAi used to silence the wild-type gene expression, and therefore was used to model CDKL5a when expressed solely in the cytoplasm. After using the GFP tag to confirm that this CDKL5 variant was exclusively present in the cytoplasm, an increase in both the length of neurites and number of neurite branches was seen. The ability of NES-GFP-CDKL5a to partially rescue the disease phenotype observed when RNAi was used to knockdown the endogenous CDKL5 expression suggests that the expression of CDKL5 in cytoplasm in an important factor in the development and growth of neurites.

Human mutations in CDKL5 are associated with a phenotype similar to Rett syndrome, and individuals with CDKL5 mutations also present with early-onset seizures. This onset of seizures differs from the classical Rett syndrome phenotype in which there is an early normal period of development before the onset of Rett symptoms. Patients with classical Rett syndrome (RTT) appear to develop normally until 6-18 months of age, and then they begin to present neurological symptoms including loss of speech and movement. Autopsies of RTT brains show smaller and more densely packed neurons with shorter dendrites in the motor and frontal cortex, suggesting that neuronal development is impaired. The majority of Classical RTT cases are due to mutations in the MECP2 gene, which is an X-linked gene encoding a nuclear protein that selectively binds to CpG dinucleotides in the mammalian genome and regulates transcription through the recruitment of complexes. Although poorly understood, it is generally thought that the dysregulation of gene expression caused by mutations in MECP2 is the underlying cause of Rett Syndrome. Approximately 20% of Classic Rett syndrome cases and 60-80% of other Rett syndrome variants carry no mutations in MECP2, suggesting an alternate genetic cause for pathogenesis. Recently, some CDKL5 mutations have been identified in patients with certain variants of RTT and other severe encephalopathies, and CDKL5 has been shown to interact with MeCP2 both in vivo and in vitro. Beyond MeCP2, CDKL5 has been shown to interact with and phosphorylate a number of downstream targets, including NGL-1. When phosphorylated, NGL-1 interacts with PSD95 and is critical for the correct genesis and development of dendritic spines and synapse formation (Ricciardi S, et al. “CDKL5 ensures excitatory synapse stability by reinforcing NGL-1-PSD95 interaction in the postsynaptic compartment and is impaired in patient iPSC-derived neurons.” Nat Cell Biol 14(9):911-923).

CDKL5 has also been shown to phosphorylate the protein DNA methyltransferase 1 (DNMT1) (Kameshita I, et al. “Cyclin-dependent kinase-like 5 binds and phosphorylates DNA methyltransferase 1.” Biochem Biophys Res Commun 377:1162-1167). This phosphorylation leads to activation of DNMT1, which is a maintenance-type methylation protein that preferentially methylates hemimethylated DNA. This process is useful for maintenance of DNA methylation patterns during DNA replication, so that newly synthesized daughter DNA strands are able to maintain the methylation pattern of the parent strand it replaced. As methylation of DNA is generally thought to be an epigenetic mechanism to silence gene expression, this maintenance function of DNMT1 is crucial in preserving gene expression patterns across cell generations.

Current models suggest that the CDKL5 kinase domain phosphorylates GSK-3β, and that phosphorylation of GSK-3β leads to its inactivation. Individuals who are deficient in CDKL5 activity therefore seem to exhibit increased GSK-3β activity. Previous studies have shown that GSK-3β modulates hippocampal neurogenesis, and that an increased activity of GSK-3β severely impairs dendritic morphology of newborn hippocampal neurons. Furthermore, GSK-3β seems to act as a negative regulator of key developmental events such as neuron survival and maturation. A study conducted using CDKL5 KO mice demonstrated that treatment with a GSK-3β inhibitor could almost fully rescue hippocampal development and behavioral deficits in mice deficient in CDKL5 activity (Fuchs et al. “Inhibition of GSK3β Rescues Hippocampal Development and Learning in a Mouse Model of CDKL5 Disorder.” Neurobiology of Disease 82: 298-310). This developmental rescue also seemed to persist beyond treatment.

### CDKL5₁₀₇ Polypeptide Constructs

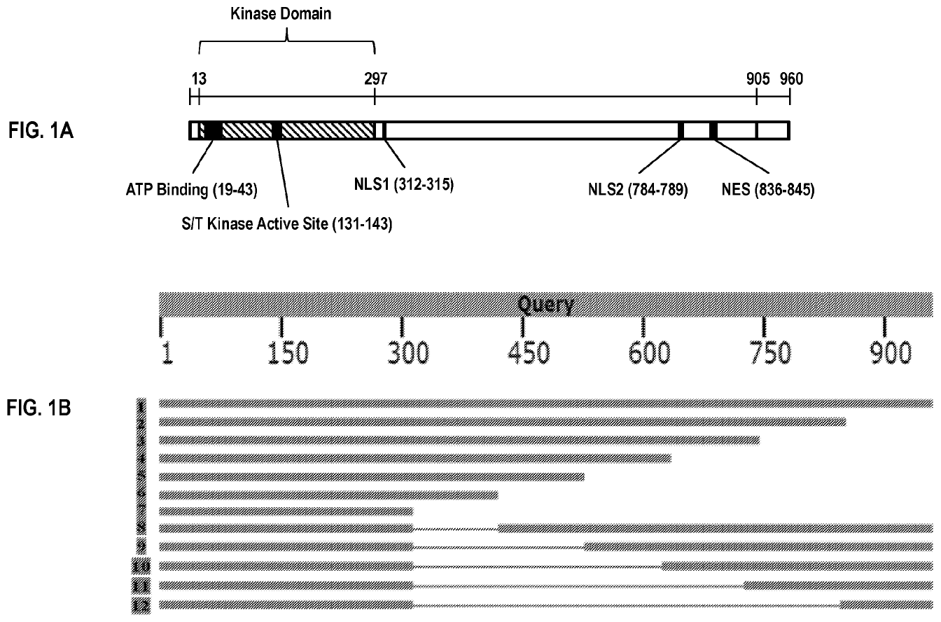

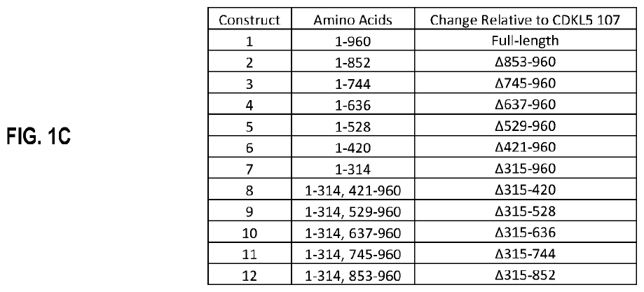

FIG. 1A displays a polypeptide map of CDKL5₁₀₇. The amino acid sequence of the wild-type full-length human CDKL5₁₀₇ isoform is provided in SEQ ID NO: 1. The CDKL5₁₀₇ protein consists of 960 amino acids, and the kinase domain is contained in the first ˜300 amino acids. Residue 42 of 960 is a key lysine residue located within the kinase domain that participates in ATP binding during a phosphorylation reaction, and mutation of this residue generally leads to loss of kinase activity (“Kinase dead”). Additionally, two nuclear localization signals are present spanning residues 312-315 (NLS1) and 784-789 (NLS2), and a nuclear export signal (NES) is present spanning residues 836-845. Amino acids at the C-terminus spanning from residue 905 to 960 are unique to CDKL5₁₀₇ and are not present in CDKL5₁₁₅. Amino acid residues 1-904 are identical between CDKL5₁₁₅ and CDKL5₁₀₇. The amino acid sequence of the wild-type full-length human CDKL5₁₁₅ isoform is provided in SEQ ID NO: 26.

Various embodiments of the present invention provide novel CDKL5 variants. FIGS. 1B and 1C show the polypeptides of the full-length human CDKL5₁₀₇ isoform (Construct 1) and novel CDKL5 constructs (designated as Constructs 2-12). These CDKL5 constructs generally fall into two categories: those missing some number of amino acids at the C-terminus (Constructs 2-7) and those missing some number of amino acids in the middle of the polypeptide chain (Constructs 8-12). Moreover, in those constructs wherein CDKL5 is fused C-terminally to additional N-terminal amino acid sequences, the initial methionine of CDKL5 is removed. In these constructs, the CDKL5 polypeptide begins with the second amino acid, lysine. Construct 1 contains all 960 amino acids of the full-length human CDKL5₁₀₇ isoform. Construct 2, which contains the first 851 amino acids of the entire 960 amino acid chain, represents a shortened CDKL5 polypeptide in which the tail sequence that differs between CDKL5₁₀₇ and CDKL5₁₁₅ is removed but the kinase domain, nuclear localization signals (NLS1 and NLS2), and nuclear export signal (NES) remain intact. Construct 3 is shortened further, in which the nuclear localization signal (NLS2) and the nuclear export signal (NES) are additionally removed. Constructs 4-7 are shortened even further, as shown in FIGS. 1B and 1C. Constructs 2-7 all contain the active kinase domain, while Constructs 3-7 do not contain the NLS2 or NES sequences. Construct 7 is further shortened up to the NLS1 sequence. The remaining constructs (Constructs 8-12) all have deletions in the middle portion of the polypeptide chain while retaining the C-terminal amino acids unique to CDKL5₁₀₇. Of these constructs, Construct 12 is missing the NES and NLS2 sequences. The amino acid sequences of Constructs 1-12 are provided in SEQ ID NOS: 1-12, respectively.

In one or more embodiments, the CDKL5 polypeptide has at least 98%, at least 98.5%, at least 99% or at least 99.5% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12. The CDKL5 polypeptide may contain deletions, substitutions and/or insertions relative to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12, such as having 1, 2, 3, 4, 5, 6, 7, 8, 9, 10, 11, 12, 13, 14, 15 or more deletions, substitutions and/or insertions to the amino acid sequence described by SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12.

In one or more embodiments, the CDKL5 polypeptide has at least 98%, at least 98.5%, at least 99% or at least 99.5% sequence identity to SEQ ID NO: 1 or SEQ ID NO: 26. The CDKL5 polypeptide may contain deletions, substitutions and/or insertions relative to SEQ ID NO: 1 or SEQ ID NO: 26, such as having 1, 2, 3, 4, 5, 6, 7, 8, 9, 10, 11, 12, 13, 14, 15 or more deletions, substitutions and/or insertions to the amino acid sequence described by SEQ ID NO: 1 or SEQ ID NO: 26.

In one or more embodiments, the CDKL5 polypeptide comprises one or more affinity-tags. In one or more embodiments, the affinity-tag is located on one or more of the N-terminus or the C-terminus of the CDKL5 polypeptide. Examples of tags that can be added to the fusion proteins include, but are not limited to, epitope tags (e.g. MYC, HA, V5, NE, StrepII, Twin-Strep-tag®, HPC4), glutathione S-transferase (GST), maltose-binding protein (MBP), calmodulin-binding peptide (CBP), FLAG®, 3×FLAG®, polyhistidine (His), and combinations thereof.

In one or more embodiments, the CDKL5 polypeptide comprises one or more protease cleavage sites. In some embodiments, the protease cleavage site is located on one or more of the N-terminus or the C-terminus of the CDKL5 polypeptide. Exemplary protease cleavage sites include, but are not limited to, cleavage sites sensitive to thrombin, furin, factor Xa, metalloproteases, enterokinases, cathepsin, HRV3C, TEV, and combinations thereof.

Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 98%, 98.5%, 99% or 99.5% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

One skilled in the art can readily derive a polynucleotide sequence encoding a particular polypeptide sequence. Such polynucleotide sequence can be codon optimized for expression in the target cell using commercially available products, such as using the OptimumGene™ codon optimization tool (GenScript, Piscataway, N.J.).

### CDKL5₁₀₇ N-Linked Glycosylation Variants

Various embodiments of the present invention provide novel CDKL5 variants that have one or more mutations to remove one or more N-linked glycosylation sites from the CDKL5 polypeptide. The wild-type human isoform CDKL5₁₀₇ contains 10 potential N-linked glycosylation sites and the wild-type human isoform CDKL5₁₁₅ contains 8 potential N-linked glycosylation sites. One of these glycosylation sites includes the TEY (Thr-Glu-Tyr) motif: NYTEY (Asn-Tyr-Thr-Glu-Tyr), and thus one of the glycosylation sites resides in the kinase domain. As such, there is a high likelihood that glycosylation at the Asn-Tyr-Thr-Glu-Tyr site can interfere with phosphorylation of the Thr-Glu-Tyr motif. Generally, sequences of Asn-X-Ser or Asn-X-Thr in the protein amino acid sequence indicate potential glycosylation sites, with the exception that X cannot be His or Pro. Accordingly, various embodiments of the present invention provide CDKL5 polypeptides that have one or more asparagine (aka Asn or N) residues substituted with a different amino acid such as glutamine (aka Gln or Q) residues. One potential advantage of choosing glutamine for the substitution is that this amino acid is structurally similar to asparagine, with only an additional methylene unit present in the glutamine residue. However, other amino acids can also be used as substitutions for the asparagine residue(s). Alternatively, the glycosylation site can be altered by changing the third amino acid in the Asn-X-Ser or Asn-X-Thr sequence to another amino acid that is not serine (aka S or Ser) or threonine (aka T or Thr) and/or changing the second amino acid to histidine (aka H or His) or proline (aka P or Pro).

Embodiments of the present invention also provide CDKL5 polynucleotides that encode CDKL5 polypeptides that have one or more Asn residues substituted with another amino acid such as Gln residues. For example, one or more AAC, AAT or AAU sequences (which encode Asn) can be substituted with one or more CAA or CAG sequences (which encode Gln). Again, other alterations in the CDKL5 polynucleotides can encode other changes to the glycosylation sites such as substituting the second amino acid with His or Pro and/or changing the third amino acid to be another amino acid that is not Ser or Thr.

In one or more embodiments, the CDKL5 polypeptide has at least 98%, at least 98.5%, at least 99% or at least 99.5% sequence identity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25. The CDKL5 polypeptide may contain deletions, substitutions and/or insertions relative to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25, such as having 1, 2, 3, 4, 5, 6, 7, 8, 9, 10, 11, 12, 13, 14, 15 or more deletions, substitutions and/or insertions to the amino acid sequence described by SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25.

In one or more embodiments, the CDKL5 polypeptide comprises one or more affinity-tags. In one or more embodiments, the affinity-tag is located on one or more of the N-terminus or the C-terminus of the CDKL5 polypeptide. Examples of tags that can be added to the fusion proteins include, but are not limited to, epitope tags (e.g. MYC, HA, V5, NE, StrepII, Twin-Strep-tag®, HPC4), glutathione S-transferase (GST), maltose-binding protein (MBP), calmodulin-binding peptide (CBP), FLAG®, 3×FLAG®, polyhistidine (His), and combinations thereof.

In one or more embodiments, the CDKL5 polypeptide comprises one or more protease cleavage sites. In some embodiments, the protease cleavage site is located on one or more of the N-terminus or the C-terminus of the CDKL5 polypeptide. Exemplary protease cleavage sites include, but are not limited to, cleavage sites sensitive to thrombin, furin, factor Xa, metalloproteases, enterokinases, cathepsin, HRV3C, TEV, and combinations thereof.

### Cell-Penetrating Peptides (CPPs)

A variety of viral and cellular proteins possess basic polypeptide sequences that mediate translocation across cellular membranes. The capacity to translocate across cellular membranes has become an important tool for the delivery of high molecular weight polypeptides across membranes. The phrase “protein transduction domain” (PTD) and “cell-penetrating peptides” (CPPs) are usually used to refer to short peptides (<30 amino acids) that can traverse the plasma membrane of many, if not all, mammalian cells. After studies to identify the specific properties of the domain that allow them to collectively cross the plasma membrane, researchers have observed that these domains contain a large number of basic amino acid residues such as lysine and arginine. Thus, cell-penetrating peptides fall into two classes: the first consisting of amphipathic helical peptides that contain lysine residues which contribute a positive charge, while the second class includes arginine-rich peptides. These peptides could have therapeutic potential if used in combination with other proteins that are difficult to deliver to intracellular targets. The most frequent experimental uses of PTDs are TAT, Antennapedia (Antp), and other poly-arginine peptides.

Thus far, TAT has been the best characterized of the PTDs, and has been used to successfully deliver small cargoes, such as short peptides and oligonucleotides, to intercellular targets. HIV-TAT (HIV Transactivator of Transcription) is an 86-amino acid protein involved in the replication of human immunodeficiency virus type 1 (HIV-1), and many studies have shown that TAT is able to translocate through the plasma membrane and reach the nucleus in order to activate transcription of the viral genome. Studies have also shown that TAT retains its penetration properties when coupled to several different proteins. In an effort to understand which areas of the TAT protein are critical to the translocation property, experiments have been conducted in which different length peptide fragments of TAT are synthesized and their penetration capabilities are assessed. (Lebleu et al. “A Truncated HIV-1 TAT Protein Basic Domain Rapidly Translocates through the Plasma Membrane and Accumulates in the Cell Nucleus.” J. Biol. Chem. 1997, 272:16010-16017). A region of basic amino acids has been identified as the aspect of TAT that retains this penetration property, and experiments in which a TAT protein without this basic amino acid cluster is unable to penetrate the cellular plasma membrane. In some instances, the shorter sequence cell-penetrating peptide has been modified to prevent cleavage during secretion by endoprotease enzymes such as furin. These modifications change the shortened cell-penetrating TAT amino acid sequence from YGRKKRRQRRR to YARKAARQARA, and this short peptide is referred to as TATκ.

The exact mechanism in which TAT is able to translocate across the plasma membrane remains uncertain. Recent work has explored the possibility that a special type of endocytosis is involved with TAT uptake, and a few cell lines have been identified that appear resistant to TAT penetration. The specific cargo to be delivered by TAT may also play a role in the efficacy of delivery. Previous research data have suggested that a TAT fusion protein has better cellular uptake when it is prepared in denaturing conditions, because correctly folded protein cargo likely requires much more energy (delta-G) to cross the plasma membrane due to structural constraints.

The capacity of the intracellular protein chaperones to refold the TAT cargo likely varies based on the identity and size of the protein cargo to be re-folded. In some instances, TAT-fusion proteins precipitate when placed in an aqueous environment and therefore cannot be prepared in a denatured manner nor remain stable for very long in native conformations. The design of the TAT-fusion protein must also be tailored to the specific cargo to be delivered. If the cargo protein is tightly associated at the N-terminus and the TAT domain is also found at the N-terminus, the TAT translocation domain may be buried in the cargo protein and transduction may be poor.

Numerous TAT-cargo variants have been successfully delivered into a variety of cell types, including primary culture cells, transformed cells, and cells present in mouse tissue. In culture, the TAT-fusion proteins generally diffuse easily into and out of cells, leading to a very rapid establishment of uniform concentration.

Many pharmaceutical agents such as enzymes, antibodies, other proteins, or even drug-loaded carrier particles need to be delivered intracellularly to exert their therapeutic action inside the cytoplasm, nucleus, or other specific organelles. Thus, the delivery of these different types of large molecules represents a significant challenge in the development of biologics. Current data suggest that TAT is able to cross the plasma membrane through more than one mechanism.

A TAT transduction domain has also been fused to the enzyme superoxide dismutase (SOD). (Torchilin, “Intracellular delivery of protein and peptide therapeutics.” Protein Therapeutics. 2008. 5(2-3):e95-e103). This fusion protein was used to demonstrate that it could translocate across cell membranes in order to deliver the SOD enzyme to the intracellular environment, and thus here the fusion protein has therapeutic potential in treating enzyme deficiency disorders that lead to higher accumulation of reactive oxygen species and oxidative stress on a host cell.

TAT fusion proteins have also been shown to transduce across the blood-brain barrier. A TAT domain fused to the neuroprotectant protein Bc1-xL was able to penetrate cells rapidly in culture, and when administered to mice suffering from cerebral ischemia, the fusion protein transduced brain cells within 1-2 hours. After transduction, the cerebral infarct was reduced in size in a dose-dependent manner (Cao, G. et al., “In Vivo Delivery of a Bc1-xL Fusion Protein Containing the TAT Protein Transduction Domain Protects against Ischemic Brain Injury and Neuronal Apoptosis.” J. Neurosci. 22, 5423, 2002.)

In various embodiments, the CDKL5 variants described herein are operably linked to a CPP such as TAT, modified TAT (TATκ), Transportan, Antennapedia or P97. As used herein, TAT can refer to the original TAT peptide having 11 amino acids (designated TAT11) or can refer to a TAT peptide having an additional 16 N-terminal amino acids (designated as TAT28) that are derived from the polylinker of the plasmid used for cloning. Similarly, TATκ can refer to a modified version of TAT11 (designated TATκ11) or a modified version of TAT28 (designated TATκ28). The TATκ28 can be further modified (designated TATκκ28) to remove a potential additional weak furin site. The amino acid sequences of the CPPs TAT28, TATκ28, TAT11, TATκ11, Transportan, Antennapedia, P97 and TATκκ28 are provided in SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 167, respectively.

In some embodiments, the CPP has at least 90% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37 or SEQ ID NO: 167. In some embodiments, the CPP has at least 95% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37 or SEQ ID NO: 167. In some embodiments, the CPP has 100% sequence identity to SEQ ID NO: 31, SEQ ID NO: 32, SEQ ID NO: 33, SEQ ID NO: 34, SEQ ID NO: 35, SEQ ID NO: 36, SEQ ID NO: 37 or SEQ ID NO: 167. In some embodiments, the CPP has at least 90% sequence identity to SEQ ID NO: 32, SEQ ID NO: 34, SEQ ID NO: 35, SEQ ID NO: 36, SEQ ID NO: 37 or SEQ ID NO: 167. In some embodiments, the CPP has at least 95% sequence identity to SEQ ID NO: 32, SEQ ID NO: 34, SEQ ID NO: 35, SEQ ID NO: 36, SEQ ID NO: 37 or SEQ ID NO: 167. In some embodiments, the CPP has 100% sequence identity to SEQ ID NO: 32, SEQ ID NO: 34, SEQ ID NO: 35, SEQ ID NO: 36, SEQ ID NO: 37 or SEQ ID NO: 167. In various embodiments, the CPP does not have the sequence of SEQ ID NO: 34.

In various embodiments, the CPP can have an N-terminal glycine added. For example, TATκ28 and TAT28 would otherwise have an N-terminal aspartate residue, which has a low stability. Adding an N-terminal glycine to the sequence can increase protein stability via the N-end rule. Accordingly, in some embodiments, any of the fusion proteins that have a leader signal polypeptide can have a glycine added at the C-terminal end of the leader signal polypeptide, such that upon cleavage of the leader signal polypeptide, the new N-terminus of the fusion protein will begin with glycine. In an analogous manner, those fusion proteins lacking a leader signal polypeptide can also have a glycine added between the N-terminal methionine and the remainder of the fusion protein. Also in analogous manner, those fusion proteins having a CPP other than TAT28 or TATκ28, can also have a glycine added between a leader signal polypeptide and a CPP.

In one or more embodiments, the CPP is operatively coupled to the N-terminus of the CDKL5 polypeptide. In one or more embodiments, the CPP is operatively coupled to the C-terminus of the CDKL5 polypeptide.

In one or more embodiments, the CPP comprises one or more affinity-tags. In one or more embodiments, the affinity-tag is located on one or more of the N-terminus or the C-terminus of the CPP. Examples of affinity-tags that can be added to the CPP include, but are not limited to, epitope tags (e.g. MYC, HA, V5, NE, StrepII, Twin-Strep-tag®, HPC4), glutathione S-transferase (GST), maltose-binding protein (MBP), calmodulin-binding peptide (CBP), FLAG®, 3×FLAG® polyhistidine (His), and combinations thereof.

In one or more embodiments, the CPP comprises one or more protease cleavage sites. In some embodiments, the protease cleavage site is located on one or more of the N-terminus or the C-terminus of the CPP. Exemplary protease cleavage sites include, but are not limited to, cleavage sites sensitive to thrombin, furin, factor Xa, metalloproteases, enterokinases, cathepsin, HRV3C, TEV, and combinations thereof.

### Fusion Proteins Comprising CDKL5 Variants

As described above, CDKL5 variants can be used in fusion proteins, such as proteins that also contain a CPP. Other polypeptides can also be incorporated into such fusion proteins, such as leader signal polypeptides to enhance protein secretion or affinity-tags for detecting and/or purifying the fusion proteins, as well as linker polypeptides that can be used to link functional polypeptides.

Examples of leader signal polypeptides include, but are not limited to, modified fragments of human immunoglobulin heavy chain binding protein (modified BiP, e.g. SEQ ID NO: 38, SEQ ID NO: 39, SEQ ID NO: 40, SEQ ID NO: 41 or SEQ ID NO: 168), murine Igκ chain leader polypeptide (SEQ ID NO: 42, e.g. pSecTag2 from ThermoFisher vectors) or insulin growth factor peptides (IGF2) such as the wild-type IFG2 (SEQ ID NO: 156) or variants thereof (e.g. SEQ ID NOS 157-166). Examples of modified BiP signal polypeptides include those described in U.S. Pat. No. 9,279,007, which is hereby incorporated by reference in its entirety. Other examples of modified BiP signal polypeptides include mvBIP, which has a valine added before the lysine in mBiP as shown in SEQ ID NO: 168.

In one or more embodiments, the fusion protein comprises a CDKL5 polypeptide having an N-terminal CPP, optionally with a leader signal polypeptide before the N-terminal CPP. In one or more embodiments, the fusion protein comprises a CDKL5 polypeptide having a C-terminal CPP, optionally with a leader signal polypeptide before the CDKL5 polypeptide. In one or more embodiments, the fusion protein comprises a leader signal peptide and a CDKL5 polypeptide without a CPP.

Examples of affinity-tags that can be added to the fusion proteins include, but are not limited to, epitope tags (e.g. MYC, HA, V5, NE, StrepII, Twin-Strep-tag®, HPC4), glutathione S-transferase (GST), maltose-binding protein (MBP), calmodulin-binding peptide (CBP), FLAG®, 3×FLAG®, polyhistidine (His), and combinations thereof.

Some embodiments of the fusion protein may also include a protease cleavage site. In some embodiments, the protease cleavage site is located on the N-terminus of affinity-tag. In some embodiments, the protease cleavage site is located on the C-terminus of affinity-tag. Exemplary protease cleavage sites include, but are not limited to, cleavage sites sensitive to thrombin, furin, factor Xa, metalloproteases, enterokinases, cathepsin, HRV3C, TEV and combination thereof.

### Methods of Protein Production

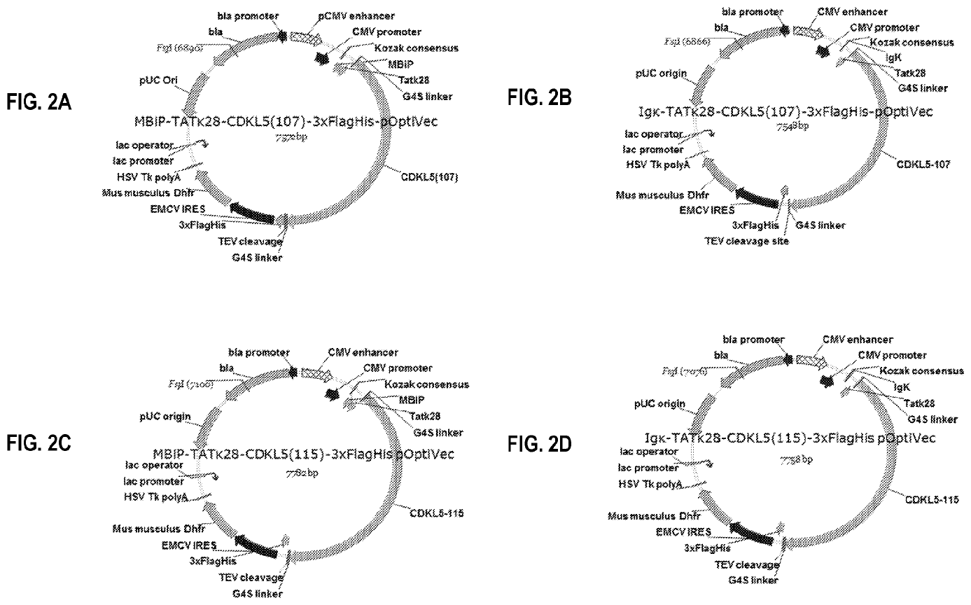

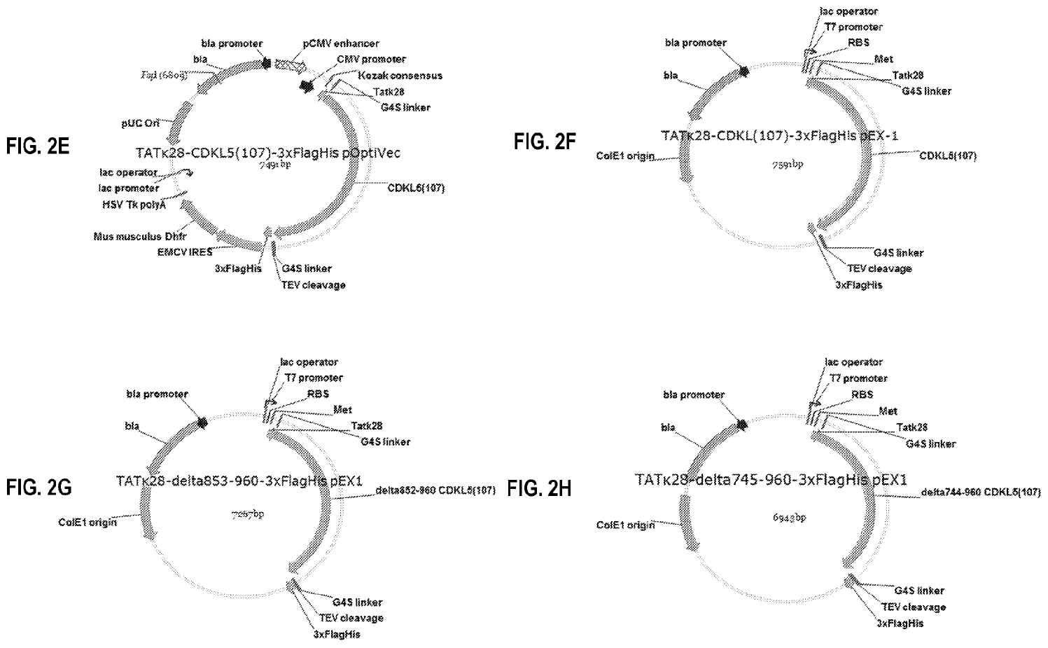

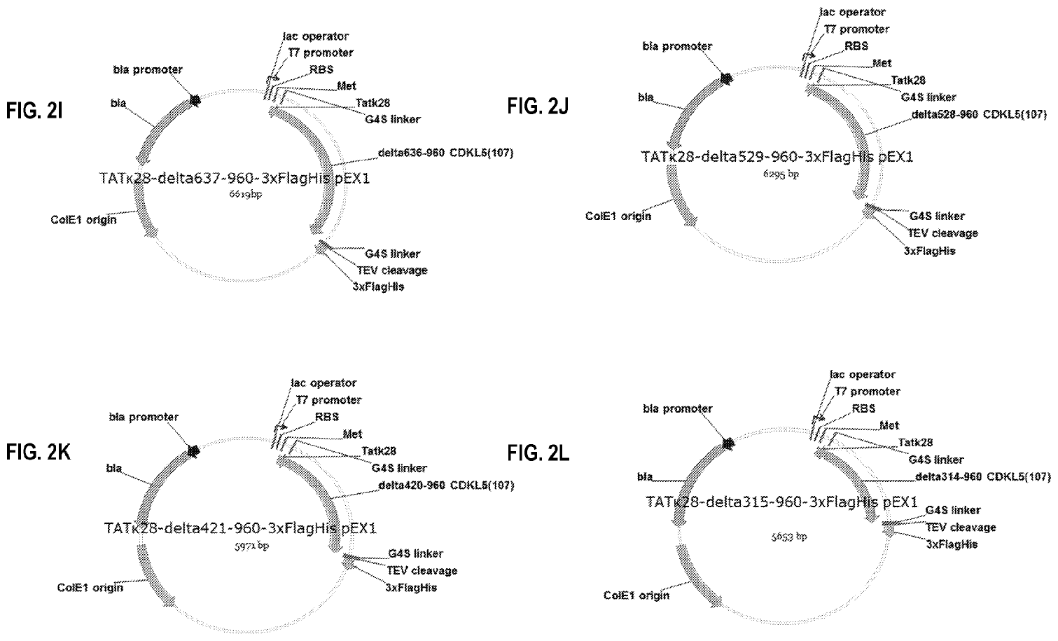

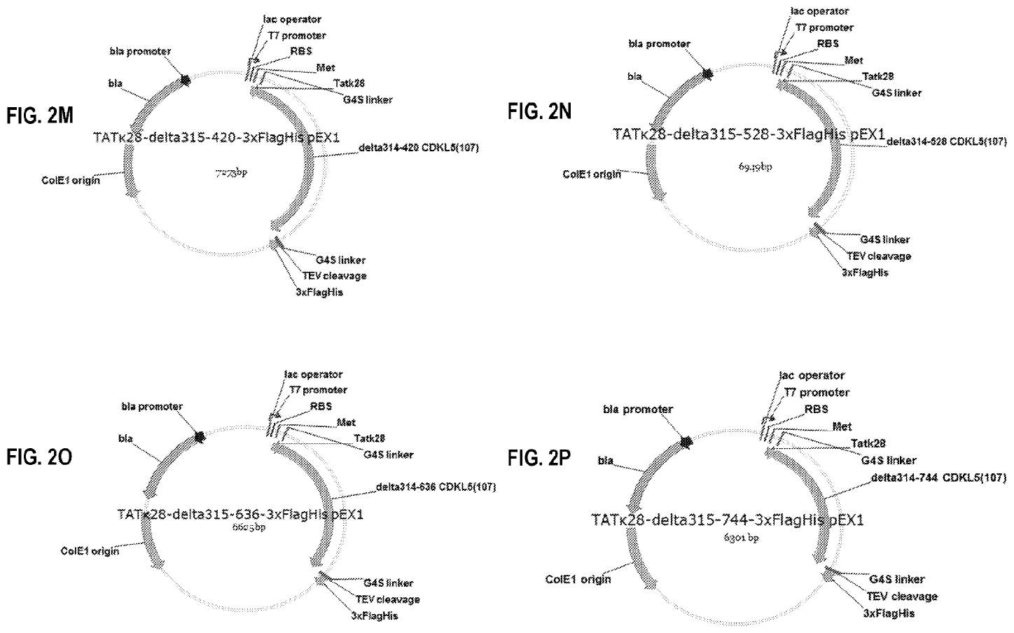

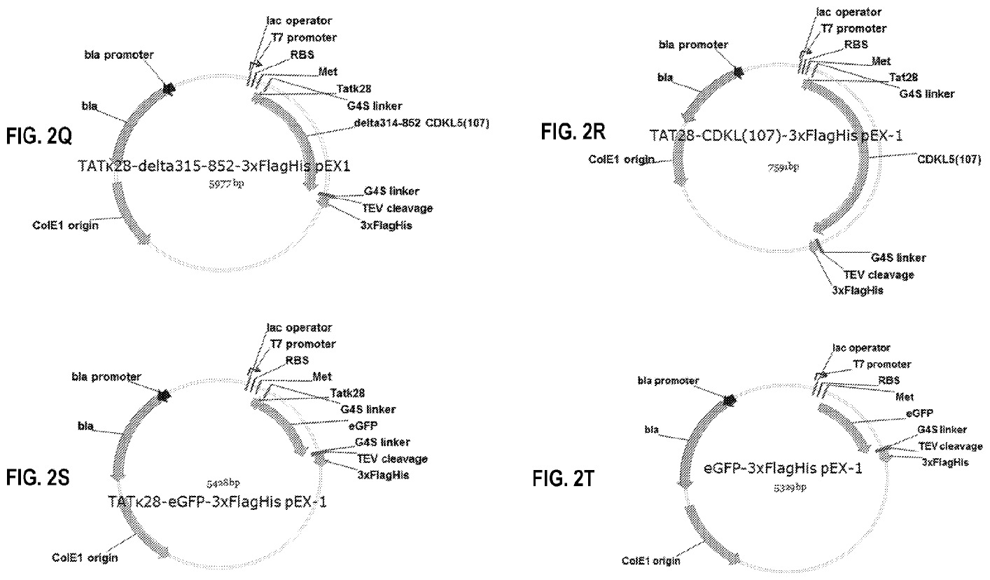

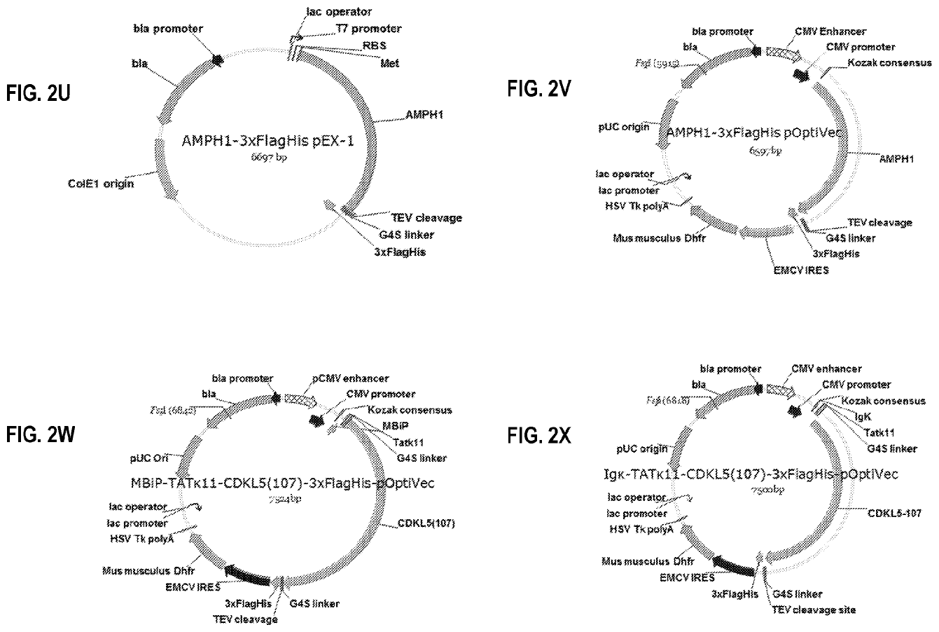

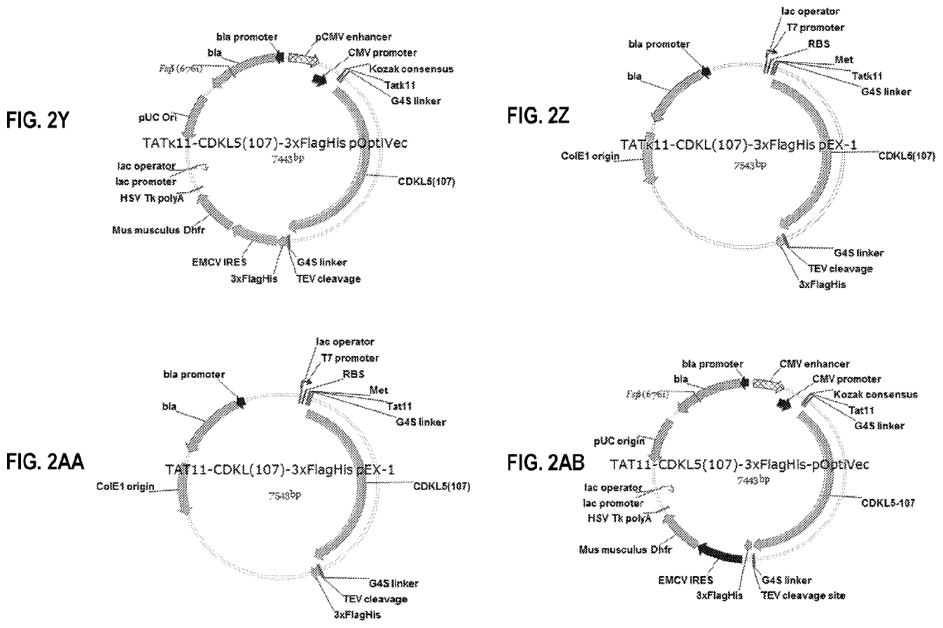

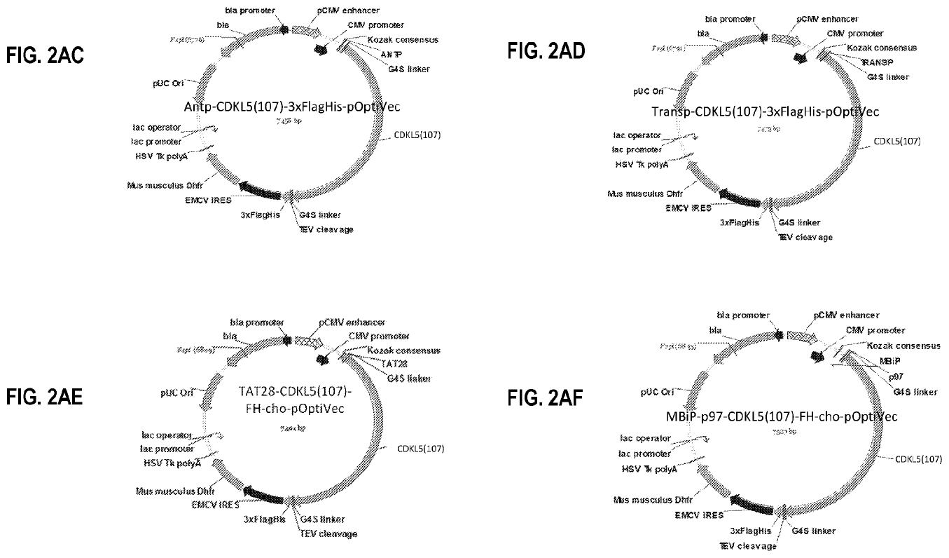

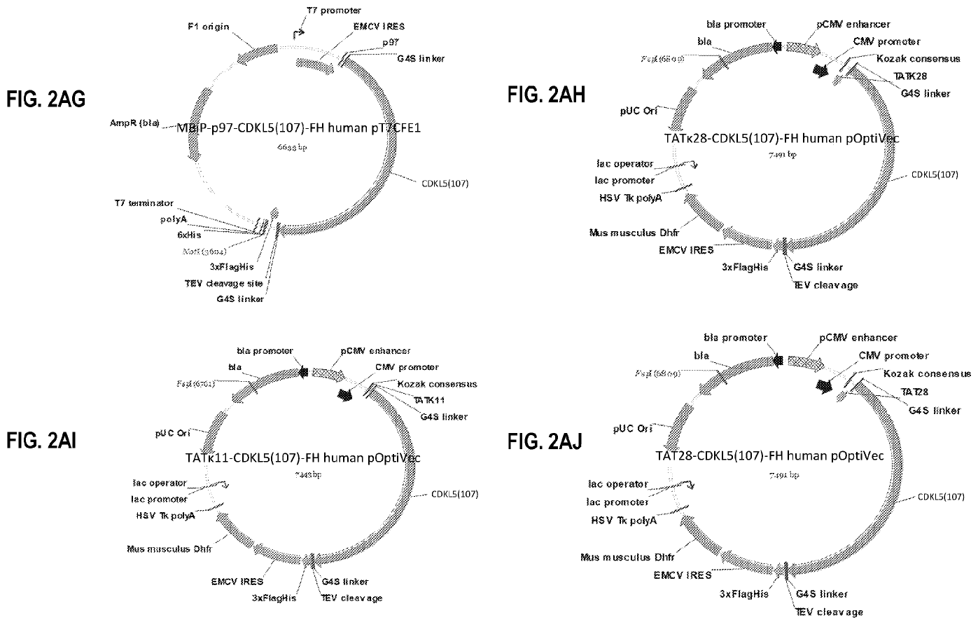

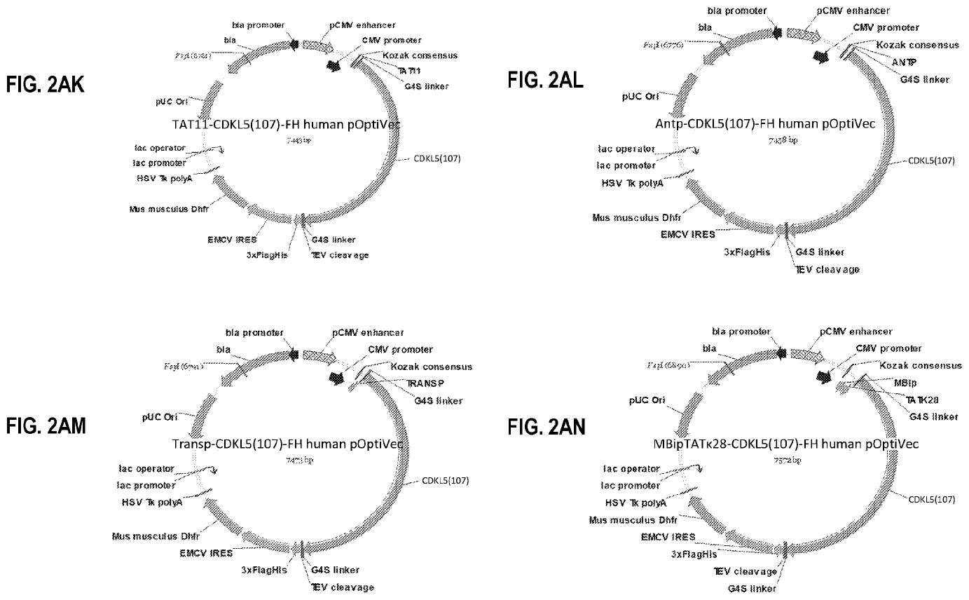

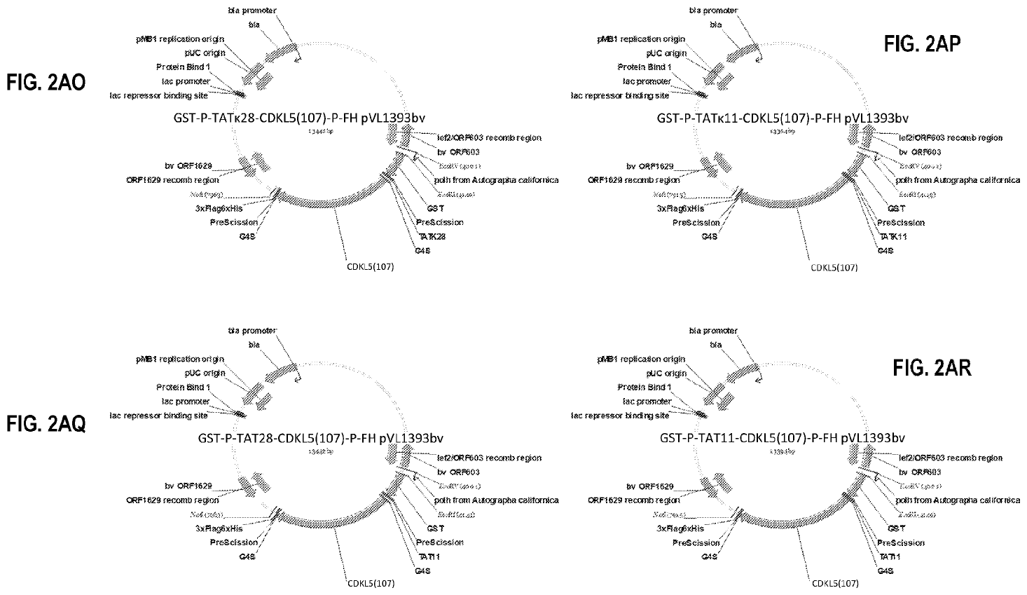

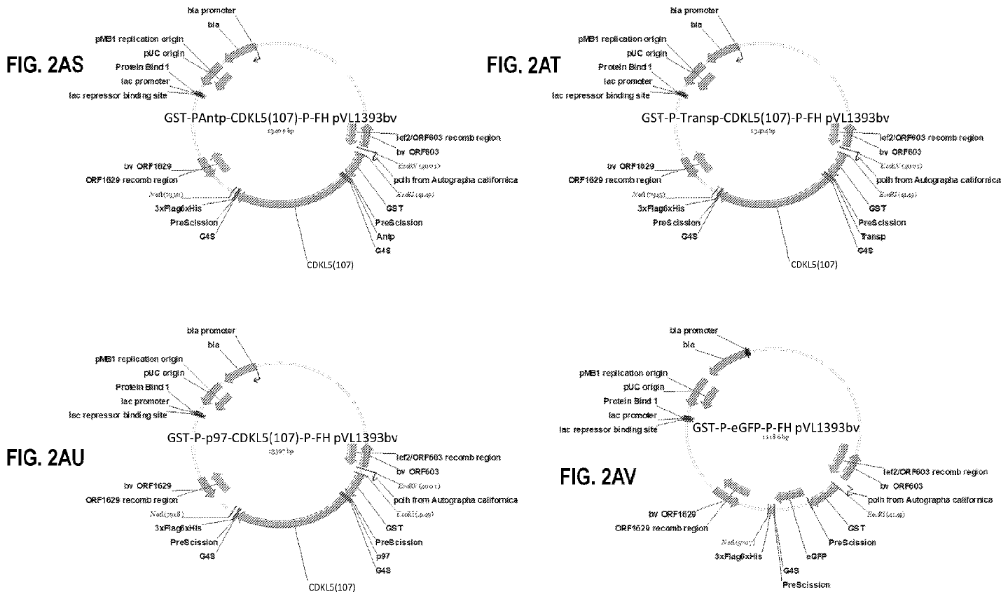

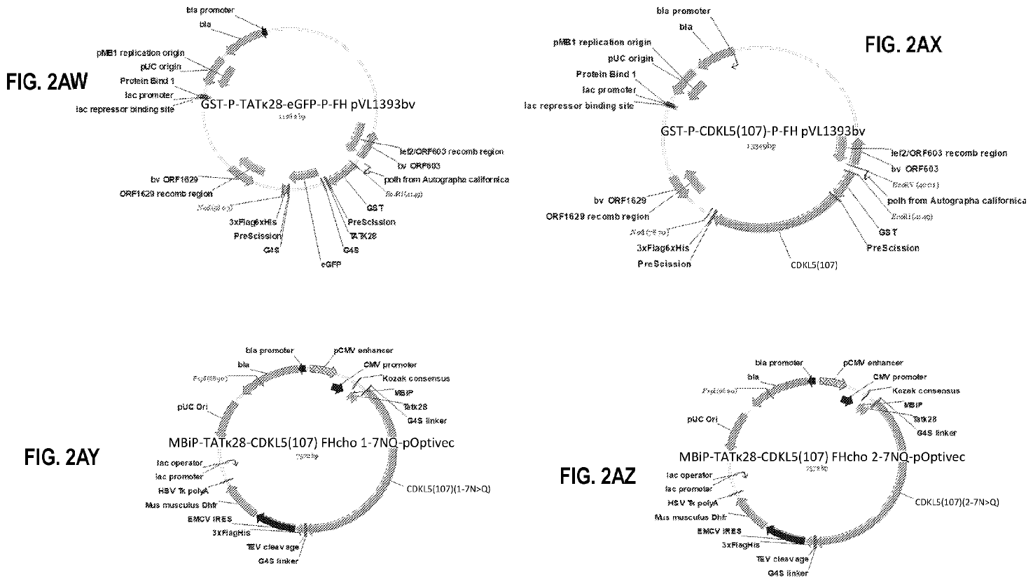

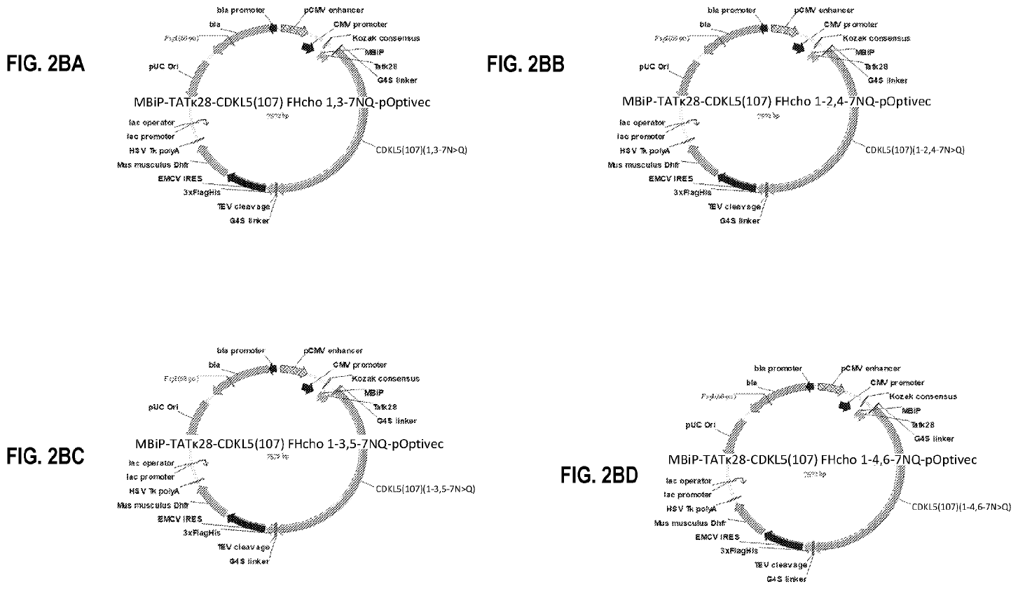

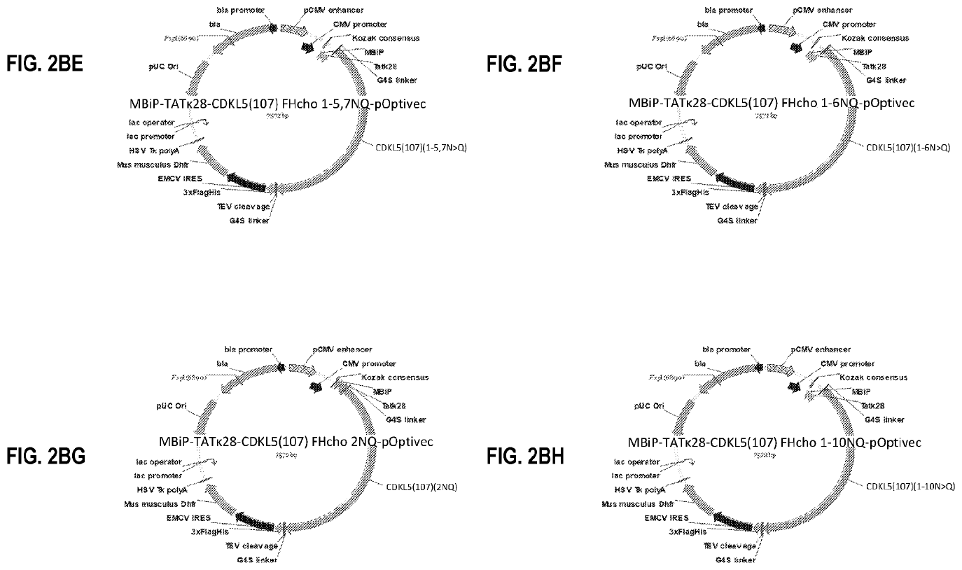

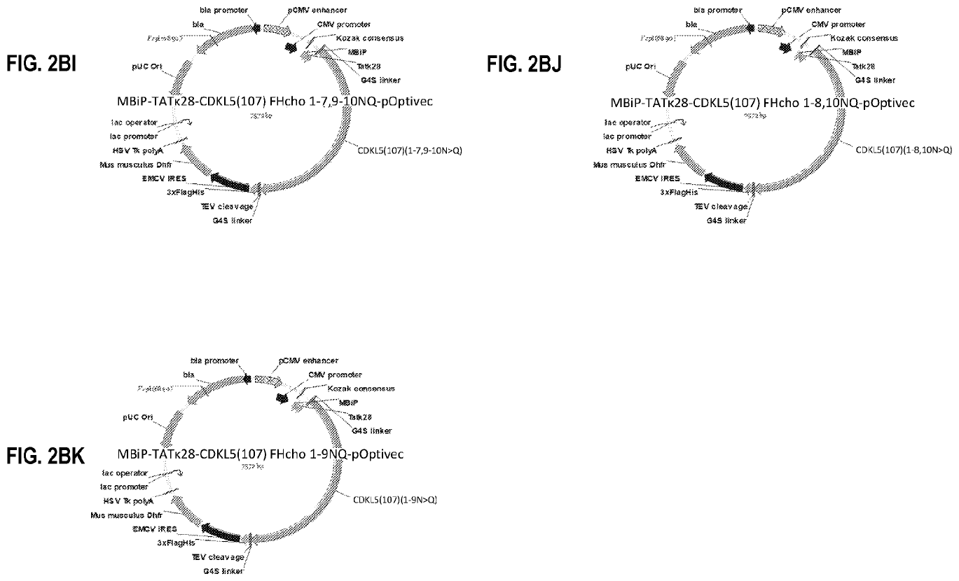

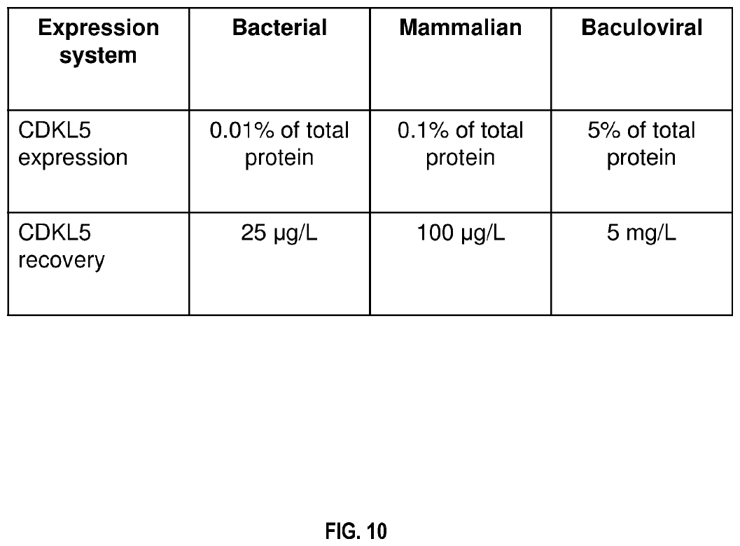

The recombinant protein (e.g. CDKL5 variant or fusion protein) can be expressed in and secreted from host cells using appropriate vectors. For example, mammalian cells (e.g., CHO, HeLa or HEK cells), insect cells (e.g. Sf9 or BTI-Tn-5B1-4) or bacterial cells (e.g., E. coli or P. haloplanktis TAC 125 cells) can be used. Exemplary plasmids are described in the examples below and shown in FIGS. 2A-2BK. Those of skill in the art can select alternative vectors suitable for transforming, transfecting, or transducing cells to produce the CDKL5 variants and fusion proteins described herein. FIG. 10 shows relative CDKL5 expression and yield in bacterial, mammalian and insect cell expression system.

After expression and secretion, recombinant protein can be recovered and purified from the surrounding cell culture media using standard techniques. Alternatively, recombinant protein can be isolated and purified directly from cells, rather than the medium.

In some embodiments, the BTI-Tn-5B1-4 cells are used to express and purify CDKL5 variant or fusion protein.

For lysis, the cells expressing the CDKL variant or fusion protein may be pelleted and subsequently resuspended into a lysis buffer. The resuspended cells may be then incubated in a cavitation chamber that is charged from about 100 PSI to about 2000 PSI with nitrogen gas. The resuspended cells may be incubated in the charged cavitation chamber for about 5 minutes to about 60 minutes. In some embodiments, the resuspended cells may be incubated in the cavitation chamber charged to 750 PSI with nitrogen gas. In some embodiments, the resuspended cells may be incubated in the charged cavitation chamber for 15 minutes. An effluent from the cavitation chamber after incubation may be then transferred on ice. A detergent may be added in the effluent followed by incubation on ice for about 5 minutes to about 60 minutes. In some embodiments, the detergent is added in the amount of about 0.1% (w/v) to about 5% (w/v). In some embodiments, the detergent is Triton X-100. The effluent with the detergent is then sonicated to lyse the cells. After lysis, soluble fractions and insoluble fractions may be separated. In some embodiments, the soluble fraction and insoluble fraction may be separated by centrifugation. The soluble material may be filtered. In some embodiments, the soluble material may be filtered through 0.45 μm filter.

For purification of the CDKL5 variants or the fusion protein, the filtered soluble material is then subject to purification. In some embodiments, the CDKL5 variants or the fusion protein is purified by a chromatography technique. In some embodiments, the chromatography technique is an affinity chromatography. In some embodiments, the CDKL5 variant or the fusion protein comprises one or more affinity tags. In some embodiments, the affinity-tag include, but are not limited to, epitope tags (e.g. MYC, HA, V5, NE, StrepII, Twin-Strep-tag®, HPC4), glutathione S-transferase (GST), maltose-binding protein (MBP), calmodulin-binding peptide (CBP), FLAG®, 3×FLAG®, polyhistidine (His) and combination thereof. In some embodiments, the CDKL5 variant or the fusion protein has a Twin-Strep-tag®. In some embodiments, the CDKL5 variant or the fusion protein with the affinity-tag is purified on a purification resin. In some embodiments of the CDKL5 variant or the fusion protein with a Twin-Strep-tag®, the purification resin is a strep-tactin resin.

Some embodiments of the CDKL5 variant or the fusion protein may also include one or more protease cleavage sites. In some embodiments, the protease cleavage site is located on the N-terminus of the CDKL5 variant or the fusion protein. In some embodiments, the protease cleavage site is located on the C-terminus of the CDKL5 variant or the fusion protein. In some embodiments, the protease cleavage site is located on N-terminus and C-terminus of the CDKL5 variant or the fusion protein. In some embodiments, the cleavage is performed when the CDKL5 variant or the fusion protein is bound to the purification resin. In some embodiments, the cleavage is performed when the CDKL5 variant or the fusion protein with the Twin-Strep-tag® is bound to the strep-tactin resin.

### Protein Replacement Therapy

In one or more embodiments, a subject may be administered with the CDKL5 protein or variants or fusion proteins. In some embodiments, the subjects may be humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the subject is a human.

In one or more embodiments, a cellular uptake of the CDKL5 protein or variants or fusion proteins is determined in cells isolated from the subject. In some embodiments, the cells may be isolated from rats. In some embodiments, the cells may be neuronal cells. In some embodiments, the cells may be embryonic primary cortical neurons. In some embodiments, the embryonic primary cortical neurons may be isolated from rats. In some embodiments, the cells may be cultured and incubated with the CDKL5 protein or variants for a duration of time. The duration of time may be at least 5 minutes, at least 10 minutes, at least 15 minutes, at least 20 minutes, at least 25 minutes, at least 30 minutes, at least 40 minutes, at least 50 minutes or at least 60 minutes. In some embodiments the duration of time may be from 5 minutes to 24 hours, 15 minutes to 24 hour, 30 minutes to 24 hour, 1 hour to 24 hour, 4 hour to 24 hour, 8 hour to 24 hour, 12 hour to 24 hour, 5 minutes to 12 hours, 15 minutes to 12 hour, 30 minutes to 12 hour, 1 hour to 12 hour, 2 hour to 12 hour, 4 hour to 12 hour, 6 hour to 12 hour, 8 hour to 12 hour, 10 hour to 12 hour, 5 minutes to 6 hours, 15 minutes to 6 hour, 30 minutes to 6 hour, 1 hour to 6 hour, 1.5 hour to 6 hour, 2 hour to 6 hour, 2.5 hour to 6 hour, 3 hour to 6 hour, 4 hour to 6 hour 5 hour to 6 hour, 5 minutes to 4 hours, 15 minutes to 4 hour, 30 minutes to 4 hour, 1 hour to 4 hour, 1.5 hour to 4 hour, 2 hour to 4 hour, 2.5 hour to 4 hour, 3 hour to 4 hour, 5 minutes to 2 hours, 15 minutes to 2 hour, 30 minutes to 2 hour, 1 hour to 2 hour, 1.5 hour to 2 hour, 5 minutes to 1 hours, 15 minutes to 1 hour or 30 minutes to 1 hour.

### Gene Therapy

Any of the CDKL5 polypeptides and/or fusion proteins described herein can be utilized in gene therapy via an appropriate polynucleotide (e.g. DNA or RNA) encoding the desired CDKL5 polypeptide and/or fusion protein.

In various embodiments, gene therapy is provided through the use of a composition comprising a gene therapy delivery system and a CDKL5 polynucleotide. Exemplary gene therapy delivery systems include, but are not limited to, viral vectors, liposomes, lipid-nucleic acid nanoparticles, exosomes and gene editing systems. For example, a gene editing system such as Clustered Regularly Interspaced Short Palindromic Repeat (CRISPR) associated protein 9 (CRISPR-Cas-9), Transcription activator-like effector nucleases (TALEN) or ZNF (Zinc finger proteins) can be used to insert the CDKL5 polynucleotide into the DNA of the host cell.

Viral vectors include, but are not limited to, adenoviral vectors, adeno-associated viral (AAV) vectors, lentiviral vectors, retroviral vectors, poxviral vectors or herpes simplex viral vectors. Viral vectors typically utilize a viral particle (virion) including an outer protein shell (capsid) and one or more DNA or RNA sequences (viral polynucleotides) encapsulated in the capsid. For example, AAV vectors typically include one or more inverted terminal repeat (ITR) sequences, a replication (Rep) gene sequence, and a capsid (Cap) gene sequence. The ITR, Rep and Cap sequences may be included in the same plasmid (in cis), or may be provided in separate plasmids (in trans). The capsid may be derived from the same serotype as the ITR sequences, or the AAV vector can be a hybrid vector utilizing ITR sequences and capsids derived from different AAV serotypes. Exemplary AAV serotypes include AAV1, AAV 2, AAV 3, AAV 4, AAV 5, AAV 6, AAV 7, AAV 8, AAV 9, AAV10, AAV11, hybrid serotypes, and synthetic serotypes. An exemplary set of ITRs is provided in SEQ ID NO: 27 (L-ITR) and SEQ ID NO: 28 (R-ITR), which are derived from AAV2.

The viral vectors also may include additional elements for increasing expression and/or stabilizing the vector such as promoters (e.g., hybrid CBA promoter (CBh) and human synapsin 1 promoter (hSyn1)), a polyadenylation signals (e.g. Bovine growth hormone polyadenylation signal (bGHpolyA)), stabilizing elements (e.g. Woodchuck Hepatitis Virus (WHP) Posttranscriptional Regulatory Element (WPRE)) and/or an SV40 intron. The DNA sequences for CBh and hSyn1 are provided in SEQ ID NO: 29 and SEQ ID NO: 30, respectively.

The gene therapy delivery system can be utilized to deliver the CDKL5 polynucleotide to the target cells so that the CDKL5 polypeptide (or fusion protein comprising the same) can be expressed in the target cells. In various embodiments, the CDKL5 polypeptide (e.g. wild-type CDKL5 polypeptides, CDKL5 variants with one or more N-linked glycosylation sites removed and/or shorter CDKL5 variants) (or fusion protein comprising the same) is expressed in the target cell and utilized in the same cell. In other embodiments, the CDKL5 polypeptide (or fusion protein comprising the same) is expressed in a first cell, secreted, and then penetrates into a second cell. In such embodiments, a leader signal polypeptide and/or a cell-penetration may be used to enhance secretion and/or penetration of the CDKL5 polypeptide. Without wishing to be bound by any particular theory, it is believed that secretion and penetration of CDKL5 polypeptide can be used to enhance the effects of gene therapy over conventional gene therapy approaches that only introduce DNA and RNA into the patient, as transduction in gene therapy may only be limited to a certain portion of the patient's cells (e.g. 10% of the target patient cells are successfully transduced with the DNA/RNA). In this way, the successfully transduced cells may be used to express the CDKL5 polypeptide (or fusion protein comprising the same) for both the transduced cells and neighboring cells that were not successfully transduced.

### Cross-Correction

Another aspect of the invention can include cross-correction. The genetherapy may not be effective to successfully transfect all defective cells. In one or more embodiments, a genetic defect in non-transfected cells can be corrected by the neighboring successfully transfected cells. For example, the CDKL5 polypeptide or fusion protein may be expressed in a successfully transfected cell, secreted from that cell, and taken up by a neighboring cell that was not successfully transfected. The defect may be cross-corrected by any of the gene therapy methods described herein via an appropriate polynucleotide (e.g. DNA or RNA) encoding the desired CDKL5 polypeptide and/or fusion protein. Any of the CDKL5 polypeptides and/or fusion proteins described herein can be utilized to cross-correct a CDKL5-related defect.

In one or more embodiments, a CDKL5 null subject is used for determining the fusion protein induced cross-correction. In some embodiments, the subject is a mouse. In some embodiments, a viral vector may be used to correct the CDKL5 defect. In a particular embodiment, AAV vector was used to correct the CDKL5 defect. In a particular embodiment, the AAV vector comprises a AAV-PHP.B.CBH.BIP-TATκ28-CDKL5.SV40. In a particular embodiment, the viral vector comprising corrective gene is administered in a dose sufficient to correct the genetic defect. In some embodiments, the sufficient dose for correcting genetic defect in mice is in a range of 10×e² GC/mice to 10×e¹⁵ GC/mice. In some embodiments, the sufficient dose for correcting genetic defect in mice may be 10×e² GC/mice, 10×e³ GC/mice, 10×e⁴ GC/mice, 10×e⁵ GC/mice, 10×e⁶ GC/mice, 10×e⁷ GC/mice, 10×e⁸ GC/mice, 10×e⁹ GC/mice, 10×e¹⁰ GC/mice, 10×e¹¹ GC/mice, 10×e¹² GC/mice, 10×e¹³ GC/mice, 10×e¹⁴ GC/mice or 10×e¹⁵ GC/mice. Exemplary routes of administration include, but are not limited to, intrathecal, intravenous, intracisternal, retro-orbital, intraperitoneal, intracerebroventrical or intraparenchymal administration.

In one or more embodiments, the CDKL5 null mice may be divided into a treatment group and a control group. Each group, the treatment group and the control group, may further be divided into two subgroups based on route of administration. More than one route can be used concurrently, if desired. In one or more embodiments, each subgroup may be administered AAV-PHP.B.CBH.BIP-TATκ28-CDKL5.SV40 dose through either intracerebroventricular (ICV) or retro orbital (RO) route of administration. Each subgroup received AAV-PHP.B.CBH.BIP-TATκ28-CDKL5.SV40 dose in an amount of 10×e⁸ GC/mice, 10×e⁹ GC/mice or 10×e¹⁰ GC/mice. Three months post-administration, the impact of the vector on behavioral endpoints may be assessed and the mice may be euthanized for transgene expression analysis.

After euthanizing mice, various section of brain may be taken including but not limited to sagittal section. The sections may be immunostained with DAPI, anti-NeuN antibody, anti-CDKL5 RNA antibody and anti-CDKL5 protein antibody. The sections may be taken from isocortex, striatum, thalamus and hippocampal formation section of brains.

The immunostained images may be analyzed using Visiopharm software. The immunostained cells may be divided into six groups: (1) DAPI stain to identify cells; (2) NeuN stain to identify neurons; (3) Neurons having CDKL5 mRNA and CDKL5 protein; (4) Neurons having CDKL5 mRNA; (5) Cross-corrected neurons; and (6) Cross-corrected non-neurons. The result of image analysis may be further subject to a statistical analysis for cross-corrected neurons and non-neurons.

### Formulations, Methods of Treatment and Use

The gene therapy compositions (e.g. comprising CDKL5 polynucleotides) or the protein replacement therapy compositions (e.g. comprising recombinant proteins including CDKL5 variants or fusion proteins), can be formulated in accordance with the routine procedures as a pharmaceutical composition adapted for administration to human beings. For example, in one or more embodiments, a composition for intravenous administration is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

Gene therapy compositions (e.g. comprising CDKL5 polynucleotides) or protein replacement therapy compositions (e.g. comprising recombinant proteins including CDKL5 variants or fusion proteins) (or a composition or medicament containing the gene therapy composition or protein replacement therapy composition) are administered by an appropriate route. In one or more embodiments, the gene therapy composition or protein replacement therapy composition is administered intravenously. In other embodiments, the gene therapy composition or protein replacement therapy composition is administered by direct administration to a target tissue, such as to heart or skeletal muscle (e.g., intramuscular; intraventricularly), or nervous system (e.g., intrathecal delivery—delivery into the space under the arachnoid membrane of the brain or spinal cord). More than one route can be used concurrently, if desired. Exemplary routes of administration include, but are not limited to, intrathecal, intravenous, intracisternal, intracerebroventrical or intraparenchymal administration.

The gene therapy composition (e.g. comprising CDKL5 polynucleotides) or protein replacement therapy composition (e.g. comprising recombinant protein including CDKL5 variants or fusion proteins) (or a composition or medicament containing such gene therapy composition or protein replacement therapy) is administered in a therapeutically effective amount (e.g., a dosage amount that, when administered at regular intervals, is sufficient to treat the disease, such as by ameliorating symptoms associated with the disease, preventing or delaying the onset of the disease, and/or lessening the severity or frequency of symptoms of the disease). The amount which will be therapeutically effective in the treatment of the disease will depend on the nature and extent of the disease's effects. In addition, in vitro or in vivo assays may optionally be employed to help identify optimal dosage ranges. The precise dose to be employed will also depend on the route of administration, and the seriousness of the disease, and should be decided according to the judgment of a practitioner and each patient's circumstances. Effective doses may be extrapolated from dose-response curves derived from in vitro or animal model test systems.

The therapeutically effective amount of gene therapy composition (e.g. comprising CDKL5 polynucleotides) or protein replacement therapy composition (e.g. comprising recombinant protein including CDKL5 variants or fusion proteins) (or a composition or medicament containing such gene therapy composition or protein replacement therapy) can be administered at regular intervals, depending on the nature and extent of the disease's effects, and/or on an ongoing basis. Administration at a “regular interval,” as used herein, indicates that the therapeutically effective amount is administered periodically (as distinguished from a one-time dose). The administration interval for a single individual need not be a fixed interval, but can be varied over time, depending on the needs of the individual.

The gene therapy composition (e.g. comprising CDKL5 polynucleotides) or protein replacement therapy composition (e.g. comprising recombinant protein including CDKL5 variants or fusion proteins) (or a composition or medicament containing such gene therapy composition or protein replacement therapy composition) may be prepared for later use, such as in a unit dose vial or syringe, or in a bottle or bag for intravenous administration. Kits containing the gene therapy composition (e.g. comprising CDKL5 polynucleotides) or protein replacement therapy composition (e.g. comprising recombinant protein including CDKL5 variants or fusion proteins) (or a composition or medicament containing such gene therapy composition or protein replacement therapy composition), as well as optional excipients or other active ingredients, such as other drugs, may be enclosed in packaging material and accompanied by instructions for reconstitution, dilution or dosing for treating a subject in need of treatment, such as a patient having a CDKL5 deficiency, Rett syndrome, or a Rett syndrome variant.

### EXAMPLES

### Examples 1-12—CDKL5 Fusion Proteins

FIGS. 2A-2BK show plasmids for expressing fusion proteins in suitable cells, such as mammalian cells (e.g., CHO cells or HEK cells), insect cells (e.g. Sf9 cells) or bacterial cells (e.g., E. coli cells). These proteins have the amino acid sequences set forth in SEQ ID NOS: 43-105. The numbering of the deletions or truncations for the fusion proteins of SEQ ID NOS: 49-59 comprising CDKL5 truncation variants is relative to the full-length CDKL5₁₀₇ polypeptide (1-960). The fusion proteins of SEQ ID NOS: 93-105 comprising CDKL5 glycosylation variants have the specified N-linked glycosylation sites altered by substitutions of Asn for Gln, e.g. “1-10NQ” indicates that all 10 N-linked glycosylation sites have been altered by substituting Asn for Gln and “2NQ” indicates that only the second N-linked glycosylation site has been altered by substituting Asn for Gln. Also, some N-linked glycosylation sites were predicted to have a higher likelihood of glycosylation than other sites, and thus these sites were investigated first. Based on this, the first 7 N-linked glycosylation sites investigated are labeled as sites 1-7 and are indicated in bold font in the amino acid sequences, and the next 3 N-linked glycosylation sites investigated are labeled as sites 8-10 and are indicated in bold and underlined font in the amino acid sequences. Therefore, the order of the N-linked glycosylation sites from the N-terminus to the C-terminus are 1, 2, 3, 8, 4, 9, 10, 5, 6 and 7. The numbering of the N-linked glycosylation sites relative to the full-length CDKL5-107 polypeptide (1-960) and motif sequence are as follows: 1=Asn159, NLS; 2=Asn167, NYT; 3=Asn348, NLS; 4=Asn500, NLS; 5=Asn764, NIS; 6=Asn942, NRT; 7=Asn945, NRS; 8=Asn363, NES; 9=Asn731, NVS; 10=Asn748, NHS.

In those constructs wherein CDKL5 is fused C-terminally to additional N-terminal amino acid sequences, the initial methionine (amino acid 1) of CDKL5 is removed. In these constructs, the CDKL5 polypeptide begins with the second amino acid, lysine. Although specific reference is made to N-terminal amino acid sequences (e.g. N-terminal CPPs), C-terminal amino acid sequences (e.g. C-terminal CPPs) are also encompassed by the present disclosure.

The abbreviations used in FIGS. 2A-2BK and SEQ ID NOS: 43-105 are summarized in Table 1 below:

TABLE 1

| Features | Description |
|---|---|
| pOptiVec | expression vector for CHO DG44 cells, using pCMV promoter for |
| | high expression of recombinant protein; from ThermoFisher Scientific |
| | Inc. |
| pEX-1 | expression vector for bacterial cells, using T7 promoter for high |
| | expression of recombinant protein; from OriGene Technologies, Inc |
| pT7CFE1 | expression vector for human cells, using T7 promoter for high |
| | expression of recombinant protein; from ThermoFisher Scientific Inc. |
| pVL1393 | expression vector for insect cells, using polyhedron promoter for high |
| | expression of recombinant protein; from Expression Systems, LLC |
| pCMV | allows high expression level of recombinant protein |
| enhancer and | |
| promoter | |
| Kozak | for proper initiation of translation |
| consensus | |
| MBiP | modified BiP leader signal polypeptide (from U.S. Pat. No. 9,279,007; |
| | SEQ ID NO. 20) for secretion of recombinant protein; |
| | MKLSLVAAMLLLLSLVAAMLLLLSAARA |
| mvBIP | further modified BiP leader signal polypeptide including valine before |
| | lysine, MVKLSLVAAMLLLLSLVAAMLLLLSAARA |
| Igκ | murine Igκ chain leader polypeptide for secretion of recombinant |
| | protein (from ThermoFisher vectors; e.g. pSecTag2); |
| | METDTLLLWVLLLWVPGSTG |
| TATκ28, | TATκ28 peptide, GDAAQPARRARRTKLAAYARKAARQARA |
| TATκ28p, | |
| Tκ28p | |
| TATκκ28 | TATκκ28 peptide, GDAAQPAARARRTKLAAYARKAARQARA |
| TAT28, | TAT28 peptide, GDAAQPARRARRTKLAAYGRKKRRQRRR |
| TAT28p, | |
| TT28p | |
| TATκ11 | TATκ11 peptide, YARKAARQARA |
| TAT11 | TAT11 peptide, YGRKKRRQRRR |
| Antp | Antennapedia peptide, RQIKIWFQNRRMKWKK |
| Transp | Transportan peptide, AGYLLGKINLKALAALAKKIL |
| P97 | P97 peptide, DSSHAFTLDELR |
| G4S linker | a short linker consisting of 4 glycine and 1 serine |
| CDKL5(107) | human CDKL5-107 isoform |
| CDKL5_107 | |
| CDKL5(115) | human CDKL5-115 isoform |
| CDKL5_115 | |
| delta###-### | refers to the deletion of ###-### amino acids to form truncated forms |
| | of protein |
| ##-##NQ | refers to the substitution of Asn to Gln at ##-## N-linked glycosylation |
| | sites |
| AMPH1 | gene encoding human Amphiphysin1 |
| eGFP | gene encoding the enhanced Green Fluorescent Protein; allows for |
| | detection using anti-GFP or fluorescence |
| NLS | gene encoding a nuclear localization signal |
| GST | glutathione S-transferase |
| PreScission, | PreScission protease cleavage site |
| P | |
| TEV | TEV protease cleavage recognition site; allows removal of 3XFLAG- |
| cleavage | HIS tag (or other tags) after initial purification |
| 3XFlagHis, | 3XFLAG tag, followed by Glycine-Alanine-Proline (a short linker), |
| FH | and 6xHis tag; Flag and His tag allows detection of fusion protein with |
| | anti-Flag and anti-His and allows purification |
| EMCV IRES | Internal Ribosome Entry Site from the Encephalomyocarditis Virus |
| | allows for cap-independent translation of DHFR |
| DHFR | Mus musculus (mouse) DHFR allows auxotrophic selection of |
| | transfected DG44 cells and for genomic amplification of stable cell |
| | lines using methotrexate (Mtx) |
| HSV Tk | Herpes Simplex Virus Thymidine Kinase polyadenylation signal |
| polyA | allows for efficient transcription termination and polyadenylation of |
| | mRNA |
| pUC Ori | pUC origin allows for high-copy number replication and growth in |
| | E. coli cells |
| bla promoter | promoter for ampicillin (bla) resistance gene |
| Bla | ampicillin resistance gene (β-lactamase) |

FIG. 2A shows an exemplary plasmid for expressing the fusion protein of SEQ ID NO: 43 in CHO cells. This fusion protein comprises the modified BiP leader signal polypeptide, TATκ28 and the full-length human CDKL5₁₀₇ isoform.

FIG. 2B shows an exemplary plasmid for expressing the fusion protein of SEQ ID NO: 44 in CHO cells. This fusion protein comprises the murine Igκ chain leader polypeptide, TATκ28 and the full-length human CDKL5₁₀₇ isoform.

FIG. 2C shows an exemplary plasmid for expressing the fusion protein of SEQ ID NO: 45 in CHO cells. This fusion protein comprises the modified BiP leader signal polypeptide, TATκ28 and the full-length human CDKL5₁₁₅ isoform.

FIG. 2D shows an exemplary plasmid for expressing the fusion protein of SEQ ID NO: 46 in CHO cells. This fusion protein comprises the murine Igκ chain leader polypeptide, TATκ28 and the full-length human CDKL5₁₁₅ isoform.

FIG. 2E shows an exemplary plasmid for expressing the fusion protein of SEQ ID NO: 47 in CHO cells. This fusion protein comprises TATκ28 and the full-length human CDKL5₁₀₇ isoform.

FIG. 2F shows an exemplary plasmid for expressing the fusion protein of SEQ ID NO: 48 in E. coli cells. This fusion protein comprises TATκ28 and the full-length human CDKL5₁₀₇ isoform.

FIG. 2G shows an exemplary plasmid for expressing the fusion protein of SEQ ID NO: 49 in E. coli cells. This fusion protein comprises TATκ28 and the CDKL5₁₀₇ variant of Construct 2.

FIG. 2H shows an exemplary plasmid for expressing the fusion protein of SEQ ID NO: 50 in E. coli cells. This fusion protein comprises TATκ28 and the CDKL5₁₀₇ variant of Construct 3.

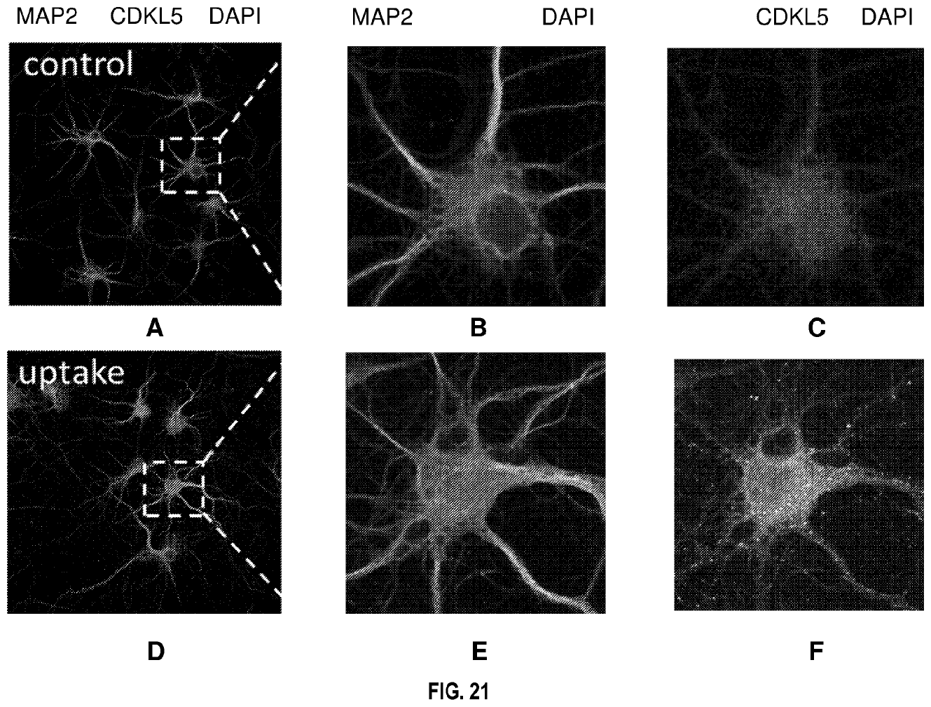

FIG. 21 shows an exemplary plasmid for expressing the fusion protein of SEQ ID NO: 51 in E. coli cells. This fusion protein comprises TATκ28 and the CDKL5₁₀₇ variant of Construct 4.

FIG. 2J shows an exemplary plasmid for expressing the fusion protein of SEQ ID NO: 52 in E. coli cells. This fusion protein comprises TATκ28 and the CDKL5₁₀₇ variant of Construct 5.

FIG. 2K shows an exemplary plasmid for expressing the fusion protein of SEQ ID NO: 53 in E. coli cells. This fusion protein comprises TATκ28 and the CDKL5₁₀₇ variant of Construct 6.

FIG. 2L shows an exemplary plasmid for expressing the fusion protein of SEQ ID NO: 54 in E. coli cells. This fusion protein comprises TATκ28 and the CDKL5₁₀₇ variant of Construct 7.

FIG. 2M shows an exemplary plasmid for expressing the fusion protein of SEQ ID NO: 55 in E. coli cells. This fusion protein comprises TATκ28 and the CDKL5₁₀₇ variant of Construct 8.

FIG. 2N shows an exemplary plasmid for expressing the fusion protein of SEQ ID NO: 56 in E. coli cells. This fusion protein comprises TATκ28 and the CDKL5₁₀₇ variant of Construct 9.

FIG. 2O shows an exemplary plasmid for expressing the fusion protein of SEQ ID NO: 57 in E. coli cells. This fusion protein comprises TATκ28 and the CDKL5₁₀₇ variant of Construct 10.

FIG. 2P shows an exemplary plasmid for expressing the fusion protein of SEQ ID NO: 58 in E. coli cells. This fusion protein comprises TATκ28 and the CDKL5₁₀₇ variant of Construct 11.

FIG. 2Q shows an exemplary plasmid for expressing the fusion protein of SEQ ID NO: 59 in E. coli cells. This fusion protein comprises TATκ28 and the CDKL5₁₀₇ variant of Construct 12.

FIG. 2R shows an exemplary plasmid for expressing the fusion protein of SEQ ID NO: 60 in E. coli cells. This fusion protein comprises TAT28 and the full-length human CDKL5₁₀₇ isoform.

FIG. 2S shows an exemplary plasmid for expressing the fusion protein of SEQ ID NO: 61 in E. coli cells. This fusion protein comprises TATκ28 and enhanced Green Fluorescent Protein (eGFP).

FIG. 2T shows an exemplary plasmid for expressing the fusion protein of SEQ ID NO: 62 in E. coli cells. This fusion protein comprises eGFP without a CPP.

FIG. 2U shows an exemplary plasmid for expressing the fusion protein of SEQ ID NO: 63 in E. coli cells. This fusion protein comprises human Amphiphysin1 (AMPH1).

FIG. 2V shows an exemplary plasmid for expressing the fusion protein of SEQ ID NO: 64 in CHO cells. This fusion protein comprises human Amphiphysin1 (AMPH1).

FIG. 2W shows an exemplary plasmid for expressing the fusion protein of SEQ ID NO: 65 in CHO cells. This fusion protein comprises the modified BiP leader signal polypeptide, TATκ11 and the full-length human CDKL5₁₀₇ isoform.

FIG. 2X shows an exemplary plasmid for expressing the fusion protein of SEQ ID NO: 66 in CHO cells. This fusion protein comprises the murine Igκ chain leader polypeptide, TATκ11 and the full-length human CDKL5₁₀₇ isoform.

FIG. 2Y shows an exemplary plasmid for expressing the fusion protein of SEQ ID NO: 67 in CHO cells. This fusion protein comprises TATκ11 and the full-length human CDKL5₁₀₇ isoform without a leader signal polypeptide.

FIG. 2Z shows an exemplary plasmid for expressing the fusion protein of SEQ ID NO: 68 in E. coli cells. This fusion protein comprises TATκ11 and the full-length human CDKL5₁₀₇ isoform without a leader signal polypeptide.

FIG. 2AA shows an exemplary plasmid for expressing the fusion protein of SEQ ID NO: 69 in E. coli cells. This fusion protein comprises TAT11 and the full-length human CDKL5₁₀₇ isoform without a leader signal polypeptide.

FIG. 2AB shows an exemplary plasmid for expressing the fusion protein of SEQ ID NO: 70 in CHO cells. This fusion protein comprises TAT11 and the full-length human CDKL5₁₀₇ isoform without a leader signal polypeptide.

FIG. 2AC shows an exemplary plasmid for expressing the fusion protein of SEQ ID NO: 71 in CHO cells. This fusion protein comprises the Antennapedia CPP and the full-length human CDKL5₁₀₇ isoform without a leader signal polypeptide.

FIG. 2AD shows an exemplary plasmid for expressing the fusion protein of SEQ ID NO: 72 in CHO cells. This fusion protein comprises the Transportan CPP and the full-length human CDKL5₁₀₇ isoform without a leader signal polypeptide.

FIG. 2AE shows an exemplary plasmid for expressing the fusion protein of SEQ ID NO: 73 in CHO cells. This fusion protein comprises TAT28 and the full-length human CDKL5₁₀₇ isoform without a leader signal polypeptide.

FIG. 2AF shows an exemplary plasmid for expressing the fusion protein of SEQ ID NO: 74 in CHO cells. This fusion protein comprises the modified BiP leader signal polypeptide, the P97 CPP and the full-length human CDKL5₁₀₇ isoform.

FIG. 2AG shows an exemplary plasmid for expressing the fusion protein of SEQ ID NO: 75 in human cells. This fusion protein comprises the P97 CPP and the full-length human CDKL5₁₀₇ isoform without a leader signal polypeptide.

FIG. 2AH shows an exemplary plasmid for expressing the fusion protein of SEQ ID NO: 76 in human cells. This fusion protein comprises TATκ28 and the full-length human CDKL5₁₀₇ isoform without a leader signal polypeptide.

FIG. 2AI shows an exemplary plasmid for expressing the fusion protein of SEQ ID NO: 77 in human cells. This fusion protein comprises TATκ11 and the full-length human CDKL5₁₀₇ isoform without a leader signal polypeptide.

FIG. 2AJ shows an exemplary plasmid for expressing the fusion protein of SEQ ID NO: 78 in human cells. This fusion protein comprises TAT28 and the full-length human CDKL5₁₀₇ isoform without a leader signal polypeptide.

FIG. 2AK shows an exemplary plasmid for expressing the fusion protein of SEQ ID NO: 79 in human cells. This fusion protein comprises TAT11 and the full-length human CDKL5₁₀₇ isoform without a leader signal polypeptide.

FIG. 2AL shows an exemplary plasmid for expressing the fusion protein of SEQ ID NO: 80 in human cells. This fusion protein comprises the Antennapedia CPP and the full-length human CDKL5₁₀₇ isoform without a leader signal polypeptide.

FIG. 2AM shows an exemplary plasmid for expressing the fusion protein of SEQ ID NO: 81 in human cells. This fusion protein comprises the Transportan CPP and the full-length human CDKL5₁₀₇ isoform without a leader signal polypeptide.

FIG. 2AN shows an exemplary plasmid for expressing the fusion protein of SEQ ID NO: 82 in human cells. This fusion protein comprises the modified BiP leader signal polypeptide, TATκ28 and the full-length human CDKL5₁₁₅ isoform.

FIG. 2AO shows an exemplary plasmid for expressing the fusion protein of SEQ ID NO: 83 in insect cells. This fusion protein comprises TATκ28 and the full-length human CDKL5₁₀₇ isoform without a leader signal polypeptide.

FIG. 2AP shows an exemplary plasmid for expressing the fusion protein of

SEQ ID NO: 84 in insect cells. This fusion protein comprises TATκ11 and the full-length human CDKL5₁₀₇ isoform without a leader signal polypeptide.

FIG. 2AQ shows an exemplary plasmid for expressing the fusion protein of SEQ ID NO: 85 in insect cells. This fusion protein comprises TAT28 and the full-length human CDKL5₁₀₇ isoform without a leader signal polypeptide.

FIG. 2AR shows an exemplary plasmid for expressing the fusion protein of SEQ ID NO: 86 in insect cells. This fusion protein comprises TAT11 and the full-length human CDKL5₁₀₇ isoform without a leader signal polypeptide.

FIG. 2AS shows an exemplary plasmid for expressing the fusion protein of SEQ ID NO: 87 in insect cells. This fusion protein comprises the Antennapedia CPP and the full-length human CDKL5₁₀₇ isoform without a leader signal polypeptide.

FIG. 2AT shows an exemplary plasmid for expressing the fusion protein of SEQ ID NO: 88 in insect cells. This fusion protein comprises the Transportan CPP and the full-length human CDKL5₁₀₇ isoform without a leader signal polypeptide.

FIG. 2AU shows an exemplary plasmid for expressing the fusion protein of SEQ ID NO: 89 in insect cells. This fusion protein comprises the P97 CPP and the full-length human CDKL5₁₀₇ isoform without a leader signal polypeptide.

FIG. 2AV shows an exemplary plasmid for expressing the fusion protein of SEQ ID NO: 90 in insect cells. This fusion protein comprises eGFP without a CPP.

FIG. 2AW shows an exemplary plasmid for expressing the fusion protein of SEQ ID NO: 91 in insect cells. This fusion protein comprises TATκ28 and eGFP.

FIG. 2AX shows an exemplary plasmid for expressing the fusion protein of SEQ ID NO: 92 in insect cells. This fusion protein comprises the full-length human CDKL5₁₀₇ isoform without a leader signal polypeptide or CPP.

FIG. 2AY shows an exemplary plasmid for expressing the fusion protein of SEQ ID NO: 93 in CHO cells. This fusion protein comprises the modified BiP leader signal polypeptide, TATκ28 and the 1-7NQ CDKL5₁₀₇ glycosylation variant.

FIG. 2AZ shows an exemplary plasmid for expressing the fusion protein of SEQ ID NO: 94 in CHO cells. This fusion protein comprises the modified BiP leader signal polypeptide, TATκ28 and the 2-7NQ CDKL5₁₀₇ glycosylation variant.

FIG. 2BA shows an exemplary plasmid for expressing the fusion protein of SEQ ID NO: 95 in CHO cells. This fusion protein comprises the modified BiP leader signal polypeptide, TATκ28 and the 1,3-7NQ CDKL5₁₀₇ glycosylation variant.

FIG. 2BB shows an exemplary plasmid for expressing the fusion protein of SEQ ID NO: 96 in CHO cells. This fusion protein comprises the modified BiP leader signal polypeptide, TATκ28 and the 1-2,4-7NQ CDKL5₁₀₇ glycosylation variant.

FIG. 2BC shows an exemplary plasmid for expressing the fusion protein of SEQ ID NO: 97 in CHO cells. This fusion protein comprises the modified BiP leader signal polypeptide, TATκ28 and the 1-3,5-7NQ CDKL5₁₀₇ glycosylation variant.

FIG. 2BD shows an exemplary plasmid for expressing the fusion protein of SEQ ID NO: 98 in CHO cells. This fusion protein comprises the modified BiP leader signal polypeptide, TATκ28 and the 1-4,6-7NQ CDKL5₁₀₇ glycosylation variant.

FIG. 2BE shows an exemplary plasmid for expressing the fusion protein of SEQ ID NO: 99 in CHO cells. This fusion protein comprises the modified BiP leader signal polypeptide, TATκ28 and the 1-5,7NQ CDKL5₁₀₇ glycosylation variant.

FIG. 2BF shows an exemplary plasmid for expressing the fusion protein of SEQ ID NO: 100 in CHO cells. This fusion protein comprises the modified BiP leader signal polypeptide, TATκ28 and the 1-6NQ CDKL5₁₀₇ glycosylation variant.

FIG. 2BG shows an exemplary plasmid for expressing the fusion protein of SEQ ID NO: 101 in CHO cells. This fusion protein comprises the modified BiP leader signal polypeptide, TATκ28 and the 2NQ CDKL5₁₀₇ glycosylation variant.

FIG. 2BH shows an exemplary plasmid for expressing the fusion protein of SEQ ID NO: 102 in CHO cells. This fusion protein comprises the modified BiP leader signal polypeptide, TATκ28 and the 1-10NQ CDKL5₁₀₇ glycosylation variant.

FIG. 2BI shows an exemplary plasmid for expressing the fusion protein of SEQ ID NO: 103 in CHO cells. This fusion protein comprises the modified BiP leader signal polypeptide, TATκ28 and the 1-7,9-10NQ CDKL5₁₀₇ glycosylation variant.

FIG. 2BJ shows an exemplary plasmid for expressing the fusion protein of SEQ ID NO: 104 in CHO cells. This fusion protein comprises the modified BiP leader signal polypeptide, TATκ28 and the 1-8,10NQ CDKL5₁₀₇ glycosylation variant.

FIG. 2BK shows an exemplary plasmid for expressing the fusion protein of SEQ ID NO: 105 in CHO cells. This fusion protein comprises the modified BiP leader signal polypeptide, TATκ28 and the 1-9NQ CDKL5₁₀₇ glycosylation variant.

Various CDKL5 fusion proteins were expressed in E. coli, CHO, HEK and insect cells, as well as using in vitro transcription/translation with HeLa cell lysates, as further described below.

### Example 1—Expression of CDKL5 Truncation Variants in E. Coll Cells

Full-length and truncations of TATκ28-CDKL5_107-FH were cloned into the pET vector, pEX-1, and transformed into the E. coli strain, BL21(DE3). Colony-purified transformants were cultured in LB+100 μg/mL ampicillin at 37° C. to exponential phase. The cultures were then cooled to 20° C. and induced with (or without) 1 mM IPTG for 16 hours. Cell pellets were collected and lysed in B-Per Complete Bacterial Protein Extraction Solution (Thermo) supplemented with 1X Complete Protease Inhibitor Complex (Roche). Lysis was allowed to proceed for 30 minutes at room temperature. A soluble fraction was prepared from the lysate by centrifugation at 16,000×g for 15 minutes at 4° C. Proteins were resolved on SDS-PAGE, transferred to nitrocellulose membranes, probed with a rabbit anti-polyhistidine antibody (Thermo), and detected with a fluorescent secondary antibody.

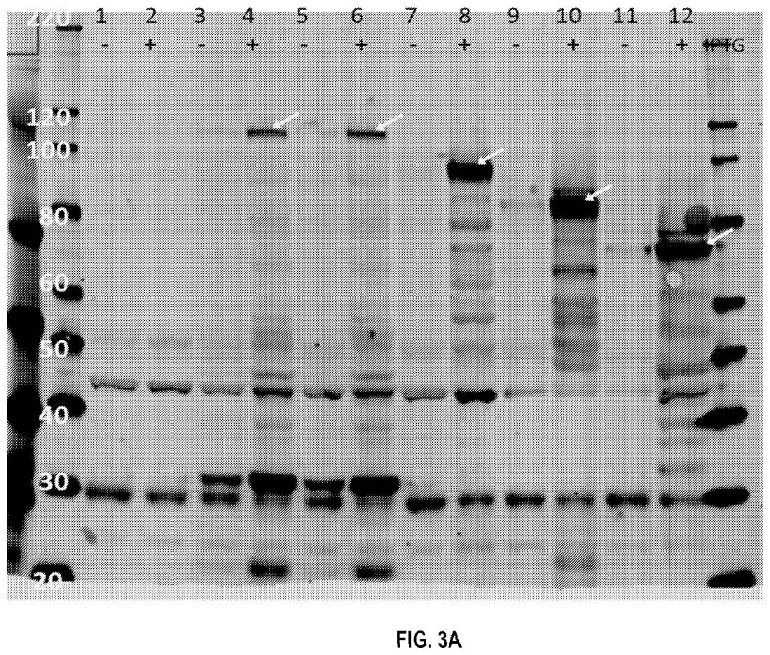

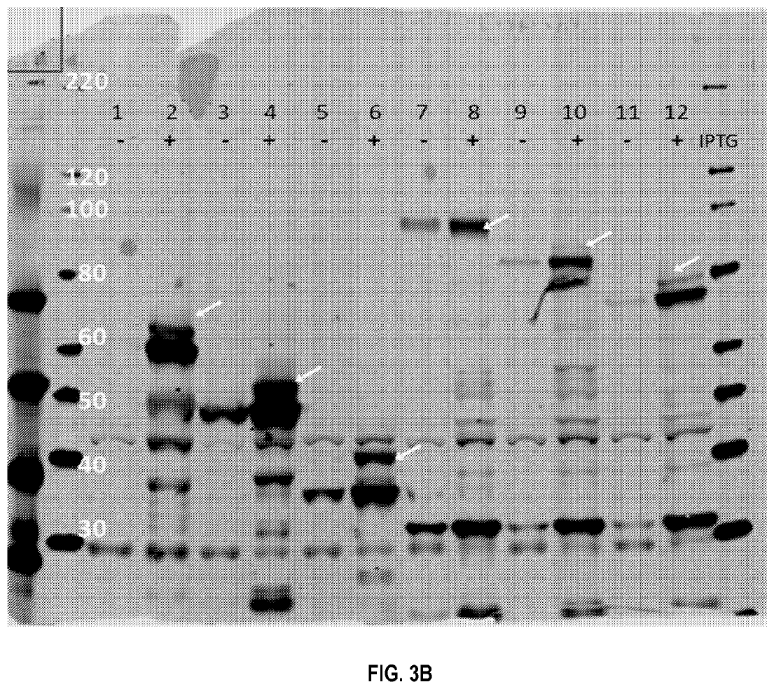

Blots shown FIGS. 3A and 3B confirmed expression of the CDKL5 truncation variants. In FIGS. 3A and 3B, the cultures without IPTG induction are the odd-numbered lanes and the cultures with IPTG induction are the even-numbered lanes, with no CDKL5 fusion protein being expressed in the lanes without IPTG induction and the CDKL5 fusion proteins being expressed in the lanes with IPTG induction.

For FIG. 3A, the lane identification is as follows:

TABLE 2
Lane Identification for Figure 3A

| | Sample | AA size | |
|---|---|---|---|
| # | Name | (difference) | Lane |
| 1 | pEX-1 empty | | 1, 2 |
| 2 | Tκ28p_107 | 1038 | 3, 4 |
| 3 | TT28p_107 | 1038 | 5, 6 |
| 4 | Δ853-960 | −108 | 7, 8 |
| 5 | Δ745-960 | −216 | 9, 10 |
| 6 | Δ637-960 | −324 | 11, 12 |

For FIG. 3B, the lane identification is as follows:

TABLE 3
Lane Identification for Figure 3B

| # | Sample Name | AA size (difference) | Lane |
|---|---|---|---|
| 7 | Δ529-960 | −432 | 1, 2 |
| 8 | Δ421-960 | −540 | 3, 4 |
| 9 | Δ315-960 | −646 | 5, 6 |
| 10 | Δ315-420 | −106 | 7, 8 |
| 11 | Δ315-528 | −214 | 9, 10 |
| 12 | Δ315-636 | −322 | 11, 12 |

### Example 2—Expression of CDKL5 Fusion Proteins in CHO Cells

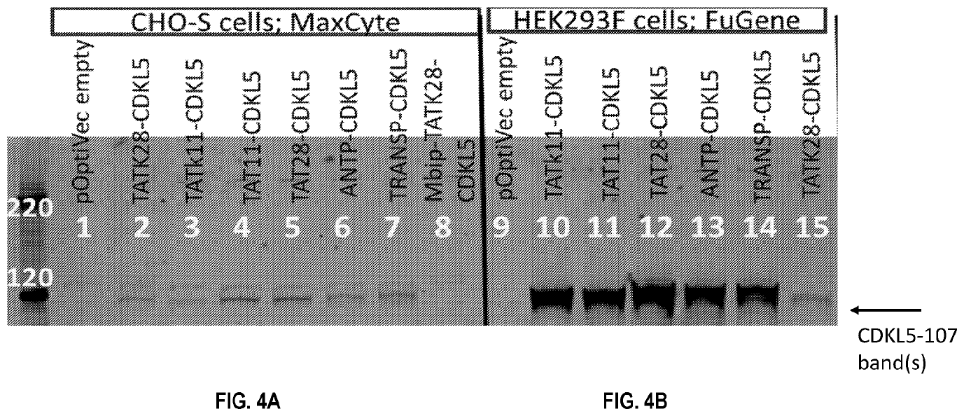

CHO-S cells (20×10\hat{ }6cells) were electroporated using Maxcyte STX with 8 plasmids: (1) pOptiVec empty vector; 2) TATκ28-CDKL5-107-3×FlagHis; 3) TATκ11-CDKL5-107-3×FlagHis; 4) TAT11-CDKL5-107-3×FlagHis; 5) TAT28-CDKL5-107-3×FlagHis; 6) ANTP-CDKL5-107-3×FlagHis; 7) TRANSP-CDKL5-107-3×FlagHis and 8) MBiP-TATκ28-CDKL5-107-3×FlagHis (coding sequences being CHO codon-optimized). Cells were recovered in culture medium, and cultured for one day. Cells were harvested and lysed. For each transfection, 20 μg lysate was subjected to 4-12% BisTris SDS-PAGE, and transferred to nitrocellulose blot using the iBlot2 system. The blot was blocked in 5% milk in 1×TBS-T. Blot was subjected to Western blot by incubating with 1:2000 dilution of rabbit anti-His antibody overnight. After a series of washes, blot was incubated with 1:10000 anti-rabbit IgG DyaLight 680 secondary antibody. Additional washes were performed. Blot was imaged on Licor Odyssey scanner. Blot shown in FIG. 4A confirmed expression of the CDKL5 fusion proteins.

### Example 3—Expression of CDKL5 Fusion Proteins in HEK Cells

HEK293F cells (8×10\hat{ }cells) were transfected with FuGeneHD (240 FuGeneHD: 8 μg DNA ratio) and 7 plasmids: 1) empty pOptiVec; 2) TATκ11-CDKL5_107-3×FlagHis; 3) TAT11-CDKL5_107-3×FlagHis; 4) TAT28-CDKL5_107-3×FlagHis; 5) ANTP-CDKL5_107-3×FlagHis; 6) TRANSP-CDKL5_107-3×FlagHis and 7) TATκ28-CDKL5_107-3×FlagHis (coding sequences being human codon-optimized). Cells were incubated and harvested 2 days post transfection. Cells were lysed, and 20 μg lysate was subjected to 4-12% BisTris SDS-PAGE, and transferred to nitrocellulose blot using the iBlot2 system. The blot was blocked in 5% milk in 1×TBS-T. Blot was subjected to Western blot by incubating with 1:2000 dilution of rabbit anti-His antibody overnight. After a series of washes, blot was incubated with 1:10000 anti-rabbit IgG DyaLight 680 secondary antibody. Additional washes were performed. Blot was imaged on Licor Odyssey scanner. Blot shown in FIG. 4B confirmed expression of the CDKL5 fusion proteins.

### Example 4—Methotrexate Amplification of CDKL5 Fusion Proteins in CHO Cells

Methotrexate amplification was used to amplify expression of CDKL5 fusion proteins in CHO-DG44 cells. TATκ28-CDKL5_107-FH (no signal sequence), Igκ-TATκ28-CDKL5_107-FH, and mBiP-TATκ28-CDKL5_107-FH were cloned into the pOptiVec vector providing the DHFR gene for methotrexate resistance. These plasmids were transfected into DG44 cells (deficient in dhfr) and selected by growth in medium deficient in hypoxanthine and thymidine. Methotrexate-resistant subcultures were obtained by culturing the cells sequentially in 0.1, 0.25, 0.5, and 1 μM Methotrexate (MTX), allowing cells to recover to 70% viability between steps. Cell pellets were lysed in 50 mM Tris-HCl with 75 mM NaCl, 1% Triton X-100, and 1.5X protease inhibitor cocktail (EDTA-free), pH 7.4. 40 μg of total protein were resolved on LDS-PAGE, transferred to nitrocellulose membranes, probed with a rabbit anti-polyhistidine antibody (Thermo), and detected with a fluorescent secondary antibody.

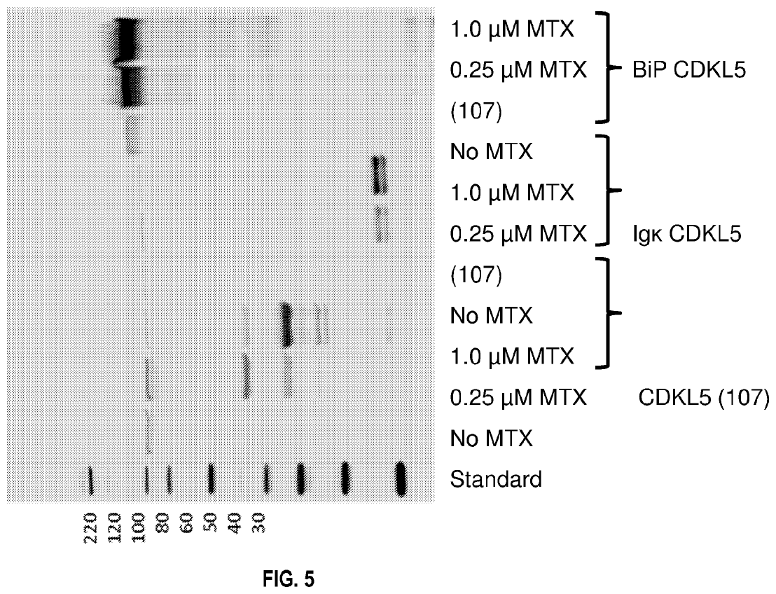

The blot shown in FIG. 5 demonstrates that as the methotrexate concentration was increased to select higher copy number variants of DHFR:CDKL5, evidence of genetic rearrangement appeared except for the mBiP construct, and only the mBiP version had increased levels of CDKL5. This pattern was replicated with both the 107 kDa (CDKL5_107) and 115 kDa (CDKL5_115) versions of CDKL5. Moreover, only with the mBiP construct was a slightly larger form of CDKL5 apparent. Without wishing to be bound by any particular theory, it is believed that the cytosolic expression of TATκ28-CDKL5 is either toxic to cells or reduces cell proliferation. Only those cells that rearranged the CDKL5 sequence, eliminating its expression, can be selected with high levels of methotrexate when a signal sequence is absent or the Igκ sequence is used. The higher mass form resulting from the mBiP signal sequence is consistent with the addition of N-linked glycans in the secretory pathway, and the lack of this larger form with the Igκ signal sequence suggests lower efficiency of translocation.

### Example 5—Comparison of CDKL5 Expression Secreted into the Medium and in Cell Lysates

In addition to the DG44 transfected cell lines noted above (TATκ28-CDKL5_107-FH without a signal sequence, Igκ-TATκ28-CDKL5_107-FH, and mBiP-TATκ28-CDKL5_107-FH), an Igκ-TATκ28-eGFP-CDKL5_107-MH plasmid stably transfected in adherent HEK293T cells were compared for secretion of CDKL5 fusion protein into the culture medium and in the cell lysates. The mBiP-TATκ28-CDKL5_107-FH cell line was represented by both 0 mM MTX and 0.5 μM MTX sub-cultures. After two days in serum-free growth, the conditioned medium was collected and concentrated 200-fold.

Cell pellets were lysed in 50 mM Tris-HCl with 75 mM NaCl, 1% Triton X-100, and 1.5X protease inhibitor cocktail (EDTA-free), pH 7.4. Cell lysates or concentrated conditioned medium were resolved on LDS-PAGE, transferred to nitrocellulose membranes, probed with a rabbit anti-polyhistidine antibody (Thermo), and detected with a fluorescent secondary antibody.

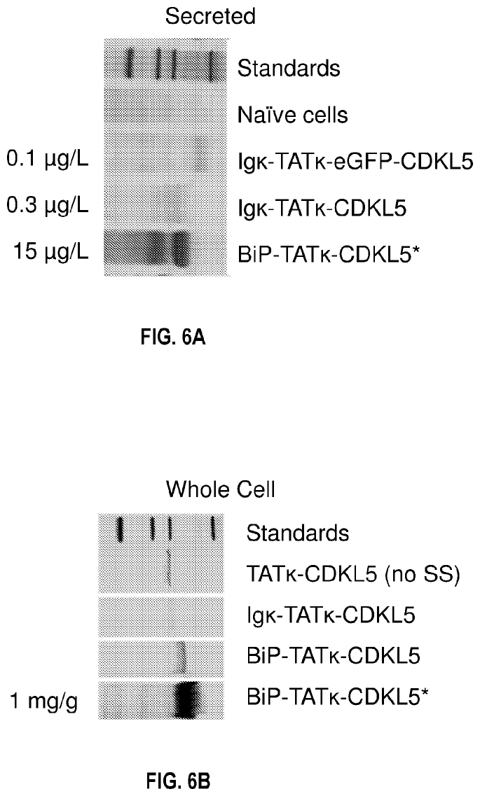

Blots shown in FIGS. 6A and 6B compare both the secreted and internal stores of CDKL5 among the various signal sequence constructs, respectively. The methotrexate amplified subculture is designated by the asterisk—Bip-TATκ-CDKL5*. Methotrexate amplified mBiP construct greatly increased the level of expressed CDKL5, and most of the protein was trapped inside the cells. The TATκ28-eGFP-CDKL5 construct only provided a secreted quantity of CDKL5 fusion protein of about 0.1 μg/L, while the mBiP-TATκ28-CDKL5 construct achieved a secreted quantity of CDKL5 fusion protein of about 15 μg/L (a 150-fold increase). Inside the same mBiP-TATκ28-CDKL5 expressing cells, the CDKL5 fusion proteins represented 0.1% (1 mg/g) of total protein.

### Example 6—Co-Expression of CDKL5 Fusion Proteins and Potential Substrates

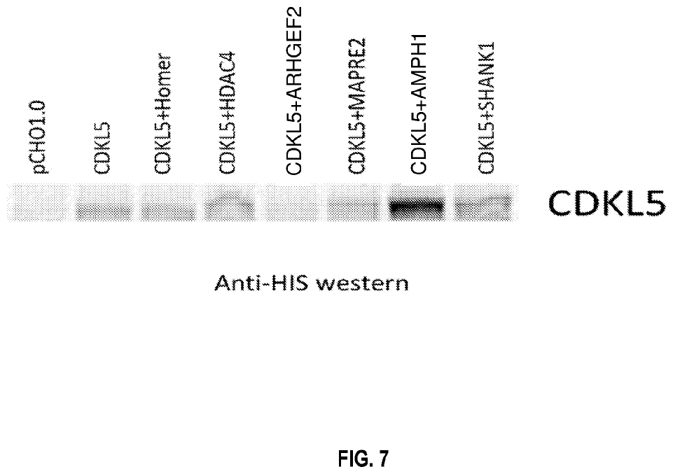

A single plasmid (pCHO 1.0) harboring both TATκ28-CDKL5-FH (no signal sequence) and one of several putative CDKL5 substrates (HOMER1, HDAC4, ARHGEF2, MAPRE2, AMPH1, or SHANK1), or no protein partner, were transiently transfected into HEK293F cells. After five days in culture, cells were harvested and lysed in 50 mM sodium phosphate, 150 mM sodium chloride, 0.5% Triton-X100, 1X Complete Protease Inhibitor Complex, EDTA-free, pH 7 for 30 minutes at 4° C. A soluble fraction was obtained by centrifugation of the lysates at 16,000×g for 15 minutes at 4° C. Soluble protein was determined by BCA assay and an equal quantity was resolved on SDS-PAGE, transferred to nitrocellulose membranes, probed with rabbit anti-polyhistidine (ThermoFisher) and mouse anti-CDKL5 antibodies (EMD Millipore), and detected with near-infrared fluorescent secondary antibodies, anti-rabbit IgG DyaLight 680 and anti-mouse IgG DyaLight 800 (Cell Signaling Technology). As shown in the blot of FIG. 7, the co-expression of AMPH1 increased the quantity of soluble TATκ28-CDKL5 while the co-expression of ARHGEF2 reduced the quantity of soluble TATκ28-CDKL5. The latter suggests that elimination of ARHGEF2 expression might increase the quantity of soluble TATκ28-CDKL5.

### Example 7—In Vitro Transcription/Translation of CDKL5 Proteins

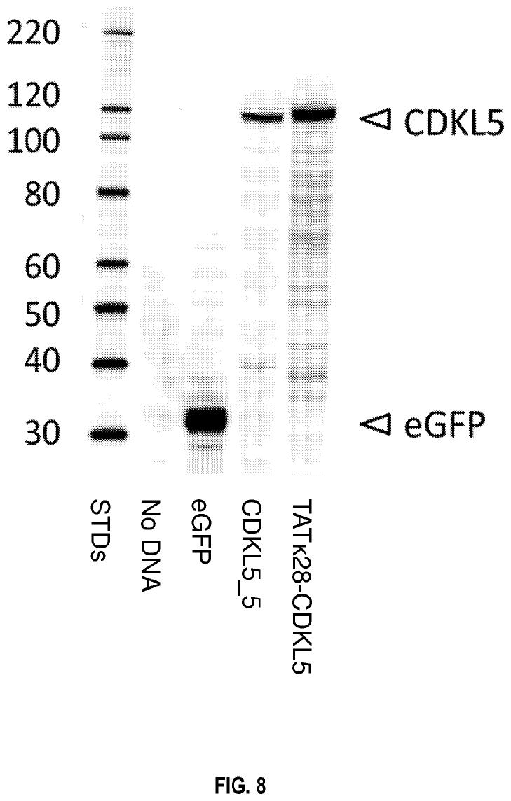

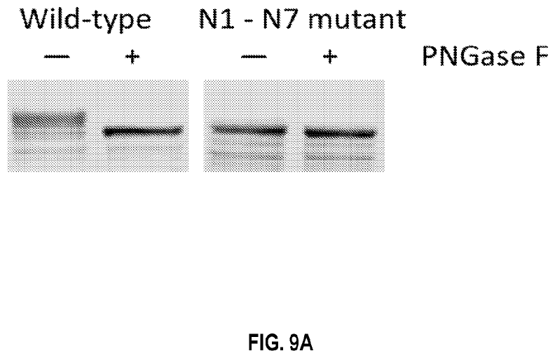

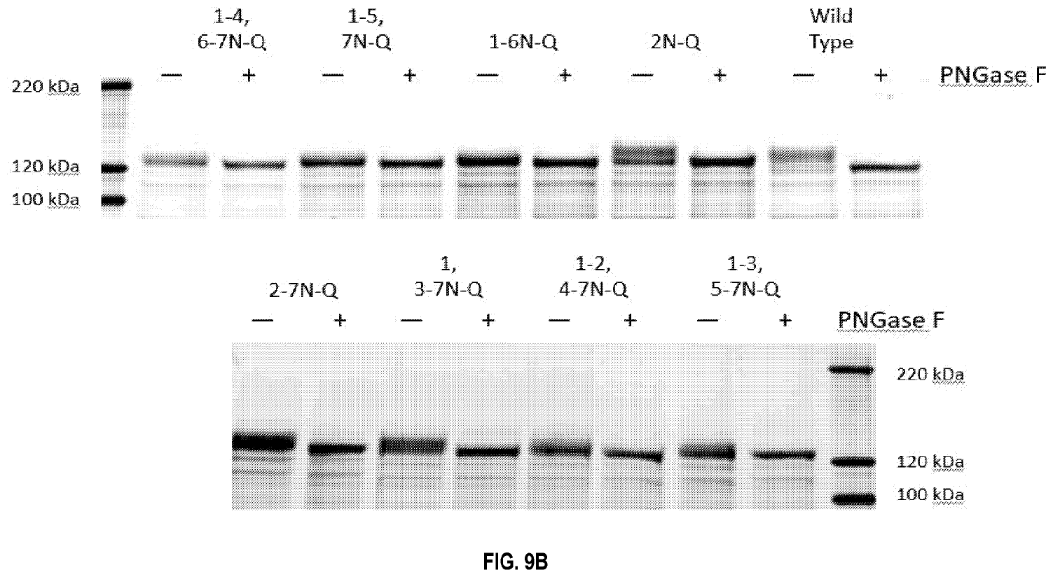

The following proteins were cloned into a T7/EMCV-IRES plasmid (pT7CFE1): eGFP, CDKL5_115 and TAT28-CDKL5_107-FH. Purified plasmid DNA was introduced into a HeLa cell-based IVT kit (Thermo) for non-CAP dependent combined in vitro transcription/translation for 5 hours at 30° C. Protein samples were resolved on SDS-PAGE, transferred to nitrocellulose membranes, probed with a rabbit anti-polyhistidine (His) antibody (Thermo), and detected with a fluorescent secondary antibody. Blot shown in FIG. 8 confirmed expression of the CDKL5 fusion proteins.

### Example 8—Glycosylation of CDKL5 Proteins

Further analysis of MBiP-TATκ28-CDKL5-107-3×FlagHis revealed that this fusion protein was glycosylated when expressed in CHO-DG44 and HEK293F cells. Plasmids were transiently transfected by electroporation into CHO-DG44 and HEK293F cells. Cell pellets were lysed and a soluble fraction was obtained by centrifugation. The soluble fraction was denatured in PNGase F buffer and incubated with PNGase F to remove N-linked glycans. Digested samples were resolved by SDS-PAGE, transferred to nitrocellulose and immunoblotted with an anti-polyhistidine antibody. Blot shown in FIG. 4A demonstrates that the fusion protein comprising the wild-type CDKL5₁₀₇ isoform is highly glycosylated when expressed in CHO-DG44 cells prior to treatment with PNGase F, whereas substituting 7 of the Asn residues of the N-linked glycosylation sites with Gln (1-7NQ) produces a fusion protein with little to no glycosylation when expressed in the CHO-DG44 cells. Further fusion proteins comprising the CDKL5 glycosylation variants 1-4, 6-7NQ; 1-5, 7NQ; 1-6NQ; 2NQ; 2-7NQ; 1, 3-7NQ; 1-2, 4-7NQ and 1-3, 5-7NQ were expressed in HEK293F cells, and untreated or treated with PNGase F and are shown in FIG. 4B. These fusion proteins comprising the other glycosylation variants had varying degrees of glycosylation and were all less glycosylated than the fusion protein comprising the wild-type CDKL5₁₀₇ isoform, thus showing that the various N-linked glycosylation sites can be glycosylated in isolation. Fusion proteins comprising the wild-type CDKL5₁₁₅ isoform were also found to be glycosylated.

### Example 9—Expression of CDKL5 Fusion Proteins in Insect Cells

Other expression systems were also investigated to improve expression, reduce glycosylation and/or enhance purification. One such system utilized the insect cells Sf9. To protect the N-terminus of TATκ28-CDKL5 and other CDKL5 fusion proteins, a GST tag was genetically fused to the N-terminus, separated from the remaining portion of the CDKL5 fusion protein by an HRV3C protease site. Another HRV3C protease site was added to the C-terminus of the CDKL5 protein to separate the FLAG and polyhistidine (His) affinity tags. Sf9 cells were co-transfected with linearized baculovirus (BV) DNA and transfer plasmids: 1) GST-P-TATκ28-eGFP-P-FH; 2) GST-P-eGFP-P-FH; 3) GST-P-TAT28-CDKL5_107-P-FH; 4) GST-P-TATκ28-CDKL5_107-P-FH; 5) GST-P-p97p-CDKL5_107-P-FH; 6) GST-P-Antp-CDKL5_107-P-FH; 7) GST-P-TAT11-CDKL5_107-P-FH and GST-P-Transp-CDKL5_107-P-FH (coding sequences being Sf9 codon-optimized). 1 μg protein run out on duplicate 4-12%, 10-well NuPage gels. Gels run at 175V for 90 minutes. Protein transferred to nitrocellulose using the iBLOT at 20v for 7 minutes. Expression of CDKL5 fusion proteins was analyzed with Sypro Ruby Red total protein stain as shown in FIG. 5.

### Example 10—Purification and Cleavage of GST-P-TATκ28-CDKL5Proteins

CDKL5 fusion proteins from insect cells were also purified to isolate the CDKL5 proteins from the cell lysate. GST-P-TATκ28-CDKL5_107-P-FH proteins were expressed in High Five (BTI-Tn-5B1-4) cells maintained as suspension cultures in Sf90011 media. Infected cell pellets were lysed with 50 mM NaPO₄, 500 mM NaCl, 10% Glycerol, pH 6) supplemented with 1X HALT Protease Inhibitor cocktail without EDTA (Thermo, 78437), 1 mM tris 2-carboxyethyl-phosphine (TCEP) and 5 mM EDTA at a ratio of 10 ml Lysis Buffer per 100 million cells. Following lysis by nitrogen cavitation using the Parr 4639 Cell Cracker at 750PSI for 15 minutes, Triton X-100 was added to 0.5%. The lysate was clarified by centrifugation at 31,000×g for 20 minutes. The soluble material was adjusted to 350 mM NaCl and applied to HiTrap SP Fast Flow resin (GE Healthcare, 17-5157-01). Bound protein was eluted with a 10 column volume (CV) NaCl gradient, 350-2000 mM. The CDKL5 protein peak, 525-1225 mM NaCl, was buffer-exchanged in to Buffer B (50 mM NaPO₄, 500 mM NaCl, 10% Glycerol, 1X HALT Protease inhibitor cocktail without EDTA, 1 mM TCEP, pH 8). Protein was applied to IMAC Sepharose 6 FF resin (GE Healthcare, 17-0921-09) that had been charged with Nickel Sulfate and pre-equilibrated with Buffer B. The resin was washed with Buffer B+60 mM imidazole. The resin with incubated with 40 U of HRV3C protease (Millipore, 71493) at 4° C. up to overnight to remove the GST, FLAG and polyhistidine (His) affinity tags. Aliquots of the cleaved material examined at 3 hours and overnight. The resin was washed with 50 mM NaPO4, 500 mM NaCl, 10% Glycerol, 1 mM TCEP+1X HALT PI-EDTA+0.5% Triton X-100+500 mM imidazole to elute the CDKL5. The eluted protein lacks the affinity tags and migrates more quickly though SDS-PAGE.

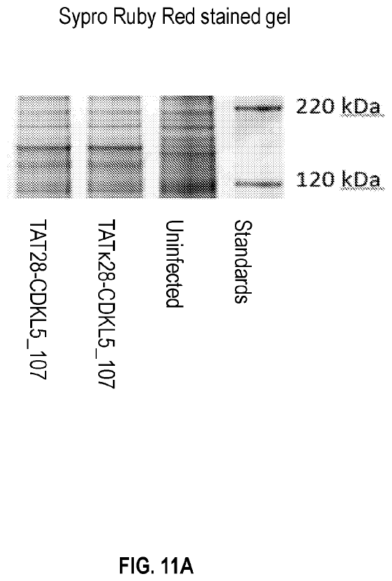

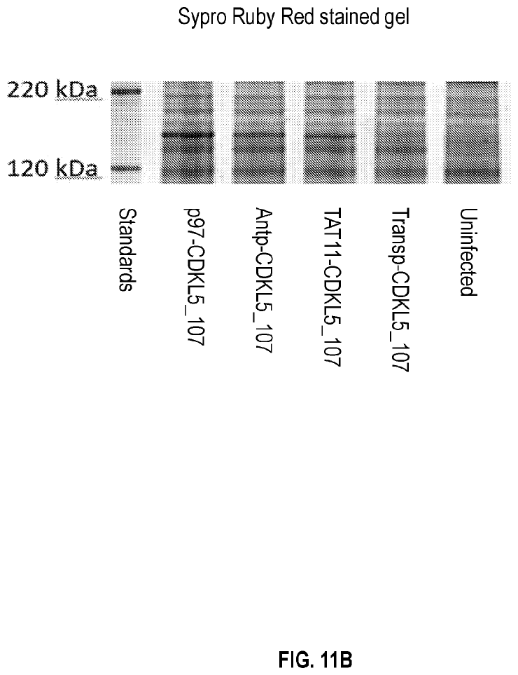

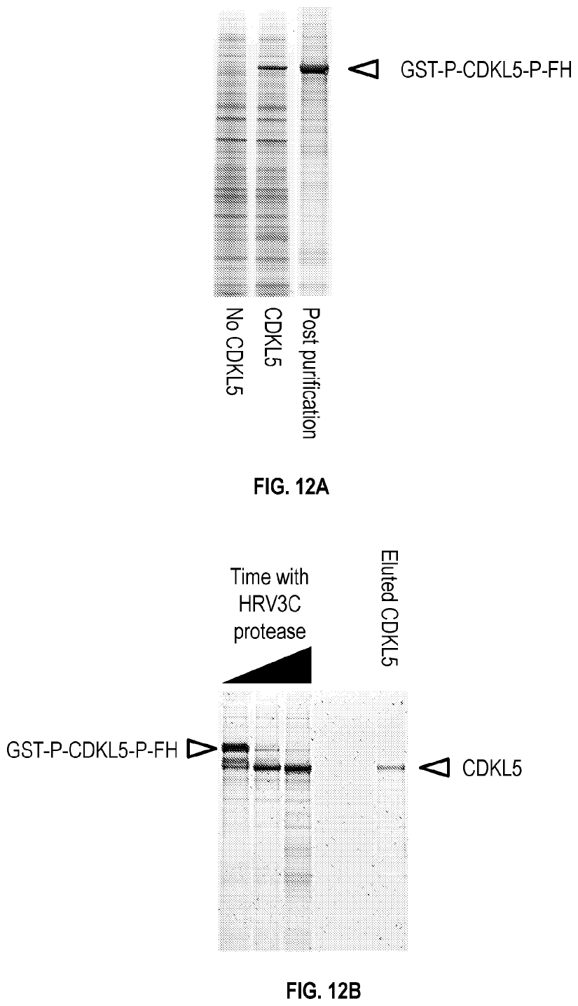

FIGS. 10A and 10B show a Sypro Ruby Red total protein stained gel analysis. FIG. 11A shows the expression of GST-P-TATκ28-CDKL5_107-P-FH in insect cells compared to uninfected control cells and the recovery of tagged protein on the IMAC resin. FIG. 11B shows the tagged CDKL5 protein prior to and post-cleavage with the eluted protein from the IMAC resin. Similarly, FIG. 12A shows a Sypro Ruby Red stained gel of a CDKL5 fusion protein in cell lysate and the purified fusion protein. FIG. 12B shows a Sypro Ruby Red stained gel demonstrating HRV3C protease cleavage of the CDKL5 fusion protein of FIG. 11A

### Example 11—Solubility of CDKL5 Proteins in Salt Solutions

GST-P-TATκ28-CDKL5_107-P-FH expressed in HighFive cells via infection with baculovirus was released from cells by lysis in 50 mM Na-phosphate, 500 mM NaCl, 10% glycerol, 1 mM TCEP, 1 mM EDTA, 1×HALT protease inhibitor cocktail, pH 6.0, using nitrogen cavitation for 15 minutes at room temperature. Following cell disruption, Triton X-100 was added to 0.5%, and incubated for 30 minutes at 4° C. The lysate was separated into soluble and insoluble fractions by centrifugation at 15,000×g for 15 minutes at room temperature. The soluble fraction was then further modified with the following conditions by dilution to the same final volume:
Maintained at 500 mM NaCl
Lowered to 350 mM NaCl
Lowered to 250 mM NaCl
(A) Supplemented with 2% Polysorbate-80, and lowered to 350 mM NaCl
(B) Supplemented with 50 mM arginine/50 mM glutamine, and lowered to 350 mM NaCl
(C) Supplemented with 100 mM betaine, and lowered to 350 mM NaCl
(D) Supplemented with 100 mM glycine, and lowered to 350 mM NaCl

Following incubation for 1 hour at room temperature under the described conditions, the solutions were again separated into soluble and insoluble fractions by centrifugation. The insoluble fraction was re-suspended in a volume equal to the soluble fraction, and both soluble and insoluble fractions were resolved on LDS-PAGE, then detected by staining with Coomassie.

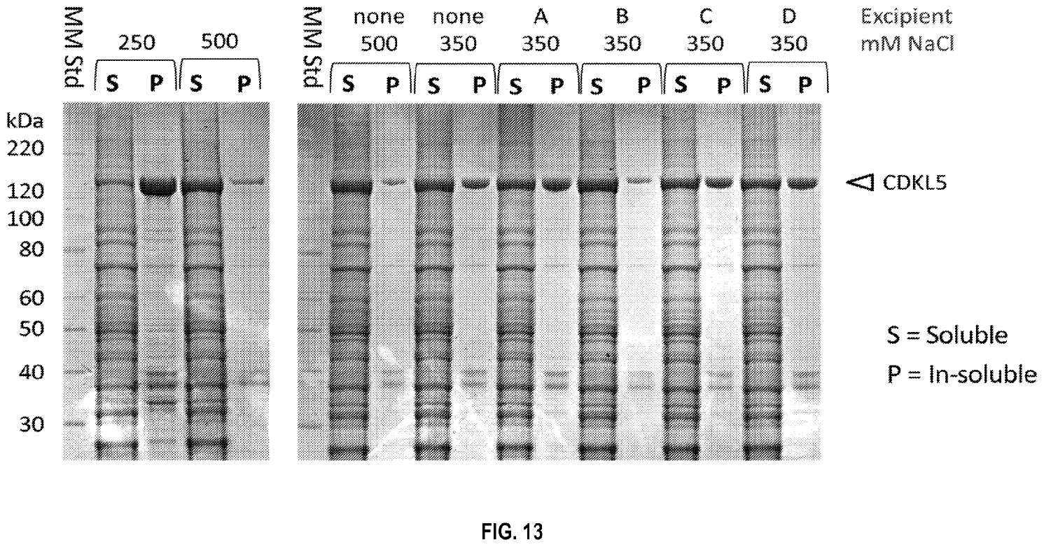

FIG. 13 shows that the CDKL5 fusion protein is soluble at high salt concentrations (e.g., at least 500 mM NaCl) and NaCl levels lower than 500 mM result in insoluble CDKL5 protein. The CDKL5 protein can be briefly exposed to NaCl concentrations as low at 350 mM, but some loss in incurred. For this reason, most purification steps described herein are carried out in high salt levels, but such high salt levels may be incompatible with in vivo administration.

### Example 12—Purification and Cleavage of TwinStrep-HRV3C-TATκ28-CDKL5-HRV3C-FLAG-His-HPC4 Proteins

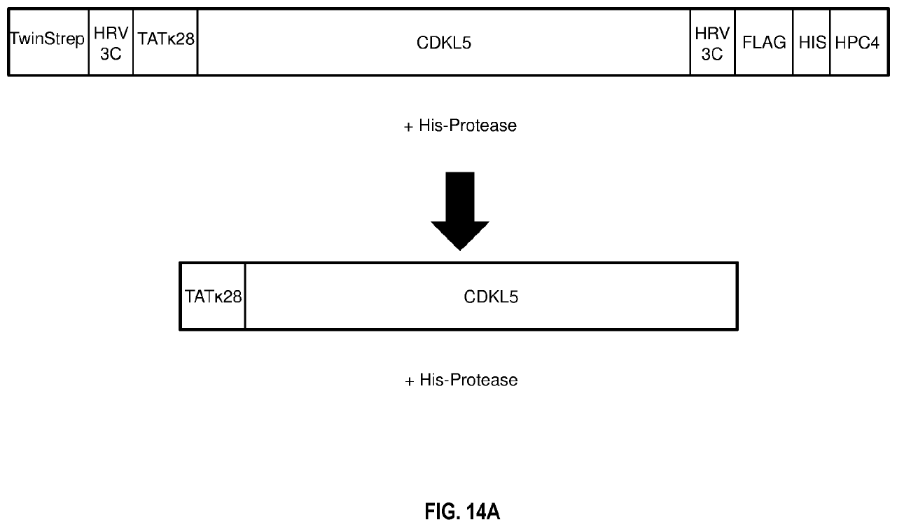

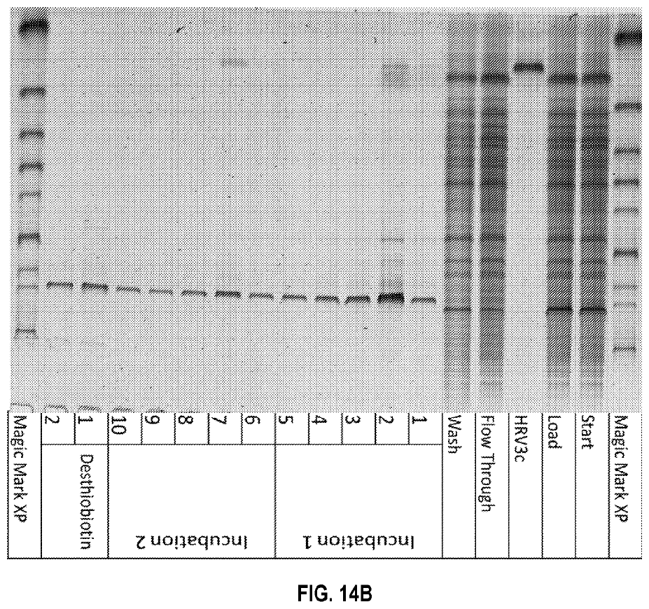

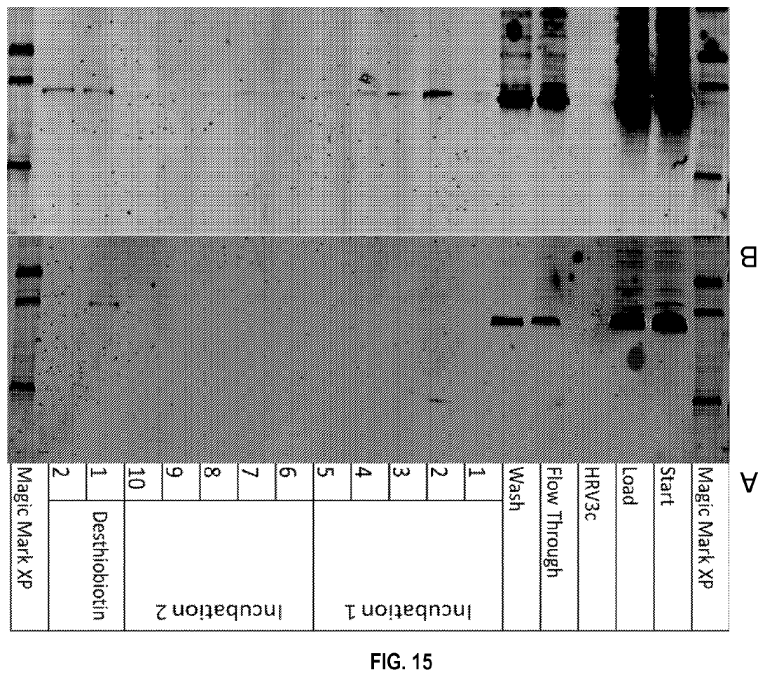

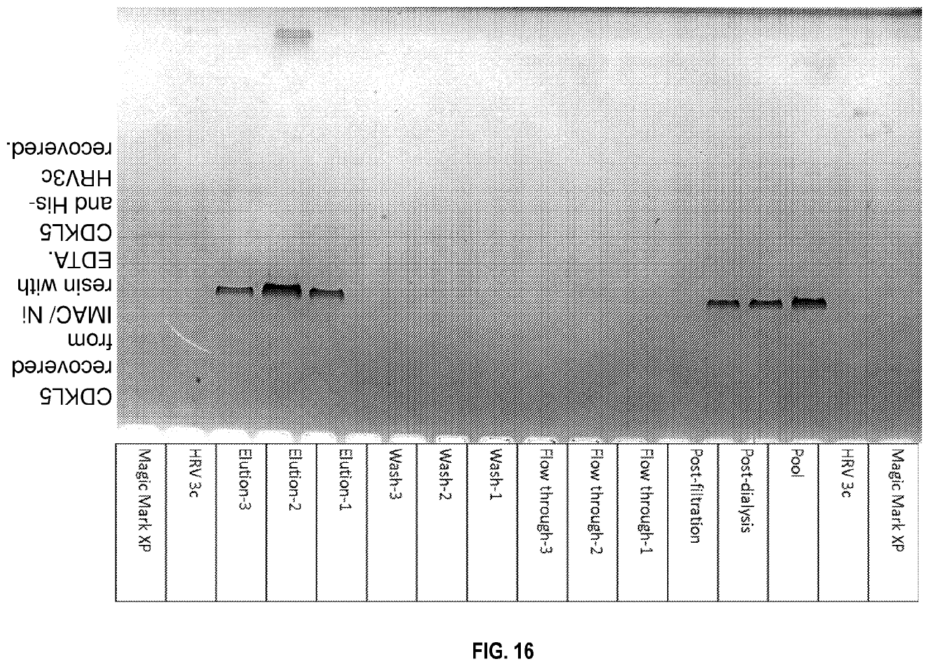

In this Example, the fusion protein TwinStrep-HRV3C-TATκ28-CDKL5-HRV3C-FLAG-His-HPC4 was expressed and purified. FIG. 14A shows the schematics of the fusion protein. The fusion protein has an amino acid sequence according to SEQ ID NO: 174. Similarly, the fusion protein has a nucleotide sequence according to SEQ ID NO: 175. FIG. 14B shows the fusion protein expression, purification, on column digestion by HRV3C protease and recovered fusion protein. FIG. 15 shows a Western blot analysis of the purification process. In FIG. 15A, the Western blot analysis was performed with anti-strep antibody and the results indicate complete digestion at the N-terminus. In contrast, the FIG. 15B shows a Western blot analysis using anti-HPC4 antibody indicating incomplete digestion at the C-terminus. FIG. 16 shows IMAC/Ni resin purification of the fusion protein and His-HRV3C protease.

### Example 13—Purification and Cleavage of TwinStrep-HRV3C-TATκ28-CDKL5-HRV3C-FLAG-His-TwinStrep Proteins

CDKL5 fusion proteins from insect cells were purified to isolate the CDKL5 proteins from the cell lysate.

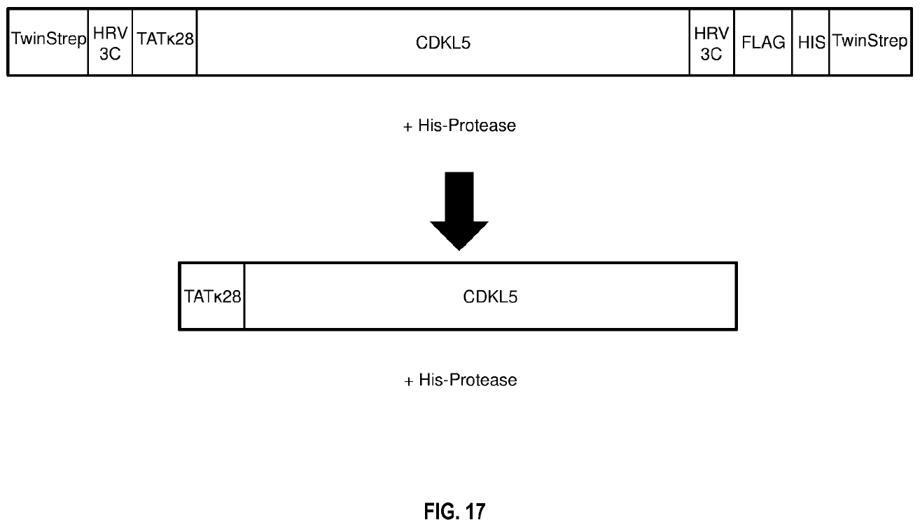

In this Example, the fusion protein was TwinStrep-HRV3C-TATκ28-CDKL5-HRV3C-FLAG-His-TwinStrep protein. The fusion protein has an amino acid sequence according the SEQ ID No: 176. Similarly, the fusion protein has a nucleotide sequence according SEQ ID No: 177. FIG. 17 shows the schematics of the fusion protein. The fusion protein was expressed in High Five (BTI-Tn-5B1-4) cells. Infected cells were pelleted and stored at −80° C.

For lysis, the cell pellet was resuspended in a lysis buffer (50 mM Tris HCl, 500 mM NaCl, 10% Glycerol, 1 mM EDTA at pH 8) supplemented with 1X HALT Protease Inhibitor cocktail without EDTA (Thermo, 78437). Following lysis by nitrogen cavitation using the Parr 4639 Cell Cracker at 750PSI for 15 minutes, Triton X-100 was added to 0.5%. The lysate was clarified by centrifugation at 31,000×g for 20 minutes. The clarified lysate was collected into a soluble fraction.

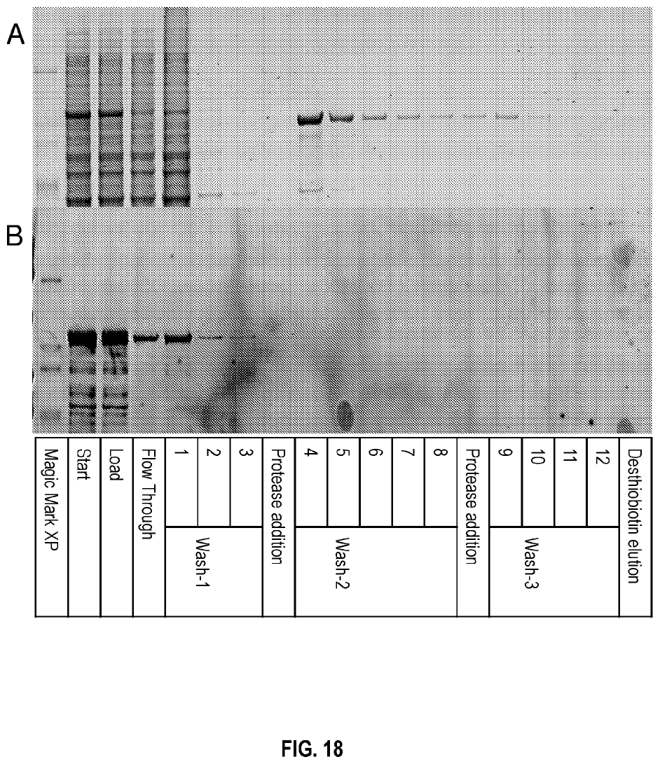

The insoluble pellet was washed with the lysis buffer. The washed insoluble pellet was then resuspended in 2 ml of the lysis buffer and sonicated. The soluble fraction after sonication was used for protein analysis. BCA assay was used to measure the protein concentration. NuPAGE was used to analyze the protein expression in insect cells. In FIG. 18, start and load shows total cellular protein and soluble fraction respectively.

For purifying the fusion protein from other soluble proteins, Strep-Tectin resin was used. The soluble fraction was loaded on a pre-equilibrated Strep-Tectin column. The affinity-tags were cleaved off on Strep-Tectin column using His-HRV3C protease. For the cleavage, the fusion protein bound to Strep-Tectin was incubated with the His-HRV3C protease for about 1 hour. After the digestion, the flow through and wash were collected. In FIG. 18, Wash-2 shows digested fusion protein. The digestion process was repeated one more time. In FIG. 18, Wash-3 shows the repeated digested fusion protein. The flow through and was were collected from the repeated digestion process. The flow-throughs and washes were pooled together in a cleavage pool. In FIG. 18, Desthiobiotin eluted fractions shows no undigested fusion protein. An analysis of imperial blue stained gel in FIG. 18A and a Western blot analysis using anti-strep antibody of FIG. 18B indicates complete digestion of the fusion protein at at N-terminus and C-terminus.

For a buffer exchange of the digested fusion protein and the His-HRV3C protease, HiPrep 26/20 Desalting column (Cytiva 17-5087-01) was used. The column was pre-equilibrated with Buffer A (50 mM Bis-Tris, 350 mM NaCl, 10% (v/v) glycerol at pH 6). The fusion protein containing the His-HRV3C protease was loaded on the column and fractions were pooled together into a desalting pool.

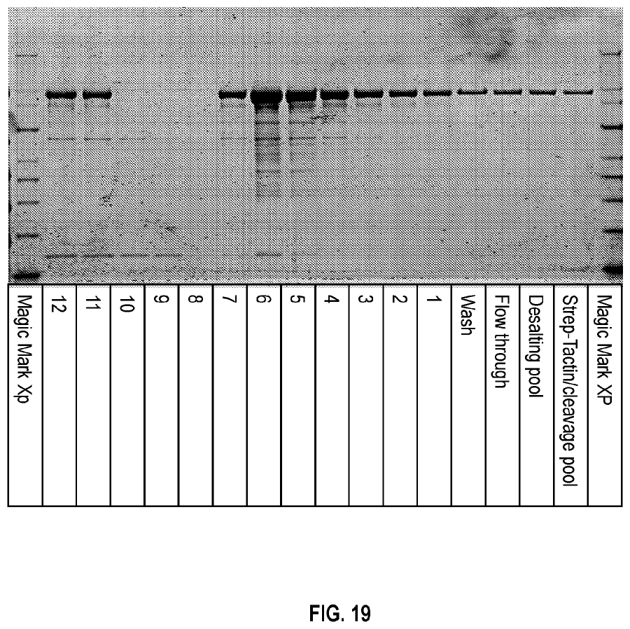

For purifying the fusion protein from the His-HRV3C protease, SP Sepharose capture column was used. The desalting pool was applied to an SP Sepharose capture, which was pre-equilibrated with the Buffer A. The TATκ28-CDKL5 protein was eluted with 55% of Buffer A and 45% of Buffer B (50 mM Bis-Tris, 2000 mM NaCl, 10% (v/v) glycerol at pH 6) to remove His-HVRc3 from the purified TATκ28-CDKL5 protein fraction. FIG. 19 shows purification process using SP Sepharose capture column.

### Example 14—Uptake of Purified TATκ28-CDKL5 Proteins in DIV14 Embryonic Primary Cortical Neurons

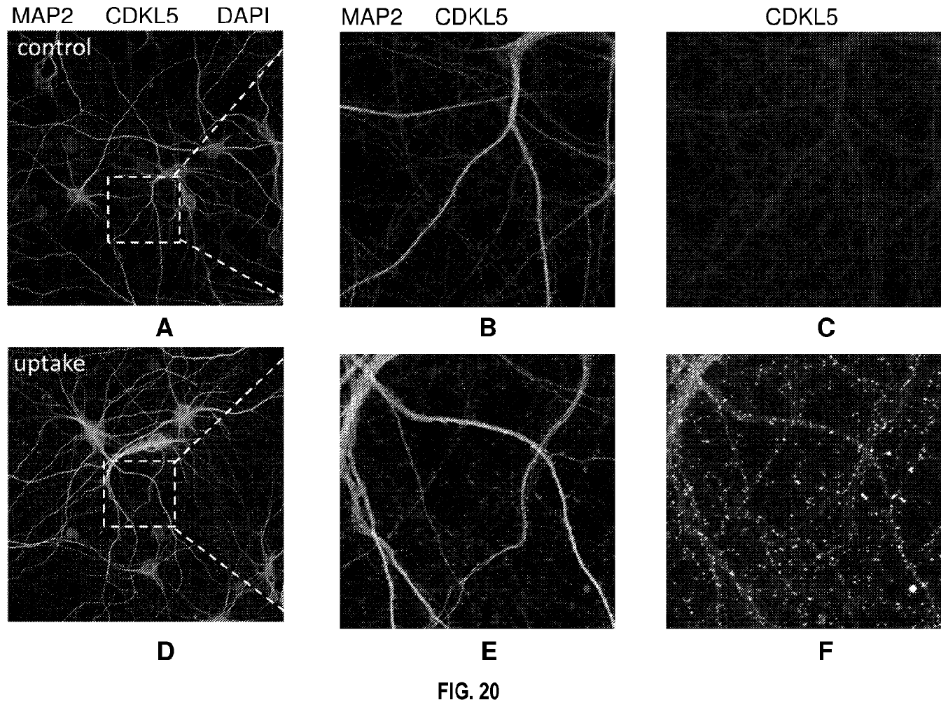

In this Example, an uptake of CDKL5 fusion proteins in embryonic primary cortical neurons was determined. The embryonic primary cortical neurons were isolated from healthy rat embryos at E15. The embryonic primary cortical neurons were seeded on poly-1-lysine coated glass coverslips and maintained for 14 days in vitro (DIV14). Recombinant TATκ28-CDKL5 was purified from a baculoviral/insect cell expression system via affinity-tag chromatography. The affinity-tags were removed by protease cleavage and the full-length protein was further isolated and concentrated via cation exchange chromatography. Cultured embryonic primary cortical neurons were treated with 10 μg/ml recombinant TATκ28-CDKL5 for 6 hours. Non-treated cultured embryonic primary cortical neurons were used as a negative control. Each sample, either treated or non-treated, were fixed in 4% PFA, permeabilized in 0.1% saponin, and stained using anti-MAP2, anti-CDKL5, and/or anti-phosphorylated (S222) EB2 antibodies. The cells were counterstained with DAPI and mounted on glass microscope slides under Prolong Diamond anti-fade mounting medium. The samples were imaged using a Leica SP8 point scanning laser confocal microscope with a 63x oil-immersion objective. The images were processed using Leica Lightning software and merged and colorized using ImageJ software. Analysis of phospho (S222) EB2 signal was performed using ImageJ software and graphed with GraphPad Prism software. FIG. 20A-20F shows the uptake of TATκ28-CDKL5 in DIV14 embryonic primary cortical neurons. FIG. 20A-20C shows images for negative controls treated with an equivalent volume of saline. FIG. 20A shows images of rat DIV14 embryonic primary cortical neurons stained with anti-DAPI and anti-MAP2 under the fluorescence microscope. FIG. 20B is an enlarged section of FIG. 20A. FIG. 20C shows FIG. 20B but only for anti-CDKL5 protein fluorescence. FIG. 20D-20F shows results of the uptake experiment, where the cells were treated with TATκ28-CDKL5. FIG. 20D shows image of rat DIV14 embryonic primary cortical neurons stained with anti-DAPI and anti-MAP2 under the fluorescence microscope. FIG. 20E is an enlarged section of FIG. 20D. FIG. 20F shows FIG. 20E but only for anti-CDKL5 fluorescence.

Similar experiments were also performed in rat DIV7 embryonic primary cortical neurons to compare the results with rat DIV14 embryonic primary cortical neurons.

FIG. 21A-21F shows the uptake of TATκ28-CDKL5 in rat DIV7 embryonic primary cortical neurons. FIG. 21A-21C are negative controls treated with an equivalent volume of saline. FIG. 21A shows image of rat DIV7 embryonic primary cortical neurons stained with anti-DAPI, anti-MAP2 and anti-CDKL5 protein under the fluorescence microscope. FIG. 21B is an enlarged section of FIG. 21A. FIG. 21C shows FIG. 21B but only for DAPI and anti-CDKL5 protein fluorescence. FIG. 21D-21F shows results of the uptake experiment, where the cells were treated with TATκ28-CDKL5. FIG. 21D shows image of rat DIV7 embryonic primary cortical neurons stained with anti-DAPI, anti-MAP2 and anti-CDKL5 protein under the fluorescence microscope. FIG. 21E is an enlarged section of FIG. 21D. FIG. 21F shows FIG. 21E but only for DAPI and anti-CDKL5 protein fluorescence.

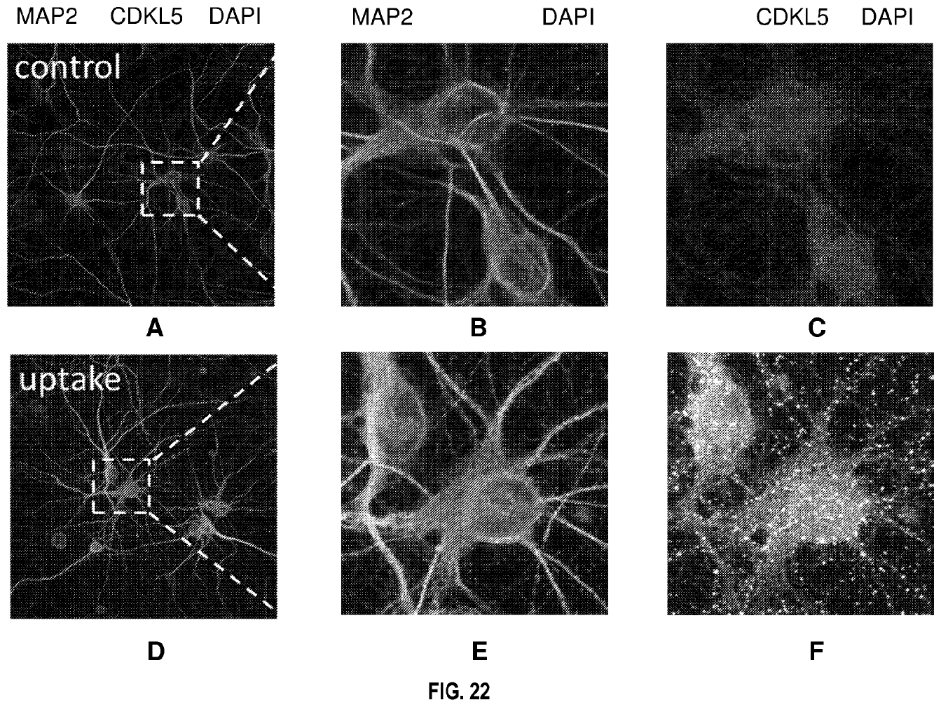

Similarly, FIG. 22A-22F shows the uptake of TATκ28-CDKL5 in rat DIV14 embryonic primary cortical neurons. FIG. 22A-22C represent images of negative controls. FIG. 22A shows image of embryonic primary cortical neurons stained with anti-DAPI, anti-MAP2 and anti-CDKL5 protein under the fluorescence microscope, FIG. 22B is an enlarged section of FIG. 22A. FIG. 22C shows FIG. 22B but only for DAPI and anti-CDKL5 protein fluorescence. FIG. 22D-22F shows results of the uptake experiment, where the cells were treated with the TATκ28-CDKL5 protein. FIG. 22D shows image of rat DIV14 embryonic primary cortical neurons stained with anti-DAPI, anti-MAP2 and anti-CDKL5 protein under the fluorescence microscope. FIG. 22E is an enlarged section of FIG. 22D. FIG. 22F shows FIG. 22E but only for DAPI and anti-CDKL5 protein fluorescence.

### Example 15—Time Dependent Uptake of Purified TATκ28-CDKL5 Proteins in DIV14 Embryonic Primary Cortical Neurons

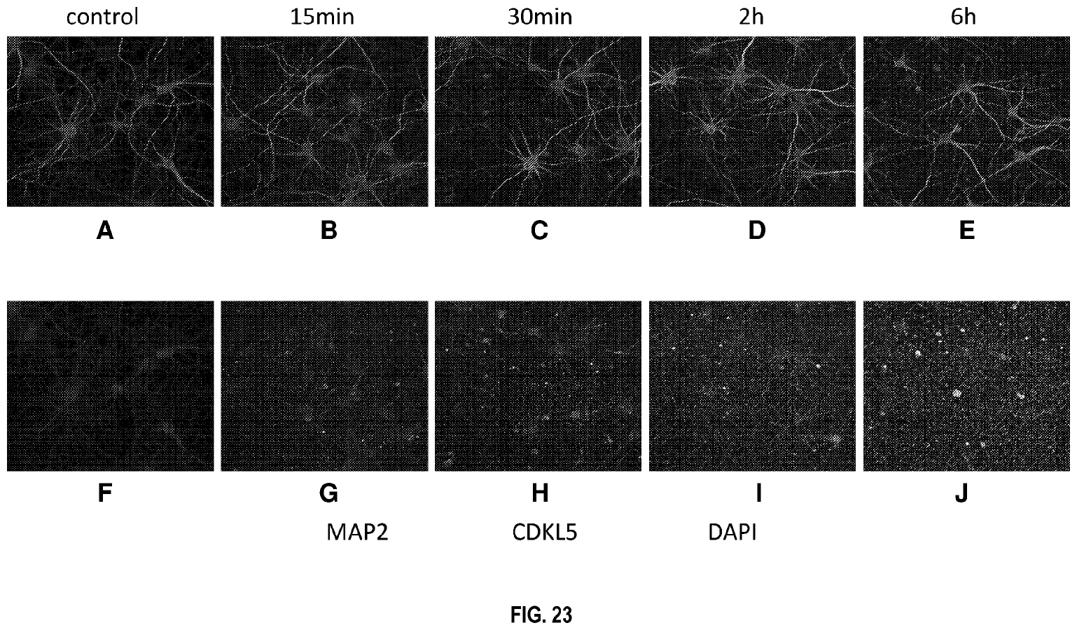

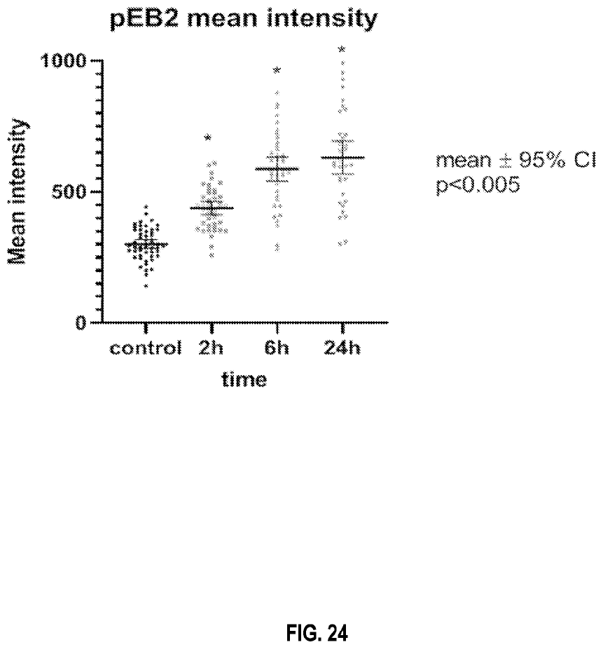

To further confirm TATκ28-CDKL5 over time, the cultured embryonic primary cortical neurons were treated with 10 μg/ml recombinant TATκ28-CDKL5 for 15 min, 30 min, 2 hr, 6 hr, or 24 hours. At each timepoint, treated coverslips were fixed in 4% PFA, permeabilized in 0.1% saponin, and stained using anti-MAP2, anti-CDKL5, and/or anti-phosphorylated (S222) EB2 antibodies. The cells were counterstained with DAPI and mounted on glass microscope slides under Prolong Diamond anti-fade mounting medium. The samples were imaged using a Leica SP8 point scanning laser confocal microscope with a 63x oil-immersion objective. The images were processed using Leica Lightning software and merged and colorized using ImageJ software. FIG. 23A-23J shows rapid uptake of TATκ28-CDKL5 protein by the cultured embryonic primary cortical neurons. FIG. 23A shows negative control with anti-DAPI, anti-MAP2 and anti-CDKL5. FIG. 23B-23E shows cortical neurons stained with anti-DAPI, anti-MAP2 and anti-CDKL5 at 15, 30, 120 and 360 minutes respectively. FIG. 23F shows FIG. 23A image but filtered for anti-CDKL5. Similarly, FIG. 23G-23J shows FIG. 23B-23E images filtered for anti-CDKL5 respectively. An analysis of FIG. 23A-23J indicates TATκ28-CDKL5 protein accumulation in cortical neurons that increases gradually increase in signal intensity over a period of at least 6 hours. Analysis of phospho (S222) EB2 signal was performed using ImageJ software and graphed with GraphPad Prism software. FIG. 24 observe an increase in intensity of phospho (S222) EB2 signal following uptake, an indication that the TATκ28-CDKL5 is active inside the cell.

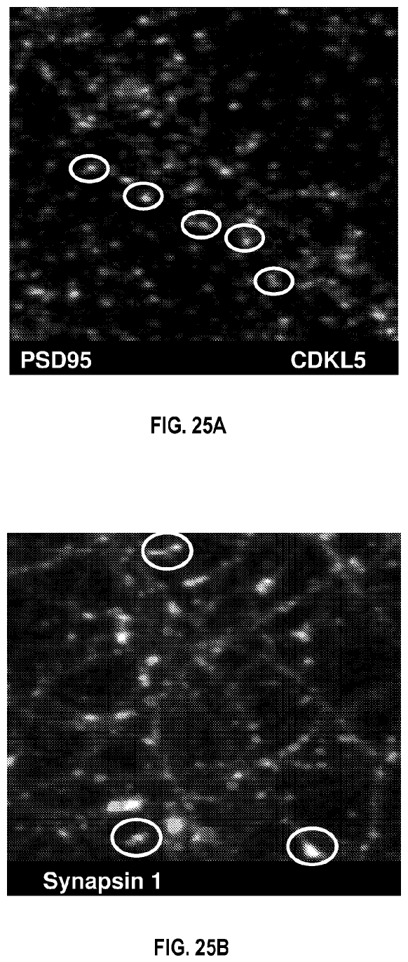

CDKL5 protein is reported to co-localize with PSD95 in neurons. In a particular embodiment, the DIV14 neurons were treated with 15 μg/ml of TATκ28-CDKL5 for 2 hours. The neurons were then stained with anti-PSD95 and anti-CDKL5. FIG. 25A and FIG. 25B shows co-localization of CDKL5 with PSD95 and Synapsin1 respectively.

### Example 16—Lentiviral Delivery of CDKL5 to Rat Neurons

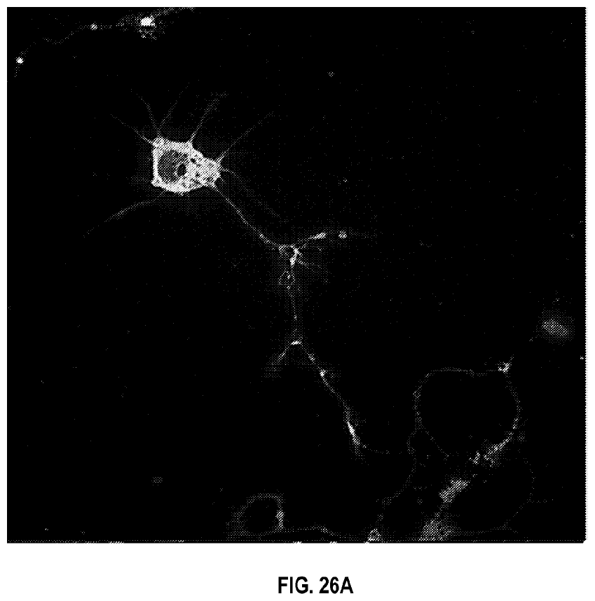

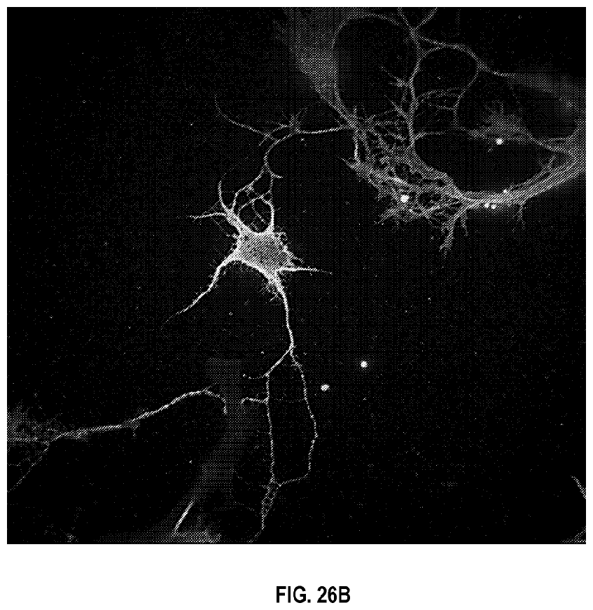

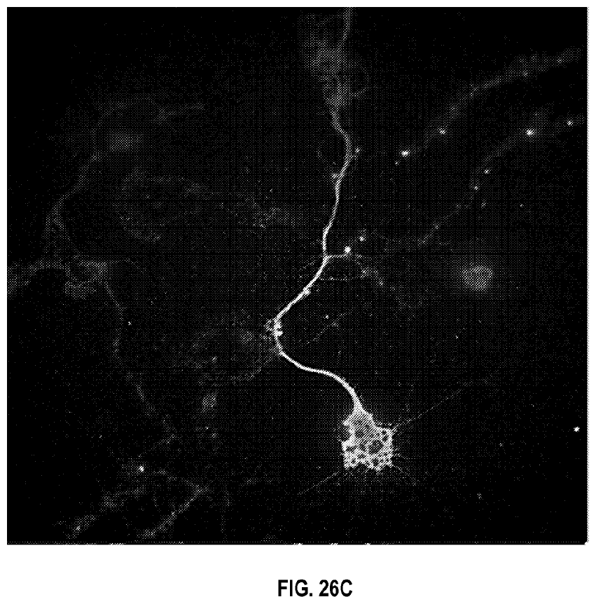

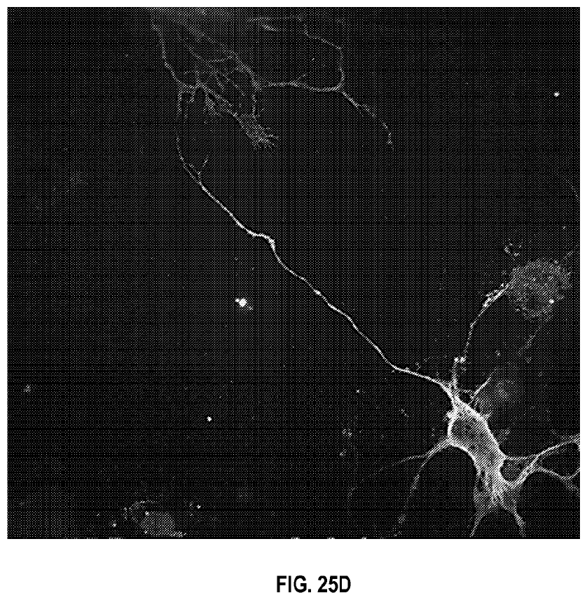

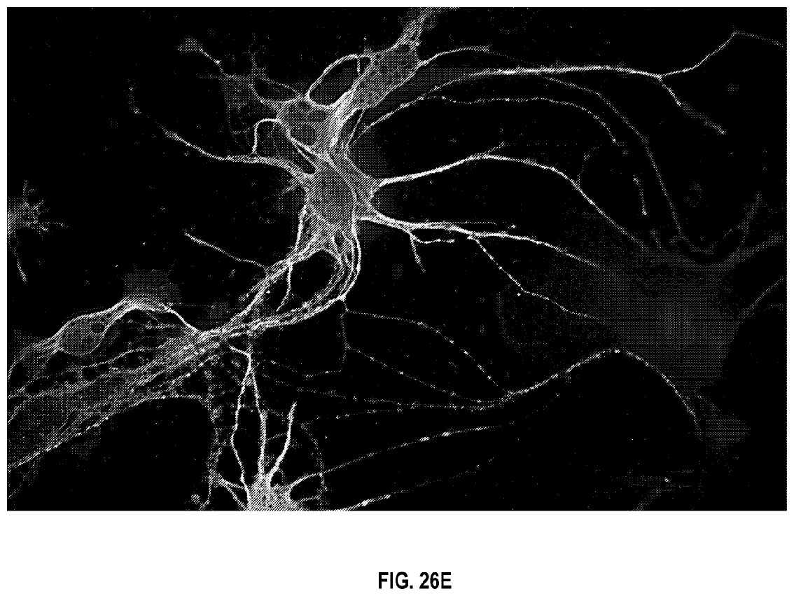

FIGS. 26A-26E show lentiviral delivery of the following to primary cdk15Δ rat neurons: untreated (13A), mBiP (12B), p97 (13C), TATκ28 (13D) and Antennapedia (13E). Cells were treated with 200 μl CPP-CKDL5 lentiviral supernatant and incubated for 24 hours, with a multiplicity of infection (MOI) of about 0.03. Packaging for the lentiviral delivery was done with the ViraPower™ Lentiviral Packaging Mix, Invitrogen K487500. After transduction, cells were fixed in PFA, permeabilized in saponin, and labeled with Ms anti-Beta III tubulin (red), Shp anti-CKDL5 (green), and DAPI (blue); imaged with 63x oil objective. These images show localization of the CDKL5 fusion protein along the neurite.

### Example 17—Cdkl5 Aav Constructs

SEQ ID NOS: 106-121 provide exemplary sequences for CDKL5 AAV vectors.

SEQ ID NO: 106 provides an exemplary sequence for a plasmid for expressing the full-length human CDKL5₁₀₇ isoform using the CBh promoter and the L-ITR and R-ITR of SEQ ID NOS: 27 and 28. The DNA sequence is codon-optimized for expression in mice.

SEQ ID NO: 107 provides an exemplary sequence for a plasmid for expressing a kinase-dead version of the full-length human CDKL5₁₀₇ isoform using the CBh promoter and the L-ITR and R-ITR of SEQ ID NOS: 27 and 28. The DNA sequence is codon-optimized for expression in mice.

SEQ ID NO: 108 provides an exemplary sequence for a plasmid for expressing eGFP using the CBh promoter and the L-ITR and R-ITR of SEQ ID NOS: 27 and 28. The DNA sequence is codon-optimized for expression in mice.

SEQ ID NO: 109 provides an exemplary sequence for a plasmid for expressing a fusion protein comprising NLS and eGFP using the CBh promoter and the L-ITR and R-ITR of SEQ ID NOS: 27 and 28. The DNA sequence is codon-optimized for expression in mice.

SEQ ID NO: 110 provides an exemplary sequence for a plasmid for expressing a fusion protein comprising a modified BiP leader signal polypeptide, TATκ28 and the full-length human CDKL5₁₀₇ isoform using the CBh promoter and the L-ITR and R-ITR of SEQ ID NOS: 27 and 28. The DNA sequence is codon-optimized for expression in mice.

SEQ ID NO: 111 provides an exemplary sequence for a plasmid for expressing a fusion protein comprising a modified BiP leader signal polypeptide, TATκ28 and a kinase-dead version of the full-length human CDKL5₁₀₇ isoform using the CBh promoter and the L-ITR and R-ITR of SEQ ID NOS: 27 and 28. The DNA sequence is codon-optimized for expression in mice.

SEQ ID NO: 112 provides an exemplary sequence for a plasmid for expressing a fusion protein comprising a modified BiP leader signal polypeptide, TATκ28 and eGFP using the CBh promoter and the L-ITR and R-ITR of SEQ ID NOS: 27 and 28. The DNA sequence is codon-optimized for expression in mice.

SEQ ID NO: 113 provides an exemplary sequence for a plasmid for expressing a fusion protein comprising a modified BiP leader signal polypeptide, TATκ28, NLS and eGFP using the CBh promoter and the L-ITR and R-ITR of SEQ ID NOS: 27 and 28. The DNA sequence is codon-optimized for expression in mice.

SEQ ID NO: 114 provides an exemplary sequence for a plasmid for expressing the full-length human CDKL5₁₀₇ isoform using the hSyn1 promoter and the L-ITR and R-ITR of SEQ ID NOS: 27 and 28. The DNA sequence is codon-optimized for expression in mice.

SEQ ID NO: 115 provides an exemplary sequence for a plasmid for expressing a kinase-dead version of the full-length human CDKL5₁₀₇ isoform using the hSyn1 promoter and the L-ITR and R-ITR of SEQ ID NOS: 27 and 28. The DNA sequence is codon-optimized for expression in mice.

SEQ ID NO: 116 provides an exemplary sequence for a plasmid for expressing eGFP using the hSyn1 promoter and the L-ITR and R-ITR of SEQ ID NOS: 27 and 28. The DNA sequence is codon-optimized for expression in mice.

SEQ ID NO: 117 provides an exemplary sequence for a plasmid for expressing a fusion protein comprising NLS and eGFP using the hSyn1 promoter and the L-ITR and R-ITR of SEQ ID NOS: 27 and 28. The DNA sequence is codon-optimized for expression in mice.

SEQ ID NO: 118 provides an exemplary sequence for a plasmid for expressing a fusion protein comprising a modified BiP leader signal polypeptide, TATκ28 and the full-length human CDKL5₁₀₇ isoform using the hSyn1 promoter and the L-ITR and R-ITR of SEQ ID NOS: 27 and 28. The DNA sequence is codon-optimized for expression in mice.

SEQ ID NO: 119 provides an exemplary sequence for a plasmid for expressing a fusion protein comprising a modified BiP leader signal polypeptide, TATκ28 and a kinase-dead version of the full-length human CDKL5₁₀₇ isoform using the hSyn1 promoter and the L-ITR and R-ITR of SEQ ID NOS: 27 and 28. The DNA sequence is codon-optimized for expression in mice.

SEQ ID NO: 120 provides an exemplary sequence for a plasmid for expressing a fusion protein comprising a modified BiP leader signal polypeptide, TATκ28 and eGFP using the hSyn1 promoter and the L-ITR and R-ITR of SEQ ID NOS: 27 and 28. The DNA sequence is codon-optimized for expression in mice.

SEQ ID NO: 121 provides an exemplary sequence for a plasmid for expressing a fusion protein comprising a modified BiP leader signal polypeptide, TATκ28, NLS and eGFP using the hSyn1 promoter and the L-ITR and R-ITR of SEQ ID NOS: 27 and 28. The DNA sequence is codon-optimized for expression in mice.

Plasmids containing SEQ ID NOS: SEQ ID NOS: 106-121 will be generated and tested in mice. Similar plasmids that are codon-optimized for rats will be tested in mice.

An exemplary DNA sequence codon-optimized for expression of a fusion protein in a human is provided in SEQ ID NO: 122. The fusion protein encoded by SEQ ID NO: 122 comprises a modified BiP leader signal polypeptide, TATκ28 and the full-length human CDKL5₁₀₇ isoform.

An exemplary DNA sequence codon-optimized for expression of the full-length human CDKL5₁₀₇ isoform in a human (but without the initiator methionine codon or the stop codon) is provided in SEQ ID NO: 123.

One skilled in the art can derive exemplary DNA sequences for human expression of the CDKL5 truncation variants described herein by deleting the relevant portions of the DNA sequence for the full-length CDKL5₁₀₇ isoform.

Exemplary DNA sequences for the glycosylation variant fusion proteins of SEQ ID NOS: 93-105 that are codon-optimized for human expression are provided in SEQ ID NOS: 124, 126, 128, 130, 132, 134, 136, 138, 140, 142, 144, 146 and 148, respectively.

Exemplary DNA sequences for the glycosylation variant CDKL5 polypeptides of SEQ ID NOS: 13-25 that are codon-optimized for human expression (but without the initiator methionine codon or the stop codon) are provided in SEQ ID NOS: 125, 127, 129, 131, 133, 135, 137, 139, 141, 143, 145, 147 and 149, respectively.

Exemplary DNA sequences for TATκ11, TATκ28, Antennapedia, Transportan and P97 that are codon-optimized for human expression (but without the initiator methionine codon or the stop codon) are provided in SEQ ID NOS: 150-154, respectively. Exemplary DNA sequences for TATκκ28 that are codon-optimized for human expression (but without the initiator methionine codon or the stop codon) using different codon optimization tools are provided in SEQ ID NOS: 170-173

An exemplary DNA sequence for mBIP that is codon-optimized for human expression (including the initiator methionine codon but without the stop codon) is provided in SEQ ID NO: 155. An exemplary DNA sequence for mvBIP that is codon-optimized for human expression (including the initiator methionine codon but without the stop codon) is provided in SEQ ID NO: 169.

### Example 18—CDKL5 Cross-Correction

In this Example, CDKL5 null mice were used for determining BIP-TATκ28-CDKL5 induced cross-correction. The CDKL5 null mice were divided into a treatment group and a control group. The treatment group was administered AAV-PHP.B.CBH.BIP-TATκ28-CDKL5.SV40 through intracerebroventricular (ICV) injection in an amount of 10×e⁹ GC/mice or 10×e¹⁰ GC/mice. The control group mice were administered PBS. Three months post-administration, the impact of the vector on behavioral endpoints was assessed and the mice were euthanized for transgene expression analysis.

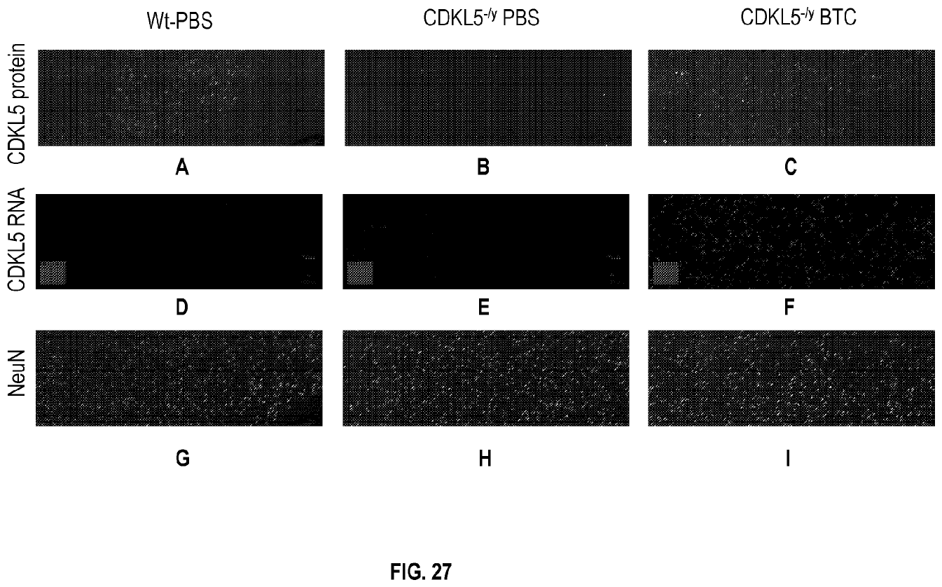

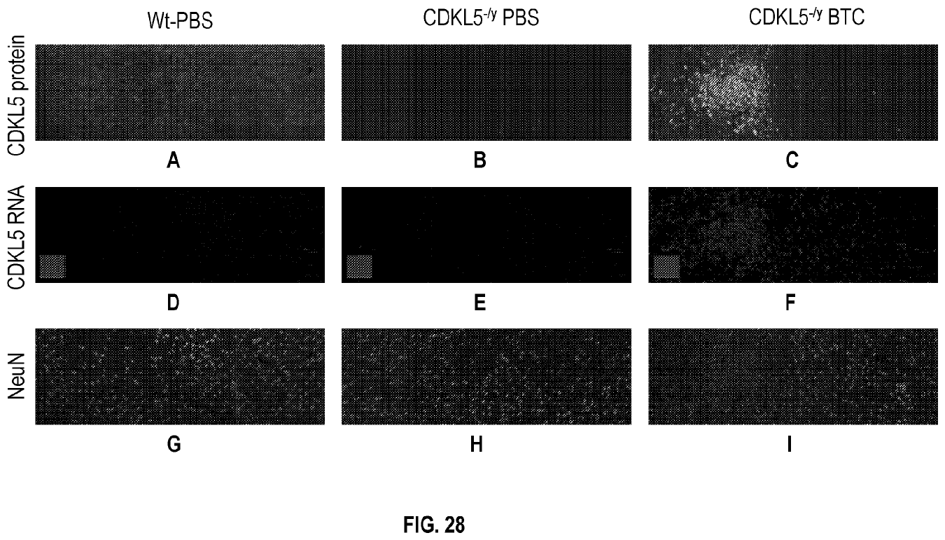

After euthanizing mice, sections of brain were taken. The sections were stained with DAPI, anti-NeuN antibody, anti-CDKL5 RNA riboprobe and anti-CDKL5 protein antibody. FIG. 27-29 shows anti-NeuN antibody, anti-CDKL5 RNA riboprobe and anti-CDKL5 protein antibody stained images of striatum, thalamus and hippocampal formation regions of brains, respectively.

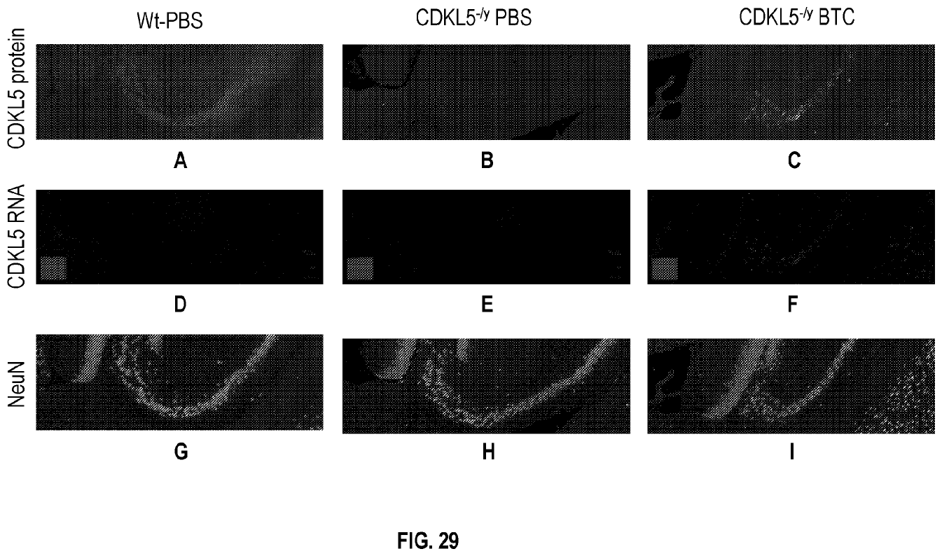

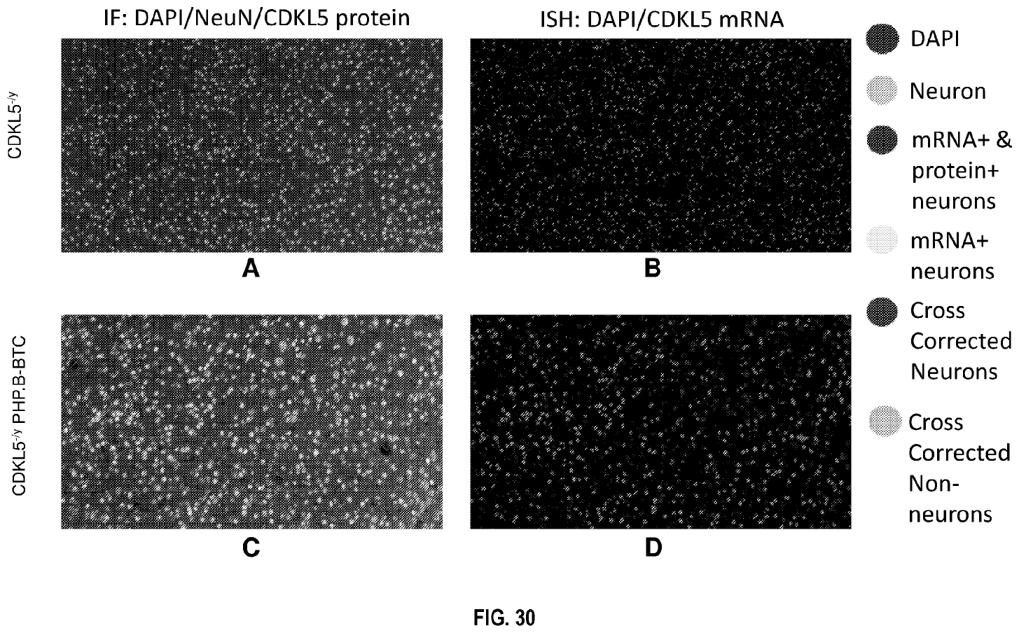

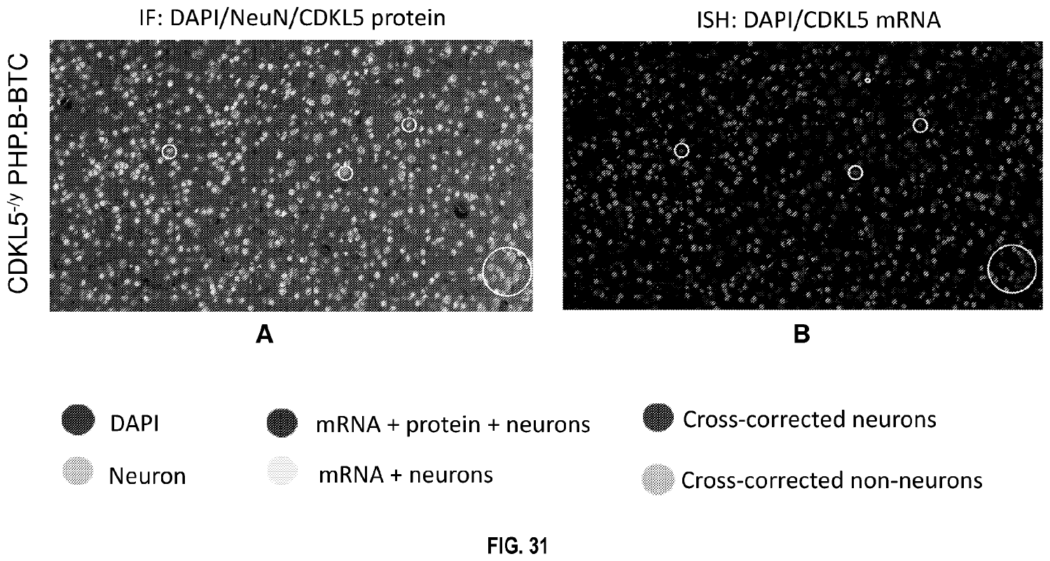

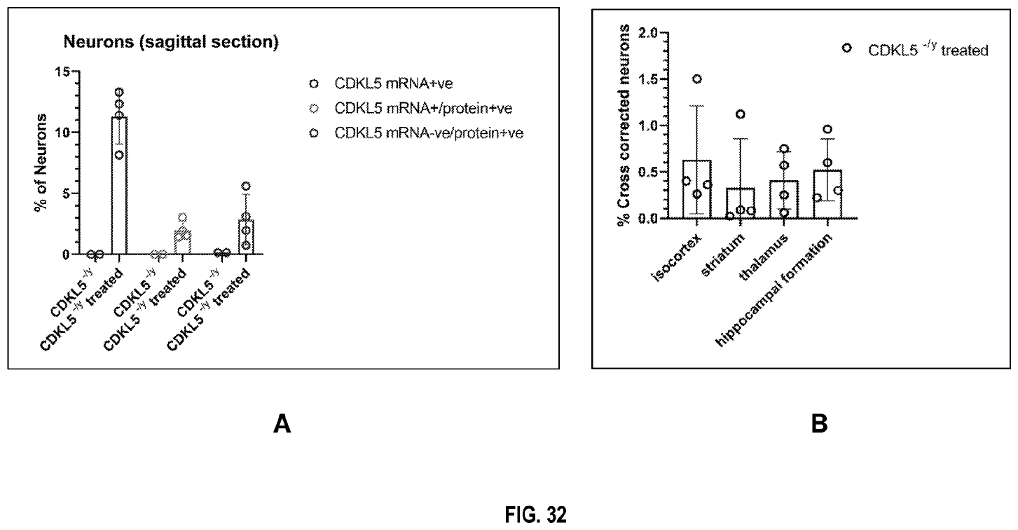

An image analysis was performed using Visiopharm software and the cells were divided into six groups: (1) DAPI stain to identify cells; (2) NeuN stain to identify neurons; (3) Neurons having CDKL5 mRNA and CDKL5 protein; (4) Neurons having CDKL5 mRNA; and (5) Cross-corrected neurons. FIG. 30 shows the image of identified six groups. FIGS. 29A and 29B represents image of immunostained brain section from the control group, whereas FIGS. 29C and 29D represents image of immunostained brain section from the treatment group. FIGS. 29A and 29C represents image of brain section stained with DAPI, anti-NeuN and anti-CDKL5 protein. FIGS. 29B and 29D represents image of brain section labeled with DAPI and anti-CDKL5 mRNA. FIG. 31 shows identified cross-corrected cells. FIG. 32A shows statistical analysis of cross-corrected neurons in a sagittal section. FIG. 32B shows statistical analysis of cross-corrected neurons in the specific brain regions, isocortex, striatum, thalamus and hippocampal formation, of the sagittal section.

### Example 19—Comparison of N-Terminal and C-Terminal CPPs

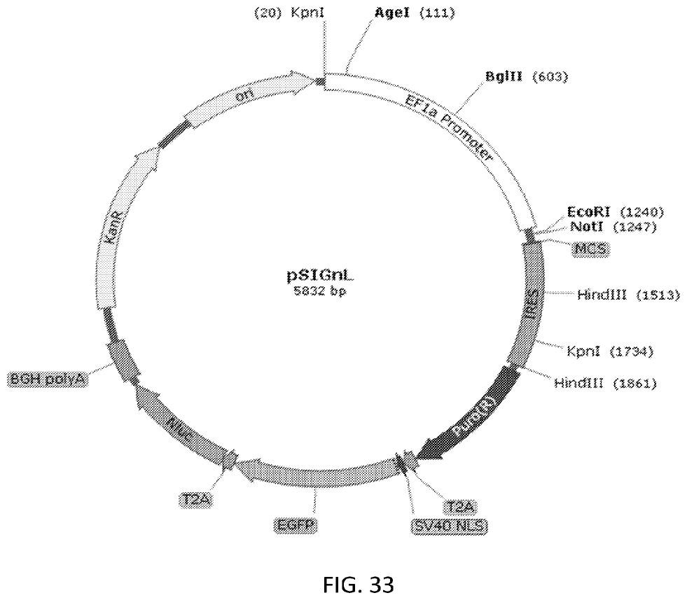

An exemplary plasmid for expressing various fusion proteins is shown in FIG. 33. This plasmid contains an EF1a promoter, a multiple cloning site (MCS), an IRES followed by Puromycin resistance, nuclear localized GFP, and nanoluciferase. The proteins after the IRES are separated by a T2A skip peptide. The plasmid will be tested for expressing the fusion proteins provided in Table 4 below:

TABLE 4

| Plasmid | Leader Signal | | CDKL5 | C-terminal | Codon |
|---|---|---|---|---|---|
| No. | Polypeptide | N-terminal CPP | Polypeptide | CPP | Optimization |
| 1 | mBIP | TATκ28 | CDKL5(107) | None | Gene Art |
| 2 | mBIP | TATκ11 | CDKL5(107) | None | Gene Art |
| 3 | mBIP | Transportan | CDKL5(107) | None | Gene Art |
| 4 | mBIP | Antennapedia | CDKL5(107) | None | Gene Art |
| 5 | mBIP | Melanotranferrin | CDKL5(107) | None | Gene Art |
| | | p97 | | | |
| 6 | mBIP | None | CDKL5(107) | TATκ28 | Gene Art |
| 7 | mBIP | None | CDKL5(107) | TATκ11 | Gene Art |
| 8 | mBIP | None | CDKL5(107) | Transportan | Gene Art |
| 9 | mBIP | None | CDKL5(107) | Antennapedia | Gene Art |
| 10 | mBIP | None | CDKL5(107) | Melanotranferrin | Gene Art |
| | | | | p97 | |
| 11 | mBIP | None | CDKL5(107) | None | Gene Art |
| 12 | mBIP | TATκ28 | CDKL5(107) | None | GenScript |
| 13 | mBIP | TATκ11 | CDKL5(107) | None | GenScript |
| 14 | mBIP | Transportan | CDKL5(107) | None | GenScript |
| 15 | mBIP | Antennapedia | CDKL5(107) | None | GenScript |
| 16 | mBIP | Melanotranferrin | CDKL5(107) | None | GenScript |
| | | p97 | | | |
| 17 | mBIP | None | CDKL5(107) | TATκ28 | GenScript |
| 18 | mBIP | None | CDKL5(107) | TATκ11 | GenScript |
| 19 | mBIP | None | CDKL5(107) | Transportan | GenScript |
| 20 | mBIP | None | CDKL5(107) | Antennapedia | GenScript |
| 21 | mBIP | None | CDKL5(107) | Melanotranferrin | GenScript |
| | | | | p97 | |
| 22 | mBIP | None | CDKL5(107) | None | GenScript |
| 23 | mBIP | TATκ28 | CDKL5(107) | None | SnapGene |
| 24 | mBIP | TATκ11 | CDKL5(107) | None | SnapGene |
| 25 | mBIP | Transportan | CDKL5(107) | None | SnapGene |
| 26 | mBIP | Antennapedia | CDKL5(107) | None | SnapGene |
| 27 | mBIP | Melanotranferrin | CDKL5(107) | None | SnapGene |
| | | p97 | | | |
| 28 | mBIP | None | CDKL5(107) | TATκ28 | SnapGene |
| 29 | mBIP | None | CDKL5(107) | TATκ11 | SnapGene |
| 30 | mBIP | None | CDKL5(107) | Transportan | SnapGene |
| 31 | mBIP | None | CDKL5(107) | Antennapedia | SnapGene |
| 32 | mBIP | None | CDKL5(107) | Melanotranferrin | SnapGene |
| | | | | p97 | |
| 33 | mBIP | None | CDKL5(107) | None | SnapGene |
| 34 | mBIP | TATκ28 | CDKL5(107) | None | COOL |
| 35 | mBIP | TATκ11 | CDKL5(107) | None | COOL |
| 36 | mBIP | Transportan | CDKL5(107) | None | COOL |
| 37 | mBIP | Antennapedia | CDKL5(107) | None | COOL |
| 38 | mBIP | Melanotranferrin | CDKL5(107) | None | COOL |
| | | p97 | | | |
| 39 | mBIP | None | CDKL5(107) | TATκ28 | COOL |
| 40 | mBIP | None | CDKL5(107) | TATκ11 | COOL |
| 41 | mBIP | None | CDKL5(107) | Transportan | COOL |
| 42 | mBIP | None | CDKL5(107) | Antennapedia | COOL |
| 43 | mBIP | None | CDKL5(107) | Melanotranferrin | COOL |
| | | | | p97 | |
| 44 | mBIP | None | CDKL5(107) | None | COOL |
| 45 | mBIP | None | CDKL5(107) | TATκκ28 | Gene Art |
| 46 | mBIP | None | CDKL5(107) | TATκκ28 | GenScript |
| 47 | mBIP | None | CDKL5(107) | TATκκ28 | SnapGene |
| 48 | mBIP | None | CDKL5(107) | TATκκ28 | COOL |
| 49 | mvBIP | TATκ28 | CDKL5(107) | None | SnapGene |
| 50 | mvBIP | None | CDKL5(107) | TATκ28 | SnapGene |

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

```
SEQUENCE LISTING
CDKL5₁₀₇ isoform polypeptide 1-960 (full-length)
SEQ ID NO: 1
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPENISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYNRTN
RSRMPNLNDLKETAL
CDKL5₁₀₇ Variant Δ853-960
SEQ ID NO: 2
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPENISHSEQLKEKEKQGFFRSMKKKKKKSQTVPNSDSPDLLTLQKSIHSASTPSSRPK
EWRPEKISDLQTQSQPLKSLRKLLHLSSASNHP
CDKL5₁₀₇ Variant Δ745-960
SEQ ID NO: 3
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESS
CDKL5₁₀₇ Variant Δ637-960
SEQ ID NO: 4
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMA
CDKL5₁₀₇ Variant Δ529-960
SEQ ID NO: 5
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPT
CDKL5₁₀₇ Variant Δ421-960
SEQ ID NO: 6
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFN
CDKL5₁₀₇ Variant Δ315-960
SEQ ID NO: 7
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRK
CDKL5₁₀₇ Variant Δ315-420
SEQ ID NO: 8
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKI
DPKPSEGPGTKYLKSNSRSQQNRHSFMESSQSKAGTLQPNEKQSRHSYIDTIPQSSRSPSYRT
KAKSHGALSDSKSVSNLSEARAQIAEPSTSRYFPSSCLDLNSPTSPTPTRHSDTRTLLSPSGR
NNRNEGTLDSRRTTTRHSKTMEELKLPEHMDSSHSHSLSAPHESFSYGLGYTSPFSSQQRPHR
HSMYVTRDKVRAKGLDGSLSIGQGMAARANSLQLLSPQPGEQLPPEMTVARSSVKETSREGTS
SFHTRQKSEGGVYHDPHSDDGTAPKENRHLYNDPVPRRVGSFYRVPSPRPDNSFHENNVSTRV
SSLPSESSSGTNHSKRQPAFDPWKSPENISHSEQLKEKEKQGFFRSMKKKKKKSQTVPNSDSP
DLLTLQKSIHSASTPSSRPKEWRPEKISDLQTQSQPLKSLRKLLHLSSASNHPASSDPRFQPL
TAQQTKNSFSEIRIHPLSQASGGSSNIRQEPAPKGRPALQLPGQMDPGWHVSSVTRSATEGPS
YSEQLGAKSGPNGHPYNRTNRSRMPNLNDLKETAL
CDKL5₁₀₇ Variant Δ315-528
SEQ ID NO: 9
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKS
PTPTRHSDTRTLLSPSGRNNRNEGTLDSRRTTTRHSKTMEELKLPEHMDSSHSHSLSAPHESF
SYGLGYTSPFSSQQRPHRHSMYVTRDKVRAKGLDGSLSIGQGMAARANSLQLLSPQPGEQLPP
EMTVARSSVKETSREGTSSFHTRQKSEGGVYHDPHSDDGTAPKENRHLYNDPVPRRVGSFYRV
PSPRPDNSFHENNVSTRVSSLPSESSSGTNHSKRQPAFDPWKSPENISHSEQLKEKEKQGFFR
SMKKKKKKSQTVPNSDSPDLLTLQKSIHSASTPSSRPKEWRPEKISDLQTQSQPLKSLRKLLH
LSSASNHPASSDPRFQPLTAQQTKNSFSEIRIHPLSQASGGSSNIRQEPAPKGRPALQLPGQM
DPGWHVSSVTRSATEGPSYSEQLGAKSGPNGHPYNRTNRSRMPNLNDLKETAL
CDKL5₁₀₇ Variant Δ315-636
SEQ ID NO: 10
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKA
RANSLQLLSPQPGEQLPPEMTVARSSVKETSREGTSSFHTRQKSEGGVYHDPHSDDGTAPKEN
RHLYNDPVPRRVGSFYRVPSPRPDNSFHENNVSTRVSSLPSESSSGTNHSKRQPAFDPWKSPE
NISHSEQLKEKEKQGFFRSMKKKKKKSQTVPNSDSPDLLTLQKSIHSASTPSSRPKEWRPEKI
SDLQTQSQPLKSLRKLLHLSSASNHPASSDPRFQPLTAQQTKNSFSEIRIHPLSQASGGSSNI
RQEPAPKGRPALQLPGQMDPGWHVSSVTRSATEGPSYSEQLGAKSGPNGHPYNRTNRSRMPNL
NDLKETAL
CDKL5₁₀₇ Variant Δ315-744
SEQ ID NO: 11
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKS
GTNHSKRQPAFDPWKSPENISHSEQLKEKEKQGFFRSMKKKKKKSQTVPNSDSPDLLTLQKSI
HSASTPSSRPKEWRPEKISDLQTQSQPLKSLRKLLHLSSASNHPASSDPRFQPLTAQQTKNSF
SEIRIHPLSQASGGSSNIRQEPAPKGRPALQLPGQMDPGWHVSSVTRSATEGPSYSEQLGAKS
GPNGHPYNRTNRSRMPNLNDLKETAL
CDKL5₁₀₇ Variant Δ315-852
SEQ ID NO: 12
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKA
SSDPRFQPLTAQQTKNSFSEIRIHPLSQASGGSSNIRQEPAPKGRPALQLPGQMDPGWHVSSV
TRSATEGPSYSEQLGAKSGPNGHPYNRTNRSRMPNLNDLKETAL
CDKL5₁₀₇ Variant 1-7NQ
SEQ ID NO: 13
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant 2-7NQ
SEQ ID NO: 14
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant 1,3-7NQ
SEQ ID NO: 15
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant l-2,4-7NQ
SEQ ID NO: 16
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant l-3,5-7NQ
SEQ ID NO: 17
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant l-4,6-7NQ
SEQ ID NO: 18
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPENISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant 1-5,7NQ
SEQ ID NO: 19
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYNRTQ
RSRMPNLNDLKETAL
CDKL5₁₀₇ Variant 1-6NQ
SEQ ID NO: 20
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTN
RSRMPNLNDLKETAL
CDKL5₁₀₇ Variant 2NQ
SEQ ID NO: 21
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPENISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYNRTN
RSRMPNLNDLKETAL
CDKL5₁₀₇ Variant 1-10NQ
SEQ ID NO: 22
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLETIQKVLGPLPSEQMKLFYSNPREHGLREPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPA [symbol] ESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHEN [symbol] VSTRVSSLPSESSSGT [symbol] HSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant 1-7, 9-10NQ
SEQ ID NO: 23
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPA [symbol] ESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHEN [symbol] VSTRVSSLPSESSSGT [symbol] HSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant 1-8, 10NQ
SEQ ID NO: 24
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPA [symbol] ESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHEN [symbol] VSTRVSSLPSESSSGT [symbol] HSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5₁₀₇ Variant 1-9NQ
SEQ ID NO: 25
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARQLSEGNNAQYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQQLSVGLPRADEGLPA [symbol] ESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSQLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHEN [symbol] VSTRVSSLPSESSSGT [symbol] HSKRQPAF
DPWKSPEQISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYQRTQ
RSRMPNLNDLKETAL
CDKL5115 isoform polypeptide 1-1030 (full-length)
SEQ ID NO: 26
MKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPENISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPDGGCDGRRQRHHSGPQDRRFMLRTTEQQGEYFCCGDPKKPH
TPCVPNRALHRPISSPAPYPVLQVRGTSMCPTLQVRGTDAFSCPTQQSGFSFFVRHVMREALI
HRAQVNQAALLTYHENAALTGK
AAV2 L-ITR
SEQ ID NO: 27
CCTGCAGGCAGCTGCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGAGTGGCCAACTCCA
TCACTAGGGGTTCCT
AAV2 R-ITR
SEQ ID NO: 28
AGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGC
GCAGCTGCCTGCAGG
CBh
SEQ ID NO: 29
TTAATAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCAATAAT
GACGTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTATTGA
CGTCAATGACGGTAAATGGCCCGCCTGGCATTATGCCCAGTACATGACCTTACGGGACTTTCC
TACTTGGCAGTACATCTCCACGTTCTGCTTCACTCTCCCCATCTCCCCCCCCTCCCCACCCCC
AATTTTGTATTTATTTATTTTTTAATTATTTTGTGCAGCGATGGGGGCGGGGGGGGGGGGGGC
GCGCGCCAGGCGGGGCGGGGCGGGGCGAGGGGCGGGGCGGGGCGAGGCGGAGAGGTGCGGCGG
CAGCCAATCAGAGCGGCGCGCTCCGAAAGTTTCCTTTTATGGCGAGGCGGCGGCGGCGGCGGC
CCTATAAAAAGCGAAGCGCGCGGCGGGGAGTCGCTGCGTTGCCTTCGCCCCGTGCCCCGCTCC
GCGCCGCCTCGCGCCGCCCGCCCCGGCTCTGACTGACCGCGTTACTCCCACAGGTGAGCGGGC
GGGACGGCCCTTCTCCTCCGGGCTGTAATTAGCAAGAGGTAAGGGTTTAAGGGATGGTTGGTT
GGTGGGGTATTAATGTTTAATTACCTGTTTTACAGGCCTGAAATCACTTGGTTTTAGGTTGG
hSyn1
SEQ ID NO: 30
ACTACAAACCGAGTATCTGCAGAGGGCCCTGCGTATGAGTGCAAGTGGGTTTTAGGACCAGGA
TGAGGCGGGGTGGGGGTGCCTACCTGACGACCGACCCCGACCCACTGGACAAGCACCCAACCC
CCATTCCCCAAATTGCGCATCCCCTATCAGAGAGGGGGAGGGGAAACAGGATGCGGCGAGGCG
CGTGCGCACTGCCAGCTTCAGCACCGCGGACAGTGCCTTCGCCCCCGCCTGGCGGCGCGCGCC
ACCGCCGCCTCAGCACTGAAGGCGCGCTGACGTCACTCGCCGGTCCCCCGCAAACTCCCCTTC
CCGGCCACCTTGGTCGCGTCCGCGCCGCCGCCGGCCCAGCCGGACCGCACCACGCGAGGCGCG
AGATAGGGGGGCACGGGCGCGACCATCTGCGCTGCGGCGCCGGCGACTCAGCGCTGCCTCAGT
CTGCGGTGGGCAGCGGAGGAGTCGTGTCGTGCCTGAGAGCGCAGCTGTGCTCCTGGGCACCGC
GCAGTCCGCCCCCGCGGCTCCTGGCCAGACCACCCCTAGGACCCCCTGCCCCAAGTCGCAGCC
TTCGA
TAT28 CPP
SEQ ID NO: 31
DAAQPARRARRTKLAAYGRKKRRQRRR
TATκ28 CPP
SEQ ID NO: 32
DAAQPARRARRTKLAAYARKAARQARA
TAT11 CPP
SEQ ID NO: 33
YGRKKRRQRRR
TATκ11 CPP
SEQ ID NO: 34
YARKAARQARA
Transportan CPP
SEQ ID NO: 35
AGYLLGKINLKALAALAKKIL
Antennapedia CPP
SEQ ID NO: 36
RQIKIWFQNRRMKWKK
P97 CPP
SEQ ID NO: 37
DSSHAFTLDELR
MBiP
SEQ ID NO: 38
MKLSLVAAMLLLLSLVAAMLLLLSAARA
MBiP2
SEQ ID NO: 39
MKLSLVAAMLLLLWVALLLLSAARA
MBiP3
SEQ ID NO: 40
MKLSLVAAMLLLLSLVALLLLSAARA
MBiP4
SEQ ID NO: 41
MKLSLVAAMLLLLALVALLLLSAARA
Murine Igκ
SEQ ID NO: 42
METDTLLLWVLLLWVPGSTG
MBip_Tκ28p_107_3xFlagHis_cho-opt in pOptiVec
SEQ ID NO: 43
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARNLSEGNNANYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQNLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSNLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPENISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYNRTNRSR

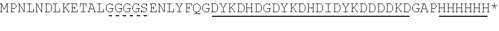

Igκ_Tκ28p_107_3xFlagHis_cho-opt in pOptiVec
SEQ ID NO: 44
METDTLLLWVLLLWVPGSTGGDAAQPARRARRTKLAAYARKAARQARAGGGGSKIPNIGNVMN
KFEILGVVGEGAYGVVLKCRHKETHEIVAIKKFKDSEENEEVKETTLRELKMLRTLKQENIVE
LKEAFRRRGKLYLVFEYVEKNMLELLEEMPNGVPPEKVKSYIYQLIKAIHWCHKNDIVHRDIK
PENLLISHNDVLKLCDFGFARNLSEGNNANYTEYVATRWYRSPELLLGAPYGKSVDMWSVGCI
LGELSDGQPLFPGESEIDQLFTIQKVLGPLPSEQMKLFYSNPRFHGLRFPAVNHPQSLERRYL
GILNSVLLDLMKNLLKLDPADRYLTEQCLNHPTFQTQRLLDRSPSRSAKRKPYHVESSTLSNR
NQAGKSTALQSHHRSNSKDIQNLSVGLPRADEGLPANESFLNGNLAGASLSPLHTKTYQASSQ
PGSTSKDLTNNNIPHLLSPKEAKSKTEFDFNIDPKPSEGPGTKYLKSNSRSQQNRHSFMESSQ
SKAGTLQPNEKQSRHSYIDTIPQSSRSPSYRTKAKSHGALSDSKSVSNLSEARAQIAEPSTSR
YFPSSCLDLNSPTSPTPTRHSDTRTLLSPSGRNNRNEGTLDSRRTTTRHSKTMEELKLPEHMD
SSHSHSLSAPHESFSYGLGYTSPFSSQQRPHRHSMYVTRDKVRAKGLDGSLSIGQGMAARANS
LQLLSPQPGEQLPPEMTVARSSVKETSREGTSSFHTRQKSEGGVYHDPHSDDGTAPKENRHLY
NDPVPRRVGSFYRVPSPRPDNSFHENNVSTRVSSLPSESSSGTNHSKRQPAFDPWKSPENISH
SEQLKEKEKQGFFRSMKKKKKKSQTVPNSDSPDLLTLQKSIHSASTPSSRPKEWRPEKISDLQ
TQSQPLKSLRKLLHLSSASNHPASSDPRFQPLTAQQTKNSFSEIRIHPLSQASGGSSNIRQEP
APKGRPALQLPGQMDPGWHVSSVTRSATEGPSYSEQLGAKSGPNGHPYNRTNRSRMPNLNDLK

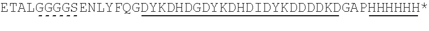

MBiP_Tκ28p_115_3xFlagHis_cho-opt in pOptiVec
SEQ ID NO: 45
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARNLSEGNNANYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQNLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSNLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPENISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPDGGCDGRRQRHHSGPQDRRFMLRTTEQQGEYFCCGDPKKPHTPC
VPNRALHRPISSPAPYPVLQVRGTSMCPTLQVRGTDAFSCPTQQSGFSFFVRHVMREALIHRA

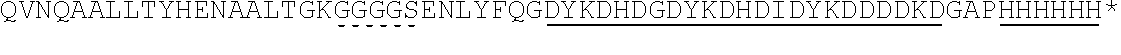

Igκ_Tκ28p_115_3xFlagHis_cho-opt in pOptiVec
SEQ ID NO: 46
METDTLLLWVLLLWVPGSTGGDAAQPARRARRTKLAAYARKAARQARAGGGGSKIPNIGNVMN
KFEILGVVGEGAYGVVLKCRHKETHEIVAIKKFKDSEENEEVKETTLRELKMLRTLKQENIVE
LKEAFRRRGKLYLVFEYVEKNMLELLEEMPNGVPPEKVKSYIYQLIKAIHWCHKNDIVHRDIK
PENLLISHNDVLKLCDFGFARNLSEGNNANYTEYVATRWYRSPELLLGAPYGKSVDMWSVGCI
LGELSDGQPLFPGESEIDQLFTIQKVLGPLPSEQMKLFYSNPRFHGLRFPAVNHPQSLERRYL
GILNSVLLDLMKNLLKLDPADRYLTEQCLNHPTFQTQRLLDRSPSRSAKRKPYHVESSTLSNR
NQAGKSTALQSHHRSNSKDIQNLSVGLPRADEGLPANESFLNGNLAGASLSPLHTKTYQASSQ
PGSTSKDLTNNNIPHLLSPKEAKSKTEFDFNIDPKPSEGPGTKYLKSNSRSQQNRHSFMESSQ
SKAGTLQPNEKQSRHSYIDTIPQSSRSPSYRTKAKSHGALSDSKSVSNLSEARAQIAEPSTSR
YFPSSCLDLNSPTSPTPTRHSDTRTLLSPSGRNNRNEGTLDSRRTTTRHSKTMEELKLPEHMD
SSHSHSLSAPHESFSYGLGYTSPFSSQQRPHRHSMYVTRDKVRAKGLDGSLSIGQGMAARANS
LQLLSPQPGEQLPPEMTVARSSVKETSREGTSSFHTRQKSEGGVYHDPHSDDGTAPKENRHLY
NDPVPRRVGSFYRVPSPRPDNSFHENNVSTRVSSLPSESSSGTNHSKRQPAFDPWKSPENISH
SEQLKEKEKQGFFRSMKKKKKKSQTVPNSDSPDLLTLQKSIHSASTPSSRPKEWRPEKISDLQ
TQSQPLKSLRKLLHLSSASNHPASSDPRFQPLTAQQTKNSFSEIRIHPLSQASGGSSNIRQEP
APKGRPALQLPDGGCDGRRQRHHSGPQDRRFMLRTTEQQGEYFCCGDPKKPHTPCVPNRALHR
PISSPAPYPVLQVRGTSMCPTLQVRGTDAFSCPTQQSGFSFFVRHVMREALIHRAQVNQAALL

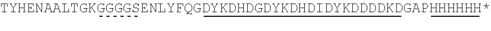

Tκ28p_107_3xFlagHis_cho-opt in pOptiVec
SEQ ID NO: 47
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG
YTSPFSSQQRPHRHSMYVTRDKVRAKGLDGSLSIGQGMAARANSLQLLSPQPGEQLPPEMTVA
RSSVKETSREGTSSFHTRQKSEGGVYHDPHSDDGTAPKENRHLYNDPVPRRVGSFYRVPSPRP
DNSFHENNVSTRVSSLPSESSSGTNHSKRQPAFDPWKSPENISHSEQLKEKEKQGFFRSMKKK
KKKSQTVPNSDSPDLLTLQKSIHSASTPSSRPKEWRPEKISDLQTQSQPLKSLRKLLHLSSAS
NHPASSDPRFQPLTAQQTKNSFSEIRIHPLSQASGGSSNIRQEPAPKGRPALQLPGQMDPGWH

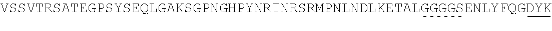

DHDGDYKDHDIDYKDDDDKDGAPHHHHHH*
Tκ28p_107_3xFlagHis_ecoli-opt in pEX-1
SEQ ID NO: 48
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG
YTSPFSSQQRPHRHSMYVTRDKVRAKGLDGSLSIGQGMAARANSLQLLSPQPGEQLPPEMTVA
RSSVKETSREGTSSFHTRQKSEGGVYHDPHSDDGTAPKENRHLYNDPVPRRVGSFYRVPSPRP
DNSFHENNVSTRVSSLPSESSSGTNHSKRQPAFDPWKSPENISHSEQLKEKEKQGFFRSMKKK
KKKSQTVPNSDSPDLLTLQKSIHSASTPSSRPKEWRPEKISDLQTQSQPLKSLRKLLHLSSAS
NHPASSDPRFQPLTAQQTKNSFSEIRIHPLSQASGGSSNIRQEPAPKGRPALQLPGQMDPGWH

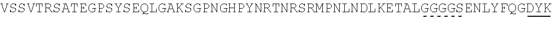

DHDGDYKDHDIDYKDDDDKDGAPHHHHHH*
Δ853-960 in pEX-1
SEQ ID NO: 49
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG
YTSPFSSQQRPHRHSMYVTRDKVRAKGLDGSLSIGQGMAARANSLQLLSPQPGEQLPPEMTVA
RSSVKETSREGTSSFHTRQKSEGGVYHDPHSDDGTAPKENRHLYNDPVPRRVGSFYRVPSPRP
DNSFHENNVSTRVSSLPSESSSGTNHSKRQPAFDPWKSPENISHSEQLKEKEKQGFFRSMKKK
KKKSQTVPNSDSPDLLTLQKSIHSASTPSSRPKEWRPEKISDLQTQSQPLKSLRKLLHLSSAS

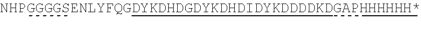

Δ745-960 in pEX-1
SEQ ID NO: 50
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG
YTSPFSSQQRPHRHSMYVTRDKVRAKGLDGSLSIGQGMAARANSLQLLSPQPGEQLPPEMTVA
RSSVKETSREGTSSFHTRQKSEGGVYHDPHSDDGTAPKENRHLYNDPVPRRVGSFYRVPSPRP

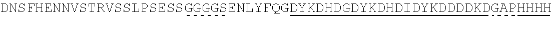

HH*
Δ637-960 in pEX-1
SEQ ID NO: 51
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG

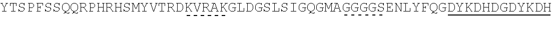

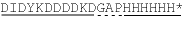


Δ529-960 in pEX-1
SEQ ID NO: 52
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID

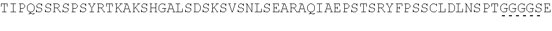

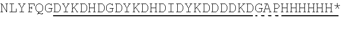


Δ421-960 in pEX-1
SEQ ID NO: 53
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP

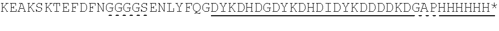

Δ315-960 in pEX-1
SEQ ID NO: 54
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP

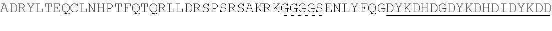

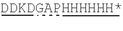


Δ315-420 in pEX-1
SEQ ID NO: 55
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKIDPKPSEGPGTKYLKSNSRSQQNRHSFMESS
QSKAGTLQPNEKQSRHSYIDTIPQSSRSPSYRTKAKSHGALSDSKSVSNLSEARAQIAEPSTS
RYFPSSCLDLNSPTSPTPTRHSDTRTLLSPSGRNNRNEGTLDSRRTTTRHSKTMEELKLPEHM
DSSHSHSLSAPHESFSYGLGYTSPFSSQQRPHRHSMYVTRDKVRAKGLDGSLSIGQGMAARAN
SLQLLSPQPGEQLPPEMTVARSSVKETSREGTSSFHTRQKSEGGVYHDPHSDDGTAPKENRHL
YNDPVPRRVGSFYRVPSPRPDNSFHENNVSTRVSSLPSESSSGTNHSKRQPAFDPWKSPENIS
HSEQLKEKEKQGFFRSMKKKKKKSQTVPNSDSPDLLTLQKSIHSASTPSSRPKEWRPEKISDL
QTQSQPLKSLRKLLHLSSASNHPASSDPRFQPLTAQQTKNSFSEIRIHPLSQASGGSSNIRQE
PAPKGRPALQLPGQMDPGWHVSSVTRSATEGPSYSEQLGAKSGPNGHPYNRTNRSRMPNLNDL

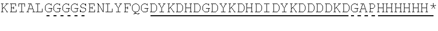

Δ315-528 in pEX-1
SEQ ID NO: 56
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKSPTPTRHSDTRTLLSPSGRNNRNEGTLDSRR
TTTRHSKTMEELKLPEHMDSSHSHSLSAPHESFSYGLGYTSPFSSQQRPHRHSMYVTRDKVRA
KGLDGSLSIGQGMAARANSLQLLSPQPGEQLPPEMTVARSSVKETSREGTSSFHTRQKSEGGV
YHDPHSDDGTAPKENRHLYNDPVPRRVGSFYRVPSPRPDNSFHENNVSTRVSSLPSESSSGTN
HSKRQPAFDPWKSPENISHSEQLKEKEKQGFFRSMKKKKKKSQTVPNSDSPDLLTLQKSIHSA
STPSSRPKEWRPEKISDLQTQSQPLKSLRKLLHLSSASNHPASSDPRFQPLTAQQTKNSFSEI
RIHPLSQASGGSSNIRQEPAPKGRPALQLPGQMDPGWHVSSVTRSATEGPSYSEQLGAKSGPN

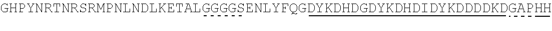

HHHH*
Δ315-636 in pEX-1
SEQ ID NO: 57
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKARANSLQLLSPQPGEQLPPEMTVARSSVKET
SREGTSSFHTRQKSEGGVYHDPHSDDGTAPKENRHLYNDPVPRRVGSFYRVPSPRPDNSFHEN
NVSTRVSSLPSESSSGTNHSKRQPAFDPWKSPENISHSEQLKEKEKQGFFRSMKKKKKKSQTV
PNSDSPDLLTLQKSIHSASTPSSRPKEWRPEKISDLQTQSQPLKSLRKLLHLSSASNHPASSD
PRFQPLTAQQTKNSFSEIRIHPLSQASGGSSNIRQEPAPKGRPALQLPGQMDPGWHVSSVTRS

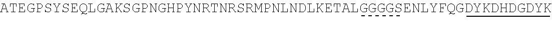

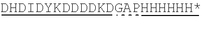


Δ315-744 in pEX-1
SEQ ID NO: 58
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKSGTNHSKRQPAFDPWKSPENISHSEQLKEKE
KQGFFRSMKKKKKKSQTVPNSDSPDLLTLQKSIHSASTPSSRPKEWRPEKISDLQTQSQPLKS
LRKLLHLSSASNHPASSDPRFQPLTAQQTKNSFSEIRIHPLSQASGGSSNIRQEPAPKGRPAL

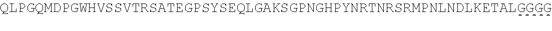

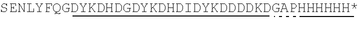


Δ315-852 in pEX-1
SEQ ID NO: 59
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKASSDPRFQPLTAQQTKNSFSEIRIHPLSQAS
GGSSNIRQEPAPKGRPALQLPGQMDPGWHVSSVTRSATEGPSYSEQLGAKSGPNGHPYNRTNR

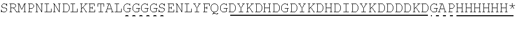

TT28p_107_3xFlagHis_ecoli-opt in pEX-1
SEQ ID NO: 60
MGDAAQPARRARRTKLAAYGRKKRRQRRRGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG
YTSPFSSQQRPHRHSMYVTRDKVRAKGLDGSLSIGQGMAARANSLQLLSPQPGEQLPPEMTVA
RSSVKETSREGTSSFHTRQKSEGGVYHDPHSDDGTAPKENRHLYNDPVPRRVGSFYRVPSPRP
DNSFHENNVSTRVSSLPSESSSGTNHSKRQPAFDPWKSPENISHSEQLKEKEKQGFFRSMKKK
KKKSQTVPNSDSPDLLTLQKSIHSASTPSSRPKEWRPEKISDLQTQSQPLKSLRKLLHLSSAS
NHPASSDPRFQPLTAQQTKNSFSEIRIHPLSQASGGSSNIRQEPAPKGRPALQLPGQMDPGWH

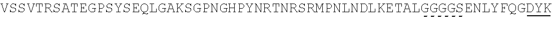

DHDGDYKDHDIDYKDDDDKDGAPHHHHHH*
Tκ28p_eGFP_ecoli-opt_3xFlagHis in pEX-1
SEQ ID NO: 61
MGDAAQPARRARRTKLAAYARKAARQARAGGGGSVSKGEELFTGVVPILVELDGDVNGHKFSV
SGEGEGDATYGKLTLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALSKDPNEKRDHM

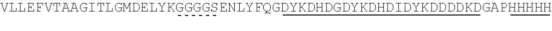

H*
eGFP_3xFlagHis_ecoli-opt in pEX-1
SEQ ID NO: 62
MVSKGEELFTGVVPILVELDGDVNGHKFSVSGEGEGDATYGKLTLKFICTTGKLPVPWPTLVT
TLTYGVQCFSRYPDHMKQHDFFKSAMPEGYVQERTIFFKDDGNYKTRAEVKFEGDTLVNRIEL
KGIDFKEDGNILGHKLEYNYNSHNVYIMADKQKNGIKVNFKIRHNIEDGSVQLADHYQQNTPI

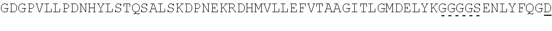

YKDHDGDYKDHDIDYKDDDDKDGAPHHHHHH*
AMPH1-3xFlagHis in pEX-1 (ecoli-opt)
SEQ ID NO: 63
MADIKTGIFAKNVQKRLNRAQEKVLQKLGKADETKDEQFEEYVQNFKRQEAEGTRLQRELRGY
LAAIKGMQEASMKLTESLHEVYEPDWYGREDVKMVGEKCDVLWEDFHQKLVDGSLLTLDTYLG
QFPDIKNRIAKRSRKLVDYDSARHHLEALQSSKRKDESRISKAEEEFQKAQKVFEEFNVDLQE
ELPSLWSRRVGFYVNTFKNVSSLEAKFHKEIAVLCHKLYEVMTKLGDQHADKAFTIQGAPSDS
GPLRIAKTPSPPEEPSPLPSPTASPNHTLAPASPAPARPRSPSQTRKGPPVPPLPKVTPTKEL
QQENIISFFEDNFVPEISVTTPSQNEVPEVKKEETLLDLDFDPFKPEVTPAGSAGVTHSPMSQ
TLPWDLWTTSTDLVQPASGGSFNGFTQPQDTSLFTMQTDQSMICNLAESEQAPPTEPKAEEPL
AAVTPAVGLDLGMDTRAEEPVEEAVIIPGADADAAVGTLVSAAEGAPGEEAEAEKATVPAGEG
VSLEEAKIGTETTEGAESAQPEAEELEATVPQEKVIPSVVIEPASNHEEEGENEITIGAEPKE
TTEDAAPPGPTSETPELATEQKPIQDPQPTPSAPAMGAADQLASAREASQELPPGFLYKVETL
HDFEAANSDELTLQRGDVVLVVPSDSEADQDAGWLVGVKESDWLQYRDLATYKGLFPENFTRR

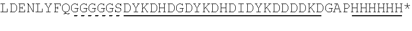

AMPH1-3xFlagHis cho-opt in pOptiVec
SEQ ID NO: 64
MADIKTGIFAKNVQKRLNRAQEKVLQKLGKADETKDEQFEEYVQNFKRQEAEGTRLQRELRGY
LAAIKGMQEASMKLTESLHEVYEPDWYGREDVKMVGEKCDVLWEDFHQKLVDGSLLTLDTYLG
QFPDIKNRIAKRSRKLVDYDSARHHLEALQSSKRKDESRISKAEEEFQKAQKVFEEFNVDLQE
ELPSLWSRRVGFYVNTFKNVSSLEAKFHKEIAVLCHKLYEVMTKLGDQHADKAFTIQGAPSDS
GPLRIAKTPSPPEEPSPLPSPTASPNHTLAPASPAPARPRSPSQTRKGPPVPPLPKVTPTKEL
QQENIISFFEDNFVPEISVTTPSQNEVPEVKKEETLLDLDFDPFKPEVTPAGSAGVTHSPMSQ
TLPWDLWTTSTDLVQPASGGSFNGFTQPQDTSLFTMQTDQSMICNLAESEQAPPTEPKAEEPL
AAVTPAVGLDLGMDTRAEEPVEEAVIIPGADADAAVGTLVSAAEGAPGEEAEAEKATVPAGEG
VSLEEAKIGTETTEGAESAQPEAEELEATVPQEKVIPSVVIEPASNHEEEGENEITIGAEPKE
TTEDAAPPGPTSETPELATEQKPIQDPQPTPSAPAMGAADQLASAREASQELPPGFLYKVETL
HDFEAANSDELTLQRGDVVLVVPSDSEADQDAGWLVGVKESDWLQYRDLATYKGLFPENFTRR

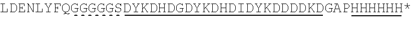

MBip_TATκ11_107__3xFlagHis_cho-opt in pOptiVec
SEQ ID NO: 65
MKLSLVAAMLLLLSLVAAMLLLLSAARAGYARKAARQARAGGGGSKIPNIGNVMNKFEILGW
GEGAYGVVLKCRHKETHEIVAIKKFKDSEENEEVKETTLRELKMLRTLKQENIVELKEAFRRR
GKLYLVFEYVEKNMLELLEEMPNGVPPEKVKSYIYQLIKAIHWCHKNDIVHRDIKPENLLISH
NDVLKLCDFGFARNLSEGNNANYTEYVATRWYRSPELLLGAPYGKSVDMWSVGCILGELSDGQ
PLFPGESEIDQLETIQKVLGPLPSEQMKLFYSNPRFHGLREPAVNHPQSLERRYLGILNSVLL
DLMKNLLKLDPADRYLTEQCLNHPTFQTQRLLDRSPSRSAKRKPYHVESSTLSNRNQAGKSTA
LQSHHRSNSKDIQNLSVGLPRADEGLPANESFLNGNLAGASLSPLHTKTYQASSQPGSTSKDL
TNNNIPHLLSPKEAKSKTEFDFNIDPKPSEGPGTKYLKSNSRSQQNRHSFMESSQSKAGTLQP
NEKQSRHSYIDTIPQSSRSPSYRTKAKSHGALSDSKSVSNLSEARAQIAEPSTSRYFPSSCLD
LNSPTSPTPTRHSDTRTLLSPSGRNNRNEGTLDSRRTTTRHSKTMEELKLPEHMDSSHSHSLS
APHESFSYGLGYTSPFSSQQRPHRHSMYVTRDKVRAKGLDGSLSIGQGMAARANSLQLLSPQP
GEQLPPEMTVARSSVKETSREGTSSFHTRQKSEGGVYHDPHSDDGTAPKENRHLYNDPVPRRV
GSFYRVPSPRPDNSFHENNVSTRVSSLPSESSSGTNHSKRQPAFDPWKSPENISHSEQLKEKE
KQGFFRSMKKKKKKSQTVPNSDSPDLLTLQKSIHSASTPSSRPKEWRPEKISDLQTQSQPLKS
LRKLLHLSSASNHPASSDPRFQPLTAQQTKNSFSEIRIHPLSQASGGSSNIRQEPAPKGRPAL

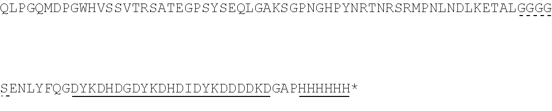

Igκ_TATκ11_107_ 3xFlagHis_cho-opt in pOptiVec
SEQ ID NO: 66
METDTLLLWVLLLWVPGSTGGYARKAARQARAGGGGSKIPNIGNVMNKFEILGVVGEGAYGVV
LKCRHKETHEIVAIKKFKDSEENEEVKETTLRELKMLRTLKQENIVELKEAFRRRGKLYLVFE
YVEKNMLELLEEMPNGVPPEKVKSYIYQLIKAIHWCHKNDIVHRDIKPENLLISHNDVLKLCD
FGFARNLSEGNNANYTEYVATRWYRSPELLLGAPYGKSVDMWSVGCILGELSDGQPLFPGESE
IDQLFTIQKVLGPLPSEQMKLFYSNPRFHGLRFPAVNHPQSLERRYLGILNSVLLDLMKNLLK
LDPADRYLTEQCLNHPTFQTQRLLDRSPSRSAKRKPYHVESSTLSNRNQAGKSTALQSHHRSN
SKDIQNLSVGLPRADEGLPANESFLNGNLAGASLSPLHTKTYQASSQPGSTSKDLTNNNIPHL
LSPKEAKSKTEFDFNIDPKPSEGPGTKYLKSNSRSQQNRHSFMESSQSKAGTLQPNEKQSRHS
YIDTIPQSSRSPSYRTKAKSHGALSDSKSVSNLSEARAQIAEPSTSRYFPSSCLDLNSPTSPT
PTRHSDTRTLLSPSGRNNRNEGTLDSRRTTTRHSKTMEELKLPEHMDSSHSHSLSAPHESFSY
GLGYTSPFSSQQRPHRHSMYVTRDKVRAKGLDGSLSIGQGMAARANSLQLLSPQPGEQLPPEM
TVARSSVKETSREGTSSFHTRQKSEGGVYHDPHSDDGTAPKENRHLYNDPVPRRVGSFYRVPS
PRPDNSFHENNVSTRVSSLPSESSSGTNHSKRQPAFDPWKSPENISHSEQLKEKEKQGFFRSM
KKKKKKSQTVPNSDSPDLLTLQKSIHSASTPSSRPKEWRPEKISDLQTQSQPLKSLRKLLHLS
SASNHPASSDPRFQPLTAQQTKNSFSEIRIHPLSQASGGSSNIRQEPAPKGRPALQLPGQMDP

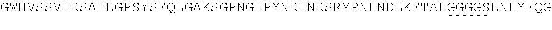

DYKDHDGDYKDHDIDYKDDDDKDGAPHHHHHH*
TATκ11_107_3xFlagHis_cho-opt in pOptiVec (leaderless)
SEQ ID NO: 67
MGYARKAARQARAGGGGSKIPNIGNVMNKFEILGVVGEGAYGVVLKCRHKETHEIVAIKKFKD
SEENEEVKETTLRELKMLRTLKQENIVELKEAFRRRGKLYLVFEYVEKNMLELLEEMPNGVPP
EKVKSYIYQLIKAIHWCHKNDIVHRDIKPENLLISHNDVLKLCDFGFARNLSEGNNANYTEYV
ATRWYRSPELLLGAPYGKSVDMWSVGCILGELSDGQPLFPGESEIDQLFTIQKVLGPLPSEQM
KLFYSNPRFHGLRFPAVNHPQSLERRYLGILNSVLLDLMKNLLKLDPADRYLTEQCLNHPTFQ
TQRLLDRSPSRSAKRKPYHVESSTLSNRNQAGKSTALQSHHRSNSKDIQNLSVGLPRADEGLP
ANESFLNGNLAGASLSPLHTKTYQASSQPGSTSKDLTNNNIPHLLSPKEAKSKTEFDFNIDPK
PSEGPGTKYLKSNSRSQQNRHSFMESSQSKAGTLQPNEKQSRHSYIDTIPQSSRSPSYRTKAK
SHGALSDSKSVSNLSEARAQIAEPSTSRYFPSSCLDLNSPTSPTPTRHSDTRTLLSPSGRNNR
NEGTLDSRRTTTRHSKTMEELKLPEHMDSSHSHSLSAPHESFSYGLGYTSPFSSQQRPHRHSM
YVTRDKVRAKGLDGSLSIGQGMAARANSLQLLSPQPGEQLPPEMTVARSSVKETSREGTSSFH
TRQKSEGGVYHDPHSDDGTAPKENRHLYNDPVPRRVGSFYRVPSPRPDNSFHENNVSTRVSSL
PSESSSGTNHSKRQPAFDPWKSPENISHSEQLKEKEKQGFFRSMKKKKKKSQTVPNSDSPDLL
TLQKSIHSASTPSSRPKEWRPEKISDLQTQSQPLKSLRKLLHLSSASNHPASSDPRFQPLTAQ
QTKNSFSEIRIHPLSQASGGSSNIRQEPAPKGRPALQLPGQMDPGWHVSSVTRSATEGPSYSE

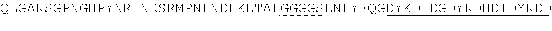

DDKDGAPHHHHHH*
TATκ11_107_3xFlagHis_ecoli-opt in pEX-1
SEQ ID NO: 68
MGYARKAARQARAGGGGSKIPNIGNVMNKFEILGVVGEGAYGVVLKCRHKETHEIVAIKKFKD
SEENEEVKETTLRELKMLRTLKQENIVELKEAFRRRGKLYLVFEYVEKNMLELLEEMPNGVPP
EKVKSYIYQLIKAIHWCHKNDIVHRDIKPENLLISHNDVLKLCDFGFARNLSEGNNANYTEYV
ATRWYRSPELLLGAPYGKSVDMWSVGCILGELSDGQPLFPGESEIDQLFTIQKVLGPLPSEQM
KLFYSNPRFHGLRFPAVNHPQSLERRYLGILNSVLLDLMKNLLKLDPADRYLTEQCLNHPTFQ
TQRLLDRSPSRSAKRKPYHVESSTLSNRNQAGKSTALQSHHRSNSKDIQNLSVGLPRADEGLP
ANESFLNGNLAGASLSPLHTKTYQASSQPGSTSKDLTNNNIPHLLSPKEAKSKTEFDFNIDPK
PSEGPGTKYLKSNSRSQQNRHSFMESSQSKAGTLQPNEKQSRHSYIDTIPQSSRSPSYRTKAK
SHGALSDSKSVSNLSEARAQIAEPSTSRYFPSSCLDLNSPTSPTPTRHSDTRTLLSPSGRNNR
NEGTLDSRRTTTRHSKTMEELKLPEHMDSSHSHSLSAPHESFSYGLGYTSPFSSQQRPHRHSM
YVTRDKVRAKGLDGSLSIGQGMAARANSLQLLSPQPGEQLPPEMTVARSSVKETSREGTSSFH
TRQKSEGGVYHDPHSDDGTAPKENRHLYNDPVPRRVGSFYRVPSPRPDNSFHENNVSTRVSSL
PSESSSGTNHSKRQPAFDPWKSPENISHSEQLKEKEKQGFFRSMKKKKKKSQTVPNSDSPDLL
TLQKSIHSASTPSSRPKEWRPEKISDLQTQSQPLKSLRKLLHLSSASNHPASSDPRFQPLTAQ
QTKNSFSEIRIHPLSQASGGSSNIRQEPAPKGRPALQLPGQMDPGWHVSSVTRSATEGPSYSE

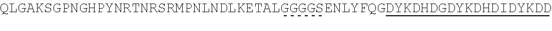

DDKDGAPHHHHHH*
TAT11_107_3xFlagHis_ecoli-opt in pEX-1
SEQ ID NO: 69
MGYGRKKRRQRRRGGGGSKIPNIGNVMNKFEILGVVGEGAYGVVLKCRHKETHEIVAIKKFKD
SEENEEVKETTLRELKMLRTLKQENIVELKEAFRRRGKLYLVFEYVEKNMLELLEEMPNGVPP
EKVKSYIYQLIKAIHWCHKNDIVHRDIKPENLLISHNDVLKLCDFGFARNLSEGNNANYTEYV
ATRWYRSPELLLGAPYGKSVDMWSVGCILGELSDGQPLFPGESEIDQLFTIQKVLGPLPSEQM
KLFYSNPRFHGLRFPAVNHPQSLERRYLGILNSVLLDLMKNLLKLDPADRYLTEQCLNHPTFQ
TQRLLDRSPSRSAKRKPYHVESSTLSNRNQAGKSTALQSHHRSNSKDIQNLSVGLPRADEGLP
ANESFLNGNLAGASLSPLHTKTYQASSQPGSTSKDLTNNNIPHLLSPKEAKSKTEFDFNIDPK
PSEGPGTKYLKSNSRSQQNRHSFMESSQSKAGTLQPNEKQSRHSYIDTIPQSSRSPSYRTKAK
SHGALSDSKSVSNLSEARAQIAEPSTSRYFPSSCLDLNSPTSPTPTRHSDTRTLLSPSGRNNR
NEGTLDSRRTTTRHSKTMEELKLPEHMDSSHSHSLSAPHESFSYGLGYTSPFSSQQRPHRHSM
YVTRDKVRAKGLDGSLSIGQGMAARANSLQLLSPQPGEQLPPEMTVARSSVKETSREGTSSFH
TRQKSEGGVYHDPHSDDGTAPKENRHLYNDPVPRRVGSFYRVPSPRPDNSFHENNVSTRVSSL
PSESSSGTNHSKRQPAFDPWKSPENISHSEQLKEKEKQGFFRSMKKKKKKSQTVPNSDSPDLL
TLQKSIHSASTPSSRPKEWRPEKISDLQTQSQPLKSLRKLLHLSSASNHPASSDPRFQPLTAQ
QTKNSFSEIRIHPLSQASGGSSNIRQEPAPKGRPALQLPGQMDPGWHVSSVTRSATEGPSYSE

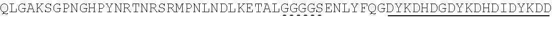

DDKDGAPHHHHHH*
TAT11_107_3xFlagHis_cho-opt in pOptiVec (leaderless)
SEQ ID NO: 70
MGYGRKKRRQRRRGGGGSKIPNIGNVMNKFEILGVVGEGAYGVVLKCRHKETHEIVAIKKFKD
SEENEEVKETTLRELKMLRTLKQENIVELKEAFRRRGKLYLVFEYVEKNMLELLEEMPNGVPP
EKVKSYIYQLIKAIHWCHKNDIVHRDIKPENLLISHNDVLKLCDFGFARNLSEGNNANYTEYV
ATRWYRSPELLLGAPYGKSVDMWSVGCILGELSDGQPLFPGESEIDQLFTIQKVLGPLPSEQM
KLFYSNPRFHGLRFPAVNHPQSLERRYLGILNSVLLDLMKNLLKLDPADRYLTEQCLNHPTFQ
TQRLLDRSPSRSAKRKPYHVESSTLSNRNQAGKSTALQSHHRSNSKDIQNLSVGLPRADEGLP
ANESFLNGNLAGASLSPLHTKTYQASSQPGSTSKDLTNNNIPHLLSPKEAKSKTEFDFNIDPK
PSEGPGTKYLKSNSRSQQNRHSFMESSQSKAGTLQPNEKQSRHSYIDTIPQSSRSPSYRTKAK
SHGALSDSKSVSNLSEARAQIAEPSTSRYFPSSCLDLNSPTSPTPTRHSDTRTLLSPSGRNNR
NEGTLDSRRTTTRHSKTMEELKLPEHMDSSHSHSLSAPHESFSYGLGYTSPFSSQQRPHRHSM
YVTRDKVRAKGLDGSLSIGQGMAARANSLQLLSPQPGEQLPPEMTVARSSVKETSREGTSSFH
TRQKSEGGVYHDPHSDDGTAPKENRHLYNDPVPRRVGSFYRVPSPRPDNSFHENNVSTRVSSL
PSESSSGTNHSKRQPAFDPWKSPENISHSEQLKEKEKQGFFRSMKKKKKKSQTVPNSDSPDLL
TLQKSIHSASTPSSRPKEWRPEKISDLQTQSQPLKSLRKLLHLSSASNHPASSDPRFQPLTAQ
QTKNSFSEIRIHPLSQASGGSSNIRQEPAPKGRPALQLPGQMDPGWHVSSVTRSATEGPSYSE

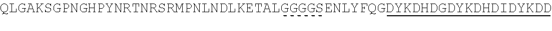

DDKDGAPHHHHHH
ANTP_107_3xFlagHis_cho-opt in pOptiVec
SEQ ID NO: 71
MGRQIKIWFQNRRMKWKKGGGGSKIPNIGNVMNKFEILGVVGEGAYGVVLKCRHKETHEIVAI
KKFKDSEENEEVKETTLRELKMLRTLKQENIVELKEAFRRRGKLYLVFEYVEKNMLELLEEMP
NGVPPEKVKSYIYQLIKAIHWCHKNDIVHRDIKPENLLISHNDVLKLCDFGFARNLSEGNNAN
YTEYVATRWYRSPELLLGAPYGKSVDMWSVGCILGELSDGQPLFPGESEIDQLFTIQKVLGPL
PSEQMKLFYSNPRFHGLRFPAVNHPQSLERRYLGILNSVLLDLMKNLLKLDPADRYLTEQCLN
HPTFQTQRLLDRSPSRSAKRKPYHVESSTLSNRNQAGKSTALQSHHRSNSKDIQNLSVGLPRA
DEGLPANESFLNGNLAGASLSPLHTKTYQASSQPGSTSKDLTNNNIPHLLSPKEAKSKTEFDF
NIDPKPSEGPGTKYLKSNSRSQQNRHSFMESSQSKAGTLQPNEKQSRHSYIDTIPQSSRSPSY
RTKAKSHGALSDSKSVSNLSEARAQIAEPSTSRYFPSSCLDLNSPTSPTPTRHSDTRTLLSPS
GRNNRNEGTLDSRRTTTRHSKTMEELKLPEHMDSSHSHSLSAPHESFSYGLGYTSPFSSQQRP
HRHSMYVTRDKVRAKGLDGSLSIGQGMAARANSLQLLSPQPGEQLPPEMTVARSSVKETSREG
TSSFHTRQKSEGGVYHDPHSDDGTAPKENRHLYNDPVPRRVGSFYRVPSPRPDNSFHENNVST
RVSSLPSESSSGTNHSKRQPAFDPWKSPENISHSEQLKEKEKQGFFRSMKKKKKKSQTVPNSD
SPDLLTLQKSIHSASTPSSRPKEWRPEKISDLQTQSQPLKSLRKLLHLSSASNHPASSDPRFQ
PLTAQQTKNSFSEIRIHPLSQASGGSSNIRQEPAPKGRPALQLPGQMDPGWHVSSVTRSATEG

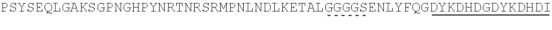

DYKDDDDKDGAPHHHHHH*
TRANSP_107_3xFlagHis_cho-opt in pOptiVec
SEQ ID NO: 72
MGAGYLLGKINLKALAALAKKILGGGGSKIPNIGNVMNKFEILGVVGEGAYGVVLKCRHKETH
EIVAIKKFKDSEENEEVKETTLRELKMLRTLKQENIVELKEAFRRRGKLYLVFEYVEKNMLEL
LEEMPNGVPPEKVKSYIYQLIKAIHWCHKNDIVHRDIKPENLLISHNDVLKLCDFGFARNLSE
GNNANYTEYVATRWYRSPELLLGAPYGKSVDMWSVGCILGELSDGQPLFPGESEIDQLFTIQK
VLGPLPSEQMKLFYSNPRFHGLRFPAVNHPQSLERRYLGILNSVLLDLMKNLLKLDPADRYLT
EQCLNHPTFQTQRLLDRSPSRSAKRKPYHVESSTLSNRNQAGKSTALQSHHRSNSKDIQNLSV
GLPRADEGLPANESFLNGNLAGASLSPLHTKTYQASSQPGSTSKDLTNNNIPHLLSPKEAKSK
TEFDFNIDPKPSEGPGTKYLKSNSRSQQNRHSFMESSQSKAGTLQPNEKQSRHSYIDTIPQSS
RSPSYRTKAKSHGALSDSKSVSNLSEARAQIAEPSTSRYFPSSCLDLNSPTSPTPTRHSDTRT
LLSPSGRNNRNEGTLDSRRTTTRHSKTMEELKLPEHMDSSHSHSLSAPHESFSYGLGYTSPFS
SQQRPHRHSMYVTRDKVRAKGLDGSLSIGQGMAARANSLQLLSPQPGEQLPPEMTVARSSVKE
TSREGTSSFHTRQKSEGGVYHDPHSDDGTAPKENRHLYNDPVPRRVGSFYRVPSPRPDNSFHE
NNVSTRVSSLPSESSSGTNHSKRQPAFDPWKSPENISHSEQLKEKEKQGFFRSMKKKKKKSQT
VPNSDSPDLLTLQKSIHSASTPSSRPKEWRPEKISDLQTQSQPLKSLRKLLHLSSASNHPASS
DPRFQPLTAQQTKNSFSEIRIHPLSQASGGSSNIRQEPAPKGRPALQLPGQMDPGWHVSSVTR

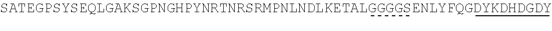

KDHDIDYKDDDDKDGAPHHHHHH*
TAT28_107_3xFlagHis_cho-opt in pOptiVec
SEQ ID NO: 73
MGDAAQPARRARRTKLAAYGRKKRRQRRRGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG
YTSPFSSQQRPHRHSMYVTRDKVRAKGLDGSLSIGQGMAARANSLQLLSPQPGEQLPPEMTVA
RSSVKETSREGTSSFHTRQKSEGGVYHDPHSDDGTAPKENRHLYNDPVPRRVGSFYRVPSPRP
DNSFHENNVSTRVSSLPSESSSGTNHSKRQPAFDPWKSPENISHSEQLKEKEKQGFFRSMKKK
KKKSQTVPNSDSPDLLTLQKSIHSASTPSSRPKEWRPEKISDLQTQSQPLKSLRKLLHLSSAS
NHPASSDPRFQPLTAQQTKNSFSEIRIHPLSQASGGSSNIRQEPAPKGRPALQLPGQMDPGWH

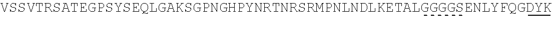

DHDGDYKDHDIDYKDDDDKDGAPHHHHHH*
MBIP_P97_107_3xFlagHis_cho-opt in pOptiVec
SEQ ID NO: 74
MKLSLVAAMLLLLSLVAAMLLLLSAARAGDSSHAFTLDELRGGGGSKIPNIGNVMNKFEILGV
VGEGAYGVVLKCRHKETHEIVAIKKFKDSEENEEVKETTLRELKMLRTLKQENIVELKEAFRR
RGKLYLVFEYVEKNMLELLEEMPNGVPPEKVKSYIYQLIKAIHWCHKNDIVHRDIKPENLLIS
HNDVLKLCDFGFARNLSEGNNANYTEYVATRWYRSPELLLGAPYGKSVDMWSVGCILGELSDG
QPLFPGESEIDQLFTIQKVLGPLPSEQMKLFYSNPRFHGLRFPAVNHPQSLERRYLGILNSVL
LDLMKNLLKLDPADRYLTEQCLNHPTFQTQRLLDRSPSRSAKRKPYHVESSTLSNRNQAGKST
ALQSHHRSNSKDIQNLSVGLPRADEGLPANESFLNGNLAGASLSPLHTKTYQASSQPGSTSKD
LTNNNIPHLLSPKEAKSKTEFDFNIDPKPSEGPGTKYLKSNSRSQQNRHSFMESSQSKAGTLQ
PNEKQSRHSYIDTIPQSSRSPSYRTKAKSHGALSDSKSVSNLSEARAQIAEPSTSRYFPSSCL
DLNSPTSPTPTRHSDTRTLLSPSGRNNRNEGTLDSRRTTTRHSKTMEELKLPEHMDSSHSHSL
SAPHESESYGLGYTSPESSQQRPHRHSMYVTRDKVRAKGLDGSLSIGQGMAARANSLQLLSPQ
PGEQLPPEMTVARSSVKETSREGTSSFHTRQKSEGGVYHDPHSDDGTAPKENRHLYNDPVPRR
VGSFYRVPSPRPDNSFHENNVSTRVSSLPSESSSGTNHSKRQPAFDPWKSPENISHSEQLKEK
EKQGFFRSMKKKKKKSQTVPNSDSPDLLTLQKSIHSASTPSSRPKEWRPEKISDLQTQSQPLK
SLRKLLHLSSASNHPASSDPRFQPLTAQQTKNSFSEIRIHPLSQASGGSSNIRQEPAPKGRPA

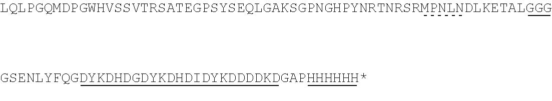

P97_107_3xFlagHis_human-opt in pT7CFEl
SEQ ID NO: 75
MGDSSHAFTLDELRGGGGSKIPNIGNVMNKFEILGVVGEGAYGVVLKCRHKETHEIVAIKKFK
DSEENEEVKETTLRELKMLRTLKQENIVELKEAFRRRGKLYLVFEYVEKNMLELLEEMPNGVP
PEKVKSYIYQLIKAIHWCHKNDIVHRDIKPENLLISHNDVLKLCDFGFARNLSEGNNANYTEY
VATRWYRSPELLLGAPYGKSVDMWSVGCILGELSDGQPLFPGESEIDQLFTIQKVLGPLPSEQ
MKLFYSNPRFHGLRFPAVNHPQSLERRYLGILNSVLLDLMKNLLKLDPADRYLTEQCLNHPTF
QTQRLLDRSPSRSAKRKPYHVESSTLSNRNQAGKSTALQSHHRSNSKDIQNLSVGLPRADEGL
PANESFLNGNLAGASLSPLHTKTYQASSQPGSTSKDLTNNNIPHLLSPKEAKSKTEFDFNIDP
KPSEGPGTKYLKSNSRSQQNRHSFMESSQSKAGTLQPNEKQSRHSYIDTIPQSSRSPSYRTKA
KSHGALSDSKSVSNLSEARAQIAEPSTSRYFPSSCLDLNSPTSPTPTRHSDTRTLLSPSGRNN
RNEGTLDSRRTTTRHSKTMEELKLPEHMDSSHSHSLSAPHESFSYGLGYTSPFSSQQRPHRHS
MYVTRDKVRAKGLDGSLSIGQGMAARANSLQLLSPQPGEQLPPEMTVARSSVKETSREGTSSF
HTRQKSEGGVYHDPHSDDGTAPKENRHLYNDPVPRRVGSFYRVPSPRPDNSFHENNVSTRVSS
LPSESSSGTNHSKRQPAFDPWKSPENISHSEQLKEKEKQGFFRSMKKKKKKSQTVPNSDSPDL
LTLQKSIHSASTPSSRPKEWRPEKISDLQTQSQPLKSLRKLLHLSSASNHPASSDPRFQPLTA
QQTKNSFSEIRIHPLSQASGGSSNIRQEPAPKGRPALQLPGQMDPGWHVSSVTRSATEGPSYS

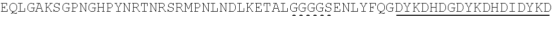

DDDKDGAPHHHHHH*
Tκ28p_107_3xFlagHis_human-opt in pOptiVec
SEQ ID NO: 76
MGDAAQPARRARRTKLAAYARKAARQARAGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG
YTSPFSSQQRPHRHSMYVTRDKVRAKGLDGSLSIGQGMAARANSLQLLSPQPGEQLPPEMTVA
RSSVKETSREGTSSFHTRQKSEGGVYHDPHSDDGTAPKENRHLYNDPVPRRVGSFYRVPSPRP
DNSFHENNVSTRVSSLPSESSSGTNHSKRQPAFDPWKSPENISHSEQLKEKEKQGFFRSMKKK
KKKSQTVPNSDSPDLLTLQKSIHSASTPSSRPKEWRPEKISDLQTQSQPLKSLRKLLHLSSAS
NHPASSDPRFQPLTAQQTKNSFSEIRIHPLSQASGGSSNIRQEPAPKGRPALQLPGQMDPGWH

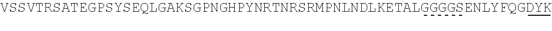

DHDGDYKDHDIDYKDDDDKDGAPHHHHHH*
TATκ11_107_3xFlagHis_human-opt in pOptiVec
SEQ ID NO: 77
MGYARKAARQARAGGGGSKIPNIGNVMNKFEILGVVGEGAYGVVLKCRHKETHEIVAIKKFKD
SEENEEVKETTLRELKMLRTLKQENIVELKEAFRRRGKLYLVFEYVEKNMLELLEEMPNGVPP
EKVKSYIYQLIKAIHWCHKNDIVHRDIKPENLLISHNDVLKLCDFGFARNLSEGNNANYTEYV
ATRWYRSPELLLGAPYGKSVDMWSVGCILGELSDGQPLFPGESEIDQLETIQKVLGPLPSEQM
KLFYSNPRFHGLRFPAVNHPQSLERRYLGILNSVLLDLMKNLLKLDPADRYLTEQCLNHPTFQ
TQRLLDRSPSRSAKRKPYHVESSTLSNRNQAGKSTALQSHHRSNSKDIQNLSVGLPRADEGLP
ANESFLNGNLAGASLSPLHTKTYQASSQPGSTSKDLTNNNIPHLLSPKEAKSKTEFDFNIDPK
PSEGPGTKYLKSNSRSQQNRHSFMESSQSKAGTLQPNEKQSRHSYIDTIPQSSRSPSYRTKAK
SHGALSDSKSVSNLSEARAQIAEPSTSRYFPSSCLDLNSPTSPTPTRHSDTRTLLSPSGRNNR
NEGTLDSRRTTTRHSKTMEELKLPEHMDSSHSHSLSAPHESFSYGLGYTSPFSSQQRPHRHSM
YVTRDKVRAKGLDGSLSIGQGMAARANSLQLLSPQPGEQLPPEMTVARSSVKETSREGTSSFH
TRQKSEGGVYHDPHSDDGTAPKENRHLYNDPVPRRVGSFYRVPSPRPDNSFHENNVSTRVSSL
PSESSSGTNHSKRQPAFDPWKSPENISHSEQLKEKEKQGFFRSMKKKKKKSQTVPNSDSPDLL
TLQKSIHSASTPSSRPKEWRPEKISDLQTQSQPLKSLRKLLHLSSASNHPASSDPRFQPLTAQ
QTKNSFSEIRIHPLSQASGGSSNIRQEPAPKGRPALQLPGQMDPGWHVSSVTRSATEGPSYSE

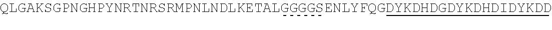

DDKDGAPHHHHHH*
TAT28_107_3xFlagHis_human-opt in pOptiVec
SEQ ID NO: 78
MGDAAQPARRARRTKLAAYGRKKRRQRRRGGGGSKIPNIGNVMNKFEILGVVGEGAYGVVLKC
RHKETHEIVAIKKFKDSEENEEVKETTLRELKMLRTLKQENIVELKEAFRRRGKLYLVFEYVE
KNMLELLEEMPNGVPPEKVKSYIYQLIKAIHWCHKNDIVHRDIKPENLLISHNDVLKLCDFGF
ARNLSEGNNANYTEYVATRWYRSPELLLGAPYGKSVDMWSVGCILGELSDGQPLFPGESEIDQ
LFTIQKVLGPLPSEQMKLFYSNPRFHGLRFPAVNHPQSLERRYLGILNSVLLDLMKNLLKLDP
ADRYLTEQCLNHPTFQTQRLLDRSPSRSAKRKPYHVESSTLSNRNQAGKSTALQSHHRSNSKD
IQNLSVGLPRADEGLPANESFLNGNLAGASLSPLHTKTYQASSQPGSTSKDLTNNNIPHLLSP
KEAKSKTEFDFNIDPKPSEGPGTKYLKSNSRSQQNRHSFMESSQSKAGTLQPNEKQSRHSYID
TIPQSSRSPSYRTKAKSHGALSDSKSVSNLSEARAQIAEPSTSRYFPSSCLDLNSPTSPTPTR
HSDTRTLLSPSGRNNRNEGTLDSRRTTTRHSKTMEELKLPEHMDSSHSHSLSAPHESFSYGLG
YTSPFSSQQRPHRHSMYVTRDKVRAKGLDGSLSIGQGMAARANSLQLLSPQPGEQLPPEMTVA
RSSVKETSREGTSSFHTRQKSEGGVYHDPHSDDGTAPKENRHLYNDPVPRRVGSFYRVPSPRP
DNSFHENNVSTRVSSLPSESSSGTNHSKRQPAFDPWKSPENISHSEQLKEKEKQGFFRSMKKK
KKKSQTVPNSDSPDLLTLQKSIHSASTPSSRPKEWRPEKISDLQTQSQPLKSLRKLLHLSSAS
NHPASSDPRFQPLTAQQTKNSFSEIRIHPLSQASGGSSNIRQEPAPKGRPALQLPGQMDPGWH

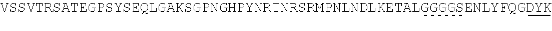

DHDGDYKDHDIDYKDDDDKDGAPHHHHHH*
TAT11_107_3xFlagHis_human-opt in pOptiVec
SEQ ID NO: 79
MGYGRKKRRQRRRGGGGSKIPNIGNVMNKFEILGVVGEGAYGVVLKCRHKETHEIVAIKKFKD
SEENEEVKETTLRELKMLRTLKQENIVELKEAFRRRGKLYLVFEYVEKNMLELLEEMPNGVPP
EKVKSYIYQLIKAIHWCHKNDIVHRDIKPENLLISHNDVLKLCDFGFARNLSEGNNANYTEYV
ATRWYRSPELLLGAPYGKSVDMWSVGCILGELSDGQPLFPGESEIDQLFTIQKVLGPLPSEQM
KLFYSNPRFHGLRFPAVNHPQSLERRYLGILNSVLLDLMKNLLKLDPADRYLTEQCLNHPTFQ
TQRLLDRSPSRSAKRKPYHVESSTLSNRNQAGKSTALQSHHRSNSKDIQNLSVGLPRADEGLP
ANESFLNGNLAGASLSPLHTKTYQASSQPGSTSKDLTNNNIPHLLSPKEAKSKTEFDFNIDPK
PSEGPGTKYLKSNSRSQQNRHSFMESSQSKAGTLQPNEKQSRHSYIDTIPQSSRSPSYRTKAK
SHGALSDSKSVSNLSEARAQIAEPSTSRYFPSSCLDLNSPTSPTPTRHSDTRTLLSPSGRNNR
NEGTLDSRRTTTRHSKTMEELKLPEHMDSSHSHSLSAPHESFSYGLGYTSPFSSQQRPHRHSM
YVTRDKVRAKGLDGSLSIGQGMAARANSLQLLSPQPGEQLPPEMTVARSSVKETSREGTSSFH
TRQKSEGGVYHDPHSDDGTAPKENRHLYNDPVPRRVGSFYRVPSPRPDNSFHENNVSTRVSSL
PSESSSGTNHSKRQPAFDPWKSPENISHSEQLKEKEKQGFFRSMKKKKKKSQTVPNSDSPDLL
TLQKSIHSASTPSSRPKEWRPEKISDLQTQSQPLKSLRKLLHLSSASNHPASSDPRFQPLTAQ
QTKNSFSEIRIHPLSQASGGSSNIRQEPAPKGRPALQLPGQMDPGWHVSSVTRSATEGPSYSE

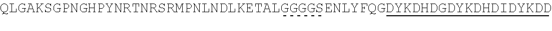

DDKDGAPHHHHHH*
ANTP_107_3xFlagHis_human-opt in pOptiVec
SEQ ID NO: 80
MGRQIKIWFQNRRMKWKKGGGGSKIPNIGNVMNKFEILGVVGEGAYGVVLKCRHKETHEIVAI
KKFKDSEENEEVKETTLRELKMLRTLKQENIVELKEAFRRRGKLYLVFEYVEKNMLELLEEMP
NGVPPEKVKSYIYQLIKAIHWCHKNDIVHRDIKPENLLISHNDVLKLCDFGFARNLSEGNNAN
YTEYVATRWYRSPELLLGAPYGKSVDMWSVGCILGELSDGQPLFPGESEIDQLFTIQKVLGPL
PSEQMKLFYSNPRFHGLRFPAVNHPQSLERRYLGILNSVLLDLMKNLLKLDPADRYLTEQCLN
HPTFQTQRLLDRSPSRSAKRKPYHVESSTLSNRNQAGKSTALQSHHRSNSKDIQNLSVGLPRA
DEGLPANESFLNGNLAGASLSPLHTKTYQASSQPGSTSKDLTNNNIPHLLSPKEAKSKTEFDF
NIDPKPSEGPGTKYLKSNSRSQQNRHSFMESSQSKAGTLQPNEKQSRHSYIDTIPQSSRSPSY
RTKAKSHGALSDSKSVSNLSEARAQIAEPSTSRYFPSSCLDLNSPTSPTPTRHSDTRTLLSPS
GRNNRNEGTLDSRRTTTRHSKTMEELKLPEHMDSSHSHSLSAPHESFSYGLGYTSPFSSQQRP
HRHSMYVTRDKVRAKGLDGSLSIGQGMAARANSLQLLSPQPGEQLPPEMTVARSSVKETSREG
TSSFHTRQKSEGGVYHDPHSDDGTAPKENRHLYNDPVPRRVGSFYRVPSPRPDNSFHENNVST
RVSSLPSESSSGTNHSKRQPAFDPWKSPENISHSEQLKEKEKQGFFRSMKKKKKKSQTVPNSD
SPDLLTLQKSIHSASTPSSRPKEWRPEKISDLQTQSQPLKSLRKLLHLSSASNHPASSDPRFQ
PLTAQQTKNSFSEIRIHPLSQASGGSSNIRQEPAPKGRPALQLPGQMDPGWHVSSVTRSATEG

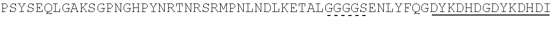

DYKDDDDKDGAPHHHHHH*
TRANSP_107_3xFlagHis_human-opt in pOptiVec
SEQ ID NO: 81
MGAGYLLGKINLKALAALAKKILGGGGSKIPNIGNVMNKFEILGVVGEGAYGVVLKCRHKETH
EIVAIKKFKDSEENEEVKETTLRELKMLRTLKQENIVELKEAFRRRGKLYLVFEYVEKNMLEL
LEEMPNGVPPEKVKSYIYQLIKAIHWCHKNDIVHRDIKPENLLISHNDVLKLCDFGFARNLSE
GNNANYTEYVATRWYRSPELLLGAPYGKSVDMWSVGCILGELSDGQPLFPGESEIDQLFTIQK
VLGPLPSEQMKLFYSNPRFHGLRFPAVNHPQSLERRYLGILNSVLLDLMKNLLKLDPADRYLT
EQCLNHPTFQTQRLLDRSPSRSAKRKPYHVESSTLSNRNQAGKSTALQSHHRSNSKDIQNLSV
GLPRADEGLPANESFLNGNLAGASLSPLHTKTYQASSQPGSTSKDLTNNNIPHLLSPKEAKSK
TEFDFNIDPKPSEGPGTKYLKSNSRSQQNRHSFMESSQSKAGTLQPNEKQSRHSYIDTIPQSS
RSPSYRTKAKSHGALSDSKSVSNLSEARAQIAEPSTSRYFPSSCLDLNSPTSPTPTRHSDTRT
LLSPSGRNNRNEGTLDSRRTTTRHSKTMEELKLPEHMDSSHSHSLSAPHESFSYGLGYTSPFS
SQQRPHRHSMYVTRDKVRAKGLDGSLSIGQGMAARANSLQLLSPQPGEQLPPEMTVARSSVKE
TSREGTSSFHTRQKSEGGVYHDPHSDDGTAPKENRHLYNDPVPRRVGSFYRVPSPRPDNSFHE
NNVSTRVSSLPSESSSGTNHSKRQPAFDPWKSPENISHSEQLKEKEKQGFFRSMKKKKKKSQT
VPNSDSPDLLTLQKSIHSASTPSSRPKEWRPEKISDLQTQSQPLKSLRKLLHLSSASNHPASS
DPRFQPLTAQQTKNSFSEIRIHPLSQASGGSSNIRQEPAPKGRPALQLPGQMDPGWHVSSVTR

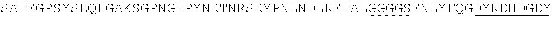

KDHDIDYKDDDDKDGAPHHHHHH*
MBip_Tκ28p_107_3xFlagHis_human-opt in pOptiVec
SEQ ID NO: 82
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARNLSEGNNANYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQNLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSNLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPENISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYNRTNRSR

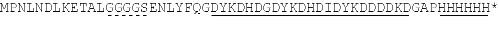

-GST-P-TATκ28-CDKL5_107-P-FH_pVL1393 (insect)
SEQ ID NO: 83
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI
DKYLKSSKYIAWPLQGWQATFGGGDHPPKSGGGGSLEVLFQGPDAAQPARRARRTKLAAYARK
AARQARAGGGGSKIPNIGNVMNKFEILGVVGEGAYGVVLKCRHKETHEIVAIKKFKDSEENEE
VKETTLRELKMLRTLKQENIVELKEAFRRRGKLYLVFEYVEKNMLELLEEMPNGVPPEKVKSY
lYQLIKAIHWCHKNDIVHRDIKPENLLISHNDVLKLCDFGFARNLSEGNNANYTEYVATRWYR
SPELLLGAPYGKSVDMWSVGCILGELSDGQPLFPGESEIDQLFTIQKVLGPLPSEQMKLFYSN
PRFHGLRFPAVNHPQSLERRYLGILNSVLLDLMKNLLKLDPADRYLTEQCLNHPTFQTQRLLD
RSPSRSAKRKPYHVESSTLSNRNQAGKSTALQSHHRSNSKDIQNLSVGLPRADEGLPANESFL
NGNLAGASLSPLHTKTYQASSQPGSTSKDLTNNNIPHLLSPKEAKSKTEFDFNIDPKPSEGPG
TKYLKSNSRSQQNRHSFMESSQSKAGTLQPNEKQSRHSYIDTIPQSSRSPSYRTKAKSHGALS
DSKSVSNLSEARAQIAEPSTSRYFPSSCLDLNSPTSPTPTRHSDTRTLLSPSGRNNRNEGTLD
SRRTTTRHSKTMEELKLPEHMDSSHSHSLSAPHESFSYGLGYTSPFSSQQRPHRHSMYVTRDK
VRAKGLDGSLSIGQGMAARANSLQLLSPQPGEQLPPEMTVARSSVKETSREGTSSFHTRQKSE
GGVYHDPHSDDGTAPKENRHLYNDPVPRRVGSFYRVPSPRPDNSFHENNVSTRVSSLPSESSS
GTNHSKRQPAFDPWKSPENISHSEQLKEKEKQGFFRSMKKKKKKSQTVPNSDSPDLLTLQKSI
HSASTPSSRPKEWRPEKISDLQTQSQPLKSLRKLLHLSSASNHPASSDPRFQPLTAQQTKNSF
SEIRIHPLSQASGGSSNIRQEPAPKGRPALQLPGQMDPGWHVSSVTRSATEGPSYSEQLGAKS

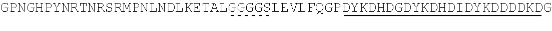

APHHHHHH*
-GST-P-TATκ11-CDKL5_107-P-FH_pVL1393 (insect)
SEQ ID NO: 84
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI
DKYLKSSKYIAWPLQGWQATFGGGDHPPKSGGGGSLEVLFQGPYARKAARQARAGGGGSKIPN
IGNVMNKFEILGVVGEGAYGVVLKCRHKETHEIVAIKKFKDSEENEEVKETTLRELKMLRTLK
QENIVELKEAFRRRGKLYLVFEYVEKNMLELLEEMPNGVPPEKVKSYIYQLIKAIHWCHKNDI
VHRDIKPENLLISHNDVLKLCDFGFARNLSEGNNANYTEYVATRWYRSPELLLGAPYGKSVDM
WSVGCILGELSDGQPLFPGESEIDQLFTIQKVLGPLPSEQMKLFYSNPRFHGLRFPAVNHPQS
LERRYLGILNSVLLDLMKNLLKLDPADRYLTEQCLNHPTFQTQRLLDRSPSRSAKRKPYHVES
STLSNRNQAGKSTALQSHHRSNSKDIQNLSVGLPRADEGLPANESFLNGNLAGASLSPLHTKT
YQASSQPGSTSKDLTNNNIPHLLSPKEAKSKTEFDFNIDPKPSEGPGTKYLKSNSRSQQNRHS
FMESSQSKAGTLQPNEKQSRHSYIDTIPQSSRSPSYRTKAKSHGALSDSKSVSNLSEARAQIA
EPSTSRYFPSSCLDLNSPTSPTPTRHSDTRTLLSPSGRNNRNEGTLDSRRTTTRHSKTMEELK
LPEHMDSSHSHSLSAPHESFSYGLGYTSPFSSQQRPHRHSMYVTRDKVRAKGLDGSLSIGQGM
AARANSLQLLSPQPGEQLPPEMTVARSSVKETSREGTSSFHTRQKSEGGVYHDPHSDDGTAPK
ENRHLYNDPVPRRVGSFYRVPSPRPDNSFHENNVSTRVSSLPSESSSGTNHSKRQPAFDPWKS
PENISHSEQLKEKEKQGFFRSMKKKKKKSQTVPNSDSPDLLTLQKSIHSASTPSSRPKEWRPE
KISDLQTQSQPLKSLRKLLHLSSASNHPASSDPRFQPLTAQQTKNSFSEIRIHPLSQASGGSS
NIRQEPAPKGRPALQLPGQMDPGWHVSSVTRSATEGPSYSEQLGAKSGPNGHPYNRTNRSRMP

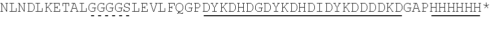

-GST-P-TAT28-CDKL5_107-P-FH_pVL1393 (insect)
SEQ ID NO: 85
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI
DKYLKSSKYIAWPLQGWQATFGGGDHPPKSGGGGSLEVLFQGPDAAQPARRARRTKLAAYGRK
KRRQRRRGGGGSKIPNIGNVMNKFEILGVVGEGAYGVVLKCRHKETHEIVAIKKFKDSEENEE
VKETTLRELKMLRTLKQENIVELKEAFRRRGKLYLVFEYVEKNMLELLEEMPNGVPPEKVKSY
IYQLIKAIHWCHKNDIVHRDIKPENLLISHNDVLKLCDFGFARNLSEGNNANYTEYVATRWYR
SPELLLGAPYGKSVDMWSVGCILGELSDGQPLFPGESEIDQLFTIQKVLGPLPSEQMKLFYSN
PRFHGLRFPAVNHPQSLERRYLGILNSVLLDLMKNLLKLDPADRYLTEQCLNHPTFQTQRLLD
RSPSRSAKRKPYHVESSTLSNRNQAGKSTALQSHHRSNSKDIQNLSVGLPRADEGLPANESFL
NGNLAGASLSPLHTKTYQASSQPGSTSKDLTNNNIPHLLSPKEAKSKTEFDFNIDPKPSEGPG
TKYLKSNSRSQQNRHSFMESSQSKAGTLQPNEKQSRHSYIDTIPQSSRSPSYRTKAKSHGALS
DSKSVSNLSEARAQIAEPSTSRYFPSSCLDLNSPTSPTPTRHSDTRTLLSPSGRNNRNEGTLD
SRRTTTRHSKTMEELKLPEHMDSSHSHSLSAPHESFSYGLGYTSPFSSQQRPHRHSMYVTRDK
VRAKGLDGSLSIGQGMAARANSLQLLSPQPGEQLPPEMTVARSSVKETSREGTSSFHTRQKSE
GGVYHDPHSDDGTAPKENRHLYNDPVPRRVGSFYRVPSPRPDNSFHENNVSTRVSSLPSESSS
GTNHSKRQPAFDPWKSPENISHSEQLKEKEKQGFFRSMKKKKKKSQTVPNSDSPDLLTLQKSI
HSASTPSSRPKEWRPEKISDLQTQSQPLKSLRKLLHLSSASNHPASSDPRFQPLTAQQTKNSF
SEIRIHPLSQASGGSSNIRQEPAPKGRPALQLPGQMDPGWHVSSVTRSATEGPSYSEQLGAKS

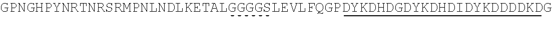

APHHHHHH*
-GST-P-TATil-CDKL5_107-P-FH_pVL1393 (insect)
SEQ ID NO: 86
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI
DKYLKSSKYIAWPLQGWQATFGGGDHPPKSGGGGSLEVLFQGPYGRKKRRQRRRGGGGSKIPN
IGNVMNKFEILGVVGEGAYGVVLKCRHKETHEIVAIKKFKDSEENEEVKETTLRELKMLRTLK
QENIVELKEAFRRRGKLYLVFEYVEKNMLELLEEMPNGVPPEKVKSYIYQLIKAIHWCHKNDI
VHRDIKPENLLISHNDVLKLCDFGFARNLSEGNNANYTEYVATRWYRSPELLLGAPYGKSVDM
WSVGCILGELSDGQPLFPGESEIDQLFTIQKVLGPLPSEQMKLEYSNPRFHGLRFPAVNHPQS
LERRYLGILNSVLLDLMKNLLKLDPADRYLTEQCLNHPTFQTQRLLDRSPSRSAKRKPYHVES
STLSNRNQAGKSTALQSHHRSNSKDIQNLSVGLPRADEGLPANESFLNGNLAGASLSPLHTKT
YQASSQPGSTSKDLTNNNIPHLLSPKEAKSKTEFDFNIDPKPSEGPGTKYLKSNSRSQQNRHS
FMESSQSKAGTLQPNEKQSRHSYIDTIPQSSRSPSYRTKAKSHGALSDSKSVSNLSEARAQIA
EPSTSRYFPSSCLDLNSPTSPTPTRHSDTRTLLSPSGRNNRNEGTLDSRRTTTRHSKTMEELK
LPEHMDSSHSHSLSAPHESFSYGLGYTSPFSSQQRPHRHSMYVTRDKVRAKGLDGSLSIGQGM
AARANSLQLLSPQPGEQLPPEMTVARSSVKETSREGTSSFHTRQKSEGGVYHDPHSDDGTAPK
ENRHLYNDPVPRRVGSFYRVPSPRPDNSFHENNVSTRVSSLPSESSSGTNHSKRQPAFDPWKS
PENISHSEQLKEKEKQGFFRSMKKKKKKSQTVPNSDSPDLLTLQKSIHSASTPSSRPKEWRPE
KISDLQTQSQPLKSLRKLLHLSSASNHPASSDPRFQPLTAQQTKNSFSEIRIHPLSQASGGSS
NIRQEPAPKGRPALQLPGQMDPGWHVSSVTRSATEGPSYSEQLGAKSGPNGHPYNRTNRSRMP

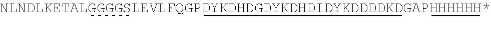

-GST-P-ANTP-CDKL5_107-P-FH_pVL1393 (insect)
SEQ ID NO: 87
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI
DKYLKSSKYIAWPLQGWQATFGGGDHPPKSGGGGSLEVLFQGPRQIKIWFQNRRMKWKKGGGG
SKIPNIGNVMNKFEILGVVGEGAYGVVLKCRHKETHEIVAIKKFKDSEENEEVKETTLRELKM
LRTLKQENIVELKEAFRRRGKLYLVFEYVEKNMLELLEEMPNGVPPEKVKSYIYQLIKAIHWC
HKNDIVHRDIKPENLLISHNDVLKLCDFGFARNLSEGNNANYTEYVATRWYRSPELLLGAPYG
KSVDMWSVGCILGELSDGQPLFPGESEIDQLFTIQKVLGPLPSEQMKLFYSNPRFHGLRFPAV
NHPQSLERRYLGILNSVLLDLMKNLLKLDPADRYLTEQCLNHPTFQTQRLLDRSPSRSAKRKP
YHVESSTLSNRNQAGKSTALQSHHRSNSKDIQNLSVGLPRADEGLPANESFLNGNLAGASLSP
LHTKTYQASSQPGSTSKDLTNNNIPHLLSPKEAKSKTEFDFNIDPKPSEGPGTKYLKSNSRSQ
QNRHSFMESSQSKAGTLQPNEKQSRHSYIDTIPQSSRSPSYRTKAKSHGALSDSKSVSNLSEA
RAQIAEPSTSRYFPSSCLDLNSPTSPTPTRHSDTRTLLSPSGRNNRNEGTLDSRRTTTRHSKT
MEELKLPEHMDSSHSHSLSAPHESFSYGLGYTSPFSSQQRPHRHSMYVTRDKVRAKGLDGSLS
IGQGMAARANSLQLLSPQPGEQLPPEMTVARSSVKETSREGTSSFHTRQKSEGGVYHDPHSDD
GTAPKENRHLYNDPVPRRVGSFYRVPSPRPDNSFHENNVSTRVSSLPSESSSGTNHSKRQPAF
DPWKSPENISHSEQLKEKEKQGFFRSMKKKKKKSQTVPNSDSPDLLTLQKSIHSASTPSSRPK
EWRPEKISDLQTQSQPLKSLRKLLHLSSASNHPASSDPRFQPLTAQQTKNSFSEIRIHPLSQA
SGGSSNIRQEPAPKGRPALQLPGQMDPGWHVSSVTRSATEGPSYSEQLGAKSGPNGHPYNRTN

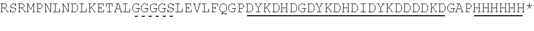

-GST-P-TRANSP-CDKL5_107-P-FH_pVL1393 (insect)
SEQ ID NO: 88
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI
DKYLKSSKYIAWPLQGWQATFGGGDHPPKSGGGGSLEVLFQGPAGYLLGKINLKALAALAKKI
LGGGGSKIPNIGNVMNKFEILGVVGEGAYGVVLKCRHKETHEIVAIKKFKDSEENEEVKETTL
RELKMLRTLKQENIVELKEAFRRRGKLYLVFEYVEKNMLELLEEMPNGVPPEKVKSYIYQLIK
AIHWCHKNDIVHRDIKPENLLISHNDVLKLCDFGFARNLSEGNNANYTEYVATRWYRSPELLL
GAPYGKSVDMWSVGCILGELSDGQPLFPGESEIDQLFTIQKVLGPLPSEQMKLFYSNPRFHGL
RFPAVNHPQSLERRYLGILNSVLLDLMKNLLKLDPADRYLTEQCLNHPTFQTQRLLDRSPSRS
AKRKPYHVESSTLSNRNQAGKSTALQSHHRSNSKDIQNLSVGLPRADEGLPANESFLNGNLAG
ASLSPLHTKTYQASSQPGSTSKDLTNNNIPHLLSPKEAKSKTEFDFNIDPKPSEGPGTKYLKS
NSRSQQNRHSFMESSQSKAGTLQPNEKQSRHSYIDTIPQSSRSPSYRTKAKSHGALSDSKSVS
NLSEARAQIAEPSTSRYFPSSCLDLNSPTSPTPTRHSDTRTLLSPSGRNNRNEGTLDSRRTTT
RHSKTMEELKLPEHMDSSHSHSLSAPHESFSYGLGYTSPFSSQQRPHRHSMYVTRDKVRAKGL
DGSLSIGQGMAARANSLQLLSPQPGEQLPPEMTVARSSVKETSREGTSSFHTRQKSEGGVYHD
PHSDDGTAPKENRHLYNDPVPRRVGSFYRVPSPRPDNSFHENNVSTRVSSLPSESSSGTNHSK
RQPAFDPWKSPENISHSEQLKEKEKQGFFRSMKKKKKKSQTVPNSDSPDLLTLQKSIHSASTP
SSRPKEWRPEKISDLQTQSQPLKSLRKLLHLSSASNHPASSDPRFQPLTAQQTKNSFSEIRIH
PLSQASGGSSNIRQEPAPKGRPALQLPGQMDPGWHVSSVTRSATEGPSYSEQLGAKSGPNGHP

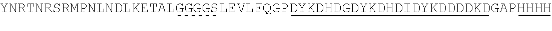

HH*
-GST-P-P97P-CDKL5_107-P-FH_pVL1393 (insect)
SEQ ID NO: 89
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI
DKYLKSSKYIAWPLQGWQATFGGGDHPPKSGGGGSLEVLFQGPDSSHAFTLDELRGGGGSKIP
NIGNVMNKFEILGVVGEGAYGVVLKCRHKETHEIVAIKKFKDSEENEEVKETTLRELKMLRTL
KQENIVELKEAFRRRGKLYLVFEYVEKNMLELLEEMPNGVPPEKVKSYIYQLIKAIHWCHKND
IVHRDIKPENLLISHNDVLKLCDFGFARNLSEGNNANYTEYVATRWYRSPELLLGAPYGKSVD
MWSVGCILGELSDGQPLFPGESEIDQLFTIQKVLGPLPSEQMKLFYSNPRFHGLRFPAVNHPQ
SLERRYLGILNSVLLDLMKNLLKLDPADRYLTEQCLNHPTFQTQRLLDRSPSRSAKRKPYHVE
SSTLSNRNQAGKSTALQSHHRSNSKDIQNLSVGLPRADEGLPANESFLNGNLAGASLSPLHTK
TYQASSQPGSTSKDLTNNNIPHLLSPKEAKSKTEFDFNIDPKPSEGPGTKYLKSNSRSQQNRH
SFMESSQSKAGTLQPNEKQSRHSYIDTIPQSSRSPSYRTKAKSHGALSDSKSVSNLSEARAQI
AEPSTSRYFPSSCLDLNSPTSPTPTRHSDTRTLLSPSGRNNRNEGTLDSRRTTTRHSKTMEEL
KLPEHMDSSHSHSLSAPHESFSYGLGYTSPFSSQQRPHRHSMYVTRDKVRAKGLDGSLSIGQG
MAARANSLQLLSPQPGEQLPPEMTVARSSVKETSREGTSSFHTRQKSEGGVYHDPHSDDGTAP
KENRHLYNDPVPRRVGSFYRVPSPRPDNSFHENNVSTRVSSLPSESSSGTNHSKRQPAFDPWK
SPENISHSEQLKEKEKQGFFRSMKKKKKKSQTVPNSDSPDLLTLQKSIHSASTPSSRPKEWRP
EKISDLQTQSQPLKSLRKLLHLSSASNHPASSDPRFQPLTAQQTKNSFSEIRIHPLSQASGGS
SNIRQEPAPKGRPALQLPGQMDPGWHVSSVTRSATEGPSYSEQLGAKSGPNGHPYNRTNRSRM

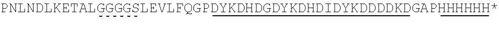

-GST-P-eGFP-P-FH_pVL1393 (insect)
SEQ ID NO: 90
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI

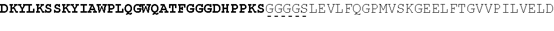

GDVNGHKFSVSGEGEGDATYGKLTLKFICTTGKLPVPWPTLVTTLTYGVQCFSRYPDHMKQHD
FFKSAMPEGYVQERTIFFKDDGNYKTRAEVKFEGDTLVNRIELKGIDFKEDGNILGHKLEYNY
NSHNVYIMADKQKNGIKVNFKIRHNIEDGSVQLADHYQQNTPIGDGPVLLPDNHYLSTQSALS

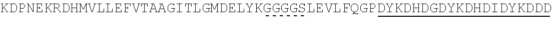

DKDGAPHHHHHH*
-GST-P-TATκ28-eGFP-P-FH_pVL1393 (insect)
SEQ ID NO: 91
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI

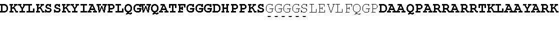

AARQARAGGGGSMVSKGEELFTGVVPILVELDGDVNGHKFSVSGEGEGDATYGKLTLKFICTT
GKLPVPWPTLVTTLTYGVQCFSRYPDHMKQHDFFKSAMPEGYVQERTIFFKDDGNYKTRAEVK
FEGDTLVNRIELKGIDFKEDGNILGHKLEYNYNSHNVYIMADKQKNGIKVNFKIRHNIEDGSV

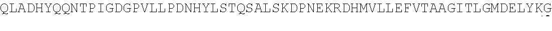

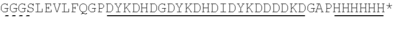


-GST-P-CDKL5_107-P-FH_pVL1393 (insect)
SEQ ID NO: 92
MSPILGYWKIKGLVQPTRLLLEYLEEKYEEHLYERDEGDKWRNKKFELGLEFPNLPYYIDGDV
KLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIPQI
DKYLKSSKYIAWPLQGWQATFGGGDHPPKSGGGGSLEVLFQGPGKIPNIGNVMNKFEILGVVG
EGAYGVVLKCRHKETHEIVAIKKFKDSEENEEVKETTLRELKMLRTLKQENIVELKEAFRRRG
KLYLVFEYVEKNMLELLEEMPNGVPPEKVKSYIYQLIKAIHWCHKNDIVHRDIKPENLLISHN
DVLKLCDFGFARNLSEGNNANYTEYVATRWYRSPELLLGAPYGKSVDMWSVGCILGELSDGQP
LFPGESEIDQLFTIQKVLGPLPSEQMKLFYSNPRFHGLRFPAVNHPQSLERRYLGILNSVLLD
LMKNLLKLDPADRYLTEQCLNHPTFQTQRLLDRSPSRSAKRKPYHVESSTLSNRNQAGKSTAL
QSHHRSNSKDIQNLSVGLPRADEGLPANESFLNGNLAGASLSPLHTKTYQASSQPGSTSKDLT
NNNIPHLLSPKEAKSKTEFDFNIDPKPSEGPGTKYLKSNSRSQQNRHSFMESSQSKAGTLQPN
EKQSRHSYIDTIPQSSRSPSYRTKAKSHGALSDSKSVSNLSEARAQIAEPSTSRYFPSSCLDL
NSPTSPTPTRHSDTRTLLSPSGRNNRNEGTLDSRRTTTRHSKTMEELKLPEHMDSSHSHSLSA
PHESFSYGLGYTSPFSSQQRPHRHSMYVTRDKVRAKGLDGSLSIGQGMAARANSLQLLSPQPG
EQLPPEMTVARSSVKETSREGTSSFHTRQKSEGGVYHDPHSDDGTAPKENRHLYNDPVPRRVG
SFYRVPSPRPDNSFHENNVSTRVSSLPSESSSGTNHSKRQPAFDPWKSPENISHSEQLKEKEK
QGFFRSMKKKKKKSQTVPNSDSPDLLTLQKSIHSASTPSSRPKEWRPEKISDLQTQSQPLKSL
RKLLHLSSASNHPASSDPRFQPLTAQQTKNSFSEIRIHPLSQASGGSSNIRQEPAPKGRPALQ

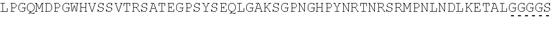

LEVLFQGPDYKDHDGDYKDHDIDYKDDDDKDGAPHHHHHH*
MBip-TATκ28-CDKL5_107-FH_cho[1-7NQ]
SEQ ID NO: 93
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

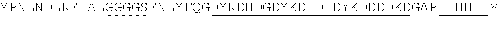

MBip-TATκ28-CDKL5_107-FH_cho[2-7NQ]
SEQ ID NO: 94
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARNLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

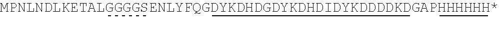

MBip-TATκ28-CDKL5_107-FH_cho[1,3-7NQ]
SEQ ID NO: 95
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNANYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

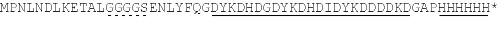

MBip-TATκ28-CDKL5_107-FH_cho[1-2,4-7NQ]
SEQ ID NO: 96
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQNLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

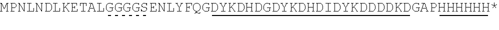

MBip-TATκ28-CDKL5_107-FH_cho[1-3,5-7NQ]
SEQ ID NO: 97
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSNLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

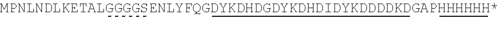

MBip-TATκ28-CDKL5_l07-FH_cho[1-4,6-7NQ]
SEQ ID NO: 98
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPENISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

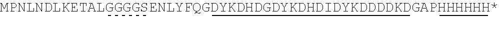

MBip-TATκ28-CDKL5_107-FH_cho[1-5,7NQ]
SEQ ID NO: 99
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYNRTQRSR

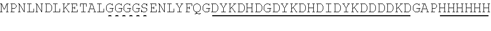

MBip-TATκ28-CDKL5_107-FH_cho[1-6NQ]
SEQ ID NO: 100
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTNRSR

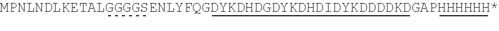

MBip-TATκ28-CDKL5_107-FH_cho[2NQ]
SEQ ID NO: 101
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARNLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQNLSVGLPRADEGLPANESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSNLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHENNVSTRVSSLPSESSSGTNHSKRQPAFDPW
KSPENISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYNRTNRSR

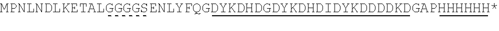

MBip-TATκ28-CDKL5_107-FH_cho[1-10NQ]
SEQ ID NO: 102
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPA [symbol] ESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHEN [symbol] VSTRVSSLPSESSSGT [symbol] HSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

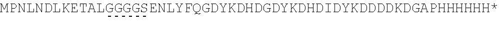

MBip-TATκ28-CDKL5_l07-FH_cho[1-7,9-10NQ]
SEQ ID NO: 103
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPA [symbol] ESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHEN [symbol] VSTRVSSLPSESSSGT [symbol] HSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

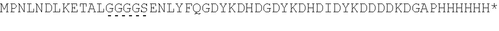

MBip-TATκ28-CDKL5_l07-FH_cho[1-8,10NQ]
SEQ ID NO: 104
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPA [symbol] ESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHEN [symbol] VSTRVSSLPSESSSGT [symbol] HSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

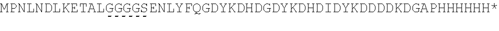

MBip-TATκ28-CDKL5_107-FH_cho[1-9NQ]
SEQ ID NO: 105
MKLSLVAAMLLLLSLVAAMLLLLSAARAGDAAQPARRARRTKLAAYARKAARQARAGGGGSKI
PNIGNVMNKFEILGVVGEGAYGVVLKCRHKETHEIVAIKKFKDSEENEEVKETTLRELKMLRT
LKQENIVELKEAFRRRGKLYLVFEYVEKNMLELLEEMPNGVPPEKVKSYIYQLIKAIHWCHKN
DIVHRDIKPENLLISHNDVLKLCDFGFARQLSEGNNAQYTEYVATRWYRSPELLLGAPYGKSV
DMWSVGCILGELSDGQPLFPGESEIDQLFTIQKVLGPLPSEQMKLFYSNPRFHGLRFPAVNHP
QSLERRYLGILNSVLLDLMKNLLKLDPADRYLTEQCLNHPTFQTQRLLDRSPSRSAKRKPYHV
ESSTLSNRNQAGKSTALQSHHRSNSKDIQQLSVGLPRADEGLPA [symbol] ESFLNGNLAGASLSPLHT
KTYQASSQPGSTSKDLTNNNIPHLLSPKEAKSKTEFDFNIDPKPSEGPGTKYLKSNSRSQQNR
HSFMESSQSKAGTLQPNEKQSRHSYIDTIPQSSRSPSYRTKAKSHGALSDSKSVSQLSEARAQ
IAEPSTSRYFPSSCLDLNSPTSPTPTRHSDTRTLLSPSGRNNRNEGTLDSRRTTTRHSKTMEE
LKLPEHMDSSHSHSLSAPHESFSYGLGYTSPFSSQQRPHRHSMYVTRDKVRAKGLDGSLSIGQ
GMAARANSLQLLSPQPGEQLPPEMTVARSSVKETSREGTSSFHTRQKSEGGVYHDPHSDDGTA
PKENRHLYNDPVPRRVGSFYRVPSPRPDNSFHEN [symbol] VSTRVSSLPSESSSGT [symbol] HSKRQPAFDPW
KSPEQISHSEQLKEKEKQGFFRSMKKKKKKSQTVPNSDSPDLLTLQKSIHSASTPSSRPKEWR
PEKISDLQTQSQPLKSLRKLLHLSSASNHPASSDPRFQPLTAQQTKNSFSEIRIHPLSQASGG
SSNIRQEPAPKGRPALQLPGQMDPGWHVSSVTRSATEGPSYSEQLGAKSGPNGHPYQRTQRSR

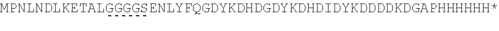

AAVC-CBh-hCDKL5-107
L-ITR: 1-141
CBh promoter: 159-976
hCDKL5-107 ORF: 1000-2885
bGHp(A): 3910-4137
R-ITR: 4149-4289
Amp(R): 5206-6063
pUC origin: 6214-6881
SEQ ID NO: 106
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGTT AATAGTAATC AATTACGGGG TCATTAGTTC ATAGGCCATA
  201 TATGGAGTTC CGCGTTACAT AACTTACGGT AAATGGCCCG CCTGGCTGAC
  251 CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG
  351 GTAAACTGCC CACTTGGCAG TACATCAAGT GTATCATATG CCAAGTACGC
  401 CCCCTATTGA CGTCAATGAC GGTAAATGGC CCGCCTGGCA TTATGCCCAG
  451 TACATGACCT TACGGGACTT TCCTACTTGG CAGTACATCT CCACGTTCTG
  501 CTTCAGTCTC CCCATCTCCC CCCCCTCCCC ACCCCCAATT TTGTATTTAT
  551 TTATTTTTTA ATTATTTTGT GCAGCGATGG GGGCGGGGGG GGGGGGGGCG
  601 CGCGCCAGGC GGGGCGGGGC GGGGCGAGGG GCGGGGCGGG GCGAGGCGGA
  651 GAGGTGCGGC GGCAGCCAAT CAGAGCGGCG CGCTCCGAAA GTTTCCTTTT
  701 ATGGCGAGGC GGCGGCGGCG GCGGCCCTAT AAAAAGCGAA GCGCGCGGCG
  751 GGGAGTCGCT GCGTTGCCTT CGCCCCGTGC CCCGCTCCGC GCCGCCTCGC
  801 GCCGCCCGCC CCGGCTCTGA CTGACCGCGT TACTCCCACA GGTGAGCGGG
  851 CGGGACGGCC CTTCTCCTCC GGGCTGTAAT TAGCAAGAGG TAAGGGTTTA
  901 AGGGATGGTT GGTTGGTGGG GTATTAATGT TTAATTACCT GTTTTACAGG
  951 CCTGAAATCA CTTGGTTTTA GGTTGGGCTA GCCAAAGCTT GCCGCCACCA
1001 TGAAAATCCC TAACATTGGT AACGTGATGA ACAAGTTTGA AATCCTCGGG
1051 GTCGTCGGAG AAGGTGCCTA CGGGGTCGTG CTGAAGTGCA GACACAAGGA
1101 GACACACGAG ATCGTGGCCA TCAAGAAGTT CAAGGATAGC GAGGAGAACG
1151 AGGAGGTGAA GGAGACAACC CTGAGAGAGC TGAAGATGCT GCGGACACTG
1201 AAGCAGGAGA ACATCGTGGA GCTGAAGGAG GCTTTCAGGA GACGGGGAAA
1251 GCTGTACCTG GTGTTTGAGT ACGTGGAGAA GAACATGCTG GAGCTGCTGG
1301 AGGAGATGCC TAACGGCGTG CCCCCTGAGA AGGTGAAGTC CTACATCTAC
1351 CAGCTGATCA AGGCCATCCA CTGGTGCCAC AAGAACGACA TCGTGCACAG
1401 AGATATCAAG CCAGAGAACC TGCTGATCTC CCACAACGAC GTGCTGAAGC
1451 TGTGCGATTT CGGCTTTGCC CGGAACCTGA GCGAGGGAAA CAACGCCAAC
1501 TACACAGAGT ACGTGGCTAC CAGATGGTAC CGGAGCCCAG AGCTGCTGCT
1551 GGGAGCTCCA TACGGAAAGA GCGTGGACAT GTGGTCCGTG GGCTGCATCC
1601 TGGGAGAGCT GTCTGACGGC CAGCCTCTGT TCCCAGGAGA GAGCGAGATC
1651 GATCAGCTGT TTACCATCCA GAAGGTGCTG GGCCCTCTGC CAAGCGAGCA
1701 GATGAAGCTG TTCTACTCCA ACCCTAGATT CCACGGACTG CGGTTTCCCG
1751 CCGTGAACCA CCCTCAGAGC CTGGAGCGCA GGTACCTGGG CATCCTGAAC
1801 TCCGTGCTGC TGGATCTGAT GAAGAACCTG CTGAAGCTGG ACCCCGCCGA
1851 TAGATACCTG ACCGAGCAGT GTCTGAACCA CCCTACATTT CAGACCCAGC
1901 GCCTGCTGGA CAGGAGCCCT TCCAGATCTG CTAAGCGGAA GCCATACCAC
1951 GTGGAGAGCT CCACCCTGTC CAACAGAAAC CAGGCCGGCA AGTCTACAGC
2001 TCTGCAGAGC CACCACCGGA GCAACTCCAA GGACATCCAG AACCTGTCTG
2051 TGGGCCTGCC TAGGGCTGAT GAGGGACTGC CAGCTAACGA GAGCTTCCTG
2101 AACGGCAACC TGGCCGGAGC TTCTCTGAGC CCACTGCACA CAAAGACCTA
2151 CCAGGCCTCT AGCCAGCCCG GCTCCACATC TAAGGACCTG ACCAACAACA
2201 ACATCCCACA CCTGCTGTCT CCCAAGGAGG CTAAGAGCAA GACCGAGTTC
2251 GACTTTAACA TCGATCCCAA GCCTAGCGAG GGCCCAGGAA CAAAGTACCT
2301 GAAGAGCAAC TCCCGCTCTC AGCAGAACAG GCACTCCTTC ATGGAGTCCT
2351 CTCAGTCTAA GGCCGGCACC CTGCAGCCCA ACGAGAAGCA GAGCAGGCAC
2401 TCCTACATCG ATACCATCCC CCAGAGCTCC AGAAGCCCTT CCTACCGGAC
2451 AAAGGCCAAG AGCCACGGCG CTCTGTCTGA CAGCAAGTCC GTGTCTAACC
2501 TGTCCGAGGC TAGGGCTCAG ATCGCTGAGC CAAGCACCTC CAGGTACTTT
2551 CCTTCTAGCT GTCTGGACCT GAACTCTCCT ACAAGCCCAA CACCCACCCG
2601 CCACTCCGAT ACAAGGACCC TGCTGTCTCC AAGCGGCAGG AACAACAGGA
2651 AGGAGGGAAC CCTGGATTCT AGACGGACCA CAACCCGCCA CAGCAAGACA
2701 ATGGAGGAGC TGAAGCTGCC AGAGCACATG GACTCCTCTC ACTCCCACTC
2751 TCTGAGCGCC CCCCACGAGT CCTTCTCTTA CGGCCTGGGA TACACCTCCC
2801 CCTTCAGGTC CCAGCAGAGG CCCCACAGGC ACTCTATGTA CGTGACACGC
2851 GACAAGGTGA GGGCCAAGGG CCTGGATGGA AGCCTGTCCA TCGGACAGGG
2901 AATGGCTGCT AGGGCTAACT CCCTGCAGCT GCTGTCTCCT CAGCCAGGAG
2951 AGCAGCTGCC ACCAGAGATG ACCGTGGCTC GCTCTAGCGT GAAGGAGACA
3001 AGCAGGGAGG GCACCTCCTC TTTCCACACA CGCCAGAAGT CCGAGGGCGG
3051 AGTGTACCAC GACCCCCACT CTGACGATGG AACAGCTCCT AAGGAGAACA
3101 GGCACCTGTA CAACGATCCC GTGCCTCGCA GGGTGGGCTC CTTCTACAGA
3151 GTGCCATCTC CCCGGCCTGA CAACAGCTTT CACGAGAACA ACGTGTCCAC
3201 CCGCGTGAGC TCCCTGCCTT CTGAGTCTAG CTCCGGAACA AACCACTCTA
3251 AGAGGCAGCC CGCCTTTGAC CCTTGGAAGA GCCCAGAGAA CATCTCTCAC
3301 AGCGAGCAGC TGAAGGAGAA GGAGAAGCAG GGCTTCTTTC GCAGCATGAA
3351 GAAGAAGAAG AAGAAGAGCC AGACCGTGCC TAACTCCGAC TCTCCAGATC
3401 TGCTGACCCT GCAGAAGTCC ATCCACAGCG CCTCCACACC ATCTAGCCGC
3451 CCTAAGGAGT GGAGGCCTGA GAAGATCAGC GATCTGCAGA CACAGAGCCA
3501 GCCACTGAAG TCCCTGAGGA AGCTGCTGCA CCTGTCCTCT GCCAGCAACC
3551 ACCCCGCTAG CTCCGACCCA AGATTCCAGC CCCTGACAGC CCAGCAGACC
3601 AAGAACTCTT TTAGCGAGAT CCGGATCCAC CCTCTGTCCC AGGCTTCTGG
3651 CGGATCTAGC AACATCAGAC AGGAGCCAGC TCCAAAGGGC CGGCCCGCTC
3701 TGCAGCTGCC TGGCCAGATG GACCCAGGAT GGCACGTGTC CTCTGTGACA
3751 AGATCCGCCA CCGAGGGACC ATCCTACTCT GAGCAGCTGG GCGCTAAGTC
3801 TGGCCCTAAC GGACACCCAT ACAATAGGAC TAATAGAAGC AGAATGCCAA
3851 ACCTCAATGA CCTCAAGGAA ACAGCACTCT GATAAGCGGC CGCAACTCGA
3901 GACTCTAGAC GACTGTGCCT TCTAGTTGCC AGCCATCTGT TGTTTGCCCC
3951 TCCCCCGTGC CTTCCTTGAC CCTGGAAGGT GCCACTCCCA CTGTCCTTTC
4001 CTAATAAAAT GAGGAAATTG CATCGCATTG TCTGAGTAGG TGTCATTCTA
4051 TTCTGGGGGG TGGGGTGGGG CAGGACAGCA AGGGGGAGGA TTGGGAAGAC
4101 AATAGCAGGC ATGCTGGGGA TGCGGTGGGC TCTATGGCCG CGGGCCGCAG
4151 GAACCCCTAG TGATGGAGTT GGCCACTCCC TCTCTGCGCG CTCGCTCGCT
4201 CACTGAGGCC GGGCGACCAA AGGTCGCCCG ACGCCCGGGC TTTGCCCGGG
4251 CGGCCTCAGT GAGCGAGCGA GCGCGCAGCT GCCTGCAGGG GCGCCTGATG
4301 CGGTATTTTC TCCTTACGCA TCTGTGCGGT ATTTCACACC GCATACGTCA
4351 AAGCAACCAT AGTACGCGCC CTGTAGCGGC GCATTAAGCG CGGCGGGTGT
4401 GGTGGTTACG CGCAGCGTGA CCGCTACACT TGCCAGCGCC CTAGCGCCCG
4451 CTCCTTTCGC TTTCTTCCCT TCCTTTCTCG CCACGTTCGC CGGCTTTCCC
4501 CGTCAAGCTC TAAATCGGGG GCTCCCTTTA GGGTTCCGAT TTAGTGCTTT
4551 ACGGCACCTC GACCCCAAAA AACTTGATTT GGGTGATGGT TCACGTAGTG
4601 GGCCATCGCC CTGATAGACG GTTTTTCGCC CTTTGACGTT GGAGTCCACG
4651 TTCTTTAATA GTGGACTCTT GTTCCAAACT GGAACAACAC TCAACCCTAT
4701 CTCGGGCTAT TCTTTTGATT TATAAGGGAT TTTGCCGATT TCGGCCTATT
4751 GGTTAAAAAA TGAGCTGATT TAACAAAAAT TTAACGCGAA TTTTAACAAA
4801 ATATTAACGT TTACAATTTT ATGGTGCACT CTCAGTACAA TCTGCTCTGA
4851 TGCCGCATAG TTAAGCCAGC CCCGACACCC GCCAACACCC GCTGACGCGC
4901 CCTGACGGGC TTGTCTGCTC CCGGCATCCG CTTACAGACA AGCTGTGACC
4951 GTCTCCGGGA GCTGCATGTG TCAGAGGTTT TCACCGTCAT CACCGAAACG
5001 CGCGAGACGA AAGGGCCTCG TGATACGCCT ATTTTTATAG GTTAATGTCA
5051 TGATAATAAT GGTTTCTTAG ACGTCAGGTG GCACTTTTCG GGGAAATGTG
5101 CGCGGAACCC CTATTTGTTT ATTTTTCTAA ATACATTCAA ATATGTATCC
5151 GCTCATGAGA CAATAACCCT GATAAATGCT TCAATAATAT TGAAAAAGGA
5201 AGAGTATGAG TATTCAACAT TTCCGTGTCG CCCTTATTCC CTTTTTTGCG
5251 GCATTTTGCC TTCCTGTTTT TGCTCACCCA GAAACGCTGG TGAAAGTAAA
5301 AGATGCTGAA GATCAGTTGG GTGCACGAGT GGGTTACATC GAACTGGATC
5351 TCAACAGCGG TAAGATCCTT GAGAGTTTTC GCCCCGAAGA ACGTTTTCCA
5401 ATGATGAGCA CTTTTAAAGT TCTGCTATGT GGCGCGGTAT TATCCCGTAT
5451 TGACGCCGGG CAAGAGCAAC TCGGTCGCCG CATACACTAT TCTCAGAATG
5501 ACTTGGTTGA GTACTCACCA GTCACAGAAA AGCATCTTAC GGATGGCATG
5551 ACAGTAAGAG AATTATGCAG TGCTGCCATA ACCATGAGTG ATAACACTGC
5601 GGCCAACTTA CTTCTGACAA CGATCGGAGG ACCGAAGGAG CTAACCGCTT
5651 TTTTGCACAA CATGGGGGAT CATGTAACTC GCCTTGATCG TTGGGAACCG
5701 GAGCTGAATG AAGCCATACC AAACGACGAG CGTGACACCA CGATGCCTGT
5751 AGCAATGGCA ACAACGTTGC GCAAACTATT AACTGGCGAA CTACTTACTC
5801 TAGCTTCCCG GCAACAATTA ATAGACTGGA TGGAGGCGGA TAAAGTTGCA
5851 GGACCACTTC TGCGCTCGGC CCTTCCGGCT GGCTGGTTTA TTGCTGATAA
5901 ATCTGGAGCC GGTGAGCGTG GGTCTCGCGG TATCATTGCA GCACTGGGGC
5951 CAGATGGTAA GCCCTCCCGT ATCGTAGTTA TCTACACGAC GGGGAGTCAG
6001 GCAACTATGG ATGAACGAAA TAGACAGATC GCTGAGATAG GTGCCTCACT
6051 GATTAAGCAT TGGTAACTGT CAGACCAAGT TTACTCATAT ATACTTTAGA
6101 TTGATTTAAA ACTTCATTTT TAATTTAAAA GGATCTAGGT GAAGATCCTT
6151 TTTGATAATC TCATGACCAA AATCCCTTAA CGTGAGTTTT CGTTCCACTG
6201 AGCGTCAGAC CCCGTAGAAA AGATCAAAGG ATCTTCTTGA GATCCTTTTT
6251 TTCTGCGCGT AATCTGCTGC TTGCAAACAA AAAAACCACC GCTACCAGCG
6301 GTGGTTTGTT TGCCGGATCA AGAGCTACCA ACTCTTTTTC CGAAGGTAAC
6351 TGGCTTCAGC AGAGCGCAGA TACCAAATAC TGTCCTTCTA GTGTAGCCGT
6401 AGTTAGGCCA CCACTTCAAG AACTCTGTAG CACCGCCTAC ATACCTCGCT
6451 CTGCTAATCC TGTTACCAGT GGCTGCTGCC AGTGGCGATA AGTCGTGTCT
6501 TACCGGGTTG GACTCAAGAC GATAGTTACC GGATAAGGCG CAGCGGTCGG
6551 GCTGAACGGG GGGTTCGTGC ACACAGCCCA GCTTGGAGCG AACGACCTAC
6601 ACCGAACTGA GATACCTACA GCGTGAGCTA TGAGAAAGCG CCACGCTTCC
6651 CGAAGGGAGA AAGGCGGACA GGTATCCGGT AAGCGGCAGG GTCGGAACAG
6701 GAGAGCGCAC GAGGGAGCTT CCAGGGGGAA ACGCCTGGTA TCTTTATAGT
6751 CCTGTCGGGT TTCGCCACCT CTGACTTGAG CGTCGATTTT TGTGATGCTC
6801 GTCAGGGGGG CGGAGCCTAT GGAAAAACGC CAGCAACGCG GCCTTTTTAC
6851 GGTTCCTGGC CTTTTGCTGG CCTTTTGCTC ACATGT
L-ITR: 1-141
CBh promoter: 159-976
hCDKL5-107 (dead Kinase) ORF: 1000-2885
bGHp(A): 3910-4137
R-ITR: 4149-4289
Amp(R): 5206-6063
pUC origin: 6214-6881
SEQ ID NO: 107
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGTT AATAGTAATC AATTACGGGG TCATTAGTTC ATAGCCCATA
  201 TATGGAGTTC CGCGTTACAT AACTTACGGT AAATGGCCCG CCTGGCTGAC
  251 CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG
  351 GTAAACTGCC CACTTGGCAG TACATCAAGT GTATCATATG CCAAGTACGC
  401 CCCCTATTGA CGTCAATGAC GGTAAATGGC CCGCCTGGCA TTATGCCCAG
  451 TACATGACCT TACGGGACTT TCCTACTTGG CAGTACATCT CCACGTTCTG
  501 CTTCAGTCTC CCCATCTCCC CCCCCTCCCC ACCCCCAATT TTGTATTTAT
  551 TTATTTTTTA ATTATTTTGT GCAGCGATGG GGGCGGGGGG GGGGGGGGCG
  601 CGCGCCAGGC GGGGCGGGGC GGGGCGAGGG GCGGGGCGGG GCGAGGCGGA
  651 GAGGTGCGGC GGCAGCCAAT CAGAGCGGCG CGCTCCGAAA GTTTCCTTTT
  701 ATGGCGAGGC GGCGGCGGCG GCGGCCCTAT AAAAAGCGAA GCGCGCGGCG
  751 GGGAGTCGCT GCGTTGCCTT CGCCCCGTGC CCCGCTCCGC GCCGCCTCGC
  801 GCCGCCCGCC CCGGCTCTGA CTGACCGCGT TACTCCCACA GGTGAGCGGG
  851 CGGGACGGCC CTTCTCCTCC GGGCTGTAAT TAGCAAGAGG TAAGGGTTTA
  901 AGGGATGGTT GGTTGGTGGG GTATTAATGT TTAATTACCT GTTTTACAGG
  951 CCTGAAATCA CTTGGTTTTA GGTTGGGCTA GCCAAAGCTT GCCGCCACCA
1001 TGAAAATCCC TAATATCGGA AATGTGATGA ATAAGTTTGA AATCCTCGGG
1051 GTCGTCGGAG AAGGTGCCTA CGGGGTCGTC CTGAAATGCA GACACAAGGA
1101 GACACACGAG ATCGTGGCCA TCAGGAGATT CAAGGATAGC GAGGAGAACG
1151 AGGAGGTGAA GGAGACAACC CTGCGCGAGC TGAAGATGCT GAGGACACTG
1201 AAGCAGGAGA ACATCGTGGA GCTGAAGGAG GCTTTCCGGC GCAGGGGAAA
1251 GCTGTACCTG GTGTTTGAGT ACGTGGAGAA GAACATGCTG GAGCTGCTGG
1301 AGGAGATGCC TAACGGCGTG CCCCCTGAGA AGGTGAAGTC CTACATCTAC
1351 CAGCTGATCA AGGCCATCCA CTGGTGCCAC AAGAACGACA TCGTGCACCG
1401 CGATATCAAG CCAGAGAACC TGCTGATCTC CCACAACGAC GTGCTGAAGC
1451 TGTGCGATTT CGGCTTTGCC AGGAACCTGA GCGAGGGAAA CAACGCCAAC
1501 TACACAGAGT ACGTGGCTAC CCGCTGGTAC AGGAGCCCAG AGCTGCTGCT
1551 GGGAGCTCCA TACGGAAAGA GCGTGGACAT GTGGTCCGTG GGCTGCATCC
1601 TGGGAGAGCT GTCTGACGGC CAGCCTCTGT TCCCAGGAGA GAGCGAGATC
1651 GATCAGCTGT TTACCATCCA GAAGGTGCTG GGCCCTCTGC CAAGCGAGCA
1701 GATGAAGCTG TTCTACTCCA ACCCTCGCTT CCACGGACTG AGGTTTCCCG
1751 CCGTGAACCA CCCTCAGAGC CTGGAGAGAC GGTACCTGGG CATCCTGAAC
1801 TCCGTGCTGC TGGATCTGAT GAAGAACCTG CTGAAGCTGG ACCCCGCCGA
1851 TCGCTACCTG ACCGAGCAGT GTCTGAACCA CCCTACATTT CAGACCCAGA
1901 GACTGCTGGA CCGGAGCCCT TCCCGCTCTG CTAAGAGGAA GCCATACCAC
1951 GTGGAGAGCT CCACCCTGTC CAACCGCAAC CAGGCCGGCA AGTCTACAGC
2001 TCTGCAGAGC CACCACAGGA GCAACTCCAA GGACATCCAG AACCTGTCTG
2051 TGGGCCTGCC TAGGGCTGAT GAGGGACTGC CAGCTAACGA GAGCTTCCTG
2101 AACGGCAACC TGGCCGGAGC TTCTCTGAGC CCACTGCACA CAAAGACCTA
2151 CCAGGCCTCT AGCCAGCCCG GCTCCACATC TAAGGACCTG ACCAACAACA
2201 ACATCCCACA CCTGCTGTCT CCCAAGGAGG CTAAGAGGAA GACCGAGTTC
2251 GACTTTAACA TCGATCCCAA GCCTAGCGAG GGCCCAGGAA CAAAGTACCT
2301 GAAGAGCAAC TCCAGATCTC AGCAGAACCG GCACTCCTTC ATGGAGTCCT
2351 CTCAGTCTAA GGCCGGCACC CTGCAGCCCA ACGAGAAGCA GAGCAGGCAC
2401 TCCTACATCG ATACCATCCC CCAGAGCTCC CGCAGCCCTT CCTACAGGAC
2451 AAAGGCCAAG AGCCACGGCG CTCTGTCTGA CAGCAAGTCC GTGTCTAACC
2501 TGTCCGAGGC CAGAGCTCAG ATCGCTGAGC CCAGCACCTC CCGGTACTTT
2551 CCTTCTAGCT GTCTGGACCT GAACTCTCCT ACAAGCCCAA CACCCACCAG
2601 ACACTCCGAT ACACGGACCC TGCTGTCTCC AAGCGGCAGA AACAACCGGA
2651 AGGAGGGAAC CCTGGATTCT CGCAGGACCA CAACCAGACA CAGCAAGACA
2701 ATGGAGGAGC TGAAGCTGCC AGAGCACATG GACTCCTCTC ACTCCCACTC
2751 TCTGAGCGCC CCCCACGAGT CCTTCTCTTA CGGCCTGGGA TACACCTCCC
2801 CCTTCAGCTC CCAGCAGCGC CCCCACAGGC ACTCTATGTA CGTGACAAGA
2851 GACAAGGTGC GGGCCAAGGG CCTGGATGGA AGCCTGTCCA TCGGCCAGGG
2901 AATGGCCGCT AGGGCTAACT CCCTGCAGCT GCTGTCTCCT CAGCCAGGAG
2951 AGCAGCTGCC ACCCGAGATG ACCGTGGCCA GATCTAGCGT GAAGGAGACA
3001 AGCCGGGAGG GCACCTCCTC TTTCCACACA AGACAGAAGT CCGAGGGCGG
3051 AGTGTACCAC GACCCCCACT CTGACGATGG AACAGCTCCT AAGGAGAACC
3101 GGCACCTGTA CAACGATCCC GTGCCTAGAC GGGTGGGCTC CTTCTACCGC
3151 GTGCCATCTC CCAGGCCTGA CAACAGCTTT CACGAGAACA ACGTGTCCAC
3201 CAGAGTGAGC TCCCTGCCTT CTGAGTCTAG CTCCGGAACA AACCACTCTA
3251 AGCGGCAGCC CGCCTTTGAC CCTTGGAAGA GCCCAGAGAA CATCTCTCAC
3301 AGCGAGCAGC TGAAGGAGAA GGAGAAGCAG GGCTTCTTTA GAAGCATGAA
3351 GAAGAAGAAG AAGAAGAGCC AGACCGTGCC TAACTCCGAC TCTCCAGATC
3401 TGCTGACCCT GCAGAAGTCC ATCCACAGCG CCTCCACACC CTCTAGCAGA
3451 CCTAAGGAGT GGCGGCCTGA GAAGATCAGC GATCTGCAGA CACAGAGCCA
3501 GCCACTGAAG TCCCTGCGGA AGCTGCTGCA CCTGTCCTCT GCCAGCAACC
3551 ACCCAGCTAG CTCCGACCCA AGGTTCCAGC CACTGACAGC TCAGCAGACC
3601 AAGAACTCTT TTAGCGAGAT CAGGATCCAC CCTCTGTCCC AGGCTTCTGG
3651 CGGATCTAGC AACATCAGGC AGGAGCCAGC TCCAAAGGGC AGGCCCGCTC
3701 TGCAGCTGCC TGGACAGATG GACCCAGGAT GGCACGTGTC CTCTGTGACA
3751 AGATCCGCCA CCGAGGGACC ATCCTACTCT GAGCAGCTGG GCGCTAAGTC
3801 TGGCCCTAAC GGACACCCAT ACAACAGAAC AAACAGAAGC AGAATGCCCA
3851 ACCTCAATGA CCTCAAAGAA ACAGCACTCT GATAAGCGGC CGCAACTCGA
3901 GACTCTAGAC GACTGTGCCT TCTAGTTGCC AGCCATCTGT TGTTTGCCCC
3951 TCCCCCGTGC CTTCCTTGAC CCTGGAAGGT GCCACTCCCA CTGTCCTTTC
4001 CTAATAAAAT GAGGAAATTG CATCGCATTG TCTGAGTAGG TGTCATTCTA
4051 TTCTGGGGGG TGGGGTGGGG CAGGACAGCA AGGGGGAGGA TTGGGAAGAC
4101 AATAGCAGGC ATGCTGGGGA TGCGGTGGGC TCTATGGCCG CGGGCCGCAG
4151 GAACCCCTAG TGATGGAGTT GGCCACTCCC TCTCTGCGCG CTCGCTCGCT
4201 CACTGAGGCC GGGCGACCAA AGGTCGCCCG ACGCCCGGGC TTTGCCCGGG
4251 CGGCCTCAGT GAGCGAGCGA GCGCGCAGCT GCCTGCAGGG GCGCCTGATG
4301 CGGTATTTTC TCCTTACGCA TCTGTGCGGT ATTTCACACC GCATACGTCA
4351 AAGCAACCAT AGTACGCGCC CTGTAGCGGC GCATTAAGCG CGGCGGGTGT
4401 GGTGGTTACG CGCAGCGTGA CCGCTACACT TGCCAGCGCC CTAGCGCCCG
4451 CTCCTTTCGC TTTCTTCCCT TCCTTTCTCG CCACGTTCGC CGGCTTTCCC
4501 CGTCAAGCTC TAAATCGGGG GCTCCCTTTA GGGTTCCGAT TTAGTGCTTT
4551 ACGGCACCTC GACCCCAAAA AACTTGATTT GGGTGATGGT TCACGTAGTG
4601 GGCCATCGCC CTGATAGACG GTTTTTCGCC CTTTGACGTT GGAGTCCACG
4651 TTCTTTAATA GTGGACTCTT GTTCCAAACT GGAACAACAC TCAACCCTAT
4701 CTCGGGCTAT TCTTTTGATT TATAAGGGAT TTTGCCGATT TCGGCCTATT
4751 GGTTAAAAAA TGAGCTGATT TAACAAAAAT TTAACGCGAA TTTTAACAAA
4801 ATATTAACGT TTACAATTTT ATGGTGCACT CTCAGTACAA TCTGCTCTGA
4851 TGCCGCATAG TTAAGCCAGC CCCGACACCC GCCAACACCC GCTGACGCGC
4901 CCTGACGGGC TTGTCTGCTC CCGGCATCCG CTTACAGACA AGCTGTGACC
4951 GTCTCCGGGA GCTGCATGTG TCAGAGGTTT TCACCGTCAT CACCGAAACG
5001 CGCGAGACGA AAGGGCCTCG TGATACGCCT ATTTTTATAG GTTAATGTCA
5051 TGATAATAAT GGTTTCTTAG ACGTCAGGTG GCACTTTTCG GGGAAATGTG
5101 CGCGGAACCC CTATTTGTTT ATTTTTCTAA ATACATTCAA ATATGTATCC
5151 GCTCATGAGA CAATAACCCT GATAAATGCT TCAATAATAT TGAAAAAGGA
5201 AGAGTATGAG TATTCAACAT TTCCGTGTCG CCCTTATTCC CTTTTTTGCG
5251 GCATTTTGCC TTCCTGTTTT TGCTCACCCA GAAACGCTGG TGAAAGTAAA
5301 AGATGCTGAA GATCAGTTGG GTGCACGAGT GGGTTACATC GAACTGGATC
5351 TCAACAGCGG TAAGATCCTT GAGAGTTTTC GCCCCGAAGA ACGTTTTCCA
5401 ATGATGAGCA CTTTTAAAGT TCTGCTATGT GGCGCGGTAT TATCCCGTAT
5451 TGACGCCGGG CAAGAGCAAC TCGGTCGCCG CATACACTAT TCTCAGAATG
5501 ACTTGGTTGA GTACTCACCA GTCACAGAAA AGCATCTTAC GGATGGCATG
5551 ACAGTAAGAG AATTATGCAG TGCTGCCATA ACCATGAGTG ATAACACTGC
5601 GGCCAACTTA CTTCTGACAA CGATCGGAGG ACCGAAGGAG CTAACCGCTT
5651 TTTTGCACAA CATGGGGGAT CATGTAACTC GCCTTGATCG TTGGGAACCG
5701 GAGCTGAATG AAGCCATACC AAACGACGAG CGTGACACCA CGATGCCTGT
5751 AGCAATGGCA ACAACGTTGC GCAAACTATT AACTGGCGAA CTACTTACTC
5801 TAGCTTCCCG GCAACAATTA ATAGACTGGA TGGAGGCGGA TAAAGTTGCA
5851 GGACCACTTC TGCGCTCGGC CCTTCCGGCT GGCTGGTTTA TTGCTGATAA
5901 ATCTGGAGCC GGTGAGCGTG GGTCTCGCGG TATCATTGCA GCACTGGGGC
5951 CAGATGGTAA GCCCTCCCGT ATCGTAGTTA TCTACACGAC GGGGAGTCAG
6001 GCAACTATGG ATGAACGAAA TAGACAGATC GCTGAGATAG GTGCCTCACT
6051 GATTAAGCAT TGGTAACTGT CAGACCAAGT TTACTCATAT ATACTTTAGA
6101 TTGATTTAAA ACTTCATTTT TAATTTAAAA GGATCTAGGT GAAGATCCTT
6151 TTTGATAATC TCATGACCAA AATCCCTTAA CGTGAGTTTT CGTTCCACTG
6201 AGCGTCAGAC CCCGTAGAAA AGATCAAAGG ATCTTCTTGA GATCCTTTTT
6251 TTCTGCGCGT AATCTGCTGC TTGCAAACAA AAAAACCACC GCTACCAGCG
6301 GTGGTTTGTT TGCCGGATCA AGAGCTACCA ACTCTTTTTC CGAAGGTAAC
6351 TGGCTTCAGC AGAGCGCAGA TACCAAATAC TGTCCTTCTA GTGTAGCCGT
6401 AGTTAGGCCA CCACTTCAAG AACTCTGTAG CACCGCCTAC ATACCTCGCT
6451 CTGCTAATCC TGTTACCAGT GGCTGCTGCC AGTGGCGATA AGTCGTGTCT
6501 TACCGGGTTG GACTCAAGAC GATAGTTACC GGATAAGGCG CAGCGGTCGG
6551 GCTGAACGGG GGGTTCGTGC ACACAGCCCA GCTTGGAGCG AACGACCTAC
6601 ACCGAACTGA GATACCTACA GCGTGAGCTA TGAGAAAGCG CCACGCTTCC
6651 CGAAGGGAGA AAGGCGGACA GGTATCCGGT AAGCGGCAGG GTCGGAACAG
6701 GAGAGCGCAC GAGGGAGCTT CCAGGGGGAA ACGCCTGGTA TCTTTATAGT
6751 CCTGTCGGGT TTCGCCACCT CTGACTTGAG CGTCGATTTT TGTGATGCTC
6801 GTCAGGGGGG CGGAGCCTAT GGAAAAACGC CAGCAACGCG GCCTTTTTAC
6851 GGTTCCTGGC CTTTTGCTGG CCTTTTGCTC ACATGT
AAVC-CBh-eGFP
L-ITR: 1-141
CBh promoter: 159-976
EGFP ORF: 1000-1722
bGHp(A): 1747-1974
R-ITR: 1986-2126
Amp(R): 3043-3900
pUC origin: 4051-4718
SEQ ID NO: 108
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGTT AATAGTAATC AATTACGGGG TCATTAGTTC ATAGCCCATA
  201 TATGGAGTTC CGCGTTACAT AACTTACGGT AAATGGCCCG CCTGGCTGAC
  251 CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG
  351 GTAAACTGCC CACTTGGCAG TACATCAAGT GTATCATATG CCAAGTACGC
  401 CCCCTATTGA CGTCAATGAC GGTAAATGGC CCGCCTGGCA TTATGCCCAG
  451 TACATGACCT TACGGGACTT TCCTACTTGG CAGTACATCT CCACGTTCTG
  501 CTTCAGTCTC CCCATCTCCC CCCCCTCCCC ACCCCCAATT TTGTATTTAT
  551 TTATTTTTTA ATTATTTTGT GCAGCGATGG GGGCGGGGGG GGGGGGGGCG
  601 CGCGCCAGGC GGGGCGGGGC GGGGCGAGGG GCGGGGCGGG GCGAGGCGGA
  651 GAGGTGCGGC GGCAGCCAAT CAGAGCGGCG CGCTCCGAAA GTTTCCTTTT
  701 ATGGCGAGGC GGCGGCGGCG GCGGCCCTAT AAAAAGCGAA GCGCGCGGCG
  751 GGGAGTCGCT GCGTTGCCTT CGCCCCGTGC CCCGCTCCGC GCCGCCTCGC
  801 GCCGCCCGCC CCGGCTCTGA CTGACCGCGT TACTCCCACA GGTGAGCGGG
  851 CGGGACGGCC CTTCTCCTCC GGGCTGTAAT TAGCAAGAGG TAAGGGTTTA
  901 AGGGATGGTT GGTTGGTGGG GTATTAATGT TTAATTACCT GTTTTACAGG
  951 CCTGAAATCA CTTGGTTTTA GGTTGGGCTA GCCAAAGCTT GCCGCCACCA
1001 TGGTGTCAAA GGGGGAGGAA CTGTTTACTG GAGTCGTGCC TATTCTGGTC
1051 GAACTGGATG GGGATGTCAA CGGTCATAAG TTTAGCGTGT CCGGAGAGGG
1101 AGAGGGCGAC GCTACCTACG GAAAGCTGAC ACTGAAGTTC ATCTGCACCA
1151 CAGGCAAGCT GCCCGTGCCT TGGCCAACCC TGGTGACCAC ACTGACATAC
1201 GGCGTGCAGT GTTTTAGCCG GTACCCAGAC CACATGAAGC AGCACGATTT
1251 CTTTAAGTCC GCCATGCCCG AGGGATACGT GCAGGAGAGG ACCATCTTCT
1301 TTAAGGACGA TGGCAACTAC AAGACCAGAG CTGAGGTGAA GTTCGAGGGA
1351 GACACACTGG TGAACCGGAT CGAGCTGAAG GGCATCGACT TTAAGGAGGA
1401 TGGAAACATC CTGGGCCACA AGCTGGAGTA CAACTACAAC TCTCACAACG
1451 TGTACATCAT GGCCGATAAG CAGAAGAACG GAATCAAGGT GAACTTCAAG
1501 ATCCGCCACA ACATCGAGGA CGGCAGCGTG CAGCTGGCTG ATCACTACCA
1551 GCAGAACACC CCTATCGGAG ACGGACCCGT GCTGCTGCCT GATAACCACT
1601 ACCTGAGCAC ACAGTCCGCT CTGTCTAAGG ACCCAAACGA GAAGAGGGAT
1651 CACATGGTCC TCCTGGAATT TGTCACTGCT GCTGGGATTA CTCTGGGGAT
1701 GGATGAACTC TATAAGTGAT AAGCGGCCGC AACTCGAGAC TCTAGACGAC
1751 TGTGCCTTCT AGTTGCCAGC CATCTGTTGT TTGCCCCTCC CCCGTGCCTT
1801 CCTTGACCCT GGAAGGTGCC ACTCCCACTG TCCTTTCCTA ATAAAATGAG
1851 GAAATTGCAT CGCATTGTCT GAGTAGGTGT CATTCTATTC TGGGGGGTGG
1901 GGTGGGGCAG GACAGCAAGG GGGAGGATTG GGAAGACAAT AGCAGGCATG
1951 CTGGGGATGC GGTGGGCTCT ATGGCCGCGG GCCGCAGGAA CCCCTAGTGA
2001 TGGAGTTGGC CACTCCCTCT CTGCGCGCTC GCTCGCTCAC TGAGGCCGGG
2051 CGACCAAAGG TCGCCCGACG CCCGGGCTTT GCCCGGGCGG CCTCAGTGAG
2101 CGAGCGAGCG CGCAGCTGCC TGCAGGGGCG CCTGATGCGG TATTTTCTCC
2151 TTACGCATCT GTGCGGTATT TCACACCGCA TACGTCAAAG CAACCATAGT
2201 ACGCGCCCTG TAGCGGCGCA TTAAGCGCGG CGGGTGTGGT GGTTACGCGC
2251 AGCGTGACCG CTACACTTGC CAGCGCCCTA GCGCCCGCTC CTTTCGCTTT
2301 CTTCCCTTCC TTTCTCGCCA CGTTCGCCGG CTTTCCCCGT CAAGCTCTAA
2351 ATCGGGGGCT CCCTTTAGGG TTCCGATTTA GTGCTTTACG GCACCTCGAC
2401 CCCAAAAAAC TTGATTTGGG TGATGGTTCA CGTAGTGGGC CATCGCCCTG
2451 ATAGACGGTT TTTCGCCCTT TGACGTTGGA GTCCACGTTC TTTAATAGTG
2501 GACTCTTGTT CCAAACTGGA ACAACACTCA ACCCTATCTC GGGCTATTCT
2551 TTTGATTTAT AAGGGATTTT GCCGATTTCG GCCTATTGGT TAAAAAATGA
2601 GCTGATTTAA CAAAAATTTA AGGCGAATTT TAACAAAATA TTAACGTTTA
2651 CAATTTTATG GTGCACTCTC AGTACAATCT GCTCTGATGC CGCATAGTTA
2701 AGCCAGCCCC GACACCCGCC AACACCCGCT GACGCGCCCT GACGGGCTTG
2751 TCTGCTCCCG GCATCCGCTT ACAGACAAGC TGTGACCGTC TCCGGGAGCT
2801 GCATGTGTCA GAGGTTTTCA CCGTCATCAC CGAAACGCGC GAGACGAAAG
2851 GGCCTCGTGA TACGCCTATT TTTATAGGTT AATGTCATGA TAATAATGGT
2901 TTCTTAGACG TCAGGTGGCA CTTTTCGGGG AAATGTGCGC GGAACCCCTA
2951 TTTGTTTATT TTTCTAAATA CATTCAAATA TGTATCCGCT CATGAGACAA
3001 TAACCCTGAT AAATGCTTCA ATAATATTGA AAAAGGAAGA GTATGAGTAT
3051 TCAACATTTC CGTGTCGCCC TTATTCCCTT TTTTGCGGCA TTTTGCCTTC
3101 CTGTTTTTGC TCACCCAGAA ACGCTGGTGA AAGTAAAAGA TGCTGAAGAT
3151 CAGTTGGGTG CACGAGTGGG TTACATCGAA CTGGATCTCA ACAGCGGTAA
3201 GATCCTTGAG AGTTTTCGCC CCGAAGAACG TTTTCCAATG ATGAGCACTT
3251 TTAAAGTTCT GCTATGTGGC GCGGTATTAT CCCGTATTGA CGCCGGGCAA
3301 GAGCAACTCG GTCGCCGCAT ACACTATTCT CAGAATGACT TGGTTGAGTA
3351 CTCACCAGTC ACAGAAAAGC ATCTTACGGA TGGCATGACA GTAAGAGAAT
3401 TATGCAGTGC TGCCATAACC ATGAGTGATA ACACTGCGGC CAACTTACTT
3451 CTGACAACGA TCGGAGGACC GAAGGAGCTA ACCGCTTTTT TGCACAACAT
3501 GGGGGATCAT GTAACTCGCC TTGATCGTTG GGAACCGGAG CTGAATGAAG
3551 CCATACCAAA CGACGAGCGT GACACCACGA TGCCTGTAGC AATGGCAACA
3601 ACGTTGCGCA AACTATTAAC TGGCGAACTA CTTACTCTAG CTTCCCGGCA
3651 ACAATTAATA GACTGGATGG AGGCGGATAA AGTTGCAGGA CCACTTCTGC
3701 GCTCGGCCCT TCCGGCTGGC TGGTTTATTG CTGATAAATC TGGAGCCGGT
3751 GAGCGTGGGT CTCGCGGTAT CATTGCAGCA CTGGGGCCAG ATGGTAAGCC
3801 CTCCCGTATC GTAGTTATCT ACACGACGGG GAGTCAGGCA ACTATGGATG
3851 AACGAAATAG ACAGATCGCT GAGATAGGTG CCTCACTGAT TAAGCATTGG
3901 TAACTGTCAG ACCAAGTTTA CTCATATATA CTTTAGATTG ATTTAAAACT
3951 TCATTTTTAA TTTAAAAGGA TCTAGGTGAA GATCCTTTTT GATAATCTCA
4001 TGACCAAAAT CCCTTAACGT GAGTTTTCGT TCCACTGAGC GTCAGACCCC
4051 GTAGAAAAGA TCAAAGGATC TTCTTGAGAT CCTTTTTTTC TGCGCGTAAT
4101 CTGCTGCTTG CAAACAAAAA AACCACCGCT ACCAGCGGTG GTTTGTTTGC
4151 CGGATCAAGA GCTACCAACT CTTTTTCCGA AGGTAACTGG CTTCAGCAGA
4201 GCGCAGATAC CAAATACTGT CCTTCTAGTG TAGCCGTAGT TAGGCCACCA
4251 CTTCAAGAAC TCTGTAGCAC CGCCTACATA CCTCGCTCTG CTAATCCTGT
4301 TACCAGTGGC TGCTGCCAGT GGCGATAAGT CGTGTCTTAC CGGGTTGGAC
4351 TCAAGAGGAT AGTTACCGGA TAAGGCGCAG CGGTCGGGCT GAACGGGGGG
4401 TTCGTGCACA CAGCCCAGCT TGGAGCGAAC GACCTACACC GAACTGAGAT
4451 ACCTACAGCG TGAGCTATGA GAAAGCGCCA CGCTTCCCGA AGGGAGAAAG
4501 GCGGACAGGT ATCCGGTAAG CGGCAGGGTC GGAACAGGAG AGCGCACGAG
4551 GGAGCTTCCA GGGGGAAACG CCTGGTATCT TTATAGTCCT GTCGGGTTTC
4601 GCCACCTCTG ACTTGAGCGT CGATTTTTGT GATGCTCGTC AGGGGGGCGG
4651 AGCCTATGGA AAAACGCCAG CAACGCGGCC TTTTTACGGT TCCTGGCCTT
4701 TTGCTGGCCT TTTGCTCACA
AAVC-CBh-NLS-eGFP
L-ITR: 1-141
CBh promoter: 159-976
NLS-eGFP: 1000-1782
bGHp(A): 1807-2034
R-ITR: 2046-2186
Amp(R): 3103-3960
pUC origin: 4111-4778
SEQ ID NO: 109
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGTT AATAGTAATC AATTACGGGG TCATTAGTTC ATAGCCCATA
  201 TATGGAGTTC CGCGTTACAT AACTTACGGT AAATGGCCCG CCTGGCTGAC
  251 CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG
  351 GTAAACTGCC CACTTGGCAG TACATCAAGT GTATCATATG CCAAGTACGC
  401 CCCCTATTGA CGTCAATGAC GGTAAATGGC CCGCCTGGCA TTATGCCCAG
  451 TACATGACCT TACGGGACTT TCCTACTTGG CAGTACATCT CCACGTTCTG
  501 CTTCAGTCTC CCCATCTCCC CCCCCTCCCC ACCCCCAATT TTGTATTTAT
  551 TTATTTTTTA ATTATTTTGT GCAGCGATGG GGGCGGGGGG GGGGGGGGCG
  601 CGCGCCAGGC GGGGCGGGGC GGGGCGAGGG GCGGGGCGGG GCGAGGCGGA
  651 GAGGTGCGGC GGCAGCCAAT CAGAGCGGCG CGCTCCGAAA GTTTCCTTTT
  701 ATGGCGAGGC GGCGGCGGCG GCGGCCCTAT AAAAAGCGAA GCGCGCGGCG
  751 GGGAGTCGCT GCGTTGCCTT CGCCCCGTGC CCCGCTCCGC GCCGCCTCGC
  801 GCCGCCCGCC CCGGCTCTGA CTGACCGCGT TACTCCCACA GGTGAGCGGG
  851 CGGGACGGCC CTTCTCCTCC GGGCTGTAAT TAGCAAGAGG TAAGGGTTTA
  901 AGGGATGGTT GGTTGGTGGG GTATTAATGT TTAATTACCT GTTTTACAGG
  951 CCTGAAATCA CTTGGTTTTA GGTTGGGCTA GCCAAAGCTT GCCGCCACCA
1001 TGGCCCCAAA AAAGAAAAGA AAAGTGAGGT ATCCTGCATT CCTGTACAAA
1051 GTCGCTACTA TGGTGTCAAA AGGTGAAGAG CTGTTCACCG GAGTGGTGCC
1101 CATCCTGGTG GAGCTGGACG GAGATGTGAA CGGCCACAAG TTCAGCGTGT
1151 CCGGAGAGGG AGAGGGCGAC GCCACCTACG GAAAGCTGAC ACTGAAGTTT
1201 ATCTGCACCA CAGGCAAGCT GCCCGTGCCT TGGCCAACCC TGGTGACCAC
1251 ACTGACATAC GGCGTGCAGT GTTTCTCTCG GTACCCAGAC CACATGAAGC
1301 AGCACGATTT CTTTAAGAGC GCCATGCCCG AGGGATACGT GCAGGAGAGG
1351 ACAATCTTCT TTAAGGAGGA TGGCAACTAC AAGACCAGAG CTGAGGTGAA
1401 GTTCGAGGGA GACACACTGG TGAACCGGAT CGAGCTGAAG GGCATCGACT
1451 TTAAGGAGGA TGGAAACATC CTGGGCCACA AGCTGGAGTA CAACTACAAC
1501 TCCCACAACG TGTACATCAT GGCCGATAAG CAGAAGAACG GAATCAAGGT
1551 GAACTTTAAG ATCCGCCACA ACATCGAGGA CGGCTCTGTG CAGCTGGCTG
1601 ATCACTACCA GCAGAACACC CCTATCGGAG ACGGACCCGT GCTGCTGCCT
1651 GATAACCACT ACCTGTCTAC ACAGAGCGCC CTGTCCAAGG ACCCAAACGA
1701 GAAGAGGGAT CACATGGTGC TCCTGGAATT TGTCACTGCT GCCGGTATTA
1751 CTCTCGGGAT GGATGAACTG TATAAATGAT AAGCGGCCGC AACTCGAGAC
1801 TCTAGAGGAC TGTGCCTTCT AGTTGCCAGC CATCTGTTGT TTGCCCCTCC
1851 CCCGTGCCTT CCTTGACCCT GGAAGGTGCC ACTCCCACTG TCCTTTCCTA
1901 ATAAAATGAG GAAATTGCAT CGCATTGTCT GAGTAGGTGT CATTCTATTC
1951 TGGGGGGTGG GGTGGGGCAG GACAGCAAGG GGGAGGATTG GGAAGACAAT
2001 AGCAGGCATG CTGGGGATGC GGTGGGCTCT ATGGCCGCGG GCCGCAGGAA
2051 CCCCTAGTGA TGGAGTTGGC CACTCCCTCT CTGCGCGCTC GCTCGCTCAC
2101 TGAGGCCGGG CGACCAAAGG TCGCCCGACG CCCGGGCTTT GCCCGGGCGG
2151 CCTCAGTGAG CGAGCGAGCG CGCAGCTGCC TGCAGGGGCG CCTGATGCGG
2201 TATTTTCTCC TTACGCATCT GTGCGGTATT TCACACCGCA TACGTCAAAG
2251 CAACCATAGT ACGCGCCCTG TAGCGGCGCA TTAAGCGCGG CGGGTGTGGT
2301 GGTTACGCGC AGCGTGACCG CTACACTTGC CAGCGCCCTA GCGCCCGCTC
2351 CTTTCGCTTT CTTCCCTTCC TTTCTCGCCA CGTTCGCCGG CTTTCCCCGT
2401 CAAGCTCTAA ATCGGGGGCT CCCTTTAGGG TTCCGATTTA GTGCTTTACG
2451 GCACCTCGAC CCCAAAAAAC TTGATTTGGG TGATGGTTCA CGTAGTGGGC
2501 CATCGCCCTG ATAGACGGTT TTTCGCCCTT TGACGTTGGA GTCCACGTTC
2551 TTTAATAGTG GACTCTTGTT CCAAACTGGA ACAACACTCA ACCCTATCTC
2601 GGGCTATTCT TTTGATTTAT AAGGGATTTT GCCGATTTCG GCCTATTGGT
2651 TAAAAAATGA GCTGATTTAA CAAAAATTTA ACGCGAATTT TAACAAAATA
2701 TTAACGTTTA CAATTTTATG GTGCACTCTC AGTACAATCT GCTCTGATGC
2751 CGCATAGTTA AGCCAGCCCC GACACCCGCC AACACCCGCT GACGCGCCCT
2801 GACGGGCTTG TCTGCTCCCG GCATCCGCTT ACAGACAAGC TGTGACCGTC
2851 TCCGGGAGCT GCATGTGTCA GAGGTTTTCA CCGTCATCAC CGAAACGCGC
2901 GAGACGAAAG GGCCTCGTGA TACGCCTATT TTTATAGGTT AATGTCATGA
2951 TAATAATGGT TTCTTAGACG TCAGGTGGCA CTTTTCGGGG AAATGTGCGC
3001 GGAACCCCTA TTTGTTTATT TTTCTAAATA CATTCAAATA TGTATCCGCT
3051 CATGAGACAA TAACCCTGAT AAATGCTTCA ATAATATTGA AAAAGGAAGA
3101 GTATGAGTAT TCAACATTTC CGTGTCGCCC TTATTCCCTT TTTTGCGGCA
3151 TTTTGCCTTC CTGTTTTTGC TCACCCAGAA ACGCTGGTGA AAGTAAAAGA
3201 TGCTGAAGAT CAGTTGGGTG CACGAGTGGG TTACATCGAA CTGGATCTCA
3251 ACAGCGGTAA GATCCTTGAG AGTTTTCGCC CCGAAGAACG TTTTCCAATG
3301 ATGAGCACTT TTAAAGTTCT GCTATGTGGC GCGGTATTAT CCCGTATTGA
3351 CGCCGGGCAA GAGCAACTCG GTCGCCGCAT ACACTATTCT CAGAATGACT
3401 TGGTTGAGTA CTCACCAGTC ACAGAAAAGC ATCTTACGGA TGGCATGACA
3451 GTAAGAGAAT TATGCAGTGC TGCCATAACC ATGAGTGATA ACACTGCGGC
3501 CAACTTACTT CTGACAACGA TCGGAGGACC GAAGGAGCTA ACCGCTTTTT
3551 TGCACAACAT GGGGGATCAT GTAACTCGCC TTGATCGTTG GGAACCGGAG
3601 CTGAATGAAG CCATACCAAA CGACGAGCGT GACACCACGA TGCCTGTAGC
3651 AATGGCAACA ACGTTGCGCA AACTATTAAC TGGCGAACTA CTTACTCTAG
3701 CTTCCCGGCA ACAATTAATA GACTGGATGG AGGCGGATAA AGTTGCAGGA
3751 CCACTTCTGC GCTCGGCCCT TCCGGCTGGC TGGTTTATTG CTGATAAATC
3801 TGGAGCCGGT GAGCGTGGGT CTCGCGGTAT CATTGCAGCA CTGGGGCCAG
3851 ATGGTAAGCC CTCCCGTATC GTAGTTATCT ACACGACGGG GAGTCAGGCA
3901 ACTATGGATG AACGAAATAG ACAGATCGCT GAGATAGGTG CCTCACTGAT
3951 TAAGCATTGG TAACTGTCAG ACCAAGTTTA CTCATATATA CTTTAGATTG
4001 ATTTAAAACT TCATTTTTAA TTTAAAAGGA TCTAGGTGAA GATCCTTTTT
4051 GATAATCTCA TGACCAAAAT CCCTTAACGT GAGTTTTCGT TCCACTGAGC
4101 GTCAGACCCC GTAGAAAAGA TCAAAGGATC TTCTTGAGAT CCTTTTTTTC
4151 TGCGCGTAAT CTGCTGCTTG CAAACAAAAA AACCACCGCT ACCAGCGGTG
4201 GTTTGTTTGC CGGATCAAGA GCTACCAACT CTTTTTCCGA AGGTAACTGG
4251 CTTCAGCAGA GCGCAGATAC CAAATACTGT CCTTCTAGTG TAGCCGTAGT
4301 TAGGCCACCA CTTCAAGAAC TCTGTAGCAC CGCCTACATA CCTCGCTCTG
4351 CTAATCCTGT TACCAGTGGC TGCTGCCAGT GGCGATAAGT CGTGTCTTAC
4401 CGGGTTGGAC TCAAGACGAT AGTTACCGGA TAAGGCGCAG CGGTCGGGCT
4451 GAACGGGGGG TTCGTGCACA CAGCCCAGCT TGGAGCGAAC GACCTACACC
4501 GAACTGAGAT ACCTACAGCG TGAGCTATGA GAAAGCGCCA CGCTTCCCGA
4551 AGGGAGAAAG GCGGACAGGT ATCCGGTAAG CGGCAGGGTC GGAACAGGAG
4601 AGCGCACGAG GGAGCTTCCA GGGGGAAACG CCTGGTATCT TTATAGTCCT
4651 GTCGGGTTTC GCCACCTCTG ACTTGAGCGT CGATTTTTGT GATGCTCGTC
4701 AGGGGGGCGG AGCCTATGGA AAAACGCCAG CAACGCGGCC TTTTTACGGT
4751 TCCTGGCCTT TTGCTGGCCT TTTGCTCACA TGT
AAVC-CBh-mBPIP-TATκ28-hCDKL5-107
L-ITR: 1-141
CBh promoter: 159-976
Bip-TATκ28-hCDKL5-107:1000 - 4065
bGHp(A): 4090-4317
R-ITR: 4329-4469
Amp(R): 5386-6243
pUC origin: 6394-7061
SEQ ID NO: 110
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGTT AATAGTAATC AATTACGGGG TCATTAGTTC ATAGGCCATA
  201 TATGGAGTTC CGCGTTACAT AACTTACGGT AAATGGCCCG CCTGGCTGAC
  251 CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG
  351 GTAAACTGCC CACTTGGCAG TACATCAAGT GTATCATATG CCAAGTACGC
  401 CCCCTATTGA CGTCAATGAC GGTAAATGGC CCGCCTGGCA TTATGCCCAG
  451 TACATGACCT TACGGGACTT TCCTACTTGG CAGTACATCT CCACGTTCTG
  501 CTTCACTCTC CCCATCTCCC CCCCCTCCCC ACCCCCAATT TTGTATTTAT
  551 TTATTTTTTA ATTATTTTGT GCAGCGATGG GGGCGGGGGG GGGGGGGGCG
  601 CGCGCCAGGC GGGGCGGGGC GGGGCGAGGG GCGGGGCGGG GCGAGGCGGA
  651 GAGGTGCGGC GGCAGCCAAT CAGAGCGGCG CGCTCCGAAA GTTTCCTTTT
  701 ATGGCGAGGC GGCGGCGGCG GCGGCCCTAT AAAAAGCGAA GCGCGCGGCG
  751 GGGAGTCGCT GCGTTGCCTT CGCCCCGTGC CCCGCTCCGC GCCGCCTCGC
  801 GCCGCCCGCC CCGGCTCTGA CTGACCGCGT TACTCCCACA GGTGAGCGGG
  851 CGGGACGGCC CTTCTCCTCC GGGCTGTAAT TAGCAAGAGG TAAGGGTTTA
  901 AGGGATGGTT GGTTGGTGGG GTATTAATGT TTAATTACCT GTTTTACAGG
  951 CCTGAAATCA CTTGGTTTTA GGTTGGGCTA GCCAAAGCTT GCCGCCACCA
1001 TGAAACTCTC CCTGGTCGCT GCTATGCTGC TCCTCCTGTC CCTGGTCGCT
1051 GCTATGCTCC TGCTGCTGTC TGCCGCTCGG GCTGGTGATG CTGCTCAGCC
1101 AGCTAGGAGA GCTAGGAGGA CCAAGCTGGC TGCTTACGCT AGAAAGGCTG
1151 CTAGACAGGC TAGGGCTGGA GGAGGCGGAT CCAAGATCCC CAACATCGGC
1201 AACGTGATGA ACAAGTTCGA GATCCTGGGA GTGGTGGGAG AGGGAGCTTA
1251 CGGAGTGGTG CTGAAGTGCC GGCACAAGGA GACACACGAG ATCGTGGCTA
1301 TCAAGAAGTT TAAGGACAGC GAGGAGAACG AGGAGGTGAA GGAGACAACC
1351 CTGCGCGAGC TGAAGATGCT GAGGACACTG AAGCAGGAGA ACATCGTGGA
1401 GCTGAAGGAG GCCTTCAGGA GACGGGGAAA GCTGTACCTG GTGTTTGAGT
1451 ACGTGGAGAA GAACATGCTG GAGCTGCTGG AGGAGATGCC AAACGGAGTG
1501 CCACCTGAGA AGGTGAAGTC CTACATCTAC CAGCTGATCA AGGCTATCCA
1551 CTGGTGCCAC AAGAACGACA TCGTGCACCG CGATATCAAG CCTGAGAACC
1601 TGCTGATCTC CCACAACGAC GTGCTGAAGC TGTGCGATTT CGGCTTTGCC
1651 AGGAACCTGA GCGAGGGAAA CAACGCCAAC TACACAGAGT ACGTGGCTAC
1701 CCGCTGGTAC AGGAGCCCAG AGCTGCTGCT GGGAGCTCCA TACGGAAAGA
1751 GCGTGGACAT GTGGTCCGTG GGCTGCATCC TGGGAGAGCT GTCTGACGGC
1801 CAGCCTCTGT TCCCAGGAGA GAGCGAGATC GATCAGCTGT TTACCATCCA
1851 GAAGGTGCTG GGCCCTCTGC CAAGCGAGCA GATGAAGCTG TTCTACTCCA
1901 ACCCTCGCTT CCACGGACTG AGGTTTCCCG CCGTGAACCA CCCTCAGAGC
1951 CTGGAGCGCA GGTACCTGGG CATCCTGAAC TCCGTGCTGC TGGATCTGAT
2001 GAAGAACCTG CTGAAGCTGG ACCCCGCCGA TAGATACCTG ACCGAGCAGT
2051 GTCTGAACCA CCCTACATTT CAGACCCAGA GACTGCTGGA CCGGAGCCCT
2101 TCCCGCTCTG CTAAGAGGAA GCCATACCAC GTGGAGAGCT CCACCCTGTC
2151 CAACCGCAAC CAGGCCGGCA AGTCTACAGC TCTGCAGAGC CACCACAGGA
2201 GCAACTCCAA GGACATCCAG AACCTGTCTG TGGGCCTGCC TAGGGCTGAT
2251 GAGGGACTGC CAGCTAACGA GAGCTTCCTG AACGGCAACC TGGCCGGAGC
2301 TTCTCTGAGC CCACTGCACA CAAAGACCTA CCAGGCCTCT AGCCAGCCCG
2351 GCTCCACATC TAAGGACCTG ACCAACAACA ACATCCCACA CCTGCTGTCT
2401 CCCAAGGAGG CTAAGAGGAA GACCGAGTTC GACTTTAACA TCGATCCCAA
2451 GCCTAGCGAG GGCCCTGGAA CAAAGTACCT GAAGAGCAAC TCCAGATCTC
2501 AGCAGAACCG GCACTCCTTC ATGGAGTCCT CTCAGTCTAA GGCCGGCACC
2551 CTGCAGCCAA AGGAGAAGGA GAGCCGGCAC TCCTACATCG ATACCATCCC
2601 CCAGAGCTCC CGCAGCCCTT CCTACAGGAC AAAGGCCAAG AGCCACGGCG
2651 CTCTGTCTGA CAGCAAGTCC GTGTCTAACC TGTCCGAGGC CAGAGCTCAG
2701 ATCGCTGAGC CAAGCACCTC CAGGTACTTT CCTTCTAGCT GTCTGGACCT
2751 GAACTCTCCT ACAAGCCCAA CACCCACCAG ACACAGCGAT ACACGGACCC
2801 TGCTGTCTCC AAGCGGCAGA AACAACCGGA ACGAGGGAAC CCTGGATTCT
2851 AGACGGACCA CAACCAGGCA CAGCAAGACA ATGGAGGAGC TGAAGCTGCC
2901 AGAGCACATG GACTCCTCTC ACTCCCACTC TCTGAGCGCC CCCCACGAGT
2951 CCTTCTCTTA CGGCCTGGGA TACACCTCCC CCTTCAGCTC CCAGCAGCGC
3001 CCCCACAGGC ACTCTATGTA CGTGACAAGA GACAAGGTGC GGGCCAAGGG
3051 CCTGGATGGA AGCCTGTCCA TCGGCCAGGG AATGGCCGCT AGAGCTAACT
3101 CCCTGCAGCT GCTGTCTCCT CAGCCAGGAG AGCAGCTGCC ACCCGAGATG
3151 ACCGTGGCCA GATCTAGCGT GAAGGAGACA AGCCGGGAGG GCACCTCCTC
3201 TTTCCACACA AGACAGAAGT CCGAGGGCGG AGTGTACCAC GACCCCCACT
3251 CTGACGATGG AACAGCTCCT AAGGAGAACC GGCACCTGTA CAACGATCCC
3301 GTGCCTCGCA GGGTGGGCTC CTTCTACCGC GTGCCATCTC CCAGGCCTGA
3351 CAACAGCTTT CACGAGAACA ACGTGTCCAC CAGAGTGAGC TCCCTGCCAT
3401 CTGAGTCTAG CTCCGGAACA AACCACTCTA AGCGGCAGCC CGCCTTCGAT
3451 CCTTGGAAGA GCCCAGAGAA CATCTCTCAC AGCGAGCAGC TGAAGGAGAA
3501 GGAGAAGCAG GGCTTCTTTC GCAGCATGAA GAAGAAGAAG AAGAAGAGCC
3551 AGACCGTGCC TAACTCCGAC TCTCCAGATC TGCTGACCCT GCAGAAGTCC
3601 ATCCACAGCG CCTCCACACC CTCTAGCAGA CCTAAGGAGT GGCGGCCTGA
3651 GAAGATCAGC GATCTGCAGA CACAGAGCCA GCCACTGAAG TCCCTGAGGA
3701 AGCTGCTGCA CCTGTCCTCT GCCAGCAACC ACCCAGCTAG CTCCGACCCA
3751 AGGTTCCAGC CACTGACAGC TCAGCAGACC AAGAACTCTT TTAGCGAGAT
3801 CAGGATCCAC CCTCTGTCCC AGGCTTCTGG CGGATCTAGC AACATCAGGC
3851 AGGAGCCAGC TCCAAAGGGC AGGCCCGCTC TGCAGCTGCC TGGACAGATG
3901 GACCCAGGAT GGCACGTGTC CTCTGTGACA AGGTCCGCCA CCGAGGGACC
3951 ATCCTACTCT GAGCAGCTGG GCGCTAAGTC TGGCCCTAAC GGACACCCAT
4001 ACAACCGAAC AAATAGAAGT AGGATGCCAA ACCTCAATGA CCTCAAGGAA
4051 ACTGCCCTGT GATAAGCGGC CGCAACTCGA GACTCTAGAC GACTGTGCCT
4101 TCTAGTTGCC AGCCATCTGT TGTTTGCCCC TCCCCCGTGC CTTCCTTGAC
4151 CCTGGAAGGT GCCACTCCCA CTGTCCTTTC CTAATAAAAT GAGGAAATTG
4201 CATCGCATTG TCTGAGTAGG TGTCATTCTA TTCTGGGGGG TGGGGTGGGG
4251 CAGGACAGCA AGGGGGAGGA TTGGGAAGAC AATAGGAGGC ATGCTGGGGA
4301 TGCGGTGGGC TCTATGGCCG CGGGCCGCAG GAACCCCTAG TGATGGAGTT
4351 GGCCACTCCC TCTCTGCGCG CTCGCTCGCT CACTGAGGCC GGGCGACCAA
4401 AGGTCGCCCG ACGCCCGGGC TTTGCCCGGG CGGCCTCAGT GAGCGAGCGA
4451 GCGCGCAGCT GCCTGCAGGG GCGCCTGATG CGGTATTTTC TCCTTACGCA
4501 TCTGTGCGGT ATTTCACACC GCATACGTCA AAGCAACCAT AGTACGCGCC
4551 CTGTAGCGGC GCATTAAGCG CGGCGGGTGT GGTGGTTACG CGCAGCGTGA
4601 CCGCTACACT TGCCAGCGCC CTAGCGCCCG CTCCTTTCGC TTTCTTCCCT
4651 TCCTTTCTCG CCACGTTCGC CGGCTTTCCC CGTCAAGCTC TAAATCGGGG
4701 GCTCCCTTTA GGGTTCCGAT TTAGTGCTTT ACGGCACCTC GACCCCAAAA
4751 AACTTGATTT GGGTGATGGT TCACGTAGTG GGCCATCGCC CTGATAGACG
4801 GTTTTTCGCC CTTTGACGTT GGAGTCCACG TTCTTTAATA GTGGACTCTT
4851 GTTCCAAACT GGAACAACAC TCAACCCTAT CTCGGGCTAT TCTTTTGATT
4901 TATAAGGGAT TTTGCCGATT TCGGCCTATT GGTTAAAAAA TGAGCTGATT
4951 TAACAAAAAT TTAACGCGAA TTTTAACAAA ATATTAACGT TTACAATTTT
5001 ATGGTGCACT CTCAGTACAA TCTGCTCTGA TGCCGCATAG TTAAGCCAGC
5051 CCCGACACCC GCCAACACCC GCTGACGCGC CCTGACGGGC TTGTCTGCTC
5101 CCGGCATCCG CTTACAGACA AGCTGTGACC GTCTCCGGGA GCTGCATGTG
5151 TCAGAGGTTT TCACCGTCAT CACCGAAACG CGCGAGACGA AAGGGCCTCG
5201 TGATACGCCT ATTTTTATAG GTTAATGTCA TGATAATAAT GGTTTCTTAG
5251 ACGTCAGGTG GCACTTTTCG GGGAAATGTG CGCGGAACCC CTATTTGTTT
5301 ATTTTTCTAA ATACATTCAA ATATGTATCC GCTCATGAGA CAATAACCCT
5351 GATAAATGCT TCAATAATAT TGAAAAAGGA AGAGTATGAG TATTCAACAT
5401 TTCCGTGTCG CCCTTATTCC CTTTTTTGGG GCATTTTGCC TTCCTGTTTT
5451 TGCTCACCCA GAAACGCTGG TGAAAGTAAA AGATGCTGAA GATCAGTTGG
5501 GTGCACGAGT GGGTTACATC GAACTGGATC TCAACAGCGG TAAGATCCTT
5551 GAGAGTTTTC GCCCCGAAGA ACGTTTTCCA ATGATGAGCA CTTTTAAAGT
5601 TCTGCTATGT GGCGCGGTAT TATCCCGTAT TGACGCCGGG CAAGAGCAAC
5651 TCGGTCGCCG CATACACTAT TCTCAGAATG ACTTGGTTGA GTACTCACCA
5701 GTCACAGAAA AGCATCTTAC GGATGGCATG ACAGTAAGAG AATTATGCAG
5751 TGCTGCCATA ACCATGAGTG ATAACACTGC GGCCAACTTA CTTCTGACAA
5801 CGATCGGAGG ACCGAAGGAG CTAACCGCTT TTTTGCACAA CATGGGGGAT
5851 CATGTAACTC GCCTTGATCG TTGGGAACCG GAGCTGAATG AAGCCATACC
5901 AAAGGAGGAG CGTGACACCA CGATGCCTGT AGCAATGGCA ACAACGTTGC
5951 GCAAACTATT AACTGGCGAA CTACTTACTC TAGCTTCCCG GCAACAATTA
6001 ATAGACTGGA TGGAGGCGGA TAAAGTTGCA GGACCACTTC TGCGCTCGGC
6051 CCTTCCGGCT GGCTGGTTTA TTGCTGATAA ATCTGGAGCC GGTGAGCGTG
6101 GGTCTCGCGG TATCATTGCA GCACTGGGGC CAGATGGTAA GCCCTCCCGT
6151 ATCGTAGTTA TCTACACGAC GGGGAGTCAG GCAACTATGG ATGAACGAAA
6201 TAGACAGATC GCTGAGATAG GTGCCTCACT GATTAAGCAT TGGTAACTGT
6251 CAGACCAAGT TTACTCATAT ATACTTTAGA TTGATTTAAA ACTTCATTTT
6301 TAATTTAAAA GGATCTAGGT GAAGATCCTT TTTGATAATC TCATGACCAA
6351 AATCCCTTAA CGTGAGTTTT CGTTCCACTG AGCGTCAGAC CCCGTAGAAA
6401 AGATCAAAGG ATCTTCTTGA GATCCTTTTT TTCTGCGCGT AATCTGCTGC
6451 TTGCAAACAA AAAAACCACC GCTACCAGCG GTGGTTTGTT TGCCGGATCA
6501 AGAGCTACCA ACTCTTTTTC CGAAGGTAAC TGGCTTCAGC AGAGCGCAGA
6551 TACCAAATAC TGTCCTTCTA GTGTAGCCGT AGTTAGGCCA CCACTTCAAG
6601 AACTCTGTAG CACCGCCTAC ATACCTCGCT CTGCTAATCC TGTTACCAGT
6651 GGCTGCTGCC AGTGGCGATA AGTCGTGTCT TACCGGGTTG GACTCAAGAC
6701 GATAGTTACC GGATAAGGCG CAGCGGTCGG GCTGAACGGG GGGTTCGTGC
6751 ACACAGCCCA GCTTGGAGCG AACGACCTAC ACCGAACTGA GATACCTACA
6801 GCGTGAGCTA TGAGAAAGCG CCACGCTTCC CGAAGGGAGA AAGGCGGACA
6851 GGTATCCGGT AAGCGGCAGG GTCGGAACAG GAGAGCGCAC GAGGGAGCTT
6901 CCAGGGGGAA ACGCCTGGTA TCTTTATAGT CCTGTCGGGT TTCGCCACCT
6951 CTGACTTGAG CGTCGATTTT TGTGATGCTC GTCAGGGGGG CGGAGCCTAT
7001 GGAAAAACGC CAGCAACGCG GCCTTTTTAC GGTTCCTGGC CTTTTGCTGG
7051 CCTTTTGCTC ACATGT
AAVC-CBh-mBPIP-TATκ28-hCDKL5-107
L-ITR: 1-141
CBh promoter: 159-976
Bip-TATκ28-hCDKL5-107 (kinase dead): 1000 - 4065
bGHp(A): 4090-4317
R-ITR: 4329-4469
Amp(R): 5386-6243
pUC origin: 6394-7061
SEQ ID NO: 111
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGTT AATAGTAATC AATTACGGGG TCATTAGTTC ATAGCCCATA
  201 TATGGAGTTC CGCGTTACAT AACTTACGGT AAATGGCCCG CCTGGCTGAC
  251 CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG
  351 GTAAACTGCC CACTTGGCAG TACATCAAGT GTATCATATG CCAAGTACGC
  401 CCCCTATTGA CGTCAATGAC GGTAAATGGC CCGCCTGGCA TTATGCCCAG
  451 TACATGACCT TACGGGACTT TCCTACTTGG CAGTACATCT CCACGTTCTG
  501 CTTCACTCTC CCCATCTCCC CCCCCTCCCC ACCCCCAATT TTGTATTTAT
  551 TTATTTTTTA ATTATTTTGT GCAGCGATGG GGGCGGGGGG GGGGGGGGCG
  601 CGCGCCAGGC GGGGCGGGGC GGGGCGAGGG GCGGGGCGGG GCGAGGCGGA
  651 GAGGTGCGGC GGCAGCCAAT CAGAGCGGCG CGCTCCGAAA GTTTCCTTTT
  701 ATGGGGAGGC GGCGGCGGCG GCGGCCCTAT AAAAAGCGAA GCGCGCGGCG
  751 GGGAGTCGCT GCGTTGCCTT CGCCCCGTGC CCCGCTCCGC GCCGCCTCGC
  801 GCCGCCCGCC CCGGCTCTGA CTGACCGCGT TACTCCCACA GGTGAGCGGG
  851 CGGGACGGCC CTTCTCCTCC GGGCTGTAAT TAGCAAGAGG TAAGGGTTTA
  901 AGGGATGGTT GGTTGGTGGG GTATTAATGT TTAATTACCT GTTTTACAGG
  951 CCTGAAATCA CTTGGTTTTA GGTTGGGCTA GCCAAAGCTT GCCGCCACCA
1001 TGAAACTCTC CCTCGTCGCC GCTATGCTCC TGCTCCTCTC CCTCGTCGCT
1051 GCCATGCTCC TGCTGCTCAG TGCCGCTCGG GCCGGAGATG CTGCTCAGCC
1101 AGCTAGGAGA GCTAGGAGGA CCAAGCTGGC TGCTTACGCT AGGAAGGCTG
1151 CTAGGCAGGC TAGGGCTGGC GGAGGCGGAT CCAAGATCCC CAACATCGGC
1201 AACGTGATGA ACAAGTTCGA GATCCTGGGA GTGGTGGGAG AGGGAGCTTA
1251 CGGAGTGGTG CTGAAGTGCA GGCACAAGGA GACACACGAG ATCGTGGCTA
1301 TCAGGAGGTT CAAGGACAGC GAGGAGAACG AGGAGGTGAA GGAGACAACC
1351 CTGAGAGAGC TGAAGATGCT GCGGACACTG AAGCAGGAGA ACATCGTGGA
1401 GCTGAAGGAG GCCTTCCGGC GCAGGGGAAA GCTGTACCTG GTGTTTGAGT
1451 ACGTGGAGAA GAACATGCTG GAGCTGCTGG AGGAGATGCC AAACGGAGTG
1501 CCACCTGAGA AGGTGAAGTC CTACATCTAC CAGCTGATCA AGGCTATCCA
1551 CTGGTGCCAC AAGAACGACA TCGTGCACAG AGATATCAAG CCTGAGAACC
1601 TGCTGATCTC CCACAACGAC GTGCTGAAGC TGTGCGATTT CGGCTTTGCC
1651 CGGAACCTGA GCGAGGGAAA CAACGCCAAC TACACAGAGT ACGTGGCTAC
1701 CAGATGGTAC CGGAGCCCAG AGCTGCTGCT GGGAGCTCCA TACGGAAAGA
1751 GCGTGGACAT GTGGTCCGTG GGCTGCATCC TGGGAGAGCT GTCTGACGGC
1801 CAGCCTCTGT TCCCAGGAGA GAGCGAGATC GATCAGCTGT TTACCATCCA
1851 GAAGGTGCTG GGCCCTCTGC CAAGCGAGCA GATGAAGCTG TTCTACTCCA
1901 ACCCTAGATT CCACGGACTG CGGTTTCCCG CCGTGAACCA CCCTCAGAGC
1951 CTGGAGAGAC GGTACCTGGG CATCCTGAAC TCCGTGCTGC TGGATCTGAT
2001 GAAGAACCTG CTGAAGCTGG ACCCCGCCGA TCGCTACCTG ACCGAGCAGT
2051 GTCTGAACCA CCCTACATTT CAGACCCAGC GCCTGCTGGA CAGGAGCCCT
2101 TCCAGATCTG CTAAGCGGAA GCCATACCAC GTGGAGAGCT CCACCCTGTC
2151 CAACAGAAAC CAGGCCGGCA AGTCTACAGC TCTGCAGAGC CACCACCGGA
2201 GCAACTCCAA GGACATCCAG AACCTGTCTG TGGGCCTGCC TAGAGCCGAT
2251 GAGGGACTGC CAGCTAACGA GAGCTTCCTG AACGGCAACC TGGCCGGAGC
2301 TTCTCTGAGC CCACTGCACA CAAAGACCTA CCAGGCCTCT AGCCAGCCCG
2351 GCTCCACATC TAAGGACCTG ACCAACAACA ACATCCCACA CCTGCTGTCT
2401 CCCAAGGAGG CTAAGAGCAA GACCGAGTTC GACTTTAACA TCGATCCCAA
2451 GCCTAGCGAG GGCCCTGGAA CAAAGTACCT GAAGAGCAAC TCCCGCTCTC
2501 AGCAGAACAG GCACTCCTTC ATGGAGTCCT CTCAGTCTAA GGCCGGCACC
2551 CTGCAGCCAA ACGAGAAGCA GAGCAGACAC TCCTACATCG ATACCATCCC
2601 CCAGAGCTCC AGAAGCCCTT CCTACCGGAC AAAGGCCAAG AGCCACGGCG
2651 CTCTGTCTGA CAGCAAGTCC GTGTCTAACC TGTCCGAGGC TAGGGCTCAG
2701 ATCGCTGAGC CCAGCACCTC CAGGTACTTT CCTTCTAGCT GTCTGGACCT
2751 GAACTCTCCT ACAAGCCCAA CACCCACCCG CCACAGCGAT ACAAGGACCC
2801 TGCTGTCTCC AAGCGGCAGG AACAACAGGA ACGAGGGAAC CCTGGATTCT
2851 CGCAGGACCA CAACCCGGCA CAGCAAGACA ATGGAGGAGC TGAAGCTGCC
2901 AGAGCACATG GACTCCTCTC ACTCCCACTC TCTGAGCGCC CCCCACGAGT
2951 CCTTCTCTTA CGGCCTGGGA TACACCTCCC CCTTCAGCTC CCAGCAGAGG
3001 CCCCACAGGC ACTCTATGTA CGTGACACGC GACAAGGTGA GGGCCAAGGG
3051 CCTGGATGGA AGCCTGTCCA TCGGCCAGGG AATGGCCGCT AGGGCTAACT
3101 CCCTGCAGCT GCTGTCTCCT CAGCCAGGAG AGCAGCTGCC ACCAGAGATG
3151 ACCGTGGCTC GCTCTAGCGT GAAGGAGACA AGCAGGGAGG GCACCTCCTC
3201 TTTCCACACA CGCCAGAAGT CCGAGGGCGG AGTGTACCAC GACCCCCACT
3251 CTGACGATGG AACAGCTCCT AAGGAGAACA GGCACCTGTA CAACGATCCC
3301 GTGCCTAGAC GGGTGGGCTC CTTCTACAGA GTGCCATCTC CCCGGCCTGA
3351 CAACAGCTTT CACGAGAACA ACGTGTCCAC CCGCGTGAGC TCCCTGCCAT
3401 CTGAGTCTAG CTCCGGAACA AACCACTCTA AGAGGCAGCC CGCCTTCGAT
3451 CCTTGGAAGA GCCCAGAGAA CATCTCTCAC AGCGAGCAGC TGAAGGAGAA
3501 GGAGAAGCAG GGCTTCTTTA GAAGCATGAA GAAGAAGAAG AAGAAGAGCC
3551 AGACCGTGCC TAACTCCGAC TCTCCAGATC TGCTGACCCT GCAGAAGTCC
3601 ATCCACAGCG CCTCCACACC ATCTAGCCGC CCTAAGGAGT GGAGGCCTGA
3651 GAAGATCAGC GATCTGCAGA CACAGAGCCA GCCACTGAAG TCCCTGCGGA
3701 AGCTGCTGCA CCTGTCCTCT GCCAGCAACC ACCCCGCTAG CTCCGACCCA
3751 AGATTCCAGC CCCTGACAGC CCAGCAGACC AAGAACTCTT TTAGCGAGAT
3801 CCGGATCCAC CCTCTGTCCC AGGCTTCTGG CGGATCTAGC AACATCAGAC
3851 AGGAGCCAGC TCCAAAGGGC CGGCCCGCTC TGCAGCTGCC TGGCCAGATG
3901 GACCCAGGAT GGCACGTGTC CTCTGTGACA AGGTCCGCCA CCGAGGGACC
3951 ATCCTACTCT GAGCAGCTGG GCGCTAAGTC TGGCCCTAAC GGACACCCAT
4001 ACAATAGGAC TAATCGCAGC AGAATGCCCA ACCTGAACGA CCTCAAGGAA
4051 ACAGCACTCT GATAAGCGGC CGCAACTCGA GACTCTAGAC GACTGTGCCT
4101 TCTAGTTGCC AGCCATCTGT TGTTTGCCCC TCCCCCGTGC CTTCCTTGAC
4151 CCTGGAAGGT GCCACTCCCA CTGTCCTTTC CTAATAAAAT GAGGAAATTG
4201 CATCGCATTG TCTGAGTAGG TGTCATTCTA TTCTGGGGGG TGGGGTGGGG
4251 CAGGACAGCA AGGGGGAGGA TTGGGAAGAC AATAGGAGGC ATGCTGGGGA
4301 TGCGGTGGGC TCTATGGCCG CGGGCCGCAG GAACCCCTAG TGATGGAGTT
4351 GGCCACTCCC TCTCTGCGCG CTCGCTCGCT CACTGAGGCC GGGCGACCAA
4401 AGGTCGCCCG ACGCCCGGGC TTTGCCCGGG CGGCCTCAGT GAGCGAGCGA
4451 GCGCGCAGCT GCCTGCAGGG GCGCCTGATG CGGTATTTTC TCCTTACGCA
4501 TCTGTGCGGT ATTTCACACC GCATACGTCA AAGCAACCAT AGTACGCGCC
4551 CTGTAGCGGC GCATTAAGCG CGGCGGGTGT GGTGGTTACG CGCAGCGTGA
4601 CCGCTACACT TGCCAGCGCC CTAGCGCCCG CTCCTTTCGC TTTCTTCCCT
4651 TCCTTTCTCG CCACGTTCGC CGGCTTTCCC CGTCAAGCTC TAAATCGGGG
4701 GCTCCCTTTA GGGTTCCGAT TTAGTGCTTT ACGGCACCTC GACCCCAAAA
4751 AACTTGATTT GGGTGATGGT TCACGTAGTG GGCCATCGCC CTGATAGACG
4801 GTTTTTCGCC CTTTGACGTT GGAGTCCACG TTCTTTAATA GTGGACTCTT
4851 GTTCCAAACT GGAACAACAC TCAACCCTAT CTCGGGCTAT TCTTTTGATT
4901 TATAAGGGAT TTTGCCGATT TCGGCCTATT GGTTAAAAAA TGAGCTGATT
4951 TAACAAAAAT TTAACGCGAA TTTTAACAAA ATATTAACGT TTACAATTTT
5001 ATGGTGCACT CTCAGTACAA TCTGCTCTGA TGCCGCATAG TTAAGCCAGC
5051 CCCGACACCC GCCAACACCC GCTGACGCGC CCTGACGGGC TTGTCTGCTC
5101 CCGGCATCCG CTTACAGACA AGCTGTGACC GTCTCCGGGA GCTGCATGTG
5151 TCAGAGGTTT TCACCGTCAT CACCGAAACG CGCGAGACGA AAGGGCCTCG
5201 TGATACGCCT ATTTTTATAG GTTAATGTCA TGATAATAAT GGTTTCTTAG
5251 ACGTCAGGTG GCACTTTTCG GGGAAATGTG CGCGGAACCC CTATTTGTTT
5301 ATTTTTCTAA ATACATTCAA ATATGTATCC GCTCATGAGA CAATAACCCT
5351 GATAAATGCT TCAATAATAT TGAAAAAGGA AGAGTATGAG TATTCAACAT
5401 TTCCGTGTCG CCCTTATTCC CTTTTTTGGG GCATTTTGCC TTCCTGTTTT
5451 TGCTCACCCA GAAACGCTGG TGAAAGTAAA AGATGCTGAA GATCAGTTGG
5501 GTGCACGAGT GGGTTACATC GAACTGGATC TCAACAGCGG TAAGATCCTT
5551 GAGAGTTTTC GCCCCGAAGA ACGTTTTCCA ATGATGAGCA CTTTTAAAGT
5601 TCTGCTATGT GGCGCGGTAT TATCCCGTAT TGACGCCGGG CAAGAGCAAC
5651 TCGGTCGCCG CATACACTAT TCTCAGAATG ACTTGGTTGA GTACTCACCA
5701 GTCACAGAAA AGCATCTTAC GGATGGCATG ACAGTAAGAG AATTATGCAG
5751 TGCTGCCATA ACCATGAGTG ATAACACTGC GGCCAACTTA CTTCTGACAA
5801 CGATCGGAGG ACCGAAGGAG CTAACCGCTT TTTTGCACAA CATGGGGGAT
5851 CATGTAACTC GCCTTGATCG TTGGGAACCG GAGCTGAATG AAGCCATACC
5901 AAACGACGAG CGTGACACCA CGATGCCTGT AGCAATGGCA ACAACGTTGC
5951 GCAAACTATT AACTGGCGAA CTACTTACTC TAGCTTCCCG GCAACAATTA
6001 ATAGACTGGA TGGAGGCGGA TAAAGTTGCA GGACCACTTC TGCGCTCGGC
6051 CCTTCCGGCT GGCTGGTTTA TTGCTGATAA ATCTGGAGCC GGTGAGCGTG
6101 GGTCTCGCGG TATCATTGCA GCACTGGGGC CAGATGGTAA GCCCTCCCGT
6151 ATCGTAGTTA TCTACAGGAC GGGGAGTCAG GCAACTATGG ATGAACGAAA
6201 TAGACAGATC GCTGAGATAG GTGCCTCACT GATTAAGCAT TGGTAACTGT
6251 CAGACCAAGT TTACTCATAT ATACTTTAGA TTGATTTAAA ACTTCATTTT
6301 TAATTTAAAA GGATCTAGGT GAAGATCCTT TTTGATAATC TCATGACCAA
6351 AATCCCTTAA CGTGAGTTTT CGTTCCACTG AGCGTCAGAC CCCGTAGAAA
6401 AGATCAAAGG ATCTTCTTGA GATCCTTTTT TTCTGCGCGT AATCTGCTGC
6451 TTGCAAACAA AAAAACCACC GCTACCAGCG GTGGTTTGTT TGCCGGATCA
6501 AGAGCTACCA ACTCTTTTTC CGAAGGTAAC TGGCTTCAGC AGAGCGCAGA
6551 TAGCAAATAC TGTCCTTCTA GTGTAGCCGT AGTTAGGCCA CCACTTCAAG
6601 AACTCTGTAG CACCGCCTAC ATACCTCGCT CTGCTAATCC TGTTACCAGT
6651 GGCTGCTGCC AGTGGCGATA AGTCGTGTCT TACCGGGTTG GACTCAAGAC
6701 GATAGTTACC GGATAAGGCG CAGCGGTCGG GCTGAACGGG GGGTTCGTGC
6751 ACACAGCCCA GCTTGGAGCG AACGACCTAC ACCGAACTGA GATACCTACA
6801 GCGTGAGCTA TGAGAAAGCG CCACGCTTCC CGAAGGGAGA AAGGCGGACA
6851 GGTATCCGGT AAGCGGCAGG GTCGGAACAG GAGAGCGCAC GAGGGAGCTT
6901 CCAGGGGGAA ACGCCTGGTA TCTTTATAGT CCTGTCGGGT TTCGCCACCT
6951 CTGACTTGAG CGTCGATTTT TGTGATGCTC GTCAGGGGGG CGGAGCCTAT
7001 GGAAAAACGC CAGCAACGCG GCCTTTTTAC GGTTCCTGGC CTTTTGCTGG
7051 CCTTTTGCTC ACATGT
AAVC-CBh-mBPIP-TATκ28-eGFP
L-ITR: 1-141
CBh promoter: 159-976
Bip-TATκ28-EGFP: 1000-1905
bGHp(A): 1930-2157
R-ITR: 2169-2309
Amp(R):3226-4083
pUC origin: 4234-4901
SEQ ID NO: 112
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGTT AATAGTAATC AATTACGGGG TCATTAGTTC ATAGCCCATA
  201 TATGGAGTTC CGCGTTACAT AACTTACGGT AAATGGCCCG CCTGGCTGAC
  251 CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG
  351 GTAAACTGCC CACTTGGCAG TACATCAAGT GTATCATATG CCAAGTACGC
  401 CCCCTATTGA CGTCAATGAC GGTAAATGGC CCGCCTGGCA TTATGCCCAG
  451 TACATGACCT TACGGGACTT TCCTACTTGG CAGTACATCT CCACGTTCTG
  501 CTTCAGTCTC CCCATCTCCC CCCCCTCCCC ACCCCCAATT TTGTATTTAT
  551 TTATTTTTTA ATTATTTTGT GCAGCGATGG GGGCGGGGGG GGGGGGGGCG
  601 CGCGCCAGGC GGGGCGGGGC GGGGCGAGGG GCGGGGCGGG GCGAGGCGGA
  651 GAGGTGCGGC GGCAGCCAAT CAGAGCGGCG CGCTCCGAAA GTTTCCTTTT
  701 ATGGCGAGGC GGCGGCGGCG GCGGCCCTAT AAAAAGCGAA GCGCGCGGCG
  751 GGGAGTCGCT GCGTTGCCTT CGCCCCGTGC CCCGCTCCGC GCCGCCTCGC
  801 GCCGCCCGCC CCGGCTCTGA CTGACCGCGT TACTCCCACA GGTGAGCGGG
  851 CGGGACGGCC CTTCTCCTCC GGGCTGTAAT TAGCAAGAGG TAAGGGTTTA
  901 AGGGATGGTT GGTTGGTGGG GTATTAATGT TTAATTACCT GTTTTACAGG
  951 CCTGAAATCA CTTGGTTTTA GGTTGGGCTA GCCAAAGCTT GCCGCCACCA
1001 TGAAACTGTC CCTGGTCGCC GCCATGCTGC TCCTCCTGTC ACTGGTCGCC
1051 GCTATGCTGC TCCTCCTCTC CGCTGCTCGG GCTGGGGACG CTGCTCAGCC
1101 AGCTAGGAGA GCTAGGAGGA CCAAGCTGGC TGCTTACGCT AGGAAGGCTG
1151 CTAGGCAGGC TAGAGCTGGA GGAGGCGGAT CTATGGTGAG CAAGGGAGAG
1201 GAGCTGTTCA CAGGCGTGGT GCCCATCCTG GTGGAGCTGG ACGGAGATGT
1251 GAACGGCCAC AAGTTTAGCG TGTCCGGAGA GGGAGAGGGC GACGCTACCT
1301 ACGGAAAGCT GACACTGAAG TTCATCTGCA CCACAGGCAA GCTGCCCGTG
1351 CCTTGGCCAA CCCTGGTGAC CACACTGACA TACGGCGTGC AGTGTTTTTC
1401 CAGGTACCCA GACCACATGA AGCAGCACGA TTTCTTTAAG TCTGCCATGC
1451 CCGAGGGATA CGTGCAGGAG CGGACCATCT TCTTTAAGGA CGATGGCAAC
1501 TACAAGACCC GCGCTGAGGT GAAGTTCGAG GGAGACACAC TGGTGAACAG
1551 GATCGAGCTG AAGGGCATCG ACTTTAAGGA GGATGGAAAC ATCCTGGGCC
1601 ACAAGCTGGA GTACAACTAC AACAGCCACA ACGTGTACAT CATGGCCGAT
1651 AAGCAGAAGA ACGGAATCAA GGTGAACTTC AAGATCAGAC ACAACATCGA
1701 GGACGGCTCC GTGCAGCTGG CTGATCACTA CCAGCAGAAC ACCCCTATCG
1751 GAGACGGACC CGTGCTGCTG CCTGATAACC ACTACCTGTC CACACAGTCT
1801 GCCCTGAGCA AGGACCCAAA CGAGAAGCGG GATCACATGG TGCTGCTGGA
1851 ATTTGTGACT GCTGCTGGTA TTACACTGGG TATGGATGAA CTCTATAAAT
1901 GATAAGCGGC CGCAACTCGA GACTCTAGAC GACTGTGCCT TCTAGTTGCC
1951 AGCCATCTGT TGTTTGCCCC TCCCCCGTGC CTTCCTTGAC CCTGGAAGGT
2001 GCCACTCCCA CTGTCCTTTC CTAATAAAAT GAGGAAATTG CATCGCATTG
2051 TCTGAGTAGG TGTCATTCTA TTCTGGGGGG TGGGGTGGGG CAGGACAGCA
2101 AGGGGGAGGA TTGGGAAGAC AATAGCAGGC ATGCTGGGGA TGCGGTGGGC
2151 TCTATGGCCG CGGGCCGCAG GAACCCCTAG TGATGGAGTT GGCCACTCCC
2201 TCTCTGCGCG CTCGCTCGCT CACTGAGGCC GGGCGACCAA AGGTCGCCCG
2251 ACGCCCGGGC TTTGCCCGGG CGGCCTCAGT GAGCGAGCGA GCGCGCAGCT
2301 GCCTGCAGGG GCGCCTGATG CGGTATTTTC TCCTTACGCA TCTGTGCGGT
2351 ATTTCACACC GCATACGTCA AAGCAACCAT AGTACGCGCC CTGTAGCGGC
2401 GCATTAAGCG CGGCGGGTGT GGTGGTTACG CGCAGCGTGA CCGCTACACT
2451 TGCCAGCGCC CTAGCGCCCG CTCCTTTCGC TTTCTTCCCT TCCTTTCTCG
2501 CCACGTTCGC CGGCTTTCCC CGTCAAGCTC TAAATCGGGG GCTCCCTTTA
2551 GGGTTCCGAT TTAGTGCTTT ACGGCACCTC GACCCCAAAA AACTTGATTT
2601 GGGTGATGGT TCACGTAGTG GGCCATCGCC CTGATAGACG GTTTTTCGCC
2651 CTTTGACGTT GGAGTCCACG TTCTTTAATA GTGGACTCTT GTTCCAAACT
2701 GGAACAACAC TCAACCCTAT CTCGGGCTAT TCTTTTGATT TATAAGGGAT
2751 TTTGCCGATT TCGGCCTATT GGTTAAAAAA TGAGCTGATT TAACAAAAAT
2801 TTAACGCGAA TTTTAACAAA ATATTAACGT TTACAATTTT ATGGTGCACT
2851 CTCAGTACAA TCTGCTCTGA TGCCGCATAG TTAAGCCAGC CCCGACACCC
2901 GCCAACACCC GCTGACGCGC CCTGACGGGC TTGTCTGCTC CCGGCATCCG
2951 CTTACAGACA AGCTGTGACC GTCTCCGGGA GCTGCATGTG TCAGAGGTTT
3001 TCACCGTCAT CACCGAAACG CGCGAGACGA AAGGGCCTCG TGATACGCCT
3051 ATTTTTATAG GTTAATGTCA TGATAATAAT GGTTTCTTAG ACGTCAGGTG
3101 GCACTTTTCG GGGAAATGTG CGCGGAACCC CTATTTGTTT ATTTTTCTAA
3151 ATACATTCAA ATATGTATCC GCTCATGAGA CAATAACCCT GATAAATGCT
3201 TCAATAATAT TGAAAAAGGA AGAGTATGAG TATTCAACAT TTCCGTGTCG
3251 CCCTTATTCC CTTTTTTGGG GCATTTTGCC TTCCTGTTTT TGCTCACCCA
3301 GAAACGCTGG TGAAAGTAAA AGATGCTGAA GATCAGTTGG GTGCACGAGT
3351 GGGTTACATC GAACTGGATC TCAACAGCGG TAAGATCCTT GAGAGTTTTC
3401 GCCCCGAAGA ACGTTTTCCA ATGATGAGCA CTTTTAAAGT TCTGCTATGT
3451 GGCGCGGTAT TATCCCGTAT TGACGCCGGG CAAGAGCAAC TCGGTCGCCG
3501 CATACACTAT TCTCAGAATG ACTTGGTTGA GTACTCACCA GTCACAGAAA
3551 AGCATCTTAC GGATGGCATG ACAGTAAGAG AATTATGCAG TGCTGCCATA
3601 ACCATGAGTG ATAACACTGC GGCCAACTTA CTTCTGACAA CGATCGGAGG
3651 ACCGAAGGAG CTAACCGCTT TTTTGCACAA CATGGGGGAT CATGTAACTC
3701 GCCTTGATCG TTGGGAACCG GAGCTGAATG AAGCCATACC AAACGACGAG
3751 CGTGACACCA CGATGCCTGT AGCAATGGCA ACAACGTTGC GCAAACTATT
3801 AACTGGCGAA CTACTTACTC TAGCTTCCCG GCAACAATTA ATAGACTGGA
3851 TGGAGGCGGA TAAAGTTGCA GGACCACTTC TGCGCTCGGC CCTTCCGGCT
3901 GGCTGGTTTA TTGCTGATAA ATCTGGAGCC GGTGAGCGTG GGTCTCGCGG
3951 TATCATTGCA GCACTGGGGC CAGATGGTAA GCCCTCCCGT ATCGTAGTTA
4001 TCTACACGAC GGGGAGTCAG GCAACTATGG ATGAACGAAA TAGACAGATC
4051 GCTGAGATAG GTGCCTCACT GATTAAGCAT TGGTAACTGT CAGACCAAGT
4101 TTACTCATAT ATACTTTAGA TTGATTTAAA ACTTCATTTT TAATTTAAAA
4151 GGATCTAGGT GAAGATCCTT TTTGATAATC TCATGACCAA AATCCCTTAA
4201 CGTGAGTTTT CGTTCCACTG AGCGTCAGAC CCCGTAGAAA AGATCAAAGG
4251 ATCTTCTTGA GATCCTTTTT TTCTGCGCGT AATCTGCTGC TTGCAAACAA
4301 AAAAACCACC GCTACCAGCG GTGGTTTGTT TGCCGGATCA AGAGCTACCA
4351 ACTCTTTTTC CGAAGGTAAC TGGCTTCAGC AGAGCGCAGA TACCAAATAC
4401 TGTCCTTCTA GTGTAGCCGT AGTTAGGCCA CCACTTCAAG AACTCTGTAG
4451 CACCGCCTAC ATACCTCGCT CTGCTAATCC TGTTACCAGT GGCTGCTGCC
4501 AGTGGCGATA AGTCGTGTCT TACCGGGTTG GACTCAAGAC GATAGTTACC
4551 GGATAAGGCG CAGCGGTCGG GCTGAACGGG GGGTTCGTGC ACACAGCCCA
4601 GCTTGGAGCG AACGACCTAC ACCGAACTGA GATACCTACA GCGTGAGCTA
4651 TGAGAAAGCG CCACGCTTCC CGAAGGGAGA AAGGCGGACA GGTATCCGGT
4701 AAGCGGCAGG GTCGGAACAG GAGAGCGCAC GAGGGAGCTT CCAGGGGGAA
4751 ACGCCTGGTA TCTTTATAGT CCTGTCGGGT TTCGCCACCT CTGACTTGAG
4801 CGTCGATTTT TGTGATGCTC GTCAGGGGGG CGGAGCCTAT GGAAAAACGC
4851 CAGCAACGCG GCCTTTTTAC GGTTCCTGGC CTTTTGCTGG CCTTTTGCTC
4901 ACATGT
AAVC-CBh- mBPIP-TATκ28-NLS-eGFP
L-ITR: 1-141
CBh promoter: 159-976
BIp-TATκ28-NLS-eGFP: 1000-1965
bGHp(A): 1990-2217
R-ITR: 2229-2369
Amp(R): 3286-4143
pUC origin: 4294-4961
SEQ ID NO: 113
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGTT AATAGTAATC AATTACGGGG TCATTAGTTC ATAGGCCATA
  201 TATGGAGTTC CGCGTTACAT AACTTACGGT AAATGGCCCG CCTGGCTGAC
  251 CGCCCAACGA CCCCCGCCCA TTGACGTCAA TAATGACGTA TGTTCCCATA
  301 GTAACGCCAA TAGGGACTTT CCATTGACGT CAATGGGTGG AGTATTTACG
  351 GTAAACTGCC CACTTGGCAG TACATCAAGT GTATCATATG CCAAGTACGC
  401 CCCCTATTGA CGTCAATGAC GGTAAATGGC CCGCCTGGCA TTATGCCCAG
  451 TACATGACCT TACGGGACTT TCCTACTTGG CAGTACATCT CCACGTTCTG
  501 CTTCACTCTC CCCATCTCCC CCCCCTCCCC ACCCCCAATT TTGTATTTAT
  551 TTATTTTTTA ATTATTTTGT GCAGCGATGG GGGCGGGGGG GGGGGGGGCG
  601 CGCGCCAGGC GGGGCGGGGC GGGGCGAGGG GCGGGGCGGG GCGAGGCGGA
  651 GAGGTGCGGC GGCAGCCAAT CAGAGCGGCG CGCTCCGAAA GTTTCCTTTT
  701 ATGGCGAGGC GGCGGCGGCG GCGGCCCTAT AAAAAGCGAA GCGCGCGGCG
  751 GGGAGTCGCT GCGTTGCCTT CGCCCCGTGC CCCGCTCCGC GCCGCCTCGC
  801 GCCGCCCGCC CCGGCTCTGA CTGACCGCGT TACTCCCACA GGTGAGCGGG
  851 CGGGACGGCC CTTCTCCTCC GGGCTGTAAT TAGCAAGAGG TAAGGGTTTA
  901 AGGGATGGTT GGTTGGTGGG GTATTAATGT TTAATTACCT GTTTTACAGG
  951 CCTGAAATCA CTTGGTTTTA GGTTGGGCTA GCCAAAGCTT GCCGCCACCA
1001 TGAAACTCAG TCTGGTCGCC GCTATGCTCC TGCTCCTCTC CCTGGTCGCC
1051 GCTATGCTCC TGCTCCTGTC TGCTGCCCGC GCTGGGGACG CTGCTCAGCC
1101 AGCTAGGAGA GCTAGGAGGA CCAAGCTGGC TGCTTACGCT AGAAAGGCTG
1151 CTAGGCAGGC TAGAGCTGGA GGAGGAGGAT CCATGGCTCC CAAGAAGAAG
1201 AGGAAGGTGC GCTACCCCGC CTTCCTGTAC AAGGTGGCTA CCATGGTGTC
1251 TAAGGGAGAG GAGCTGTTTA CAGGCGTGGT GCCCATCCTG GTGGAGCTGG
1301 ACGGAGATGT GAACGGCCAC AAGTTCAGCG TGTCCGGAGA GGGAGAGGGC
1351 GACGCCACCT ACGGAAAGCT GACACTGAAG TTTATCTGCA CCACAGGCAA
1401 GCTGCCCGTG CCTTGGCCAA CCCTGGTGAC CACACTGACA TACGGCGTGC
1451 AGTGTTTCTC TCGGTACCCT GACCACATGA AGCAGCACGA TTTCTTTAAG
1501 AGCGCCATGC CAGAGGGATA CGTGCAGGAG AGGACAATCT TCTTTAAGGA
1551 CGATGGCAAC TACAAGACCA GAGCTGAGGT GAAGTTCGAG GGAGACACAC
1601 TGGTGAACCG GATCGAGCTG AAGGGCATCG ACTTTAAGGA GGATGGAAAC
1651 ATCCTGGGCC ACAAGCTGGA GTACAACTAC AACAGCCACA ACGTGTACAT
1701 CATGGCCGAT AAGCAGAAGA ACGGAATCAA GGTGAACTTT AAGATCCGCC
1751 ACAACATCGA GGACGGCTCC GTGGAGCTGG CTGATCACTA CCAGCAGAAC
1801 ACCCCAATCG GAGACGGACC CGTGCTGCTG CCTGATAACC ACTACCTGTC
1851 TACACAGAGC GCCCTGTCCA AGGACCCTAA CGAGAAGAGG GATCACATGG
1901 TCCTCCTGGA ATTTGTGACT GCTGCTGGGA TTACTCTCGG TATGGATGAA
1951 CTGTATAAAT GATAAGCGGC CGCAACTCGA GACTCTAGAC GACTGTGCCT
2001 TCTAGTTGCC AGCCATCTGT TGTTTGCCCC TCCCCCGTGC CTTCCTTGAC
2051 CCTGGAAGGT GCCACTCCCA CTGTCCTTTC CTAATAAAAT GAGGAAATTG
2101 CATCGCATTG TCTGAGTAGG TGTCATTCTA TTCTGGGGGG TGGGGTGGGG
2151 CAGGACAGCA AGGGGGAGGA TTGGGAAGAC AATAGCAGGC ATGCTGGGGA
2201 TGCGGTGGGC TCTATGGCCG CGGGCCGCAG GAACCCCTAG TGATGGAGTT
2251 GGCCACTCCC TCTCTGCGCG CTCGCTCGCT CACTGAGGCC GGGCGACCAA
2301 AGGTCGCCCG ACGCCCGGGC TTTGCCCGGG CGGCCTCAGT GAGCGAGCGA
2351 GCGCGCAGCT GCCTGCAGGG GCGCCTGATG CGGTATTTTC TCCTTACGCA
2401 TCTGTGCGGT ATTTCACACC GCATACGTCA AAGCAACCAT AGTACGCGCC
2451 CTGTAGCGGC GCATTAAGCG CGGCGGGTGT GGTGGTTACG CGCAGCGTGA
2501 CCGCTACACT TGCCAGCGCC CTAGCGCCCG CTCCTTTCGC TTTCTTCCCT
2551 TCCTTTCTCG CCACGTTCGC CGGCTTTCCC CGTCAAGCTC TAAATCGGGG
2601 GCTCCCTTTA GGGTTCCGAT TTAGTGCTTT ACGGCACCTC GACCCCAAAA
2651 AACTTGATTT GGGTGATGGT TCACGTAGTG GGCCATCGCC CTGATAGACG
2701 GTTTTTCGCC CTTTGACGTT GGAGTCCACG TTCTTTAATA GTGGACTCTT
2751 GTTCCAAACT GGAACAACAC TCAACCCTAT CTCGGGCTAT TCTTTTGATT
2801 TATAAGGGAT TTTGCCGATT TCGGCCTATT GGTTAAAAAA TGAGCTGATT
2851 TAACAAAAAT TTAACGCGAA TTTTAACAAA ATATTAACGT TTACAATTTT
2901 ATGGTGCACT CTCAGTACAA TCTGCTCTGA TGCCGCATAG TTAAGCCAGC
2951 CCCGACACCC GCCAACACCC GCTGACGCGC CCTGACGGGC TTGTCTGCTC
3001 CCGGCATCCG CTTACAGACA AGCTGTGACC GTCTCCGGGA GCTGCATGTG
3051 TCAGAGGTTT TCACCGTCAT CACCGAAACG CGCGAGACGA AAGGGCCTCG
3101 TGATACGCCT ATTTTTATAG GTTAATGTCA TGATAATAAT GGTTTCTTAG
3151 ACGTCAGGTG GCACTTTTCG GGGAAATGTG CGCGGAACCC CTATTTGTTT
3201 ATTTTTCTAA ATACATTCAA ATATGTATCC GCTCATGAGA CAATAACCCT
3251 GATAAATGCT TCAATAATAT TGAAAAAGGA AGAGTATGAG TATTCAACAT
3301 TTCCGTGTCG CCCTTATTCC CTTTTTTGCG GCATTTTGCC TTCCTGTTTT
3351 TGCTCACCCA GAAACGCTGG TGAAAGTAAA AGATGCTGAA GATCAGTTGG
3401 GTGCACGAGT GGGTTACATC GAACTGGATC TCAACAGCGG TAAGATCCTT
3451 GAGAGTTTTC GCCCCGAAGA ACGTTTTCCA ATGATGAGCA CTTTTAAAGT
3501 TCTGCTATGT GGCGCGGTAT TATCCCGTAT TGACGCCGGG CAAGAGCAAC
3551 TCGGTCGCCG CATACACTAT TCTCAGAATG ACTTGGTTGA GTACTCACCA
3601 GTCACAGAAA AGCATCTTAC GGATGGCATG ACAGTAAGAG AATTATGCAG
3651 TGCTGCCATA ACCATGAGTG ATAACACTGC GGCCAACTTA CTTCTGACAA
3701 CGATCGGAGG ACCGAAGGAG CTAACCGCTT TTTTGCACAA CATGGGGGAT
3751 CATGTAACTC GCCTTGATCG TTGGGAACCG GAGCTGAATG AAGCCATACC
3801 AAACGACGAG CGTGACACCA CGATGCCTGT AGCAATGGCA ACAACGTTGC
3851 GCAAACTATT AACTGGCGAA CTACTTACTC TAGCTTCCCG GCAACAATTA
3901 ATAGACTGGA TGGAGGCGGA TAAAGTTGCA GGACCACTTC TGCGCTCGGC
3951 CCTTCCGGCT GGCTGGTTTA TTGCTGATAA ATCTGGAGCC GGTGAGCGTG
4001 GGTCTCGCGG TATCATTGCA GCACTGGGGC CAGATGGTAA GCCCTCCCGT
4051 ATCGTAGTTA TCTACACGAC GGGGAGTCAG GCAACTATGG ATGAACGAAA
4101 TAGACAGATC GCTGAGATAG GTGCCTCACT GATTAAGCAT TGGTAACTGT
4151 CAGACCAAGT TTACTCATAT ATACTTTAGA TTGATTTAAA ACTTCATTTT
4201 TAATTTAAAA GGATCTAGGT GAAGATCCTT TTTGATAATC TCATGACCAA
4251 AATCCCTTAA CGTGAGTTTT CGTTCCACTG AGCGTCAGAC CCCGTAGAAA
4301 AGATCAAAGG ATCTTCTTGA GATCCTTTTT TTCTGCGCGT AATCTGCTGC
4351 TTGCAAACAA AAAAACCACC GCTACCAGCG GTGGTTTGTT TGCCGGATCA
4401 AGAGCTACCA ACTCTTTTTC CGAAGGTAAC TGGCTTCAGC AGAGCGCAGA
4451 TACCAAATAC TGTCCTTCTA GTGTAGCCGT AGTTAGGCCA CCACTTCAAG
4501 AACTCTGTAG CACCGCCTAC ATACCTCGCT CTGCTAATCC TGTTACCAGT
4551 GGCTGCTGCC AGTGGCGATA AGTCGTGTCT TACCGGGTTG GACTCAAGAC
4601 GATAGTTACC GGATAAGGCG CAGCGGTCGG GCTGAACGGG GGGTTCGTGC
4651 ACACAGCCCA GCTTGGAGCG AACGACCTAC ACCGAACTGA GATACCTACA
4701 GCGTGAGCTA TGAGAAAGCG CCACGCTTCC CGAAGGGAGA AAGGCGGACA
4751 GGTATCCGGT AAGCGGCAGG GTCGGAACAG GAGAGCGCAC GAGGGAGCTT
4801 CCAGGGGGAA ACGCCTGGTA TCTTTATAGT CCTGTCGGGT TTCGCCACCT
4851 CTGAOTTGAG CGTCGATTTT TGTGATGCTC GTCAGGGGGG CGGAGCCTAT
4901 GGAAAAACGC CAGCAACGCG GCCTTTTTAC GGTTCCTGGC CTTTTGCTGG
4951 CCTTTTGCTC ACATGT
AAVC-Syn-hCDKL5-107
L-ITR: 1-141
Syn-1 promoter: 159-730
hCDKL5-107 ORF: 754-3639
bGHp(A): 3664-3891
R-ITR: 3903-4043
Amp(R): 4960-5817
pUC origin: 5968-6635
SEQ ID NO: 114
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGAC TACAAACCGA GTATCTGCAG AGGGCCCTGC GTATGAGTGC
  201 AAGTGGGTTT TAGGACCAGG ATGAGGCGGG GTGGGGGTGC CTACCTGACG
  251 ACCGACCCCG ACCCACTGGA CAAGCACCCA ACCCCCATTC CCCAAATTGC
  301 GCATCCCCTA TCAGAGAGGG GGAGGGGAAA CAGGATGCGG CGAGGCGCGT
  351 GCGCACTGCC AGCTTCAGCA CCGCGGACAG TGCCTTCGCC CCCGCCTGGC
  401 GGCGCGCGCC ACCGCCGCCT CAGCACTGAA GGCGCGCTGA CGTCACTCGC
  451 CGGTCCCCCG CAAACTCCCC TTCCCGGCCA CCTTGGTCGC GTCCGCGCCG
  501 CCGCCGGCCC AGCCGGACCG CACCACGCGA GGCGCGAGAT AGGGGGGCAC
  551 GGGCGCGACC ATCTGCGCTG CGGCGCCGGC GACTCAGCGC TGCCTCAGTC
  601 TGCGGTGGGC AGCGGAGGAG TCGTGTCGTG CCTGAGAGCG CAGCTGTGCT
  651 CCTGGGCACC GCGCAGTCCG CCCCCGCGGC TCCTGGCCAG ACCACCCCTA
  701 GGACCCCCTG CCCCAAGTCG CAGCCTTCGA GCTAGCCAAA GCTTGCCGCC
  751 ACCATGAAAA TCCCTAACAT TGGTAACGTG ATGAACAAGT TTGAAATCCT
  801 CGGGGTCGTC GGAGAAGGTG CCTACGGGGT CGTGCTGAAG TGCAGACACA
  851 AGGAGACACA CGAGATCGTG GCCATCAAGA AGTTCAAGGA TAGCGAGGAG
  901 AACGAGGAGG TGAAGGAGAC AACCCTGAGA GAGCTGAAGA TGCTGCGGAC
  951 ACTGAAGCAG GAGAACATCG TGGAGCTGAA GGAGGCTTTC AGGAGACGGG
1001 GAAAGCTGTA CCTGGTGTTT GAGTACGTGG AGAAGAACAT GCTGGAGCTG
1051 CTGGAGGAGA TGCCTAACGG CGTGCCCCCT GAGAAGGTGA AGTCCTACAT
1101 CTACCAGCTG ATCAAGGCCA TCCACTGGTG CCACAAGAAC GACATCGTGC
1151 ACAGAGATAT CAAGCCAGAG AACCTGCTGA TCTCCCACAA CGACGTGCTG
1201 AAGCTGTGCG ATTTCGGCTT TGCCCGGAAC CTGAGCGAGG GAAACAACGC
1251 CAACTACACA GAGTACGTGG CTACCAGATG GTACCGGAGC CCAGAGCTGC
1301 TGCTGGGAGC TCCATACGGA AAGAGCGTGG ACATGTGGTC CGTGGGCTGC
1351 ATCCTGGGAG AGCTGTCTGA CGGCCAGCCT CTGTTCCCAG GAGAGAGCGA
1401 GATCGATCAG CTGTTTACCA TCCAGAAGGT GCTGGGCCCT CTGCCAAGCG
1451 AGCAGATGAA GCTGTTCTAC TCCAACCCTA GATTCCACGG ACTGCGGTTT
1501 CCCGCCGTGA ACCACCCTCA GAGCCTGGAG CGCAGGTACC TGGGCATCCT
1551 GAACTCCGTG CTGCTGGATC TGATGAAGAA CCTGCTGAAG CTGGACCCCG
1601 CCGATAGATA CCTGACCGAG CAGTGTCTGA ACCACCCTAC ATTTCAGACC
1651 CAGCGCCTGC TGGACAGGAG CCCTTCCAGA TCTGCTAAGC GGAAGCCATA
1701 CCACGTGGAG AGCTCCACCC TGTCCAACAG AAACCAGGCC GGCAAGTCTA
1751 CAGCTCTGCA GAGCCACCAC CGGAGCAACT CCAAGGACAT CCAGAACCTG
1801 TCTGTGGGCC TGCCTAGGGC TGATGAGGGA CTGCCAGCTA ACGAGAGCTT
1851 CCTGAACGGC AACCTGGCCG GAGCTTCTCT GAGCCCACTG CACACAAAGA
1901 CCTACCAGGC CTCTAGCCAG CCCGGCTCCA CATCTAAGGA CCTGACCAAC
1951 AACAACATCC CACACCTGCT GTCTCCCAAG GAGGCTAAGA GCAAGACCGA
2001 GTTCGACTTT AACATCGATC CCAAGCCTAG CGAGGGCCCA GGAACAAAGT
2051 ACCTGAAGAG CAACTCCCGC TCTCAGCAGA ACAGGCACTC CTTCATGGAG
2101 TCCTCTCAGT CTAAGGCCGG CACCCTGCAG CCCAACGAGA AGCAGAGCAG
2151 GCACTCCTAC ATCGATACCA TCCCCCAGAG CTCCAGAAGC CCTTCCTACC
2201 GGACAAAGGC CAAGAGCCAC GGCGCTCTGT CTGACAGCAA GTCCGTGTCT
2251 AACCTGTCCG AGGCTAGGGC TCAGATCGCT GAGCCAAGCA CCTCCAGGTA
2301 CTTTCCTTCT AGCTGTCTGG ACCTGAACTC TCCTACAAGC CCAACACCCA
2351 CCCGCCACTC CGATACAAGG ACCCTGCTGT CTCCAAGCGG CAGGAACAAC
2401 AGGAACGAGG GAACCCTGGA TTCTAGACGG ACCACAACCC GCCACAGCAA
2451 GACAATGGAG GAGCTGAAGC TGCCAGAGCA CATGGACTCC TCTCACTCCC
2501 ACTCTCTGAG CGCCCCCCAC GAGTCCTTCT CTTACGGCCT GGGATACACC
2551 TCCCCCTTCA GCTCCCAGCA GAGGCCCCAC AGGCACTCTA TGTACGTGAC
2601 ACGCGACAAG GTGAGGGCCA AGGGCCTGGA TGGAAGCCTG TCCATCGGAC
2651 AGGGAATGGC TGCTAGGGCT AACTCCCTGC AGCTGCTGTC TCCTCAGCCA
2701 GGAGAGCAGC TGCCACCAGA GATGACCGTG GCTCGCTCTA GCGTGAAGGA
2751 GACAAGCAGG GAGGGCACCT CCTCTTTCCA CACACGCCAG AAGTCCGAGG
2801 GCGGAGTGTA CCACGACCCC CACTCTGACG ATGGAACAGC TCCTAAGGAG
2851 AACAGGCACC TGTACAACGA TCCCGTGCCT CGCAGGGTGG GCTCCTTCTA
2901 CAGAGTGCCA TCTCCCCGGC CTGACAACAG CTTTCACGAG AACAACGTGT
2951 CCACCCGCGT GAGCTCCCTG CCTTCTGAGT CTAGCTCCGG AACAAACCAC
3001 TCTAAGAGGC AGCCCGCCTT TGACCCTTGG AAGAGCCCAG AGAACATCTC
3051 TCACAGCGAG CAGCTGAAGG AGAAGGAGAA GCAGGGCTTC TTTCGCAGCA
3101 TGAAGAAGAA GAAGAAGAAG AGCCAGACCG TGCCTAACTC CGACTCTCCA
3151 GATCTGCTGA CCCTGCAGAA GTCCATCCAC AGCGCCTCCA CACCATCTAG
3201 CCGCCCTAAG GAGTGGAGGC CTGAGAAGAT CAGCGATCTG CAGACACAGA
3251 GCCAGCCACT GAAGTCCCTG AGGAAGCTGC TGCACCTGTC CTCTGCCAGC
3301 AACCACCCCG CTAGCTCCGA CCCAAGATTC CAGCCCCTGA CAGCCCAGCA
3351 GACCAAGAAC TCTTTTAGCG AGATCCGGAT CCACCCTCTG TCCCAGGCTT
3401 CTGGCGGATC TAGCAACATC AGACAGGAGC CAGCTCCAAA GGGCCGGCCC
3451 GCTCTGCAGC TGCCTGGCCA GATGGACCCA GGATGGCACG TGTCCTCTGT
3501 GACAAGATCC GCCACCGAGG GACCATCCTA CTCTGAGCAG CTGGGCGCTA
3551 AGTCTGGCCC TAACGGACAC CCATACAATA GGACTAATAG AAGCAGAATG
3601 CCAAACCTCA ATGACCTCAA GGAAACAGCA CTCTGATAAG CGGCCGCAAC
3651 TCGAGACTCT AGACGACTGT GCCTTCTAGT TGCCAGCCAT CTGTTGTTTG
3701 CCCCTCCCCC GTGCCTTCCT TGACCCTGGA AGGTGCCACT CCCACTGTCC
3751 TTTCCTAATA AAATGAGGAA ATTGCATCGC ATTGTCTGAG TAGGTGTCAT
3801 TCTATTCTGG GGGGTGGGGT GGGGCAGGAC AGCAAGGGGG AGGATTGGGA
3851 AGACAATAGC AGGCATGCTG GGGATGCGGT GGGCTCTATG GCCGCGGGCC
3901 GCAGGAACCC CTAGTGATGG AGTTGGCCAC TCCCTCTCTG CGCGCTCGCT
3951 CGCTCACTGA GGCCGGGCGA CCAAAGGTCG CCCGACGCCC GGGCTTTGCC
4001 CGGGCGGCCT CAGTGAGCGA GCGAGCGCGC AGCTGCCTGC AGGGGCGGCT
4051 GATGCGGTAT TTTCTCCTTA CGCATCTGTG CGGTATTTCA CACCGCATAC
4101 GTCAAAGCAA CCATAGTACG CGCCCTGTAG CGGCGCATTA AGCGCGGCGG
4151 GTGTGGTGGT TACGCGCAGC GTGACCGCTA CACTTGCCAG CGCCCTAGCG
4201 CCCGCTCCTT TCGCTTTCTT CCCTTCCTTT CTCGCCACGT TCGCCGGCTT
4251 TCCCCGTCAA GCTCTAAATC GGGGGCTCCC TTTAGGGTTC CGATTTAGTG
4301 CTTTACGGCA CCTCGACCCC AAAAAACTTG ATTTGGGTGA TGGTTCACGT
4351 AGTGGGCCAT CGCCCTGATA GACGGTTTTT CGCCCTTTGA CGTTGGAGTC
4401 CACGTTCTTT AATAGTGGAC TCTTGTTCCA AACTGGAACA ACACTCAACC
4451 CTATCTCGGG CTATTCTTTT GATTTATAAG GGATTTTGCC GATTTCGGCC
4501 TATTGGTTAA AAAATGAGCT GATTTAACAA AAATTTAACG CGAATTTTAA
4551 CAAAATATTA ACGTTTACAA TTTTATGGTG CACTCTCAGT ACAATCTGCT
4601 CTGATGCCGC ATAGTTAAGC CAGCCCCGAC ACCCGCCAAC ACCCGCTGAC
4651 GCGCCCTGAC GGGCTTGTCT GCTCCCGGCA TCCGCTTACA GACAAGCTGT
4701 GACCGTCTCC GGGAGCTGCA TGTGTCAGAG GTTTTCACCG TCATCACCGA
4751 AACGCGCGAG ACGAAAGGGC CTCGTGATAC GCCTATTTTT ATAGGTTAAT
4801 GTCATGATAA TAATGGTTTC TTAGACGTCA GGTGGCACTT TTCGGGGAAA
4851 TGTGCGCGGA ACCCCTATTT GTTTATTTTT CTAAATACAT TCAAATATGT
4901 ATCCGCTCAT GAGACAATAA CCCTGATAAA TGCTTCAATA ATATTGAAAA
4951 AGGAAGAGTA TGAGTATTCA ACATTTCCGT GTCGCCCTTA TTCCCTTTTT
5001 TGCGGCATTT TGCCTTCCTG TTTTTGCTCA CCCAGAAACG CTGGTGAAAG
5051 TAAAAGATGC TGAAGATCAG TTGGGTGCAC GAGTGGGTTA CATCGAACTG
5101 GATCTCAACA GCGGTAAGAT CCTTGAGAGT TTTCGCCCCG AAGAACGTTT
5151 TCCAATGATG AGCACTTTTA AAGTTCTGCT ATGTGGCGCG GTATTATCCC
5201 GTATTGACGC CGGGCAAGAG CAACTCGGTC GCCGCATACA CTATTCTCAG
5251 AATGACTTGG TTGAGTACTC ACCAGTCACA GAAAAGCATC TTACGGATGG
5301 CATGACAGTA AGAGAATTAT GCAGTGCTGC CATAACCATG AGTGATAACA
5351 CTGCGGCCAA CTTACTTCTG ACAACGATCG GAGGACCGAA GGAGCTAACC
5401 GCTTTTTTGC ACAACATGGG GGATCATGTA ACTCGCCTTG ATCGTTGGGA
5451 ACCGGAGCTG AATGAAGCCA TACCAAACGA CGAGCGTGAC ACCACGATGC
5501 CTGTAGCAAT GGCAACAACG TTGCGCAAAC TATTAACTGG CGAACTACTT
5551 ACTCTAGCTT CCCGGCAACA ATTAATAGAC TGGATGGAGG CGGATAAAGT
5601 TGCAGGACCA CTTCTGCGCT CGGCCCTTCC GGCTGGCTGG TTTATTGCTG
5651 ATAAATCTGG AGCCGGTGAG CGTGGGTCTC GCGGTATCAT TGCAGCACTG
5701 GGGCCAGATG GTAAGCCCTC CCGTATCGTA GTTATCTACA CGACGGGGAG
5751 TCAGGCAACT ATGGATGAAC GAAATAGACA GATCGCTGAG ATAGGTGCCT
5801 CACTGATTAA GCATTGGTAA CTGTCAGACC AAGTTTACTC ATATATACTT
5851 TAGATTGATT TAAAACTTCA TTTTTAATTT AAAAGGATCT AGGTGAAGAT
5901 CCTTTTTGAT AATCTCATGA CCAAAATCCC TTAACGTGAG TTTTCGTTCC
5951 ACTGAGCGTC AGACCCCGTA GAAAAGATCA AAGGATCTTC TTGAGATCCT
6001 TTTTTTCTGC GCGTAATCTG CTGCTTGCAA ACAAAAAAAC CACCGCTACC
6051 AGCGGTGGTT TGTTTGCCGG ATCAAGAGCT ACCAACTCTT TTTCCGAAGG
6101 TAACTGGCTT CAGCAGAGCG CAGATACCAA ATACTGTCCT TCTAGTGTAG
6151 CCGTAGTTAG GCCACCACTT CAAGAACTCT GTAGCACCGC CTACATACCT
6201 CGCTCTGCTA ATCCTGTTAC CAGTGGCTGC TGCCAGTGGC GATAAGTCGT
6251 GTCTTACCGG GTTGGACTCA AGACGATAGT TACCGGATAA GGCGCAGCGG
6301 TCGGGCTGAA CGGGGGGTTC GTGCACACAG CCCAGCTTGG AGCGAACGAC
6351 CTACACCGAA CTGAGATACC TACAGCGTGA GCTATGAGAA AGCGCCACGC
6401 TTCCCGAAGG GAGAAAGGCG GACAGGTATC CGGTAAGCGG CAGGGTCGGA
6451 ACAGGAGAGO GCACGAGGGA GCTTCCAGGG GGAAACGCCT GGTATCTTTA
6501 TAGTCCTGTC GGGTTTCGCC ACCTCTGACT TGAGCGTCGA TTTTTGTGAT
6551 GCTCGTCAGG GGGGCGGAGC CTATGGAAAA ACGCCAGCAA CGCGGCCTTT
6601 TTACGGTTCC TGGCCTTTTG CTGGCCTTTT GCTCACATGT
AAVC-Syn-hCDKL5-107(dead kinase)
L-ITR: 1-141
Syn-1 promoter: 159-730
hCDKL5-107 ORF (kinase dead): 754-3639
bGHp(A): 3664-3891
R-ITR: 3903-4043
Amp(R): 4960-5817
pUC origin: 5968-6635
SEQ ID NO: 115
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGAC TACAAACCGA GTATCTGCAG AGGGCCCTGC GTATGAGTGC
  201 AAGTGGGTTT TAGGACCAGG ATGAGGCGGG GTGGGGGTGC CTACCTGACG
  251 ACCGACCCCG ACCCACTGGA CAAGCACCCA ACCCCCATTC CCCAAATTGC
  301 GCATCCCCTA TCAGAGAGGG GGAGGGGAAA CAGGATGCGG CGAGGCGCGT
  351 GCGCACTGCC AGCTTCAGCA CCGCGGACAG TGCCTTCGCC CCCGCCTGGC
  401 GGCGCGCGCC ACCGCCGCCT CAGCACTGAA GGCGCGCTGA CGTCACTCGC
  451 CGGTCCCCCG CAAACTCCCC TTCCCGGCCA CCTTGGTCGC GTCCGCGCCG
  501 CCGCCGGCCC AGCCGGACCG CACCACGCGA GGCGCGAGAT AGGGGGGCAC
  551 GGGCGCGACC ATCTGCGCTG CGGCGCCGGC GACTCAGCGC TGCCTCAGTC
  601 TGCGGTGGGC AGCGGAGGAG TCGTGTCGTG CCTGAGAGCG CAGCTGTGCT
  651 CCTGGGCACC GCGCAGTCCG CCCCCGCGGC TCCTGGCCAG ACCACCCCTA
  701 GGACCCCCTG CCCCAAGTCG CAGCCTTCGA GCTAGCCAAA GCTTGCCGCC
  751 ACCATGAAAA TCCCTAATAT CGGAAATGTG ATGAATAAGT TTGAAATCCT
  801 CGGGGTCGTC GGAGAAGGTG CCTACGGGGT CGTCCTGAAA TGCAGACACA
  851 AGGAGACACA CGAGATCGTG GCCATCAGGA GATTCAAGGA TAGCGAGGAG
  901 AACGAGGAGG TGAAGGAGAC AACCCTGCGC GAGCTGAAGA TGCTGAGGAC
  951 ACTGAAGCAG GAGAACATCG TGGAGCTGAA GGAGGCTTTC CGGCGCAGGG
1001 GAAAGCTGTA CCTGGTGTTT GAGTACGTGG AGAAGAACAT GCTGGAGCTG
1051 CTGGAGGAGA TGCCTAACGG CGTGCCCCCT GAGAAGGTGA AGTCCTACAT
1101 CTACCAGCTG ATCAAGGCCA TCCACTGGTG CCACAAGAAC GACATCGTGC
1151 ACCGCGATAT CAAGCCAGAG AACCTGCTGA TCTCCCACAA CGACGTGCTG
1201 AAGCTGTGCG ATTTCGGCTT TGCCAGGAAC CTGAGCGAGG GAAACAACGC
1251 CAACTACACA GAGTACGTGG CTACCCGCTG GTACAGGAGC CCAGAGCTGC
1301 TGCTGGGAGC TCCATACGGA AAGAGCGTGG ACATGTGGTC CGTGGGCTGC
1351 ATCCTGGGAG AGCTGTCTGA CGGCCAGCCT CTGTTCCCAG GAGAGAGCGA
1401 GATCGATCAG CTGTTTACCA TCCAGAAGGT GCTGGGCCCT CTGCCAAGCG
1451 AGCAGATGAA GCTGTTCTAC TCCAACCCTC GCTTCCAGGG ACTGAGGTTT
1501 CCCGCCGTGA ACCACCCTCA GAGCCTGGAG AGACGGTACC TGGGCATCCT
1551 GAACTCCGTG CTGCTGGATC TGATGAAGAA CCTGCTGAAG CTGGACCCCG
1601 CCGATCGCTA CCTGACCGAG CAGTGTCTGA ACCACCCTAC ATTTCAGACC
1651 CAGAGACTGC TGGACCGGAG CCCTTCCCGC TCTGCTAAGA GGAAGCCATA
1701 CCACGTGGAG AGCTCCACCC TGTCCAACCG CAACCAGGCC GGCAAGTCTA
1751 CAGCTCTGCA GAGCCACCAC AGGAGCAACT CCAAGGACAT CCAGAACCTG
1801 TCTGTGGGCC TGCCTAGGGC TGATGAGGGA CTGCCAGCTA ACGAGAGCTT
1851 CCTGAACGGC AACCTGGCCG GAGCTTCTCT GAGCCCACTG CACACAAAGA
1901 CCTACCAGGC CTCTAGCCAG CCCGGCTCCA CATCTAAGGA CCTGACCAAC
1951 AACAACATCC CACACCTGCT GTCTCCCAAG GAGGCTAAGA GCAAGACCGA
2001 GTTCGACTTT AACATCGATC CCAAGCCTAG CGAGGGCCCA GGAACAAAGT
2051 ACCTGAAGAG CAACTCCAGA TCTCAGCAGA ACCGGCACTC CTTCATGGAG
2101 TCCTCTCAGT CTAAGGCCGG CACCCTGCAG CCCAACGAGA AGCAGAGCAG
2151 GCACTCCTAC ATCGATACCA TCCCCCAGAG CTCCCGCAGC CCTTCCTACA
2201 GGACAAAGGC CAAGAGCCAC GGCGCTCTGT CTGACAGCAA GTCCGTGTCT
2251 AACCTGTCCG AGGCCAGAGC TCAGATCGCT GAGCCCAGCA CCTCCCGGTA
2301 CTTTCCTTCT AGCTGTCTGG ACCTGAACTC TCCTACAAGC CCAACACCCA
2351 CCAGACACTC CGATACACGG ACCCTGCTGT CTCCAAGCGG CAGAAACAAC
2401 CGGAAGGAGG GAACCCTGGA TTCTCGCAGG ACCACAACCA GACACAGCAA
2451 GACAATGGAG GAGCTGAAGC TGCCAGAGCA CATGGACTCC TCTCACTCCC
2501 ACTCTCTGAG CGCCCCCCAC GAGTCCTTCT CTTACGGCCT GGGATACACC
2551 TCCCCCTTCA GCTCCCAGCA GCGCCCCCAC AGGCACTCTA TGTACGTGAC
2601 AAGAGACAAG GTGCGGGCCA AGGGCCTGGA TGGAAGCCTG TCCATCGGCC
2651 AGGGAATGGC CGCTAGGGCT AACTCCCTGC AGCTGCTGTC TCCTCAGCCA
2701 GGAGAGCAGC TGCCACCCGA GATGACCGTG GCCAGATCTA GCGTGAAGGA
2751 GACAAGCCGG GAGGGCACCT CCTCTTTCCA CACAAGACAG AAGTCCGAGG
2801 GCGGAGTGTA CCACGACCCC CACTCTGACG ATGGAACAGC TCCTAAGGAG
2851 AACCGGCACC TGTACAACGA TCCCGTGCCT AGACGGGTGG GCTCCTTCTA
2901 CCGCGTGCCA TCTCCCAGGC CTGACAACAG CTTTCACGAG AACAACGTGT
2951 CCACCAGAGT GAGCTCCCTG CCTTCTGAGT CTAGCTCCGG AACAAACCAC
3001 TCTAAGCGGC AGCCCGCCTT TGACCCTTGG AAGAGCCCAG AGAACATCTC
3051 TCACAGCGAG CAGCTGAAGG AGAAGGAGAA GCAGGGCTTC TTTAGAAGCA
3101 TGAAGAAGAA GAAGAAGAAG AGCCAGACCG TGCCTAACTC CGACTCTCCA
3151 GATCTGCTGA CCCTGCAGAA GTCCATCCAC AGCGCCTCCA CACCCTCTAG
3201 CAGACCTAAG GAGTGGCGGC CTGAGAAGAT CAGCGATCTG CAGACACAGA
3251 GCCAGCCACT GAAGTCCCTG CGGAAGCTGC TGCACCTGTC CTCTGCCAGC
3301 AACCACCCAG CTAGCTCCGA CCCAAGGTTC CAGCCACTGA CAGCTCAGCA
3351 GACCAAGAAC TCTTTTAGCG AGATCAGGAT CCACCCTCTG TCCCAGGCTT
3401 CTGGCGGATC TAGCAACATC AGGCAGGAGC CAGCTCCAAA GGGCAGGCCC
3451 GCTCTGCAGC TGCCTGGACA GATGGACCCA GGATGGCACG TGTCCTCTGT
3501 GACAAGATCC GCCACCGAGG GACCATCCTA CTCTGAGCAG CTGGGCGCTA
3551 AGTCTGGCCC TAACGGACAC CCATACAACA GAACAAACAG AAGCAGAATG
3601 CCCAACCTCA ATGACCTCAA AGAAACAGCA CTCTGATAAG CGGCCGCAAC
3651 TCGAGACTCT AGACGACTGT GCCTTCTAGT TGCCAGCCAT CTGTTGTTTG
3701 CCCCTCCCCC GTGCCTTCCT TGACCCTGGA AGGTGCCACT CCCACTGTCC
3751 TTTCCTAATA AAATGAGGAA ATTGCATCGC ATTGTCTGAG TAGGTGTCAT
3801 TCTATTCTGG GGGGTGGGGT GGGGCAGGAC AGCAAGGGGG AGGATTGGGA
3851 AGACAATAGC AGGCATGCTG GGGATGCGGT GGGCTCTATG GCCGCGGGCC
3901 GCAGGAACCC CTAGTGATGG AGTTGGCCAC TCCCTCTCTG CGCGCTCGCT
3951 CGCTCACTGA GGCCGGGCGA CCAAAGGTCG CCCGACGCCC GGGCTTTGCC
4001 CGGGCGGCCT CAGTGAGCGA GCGAGCGCGC AGCTGCCTGC AGGGGCGGCT
4051 GATGCGGTAT TTTCTCCTTA CGCATCTGTG CGGTATTTCA CACCGCATAC
4101 GTCAAAGCAA CCATAGTACG CGCCCTGTAG CGGCGCATTA AGCGCGGCGG
4151 GTGTGGTGGT TACGCGCAGC GTGACCGCTA CACTTGCCAG CGCCCTAGCG
4201 CCCGCTCCTT TCGCTTTCTT CCCTTCCTTT CTCGCCACGT TCGCCGGCTT
4251 TCCCCGTCAA GCTCTAAATC GGGGGCTCCC TTTAGGGTTC CGATTTAGTG
4301 CTTTACGGCA CCTCGACCCC AAAAAACTTG ATTTGGGTGA TGGTTCACGT
4351 AGTGGGCCAT CGCCCTGATA GACGGTTTTT CGCCCTTTGA CGTTGGAGTC
4401 CACGTTCTTT AATAGTGGAC TCTTGTTCCA AACTGGAACA ACACTCAACC
4451 CTATCTCGGG CTATTCTTTT GATTTATAAG GGATTTTGCC GATTTCGGCC
4501 TATTGGTTAA AAAATGAGCT GATTTAACAA AAATTTAACG CGAATTTTAA
4551 CAAAATATTA ACGTTTACAA TTTTATGGTG CACTCTCAGT ACAATCTGCT
4601 CTGATGCCGC ATAGTTAAGC CAGCCCCGAC ACCCGCCAAC ACCCGCTGAC
4651 GCGCCCTGAC GGGCTTGTCT GCTCCCGGCA TCCGCTTACA GACAAGCTGT
4701 GACCGTCTCC GGGAGCTGCA TGTGTCAGAG GTTTTCACCG TCATCACCGA
4751 AACGCGCGAG ACGAAAGGGC CTCGTGATAC GCCTATTTTT ATAGGTTAAT
4801 GTCATGATAA TAATGGTTTC TTAGACGTCA GGTGGCACTT TTCGGGGAAA
4851 TGTGCGCGGA ACCCCTATTT GTTTATTTTT CTAAATACAT TCAAATATGT
4901 ATCCGCTCAT GAGACAATAA CCCTGATAAA TGCTTCAATA ATATTGAAAA
4951 AGGAAGAGTA TGAGTATTCA ACATTTCCGT GTCGCCCTTA TTCCCTTTTT
5001 TGCGGCATTT TGCCTTCCTG TTTTTGCTCA CCCAGAAACG CTGGTGAAAG
5051 TAAAAGATGC TGAAGATCAG TTGGGTGCAC GAGTGGGTTA CATCGAACTG
5101 GATCTCAACA GCGGTAAGAT CCTTGAGAGT TTTCGCCCCG AAGAACGTTT
5151 TCCAATGATG AGCACTTTTA AAGTTCTGCT ATGTGGCGCG GTATTATCCC
5201 GTATTGACGC CGGGCAAGAG CAACTCGGTC GCCGCATACA CTATTCTCAG
5251 AATGACTTGG TTGAGTACTC ACCAGTCACA GAAAAGCATC TTACGGATGG
5301 CATGACAGTA AGAGAATTAT GCAGTGCTGC CATAACCATG AGTGATAACA
5351 CTGCGGCCAA CTTACTTCTG ACAACGATCG GAGGACCGAA GGAGCTAACC
5401 GCTTTTTTGC ACAACATGGG GGATCATGTA ACTCGCCTTG ATCGTTGGGA
5451 ACCGGAGCTG AATGAAGCCA TACCAAACGA CGAGCGTGAC ACCACGATGC
5501 CTGTAGCAAT GGCAACAACG TTGCGCAAAC TATTAACTGG CGAACTACTT
5551 ACTCTAGCTT CCCGGCAACA ATTAATAGAC TGGATGGAGG CGGATAAAGT
5601 TGCAGGACCA CTTCTGCGCT CGGCCCTTCC GGCTGGCTGG TTTATTGCTG
5651 ATAAATCTGG AGCCGGTGAG CGTGGGTCTC GCGGTATCAT TGCAGCACTG
5701 GGGCCAGATG GTAAGCCCTC CCGTATCGTA GTTATCTACA CGACGGGGAG
5751 TCAGGCAACT ATGGATGAAC GAAATAGACA GATCGCTGAG ATAGGTGCCT
5801 CACTGATTAA GCATTGGTAA CTGTCAGACC AAGTTTACTC ATATATACTT
5851 TAGATTGATT TAAAACTTCA TTTTTAATTT AAAAGGATCT AGGTGAAGAT
5901 CCTTTTTGAT AATCTCATGA CCAAAATCCC TTAACGTGAG TTTTCGTTCC
5951 ACTGAGCGTC AGACCCCGTA GAAAAGATCA AAGGATCTTC TTGAGATCCT
6001 TTTTTTCTGC GCGTAATCTG CTGCTTGCAA ACAAAAAAAC CACCGCTACC
6051 AGCGGTGGTT TGTTTGCCGG ATCAAGAGCT ACCAACTCTT TTTCCGAAGG
6101 TAACTGGCTT CAGCAGAGCG CAGATACCAA ATACTGTCCT TCTAGTGTAG
6151 CCGTAGTTAG GCCACCACTT CAAGAACTCT GTAGCACCGC CTACATACCT
6201 CGCTCTGCTA ATCCTGTTAC CAGTGGCTGC TGCCAGTGGC GATAAGTCGT
6251 GTCTTACCGG GTTGGACTCA AGACGATAGT TACCGGATAA GGCGCAGCGG
6301 TCGGGCTGAA CGGGGGGTTC GTGCACACAG CCCAGCTTGG AGCGAACGAC
6351 CTACACCGAA CTGAGATACC TACAGCGTGA GCTATGAGAA AGCGCCACGC
6401 TTCCCGAAGG GAGAAAGGCG GACAGGTATC CGGTAAGCGG CAGGGTCGGA
6451 ACAGGAGAGO GCACGAGGGA GCTTCCAGGG GGAAACGCCT GGTATCTTTA
6501 TAGTCCTGTC GGGTTTCGCC ACCTCTGACT TGAGCGTCGA TTTTTGTGAT
6551 GCTCGTCAGG GGGGCGGAGC CTATGGAAAA ACGCCAGCAA CGCGGCCTTT
6601 TTACGGTTCC TGGCCTTTTG CTGGCCTTTT GCTCACATGT
AAVC-Syn-eGFP
L-ITR: 1-141
Syn-1 promoter: 159-730
EGFP ORF: 754-1476
bGHp(A): 1501-1728
R-ITR: 1740-1880
Amp(R): 2797-3654
pUC origin: 3805-4472
SEQ ID NO: 116
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGAC TACAAACCGA GTATCTGCAG AGGGCCCTGC GTATGAGTGC
  201 AAGTGGGTTT TAGGACCAGG ATGAGGCGGG GTGGGGGTGC CTACCTGACG
  251 ACCGACCCCG ACCCACTGGA CAAGCACCCA ACCCCCATTC CCCAAATTGC
  301 GCATCCCCTA TCAGAGAGGG GGAGGGGAAA CAGGATGCGG CGAGGCGCGT
  351 GCGCACTGCC AGCTTCAGCA CCGCGGACAG TGCCTTCGCC CCCGCCTGGC
  401 GGCGCGCGCC ACCGCCGCCT CAGCACTGAA GGCGCGCTGA CGTCACTCGC
  451 CGGTCCCCCG CAAACTCCCC TTCCCGGCCA CCTTGGTCGC GTCCGCGCCG
  501 CCGCCGGCCC AGCCGGACCG CACCACGCGA GGCGCGAGAT AGGGGGGCAC
  551 GGGCGCGACC ATCTGCGCTG CGGCGCCGGC GACTCAGCGC TGCCTCAGTC
  601 TGCGGTGGGC AGCGGAGGAG TCGTGTCGTG CCTGAGAGCG CAGCTGTGCT
  651 CCTGGGCACC GCGCAGTCCG CCCCCGCGGC TCCTGGCCAG ACCACCCCTA
  701 GGACCCCCTG CCCCAAGTCG CAGCCTTCGA GCTAGCCAAA GCTTGCCGCC
  751 ACCATGGTGT CAAAGGGGGA GGAACTGTTT ACTGGAGTCG TGCCTATTCT
  801 GGTCGAACTG GATGGGGATG TCAACGGTCA TAAGTTTAGC GTGTCCGGAG
  851 AGGGAGAGGG CGACGCTACC TACGGAAAGC TGACACTGAA GTTCATCTGC
  901 ACCACAGGCA AGCTGCCCGT GCCTTGGCCA ACCCTGGTGA CCACACTGAC
  951 ATACGGCGTG CAGTGTTTTA GCCGGTACCC AGACCACATG AAGCAGCACG
1001 ATTTCTTTAA GTCCGCCATG CCCGAGGGAT AGGTGGAGGA GAGGACCATC
1051 TTCTTTAAGG ACGATGGCAA CTACAAGACC AGAGCTGAGG TGAAGTTCGA
1101 GGGAGACACA CTGGTGAACC GGATCGAGCT GAAGGGCATC GACTTTAAGG
1151 AGGATGGAAA CATCCTGGGC CACAAGCTGG AGTACAACTA CAACTCTCAC
1201 AACGTGTACA TCATGGCCGA TAAGCAGAAG AACGGAATCA AGGTGAACTT
1251 CAAGATCCGC CACAACATCG AGGACGGCAG CGTGCAGCTG GCTGATCACT
1301 ACCAGCAGAA CACCCCTATC GGAGACGGAC CCGTGCTGCT GCCTGATAAC
1351 CACTACCTGA GCACACAGTC CGCTCTGTCT AAGGACCCAA ACGAGAAGAG
1401 GGATCACATG GTCCTCCTGG AATTTGTCAC TGCTGCTGGG ATTACTCTGG
1451 GGATGGATGA ACTCTATAAG TGATAAGCGG CCGCAACTCG AGACTCTAGA
1501 CGACTGTGCC TTCTAGTTGC CAGCCATCTG TTGTTTGCCC CTCCCCCGTG
1551 CCTTCCTTGA CCCTGGAAGG TGCCACTCCC ACTGTCCTTT CCTAATAAAA
1601 TGAGGAAATT GCATCGCATT GTCTGAGTAG GTGTCATTCT ATTCTGGGGG
1651 GTGGGGTGGG GCAGGACAGC AAGGGGGAGG ATTGGGAAGA CAATAGCAGG
1701 CATGCTGGGG ATGCGGTGGG CTCTATGGCC GCGGGCCGCA GGAACCCCTA
1751 GTGATGGAGT TGGCCACTCC CTCTCTGCGC GCTCGCTCGC TCACTGAGGC
1801 CGGGCGACCA AAGGTCGCCC GACGCCCGGG CTTTGCCCGG GCGGCCTCAG
1851 TGAGCGAGCG AGCGCGCAGC TGCCTGCAGG GGCGCCTGAT GCGGTATTTT
1901 CTCCTTACGC ATCTGTGCGG TATTTCACAC CGCATACGTC AAAGCAACCA
1951 TAGTACGCGC CCTGTAGCGG CGCATTAAGC GCGGCGGGTG TGGTGGTTAC
2001 GCGCAGCGTG ACCGCTACAC TTGCCAGCGC CCTAGCGCCC GCTCCTTTCG
2051 CTTTCTTCCC TTCCTTTCTC GCCACGTTCG CCGGCTTTCC CCGTCAAGCT
2101 CTAAATCGGG GGCTCCCTTT AGGGTTCCGA TTTAGTGCTT TACGGCACCT
2151 CGACCCCAAA AAACTTGATT TGGGTGATGG TTCACGTAGT GGGCCATCGC
2201 CCTGATAGAC GGTTTTTCGC CCTTTGACGT TGGAGTCCAC GTTCTTTAAT
2251 AGTGGACTCT TGTTCCAAAC TGGAACAACA CTCAACCCTA TCTCGGGCTA
2301 TTCTTTTGAT TTATAAGGGA TTTTGCCGAT TTCGGCCTAT TGGTTAAAAA
2351 ATGAGCTGAT TTAACAAAAA TTTAACGCGA ATTTTAACAA AATATTAACG
2401 TTTACAATTT TATGGTGCAC TCTCAGTACA ATCTGCTCTG ATGCCGCATA
2451 GTTAAGCCAG CCCCGACACC CGCCAACACC CGCTGACGCG CCCTGACGGG
2501 CTTGTCTGCT CCCGGCATCC GCTTACAGAC AAGCTGTGAC CGTCTCCGGG
2551 AGCTGCATGT GTCAGAGGTT TTCACCGTCA TCACCGAAAC GCGCGAGACG
2601 AAAGGGCCTC GTGATACGCC TATTTTTATA GGTTAATGTC ATGATAATAA
2651 TGGTTTCTTA GACGTCAGGT GGCACTTTTC GGGGAAATGT GCGCGGAACC
2701 CCTATTTGTT TATTTTTCTA AATACATTCA AATATGTATC CGCTCATGAG
2751 ACAATAACCC TGATAAATGC TTCAATAATA TTGAAAAAGG AAGAGTATGA
2801 GTATTCAACA TTTCCGTGTC GCCCTTATTC CCTTTTTTGC GGCATTTTGC
2851 CTTCCTGTTT TTGCTCACCC AGAAACGCTG GTGAAAGTAA AAGATGCTGA
2901 AGATCAGTTG GGTGCACGAG TGGGTTACAT CGAACTGGAT CTCAACAGCG
2951 GTAAGATCCT TGAGAGTTTT CGCCCCGAAG AACGTTTTCC AATGATGAGC
3001 ACTTTTAAAG TTCTGCTATG TGGCGCGGTA TTATCCCGTA TTGACGCCGG
3051 GCAAGAGCAA CTCGGTCGCC GCATACACTA TTCTCAGAAT GACTTGGTTG
3101 AGTACTCACC AGTCACAGAA AAGCATCTTA CGGATGGCAT GACAGTAAGA
3151 GAATTATGCA GTGCTGCCAT AACCATGAGT GATAACACTG CGGCCAACTT
3201 ACTTCTGACA ACGATCGGAG GACCGAAGGA GCTAACCGCT TTTTTGCACA
3251 ACATGGGGGA TCATGTAACT CGCCTTGATC GTTGGGAACC GGAGCTGAAT
3301 GAAGCCATAC CAAACGACGA GCGTGACACC ACGATGCCTG TAGCAATGGC
3351 AACAACGTTG CGCAAACTAT TAACTGGCGA ACTACTTACT CTAGCTTCCC
3401 GGCAACAATT AATAGACTGG ATGGAGGCGG ATAAAGTTGC AGGACCACTT
3451 CTGCGCTCGG CCCTTCCGGC TGGCTGGTTT ATTGCTGATA AATCTGGAGC
3501 CGGTGAGCGT GGGTCTCGCG GTATCATTGC AGCACTGGGG CCAGATGGTA
3551 AGCCCTCCCG TATCGTAGTT ATCTACACGA CGGGGAGTCA GGCAACTATG
3601 GATGAACGAA ATAGACAGAT CGCTGAGATA GGTGCCTCAC TGATTAAGCA
3651 TTGGTAACTG TCAGACCAAG TTTACTCATA TATACTTTAG ATTGATTTAA
3701 AACTTCATTT TTAATTTAAA AGGATCTAGG TGAAGATCCT TTTTGATAAT
3751 CTCATGACCA AAATCCCTTA ACGTGAGTTT TCGTTCCACT GAGCGTCAGA
3801 CCCCGTAGAA AAGATCAAAG GATCTTCTTG AGATCCTTTT TTTCTGCGCG
3851 TAATCTGCTG CTTGCAAACA AAAAAACCAC CGCTACCAGC GGTGGTTTGT
3901 TTGCCGGATC AAGAGCTACC AACTCTTTTT CCGAAGGTAA CTGGCTTCAG
3951 CAGAGCGCAG ATACCAAATA CTGTCCTTCT AGTGTAGCCG TAGTTAGGCC
4001 ACCACTTCAA GAACTCTGTA GCACCGCCTA CATACCTCGC TCTGCTAATC
4051 CTGTTACCAG TGGCTGCTGC CAGTGGCGAT AAGTCGTGTC TTACCGGGTT
4101 GGACTCAAGA CGATAGTTAC CGGATAAGGC GCAGCGGTCG GGCTGAACGG
4151 GGGGTTCGTG CACACAGCCC AGCTTGGAGC GAACGACCTA CACCGAACTG
4201 AGATACCTAC AGCGTGAGCT ATGAGAAAGC GCCACGCTTC CCGAAGGGAG
4251 AAAGGCGGAC AGGTATCCGG TAAGCGGCAG GGTCGGAACA GGAGAGCGCA
4301 CGAGGGAGCT TCCAGGGGGA AACGCCTGGT ATCTTTATAG TCCTGTCGGG
4351 TTTCGCCACC TCTGACTTGA GCGTCGATTT TTGTGATGCT CGTCAGGGGG
4401 GCGGAGCCTA TGGAAAAACG CCAGCAACGC GGCCTTTTTA CGGTTCCTGG
4451 CCTTTTGCTG GCCTTTTGCT CACATGT
AAVC-Syn-NLS-eGFP
L-ITR: 1-141
Syn-1 promoter: 159-730
NLS-eGFP: 754-1536
bGHp(A): 1561-1788
R-ITR: 1800-1940
Amp(R): 2857-3714
pUC origin: 3865-4532
SEQ ID NO: 117
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGAC TACAAACCGA GTATCTGCAG AGGGCCCTGC GTATGAGTGC
  201 AAGTGGGTTT TAGGACCAGG ATGAGGCGGG GTGGGGGTGC CTACCTGACG
  251 ACCGACCCCG ACCCACTGGA CAAGCACCCA ACCCCCATTC CCCAAATTGC
  301 GCATCCCCTA TCAGAGAGGG GGAGGGGAAA CAGGATGCGG CGAGGCGCGT
  351 GCGCACTGCC AGCTTCAGCA CCGCGGACAG TGCCTTCGCC CCCGCCTGGC
  401 GGCGCGCGCC ACCGCCGCCT CAGCACTGAA GGCGCGCTGA CGTCACTCGC
  451 CGGTCCCCCG CAAACTCCCC TTCCCGGCCA CCTTGGTCGC GTCCGCGCCG
  501 CCGCCGGCCC AGCCGGACCG CACCACGCGA GGCGCGAGAT AGGGGGGCAC
  551 GGGCGCGACC ATCTGCGCTG CGGCGCCGGC GACTCAGCGC TGCCTCAGTC
  601 TGCGGTGGGC AGCGGAGGAG TCGTGTCGTG CCTGAGAGCG CAGCTGTGCT
  651 CCTGGGCACC GCGCAGTCCG CCCCCGCGGC TCCTGGCCAG ACCACCCCTA
  701 GGACCCCCTG CCCCAAGTCG CAGCCTTCGA GCTAGCCAAA GCTTGCCGCC
  751 ACCATGGCCC CAAAAAAGAA AAGAAAAGTG AGGTATCCTG CATTCCTGTA
  801 CAAAGTCGCT ACTATGGTGT CAAAAGGTGA AGAGCTGTTC ACCGGAGTGG
  851 TGCCCATCCT GGTGGAGCTG GACGGAGATG TGAACGGCCA CAAGTTCAGC
  901 GTGTCCGGAG AGGGAGAGGG CGACGCCACC TACGGAAAGC TGACACTGAA
  951 GTTTATCTGC ACCACAGGCA AGCTGCCCGT GCCTTGGCCA ACCCTGGTGA
1001 CCACACTGAC ATACGGCGTG CAGTGTTTCT CTCGGTACCC AGACCACATG
1051 AAGCAGCACG ATTTCTTTAA GAGCGCCATG CCCGAGGGAT ACGTGCAGGA
1101 GAGGACAATC TTCTTTAAGG ACGATGGCAA CTACAAGACC AGAGCTGAGG
1151 TGAAGTTCGA GGGAGACACA CTGGTGAACC GGATCGAGCT GAAGGGCATC
1201 GACTTTAAGG AGGATGGAAA CATCCTGGGC CACAAGCTGG AGTACAACTA
1251 CAACTCCCAC AACGTGTACA TCATGGCCGA TAAGCAGAAG AACGGAATCA
1301 AGGTGAACTT TAAGATCCGC CACAACATCG AGGACGGCTC TGTGCAGCTG
1351 GCTGATCACT ACCAGCAGAA CACCCCTATC GGAGACGGAC CCGTGCTGCT
1401 GCCTGATAAC CACTACCTGT CTACACAGAG CGCCCTGTCC AAGGACCCAA
1451 ACGAGAAGAG GGATCACATG GTGCTCCTGG AATTTGTCAC TGCTGCCGGT
1501 ATTACTCTCG GGATGGATGA ACTGTATAAA TGATAAGCGG CCGCAACTCG
1551 AGACTCTAGA CGACTGTGCC TTCTAGTTGC CAGCCATCTG TTGTTTGCCC
1601 CTCCCCCGTG CCTTCCTTGA CCCTGGAAGG TGCCACTCCC ACTGTCCTTT
1651 CCTAATAAAA TGAGGAAATT GCATCGCATT GTCTGAGTAG GTGTCATTCT
1701 ATTCTGGGGG GTGGGGTGGG GCAGGACAGC AAGGGGGAGG ATTGGGAAGA
1751 CAATAGCAGG CATGCTGGGG ATGCGGTGGG CTCTATGGCC GCGGGCCGCA
1801 GGAACCCCTA GTGATGGAGT TGGCCACTCC CTCTCTGCGC GCTCGCTCGC
1851 TCACTGAGGC CGGGCGACCA AAGGTCGCCC GACGCCCGGG CTTTGCCCGG
1901 GCGGCCTCAG TGAGCGAGCG AGCGCGCAGC TGCCTGCAGG GGCGCCTGAT
1951 GCGGTATTTT CTCCTTACGC ATCTGTGCGG TATTTCACAC CGCATACGTC
2001 AAAGCAACCA TAGTACGCGC CCTGTAGCGG CGCATTAAGC GCGGCGGGTG
2051 TGGTGGTTAC GCGCAGCGTG ACCGCTACAC TTGCCAGCGC CCTAGCGCCC
2101 GCTCCTTTCG CTTTCTTCCC TTCCTTTCTC GCCACGTTCG CCGGCTTTCC
2151 CCGTCAAGCT CTAAATCGGG GGCTCCCTTT AGGGTTCCGA TTTAGTGCTT
2201 TACGGCACCT CGACCCCAAA AAACTTGATT TGGGTGATGG TTCACGTAGT
2251 GGGCCATCGC CCTGATAGAC GGTTTTTCGC CCTTTGACGT TGGAGTCCAC
2301 GTTCTTTAAT AGTGGACTCT TGTTCCAAAC TGGAACAACA CTCAACCCTA
2351 TCTCGGGCTA TTCTTTTGAT TTATAAGGGA TTTTGCCGAT TTCGGCCTAT
2401 TGGTTAAAAA ATGAGCTGAT TTAACAAAAA TTTAACGCGA ATTTTAACAA
2451 AATATTAACG TTTACAATTT TATGGTGCAC TCTCAGTACA ATCTGCTCTG
2501 ATGCCGCATA GTTAAGGCAG CCCCGACACC CGCCAACACC CGCTGACGCG
2551 CCCTGACGGG CTTGTCTGCT CCCGGCATCC GCTTACAGAC AAGCTGTGAC
2601 CGTCTCCGGG AGCTGCATGT GTCAGAGGTT TTCACCGTCA TCACCGAAAC
2651 GCGCGAGACG AAAGGGCCTC GTGATACGCC TATTTTTATA GGTTAATGTC
2701 ATGATAATAA TGGTTTCTTA GACGTCAGGT GGCACTTTTC GGGGAAATGT
2751 GCGCGGAACC CCTATTTGTT TATTTTTCTA AATACATTCA AATATGTATC
2801 CGCTCATGAG ACAATAACCC TGATAAATGC TTCAATAATA TTGAAAAAGG
2851 AAGAGTATGA GTATTCAACA TTTCCGTGTC GCCCTTATTC CCTTTTTTGC
2901 GGCATTTTGC CTTCCTGTTT TTGCTCACCC AGAAACGCTG GTGAAAGTAA
2951 AAGATGCTGA AGATCAGTTG GGTGCACGAG TGGGTTACAT CGAACTGGAT
3001 CTCAACAGCG GTAAGATCCT TGAGAGTTTT CGCCCCGAAG AACGTTTTCC
3051 AATGATGAGC ACTTTTAAAG TTCTGCTATG TGGCGCGGTA TTATCCCGTA
3101 TTGACGCCGG GCAAGAGCAA CTCGGTCGCC GCATACACTA TTCTCAGAAT
3151 GACTTGGTTG AGTACTCACC AGTCACAGAA AAGCATCTTA CGGATGGCAT
3201 GACAGTAAGA GAATTATGCA GTGCTGCCAT AACCATGAGT GATAACACTG
3251 CGGCCAACTT ACTTCTGACA AGGATGGGAG GACCGAAGGA GCTAACCGCT
3301 TTTTTGGACA ACATGGGGGA TCATGTAACT CGCCTTGATC GTTGGGAACC
3351 GGAGCTGAAT GAAGCCATAC CAAACGACGA GCGTGACACC ACGATGCCTG
3401 TAGCAATGGC AACAACGTTG CGCAAACTAT TAACTGGCGA ACTACTTACT
3451 CTAGCTTCCC GGCAACAATT AATAGACTGG ATGGAGGCGG ATAAAGTTGC
3501 AGGACCACTT CTGCGCTCGG CCCTTCCGGC TGGCTGGTTT ATTGCTGATA
3551 AATCTGGAGC CGGTGAGCGT GGGTCTCGCG GTATCATTGC AGCACTGGGG
3601 CCAGATGGTA AGCCCTCCCG TATCGTAGTT ATCTACACGA CGGGGAGTCA
3651 GGCAACTATG GATGAACGAA ATAGACAGAT CGCTGAGATA GGTGCCTCAC
3701 TGATTAAGCA TTGGTAACTG TCAGACCAAG TTTACTCATA TATACTTTAG
3751 ATTGATTTAA AACTTCATTT TTAATTTAAA AGGATCTAGG TGAAGATCCT
3801 TTTTGATAAT CTCATGACCA AAATCCCTTA ACGTGAGTTT TCGTTCCACT
3851 GAGCGTCAGA CCCCGTAGAA AAGATCAAAG GATCTTCTTG AGATCCTTTT
3901 TTTCTGCGCG TAATCTGCTG CTTGCAAACA AAAAAACCAC CGCTACCAGC
3951 GGTGGTTTGT TTGCCGGATC AAGAGGTACC AACTCTTTTT CCGAAGGTAA
4001 CTGGCTTCAG CAGAGCGCAG ATACCAAATA CTGTCCTTCT AGTGTAGCCG
4051 TAGTTAGGCC ACCACTTCAA GAACTCTGTA GCACCGCCTA CATACCTCGC
4101 TCTGCTAATC CTGTTACCAG TGGCTGCTGC CAGTGGCGAT AAGTCGTGTC
4151 TTACCGGGTT GGACTCAAGA CGATAGTTAC CGGATAAGGC GCAGCGGTCG
4201 GGCTGAACGG GGGGTTCGTG CACACAGCCC AGCTTGGAGC GAACGACCTA
4251 CACCGAACTG AGATACCTAC AGCGTGAGCT ATGAGAAAGC GCCACGCTTC
4301 CCGAAGGGAG AAAGGCGGAC AGGTATCCGG TAAGCGGCAG GGTCGGAACA
4351 GGAGAGCGCA CGAGGGAGCT TCCAGGGGGA AACGCCTGGT ATCTTTATAG
4401 TCCTGTCGGG TTTCGCCACC TCTGACTTGA GCGTCGATTT TTGTGATGCT
4451 CGTCAGGGGG GCGGAGCCTA TGGAAAAACG CCAGCAACGC GGCCTTTTTA
4501 CGGTTCCTGG CCTTTTGCTG GCCTTTTGCT CACATGT
AAVC-Syn-mBPIP-TATκ28-hCDKL5-107
L-ITR: 1-141
Syn-1 promoter: 159-730
Bip-TATκ28-hCDKL5-107: 754-3819
bGHp(A): 3844-4071
R-ITR: 4083-4223
Amp(R): 5140-5997
pUC origin: 6148-6815
SEQ ID NO: 118
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGAC TACAAACCGA GTATCTGCAG AGGGCCCTGC GTATGAGTGC
  201 AAGTGGGTTT TAGGACCAGG ATGAGGCGGG GTGGGGGTGC CTACCTGACG
  251 ACCGACCCCG ACCCACTGGA CAAGCACCCA ACCCCCATTC CCCAAATTGC
  301 GCATCCCCTA TCAGAGAGGG GGAGGGGAAA CAGGATGCGG CGAGGCGCGT
  351 GCGCACTGCC AGCTTCAGCA CCGCGGACAG TGCCTTCGCC CCCGCCTGGC
  401 GGCGCGCGCC ACCGCCGCCT CAGCACTGAA GGCGCGCTGA CGTCACTCGC
  451 CGGTCCCCCG CAAACTCCCC TTCCCGGCCA CCTTGGTCGC GTCCGCGCCG
  501 CCGCCGGCCC AGCCGGACCG CACCACGCGA GGCGCGAGAT AGGGGGGCAC
  551 GGGCGCGACC ATCTGCGCTG CGGCGCCGGC GACTCAGCGC TGCCTCAGTC
  601 TGCGGTGGGC AGCGGAGGAG TCGTGTCGTG CCTGAGAGCG CAGCTGTGCT
  651 CCTGGGCACC GCGCAGTCCG CCCCCGCGGC TCCTGGCCAG ACCACCCCTA
  701 GGACCCCCTG CCCCAAGTCG CAGCCTTCGA GCTAGCCAAA GCTTGCCGCC
  751 ACCATGAAAC TCTCCCTGGT CGCTGCTATG CTGCTCCTCC TGTCCCTCGT
  801 CGCTGCTATG CTCCTGCTGC TGTCTGCCGC TCGGGCTGGT GATGCTGCTC
  851 AGCCAGCTAG GAGAGCTAGG AGGACCAAGC TGGCTGCTTA CGCTAGAAAG
  901 GCTGCTAGAC AGGCTAGGGC TGGAGGAGGC GGATCCAAGA TCCCCAACAT
  951 CGGCAACGTG ATGAACAAGT TCGAGATCCT GGGAGTGGTG GGAGAGGGAG
1001 CTTACGGAGT GGTGCTGAAG TGCCGGCACA AGGAGACACA CGAGATCGTG
1051 GCTATCAAGA AGTTTAAGGA CAGCGAGGAG AACGAGGAGG TGAAGGAGAC
1101 AACCCTGCGC GAGCTGAAGA TGCTGAGGAC ACTGAAGCAG GAGAACATCG
1151 TGGAGCTGAA GGAGGCCTTC AGGAGACGGG GAAAGCTGTA CCTGGTGTTT
1201 GAGTACGTGG AGAAGAACAT GCTGGAGCTG CTGGAGGAGA TGCCAAACGG
1251 AGTGCCACCT GAGAAGGTGA AGTCCTACAT CTACCAGCTG ATCAAGGCTA
1301 TCCACTGGTG CCACAAGAAC GACATCGTGC ACCGCGATAT CAAGCCTGAG
1351 AACCTGCTGA TCTCCCACAA CGACGTGCTG AAGCTGTGCG ATTTCGGCTT
1401 TGCCAGGAAC CTGAGCGAGG GAAACAACGC CAACTACACA GAGTACGTGG
1451 CTACCCGCTG GTACAGGAGC CCAGAGCTGC TGCTGGGAGC TCCATACGGA
1501 AAGAGCGTGG ACATGTGGTC CGTGGGCTGC ATCCTGGGAG AGCTGTCTGA
1551 CGGCCAGCCT CTGTTCCCAG GAGAGAGCGA GATCGATCAG CTGTTTACCA
1601 TCCAGAAGGT GCTGGGCCCT CTGCCAAGCG AGCAGATGAA GCTGTTCTAC
1651 TCCAACCCTC GCTTCCACGG ACTGAGGTTT CCCGCCGTGA ACCACCCTCA
1701 GAGCCTGGAG CGCAGGTACC TGGGCATCCT GAACTCCGTG CTGCTGGATC
1751 TGATGAAGAA CCTGCTGAAG CTGGACCCCG CCGATAGATA CCTGACCGAG
1801 CAGTGTCTGA ACCACCCTAC ATTTCAGACC CAGAGACTGC TGGACCGGAG
1851 CCCTTCCCGC TCTGCTAAGA GGAAGCCATA CCACGTGGAG AGCTCCACCC
1901 TGTCCAACCG CAACCAGGCC GGCAAGTCTA CAGCTCTGCA GAGCCACCAC
1951 AGGAGCAACT CCAAGGACAT CCAGAACCTG TCTGTGGGCC TGCCTAGGGC
2001 TGATGAGGGA CTGCCAGCTA ACGAGAGCTT CCTGAACGGC AACCTGGCCG
2051 GAGCTTCTCT GAGCCCACTG CACACAAAGA CCTACCAGGC CTCTAGCCAG
2101 CCCGGCTCCA CATCTAAGGA CCTGACCAAC AACAACATCC CACACCTGCT
2151 GTCTCCCAAG GAGGCTAAGA GCAAGACCGA GTTCGACTTT AACATCGATC
2201 CCAAGCCTAG CGAGGGCCCT GGAACAAAGT ACCTGAAGAG CAACTCCAGA
2251 TCTCAGCAGA ACCGGCACTC CTTCATGGAG TCCTCTCAGT CTAAGGCCGG
2301 CACCCTGCAG CCAAAGGAGA AGCAGAGCCG GCACTCCTAC ATCGATACCA
2351 TCCCCCAGAG CTCCCGCAGC CCTTCCTACA GGACAAAGGC CAAGAGCCAC
2401 GGCGCTCTGT CTGACAGCAA GTCCGTGTCT AACCTGTCCG AGGCCAGAGC
2451 TCAGATCGCT GAGCCAAGCA CCTCCAGGTA CTTTCCTTCT AGCTGTCTGG
2501 ACCTGAACTC TCCTACAAGC CCAACACCCA CCAGACACAG CGATACACGG
2551 ACCCTGCTGT CTCCAAGCGG CAGAAACAAC CGGAACGAGG GAACCCTGGA
2601 TTCTAGACGG ACCACAACCA GGCACAGCAA GACAATGGAG GAGCTGAAGC
2651 TGCCAGAGCA CATGGACTCC TCTCACTCCC ACTCTCTGAG CGCCCCCCAC
2701 GAGTCCTTCT CTTACGGCCT GGGATACACC TCCCCCTTCA GCTCCCAGCA
2751 GCGCCCCCAC AGGCACTCTA TGTACGTGAC AAGAGACAAG GTGCGGGCCA
2801 AGGGCCTGGA TGGAAGCCTG TTCATCGGCC AGGGAATGGC CGCTAGAGCT
2851 AACTCCCTGC AGCTGCTGTC TCCTCAGCCA GGAGAGCAGC TGCCACCCGA
2901 GATGACCGTG GCCAGATCTA GCGTGAAGGA GACAAGCCGG GAGGGCACCT
2951 CCTCTTTCCA CACAAGACAG AAGTCCGAGG GCGGAGTGTA CCACGACCCC
3001 CACTCTGACG ATGGAACAGC TCCTAAGGAG AACCGGCACC TGTACAACGA
3051 TCCCGTGCCT CGCAGGGTGG GCTCCTTCTA CCGCGTGCCA TCTCCCAGGC
3101 CTGACAACAG CTTTCACGAG AACAACGTGT CCACCAGAGT GAGCTCCCTG
3151 CCATCTGAGT CTAGCTCCGG AACAAACCAC TCTAAGCGGC AGCCCGCCTT
3201 CGATCCTTGG AAGAGCCCAG AGAACATCTC TCACAGCGAG CAGCTGAAGG
3251 AGAAGGAGAA GCAGGGCTTC TTTCGCAGCA TGAAGAAGAA GAAGAAGAAG
3301 AGCCAGACCG TGCCTAACTC CGACTCTCCA GATCTGCTGA CCCTGCAGAA
3351 GTCCATCCAC AGCGCCTCCA CACCCTCTAG CAGACCTAAG GAGTGGCGGC
3401 CTGAGAAGAT CAGCGATCTG CAGACACAGA GCCAGCCACT GAAGTCCCTG
3451 AGGAAGCTGC TGCACCTGTC CTCTGCCAGC AACCACCCAG CTAGCTCCGA
3501 CCCAAGGTTC CAGCCACTGA CAGCTCAGCA GACCAAGAAC TCTTTTAGCG
3551 AGATCAGGAT CCACCCTCTG TCCCAGGCTT CTGGCGGATC TAGCAACATC
3601 AGGCAGGAGC CAGCTCCAAA GGGCAGGCCC GCTCTGCAGC TGCCTGGACA
3651 GATGGACCCA GGATGGCACG TGTCCTCTGT GACAAGGTCC GCCACCGAGG
3701 GACCATCCTA CTCTGAGCAG CTGGGCGCTA AGTCTGGCCC TAACGGACAC
3751 CCATACAACC GAACAAATAG AAGTAGGATG CCAAACCTCA ATGACCTCAA
3801 GGAAACTGCC CTGTGATAAG CGGCCGCAAC TCGAGACTCT AGACGACTGT
3851 GCCTTCTAGT TGCCAGCCAT CTGTTGTTTG CCCCTCCCCC GTGCCTTCCT
3901 TGACCCTGGA AGGTGCCACT CCCACTGTCC TTTCCTAATA AAATGAGGAA
3951 ATTGCATCGC ATTGTCTGAG TAGGTGTCAT TCTATTCTGG GGGGTGGGGT
4001 GGGGCAGGAC AGCAAGGGGG AGGATTGGGA AGACAATAGC AGGCATGCTG
4051 GGGATGCGGT GGGCTCTATG GCCGCGGGCC GCAGGAACCC CTAGTGATGG
4101 AGTTGGCCAC TCCCTCTCTG CGCGCTCGCT CGCTCACTGA GGCCGGGCGA
4151 CCAAAGGTCG CCCGACGCCC GGGCTTTGCC CGGGCGGCCT CAGTGAGCGA
4201 GCGAGCGCGC AGCTGCCTGC AGGGGCGCCT GATGCGGTAT TTTCTCCTTA
4251 CGCATCTGTG CGGTATTTCA CACCGCATAC GTCAAAGCAA CCATAGTACG
4301 CGCCCTGTAG CGGCGCATTA AGCGCGGCGG GTGTGGTGGT TACGCGCAGC
4351 GTGACCGCTA CACTTGCCAG CGCCCTAGCG CCCGCTCCTT TCGCTTTCTT
4401 CCCTTCCTTT CTCGCCACGT TCGCCGGCTT TCCCCGTCAA GCTCTAAATC
4451 GGGGGCTCCC TTTAGGGTTC CGATTTAGTG CTTTACGGCA CCTCGACCCC
4501 AAAAAACTTG ATTTGGGTGA TGGTTCACGT AGTGGGCCAT CGCCCTGATA
4551 GACGGTTTTT CGCCCTTTGA CGTTGGAGTC CACGTTCTTT AATAGTGGAC
4601 TCTTGTTCCA AACTGGAACA ACACTCAACC CTATCTCGGG CTATTCTTTT
4651 GATTTATAAG GGATTTTGCC GATTTCGGCC TATTGGTTAA AAAATGAGCT
4701 GATTTAACAA AAATTTAACG CGAATTTTAA CAAAATATTA ACGTTTACAA
4751 TTTTATGGTG CACTCTCAGT ACAATCTGCT CTGATGCCGC ATAGTTAAGC
4801 CAGCCCCGAC ACCCGCCAAC ACCCGCTGAC GCGCCCTGAC GGGCTTGTCT
4851 GCTCCCGGCA TCCGCTTACA GACAAGCTGT GACCGTCTCC GGGAGCTGCA
4901 TGTGTCAGAG GTTTTCACCG TCATCACCGA AACGCGCGAG ACGAAAGGGC
4951 CTCGTGATAC GCCTATTTTT ATAGGTTAAT GTCATGATAA TAATGGTTTC
5001 TTAGACGTCA GGTGGCACTT TTCGGGGAAA TGTGCGCGGA ACCCCTATTT
5051 GTTTATTTTT CTAAATACAT TCAAATATGT ATCCGCTCAT GAGACAATAA
5101 CCCTGATAAA TGCTTCAATA ATATTGAAAA AGGAAGAGTA TGAGTATTCA
5151 ACATTTCCGT GTCGCCCTTA TTCCCTTTTT TGCGGCATTT TGCCTTCCTG
5201 TTTTTGCTCA CCCAGAAACG CTGGTGAAAG TAAAAGATGC TGAAGATCAG
5251 TTGGGTGCAC GAGTGGGTTA CATCGAACTG GATCTCAACA GCGGTAAGAT
5301 CCTTGAGAGT TTTCGCCCCG AAGAACGTTT TCCAATGATG AGCACTTTTA
5351 AAGTTCTGCT ATGTGGCGCG GTATTATCCC GTATTGACGC CGGGCAAGAG
5401 CAACTCGGTC GCCGCATACA CTATTCTCAG AATGACTTGG TTGAGTACTC
5451 ACCAGTCACA GAAAAGCATC TTACGGATGG CATGACAGTA AGAGAATTAT
5501 GCAGTGCTGC CATAACCATG AGTGATAACA CTGCGGCCAA CTTACTTCTG
5551 ACAACGATCG GAGGACCGAA GGAGCTAACC GCTTTTTTGC ACAACATGGG
5601 GGATCATGTA ACTCGCCTTG ATCGTTGGGA ACCGGAGCTG AATGAAGCCA
5651 TACCAAACGA CGAGCGTGAC ACCACGATGC CTGTAGCAAT GGCAACAACG
5701 TTGCGCAAAC TATTAACTGG CGAACTACTT ACTCTAGCTT CCCGGCAACA
5751 ATTAATAGAC TGGATGGAGG CGGATAAAGT TGCAGGACCA CTTCTGCGCT
5801 CGGCCCTTCC GGCTGGCTGG TTTATTGCTG ATAAATCTGG AGCCGGTGAG
5851 CGTGGGTCTC GCGGTATCAT TGCAGCACTG GGGCCAGATG GTAAGCCCTC
5901 CCGTATCGTA GTTATCTACA CGACGGGGAG TCAGGCAACT ATGGATGAAC
5951 GAAATAGACA GATCGCTGAG ATAGGTGCCT CACTGATTAA GCATTGGTAA
6001 CTGTCAGACC AAGTTTACTC ATATATACTT TAGATTGATT TAAAACTTCA
6051 TTTTTAATTT AAAAGGATCT AGGTGAAGAT CCTTTTTGAT AATCTCATGA
6101 CCAAAATCCC TTAACGTGAG TTTTCGTTCC ACTGAGCGTC AGACCCCGTA
6151 GAAAAGATCA AAGGATCTTC TTGAGATCCT TTTTTTCTGC GCGTAATCTG
6201 CTGCTTGCAA ACAAAAAAAC CACCGCTACC AGCGGTGGTT TGTTTGCCGG
6251 ATCAAGAGCT ACCAACTCTT TTTCCGAAGG TAACTGGCTT CAGCAGAGCG
6301 CAGATACCAA ATACTGTCCT TCTAGTGTAG CCGTAGTTAG GCCACCACTT
6351 CAAGAACTCT GTAGCACCGC CTACATACCT CGCTCTGCTA ATCCTGTTAC
6401 CAGTGGCTGC TGCCAGTGGC GATAAGTCGT GTCTTACCGG GTTGGACTCA
6451 AGACGATAGT TACCGGATAA GGCGCAGCGG TCGGGCTGAA CGGGGGGTTC
6501 GTGCACACAG CCCAGCTTGG AGCGAACGAC CTACACCGAA CTGAGATACC
6551 TACAGCGTGA GCTATGAGAA AGCGCCACGC TTCCCGAAGG GAGAAAGGCG
6601 GACAGGTATC CGGTAAGCGG CAGGGTCGGA ACAGGAGAGC GCACGAGGGA
6651 GCTTCCAGGG GGAAACGCCT GGTATCTTTA TAGTCCTGTC GGGTTTCGCC
6701 ACCTCTGACT TGAGCGTCGA TTTTTGTGAT GCTCGTCAGG GGGGCGGAGC
6751 CTATGGAAAA ACGCCAGCAA CGCGGCCTTT TTACGGTTCC TGGCCTTTTG
6801 CTGGCCTTTT GCTCACATGT
AAVC-Syn-mBPIP-TATκ28-hCDKL5-107 (dead kinase)
L-ITR: 1-141
Syn-1 promoter: 159-730
Bip-TATκ28-hCDKL5-107 (kinase dead): 754-3819
bGHp(A): 3844-4071
R-ITR: 4083-4223
Amp(R): 5140-5997
pUC origin: 6148-6815
SEQ ID NO: 119
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGAC TACAAACCGA GTATCTGCAG AGGGCCCTGC GTATGAGTGC
  201 AAGTGGGTTT TAGGACCAGG ATGAGGCGGG GTGGGGGTGC CTACCTGACG
  251 ACCGACCCCG ACCCACTGGA CAAGGACCCA ACCCCCATTC CCCAAATTGC
  301 GCATCCCCTA TCAGAGAGGG GGAGGGGAAA CAGGATGCGG CGAGGCGCGT
  351 GCGCACTGCC AGCTTCAGCA CCGCGGACAG TGCCTTCGCC CCCGCCTGGC
  401 GGCGCGCGCC ACCGCCGCCT CAGCACTGAA GGCGCGCTGA CGTCACTCGC
  451 CGGTCCCCCG CAAACTCCCC TTCCCGGCCA CCTTGGTCGC GTCCGCGCCG
  501 CCGCCGGCCC AGCCGGACCG CACCACGCGA GGCGCGAGAT AGGGGGGCAC
  551 GGGCGCGACC ATCTGCGCTG CGGCGCCGGC GACTCAGCGC TGCCTCAGTC
  601 TGCGGTGGGC AGCGGAGGAG TCGTGTCGTG CCTGAGAGCG CAGCTGTGCT
  651 CCTGGGCACC GCGCAGTCCG CCCCCGCGGC TCCTGGCCAG ACCACCCCTA
  701 GGACCCCCTG CCCCAAGTCG CAGCCTTCGA GCTAGCCAAA GCTTGCCGCC
  751 ACCATGAAAC TCTCCCTCGT CGCCGCTATG CTCCTGCTCC TCTCCCTCGT
  801 CGCTGCCATG CTCCTGCTGC TCAGTGCCGC TCGGGCCGGA GATGCTGCTC
  851 AGCCAGCTAG GAGAGCTAGG AGGACCAAGC TGGCTGCTTA CGCTAGGAAG
  901 GCTGCTAGGC AGGCTAGGGC TGGCGGAGGC GGATCCAAGA TCCCCAACAT
  951 CGGCAACGTG ATGAACAAGT TCGAGATCCT GGGAGTGGTG GGAGAGGGAG
1001 CTTACGGAGT GGTGCTGAAG TGCAGGCACA AGGAGACACA CGAGATCGTG
1051 GCTATCAGGA GGTTCAAGGA CAGCGAGGAG AACGAGGAGG TGAAGGAGAC
1101 AACCCTGAGA GAGCTGAAGA TGCTGCGGAC ACTGAAGCAG GAGAACATCG
1151 TGGAGCTGAA GGAGGCCTTC CGGCGCAGGG GAAAGCTGTA CCTGGTGTTT
1201 GAGTACGTGG AGAAGAACAT GCTGGAGCTG CTGGAGGAGA TGCCAAACGG
1251 AGTGCCACCT GAGAAGGTGA AGTCCTACAT CTACCAGCTG ATCAAGGCTA
1301 TCCACTGGTG CCACAAGAAC GACATCGTGC ACAGAGATAT CAAGCCTGAG
1351 AACCTGCTGA TCTCCCACAA CGACGTGCTG AAGCTGTGCG ATTTCGGCTT
1401 TGCCCGGAAC CTGAGCGAGG GAAACAAGCG CAACTACACA GAGTACGTGG
1451 CTACCAGATG GTACCGGAGC CCAGAGCTGC TGCTGGGAGC TCCATACGGA
1501 AAGAGCGTGG ACATGTGGTC CGTGGGCTGC ATCCTGGGAG AGCTGTCTGA
1551 CGGCCAGCCT CTGTTCCCAG GAGAGAGCGA GATCGATCAG CTGTTTACCA
1601 TCCAGAAGGT GCTGGGCCCT CTGCCAAGCG AGCAGATGAA GCTGTTCTAC
1651 TCCAACCCTA GATTCCACGG ACTGCGGTTT CCCGCCGTGA ACCACCCTCA
1701 GAGCCTGGAG AGACGGTACC TGGGCATCCT GAACTCCGTG CTGCTGGATC
1751 TGATGAAGAA CCTGCTGAAG CTGGACCCCG CCGATCGCTA CCTGACCGAG
1801 CAGTGTCTGA ACCACCCTAC ATTTCAGACC CAGCGCCTGC TGGACAGGAG
1851 CCCTTCCAGA TCTGCTAAGC GGAAGCCATA CCACGTGGAG AGCTCCACCC
1901 TGTCCAACAG AAACCAGGCC GGCAAGTCTA CAGCTCTGCA GAGCCACCAC
1951 CGGAGCAACT CCAAGGACAT CCAGAACCTG TCTGTGGGCC TGCCTAGAGC
2001 CGATGAGGGA CTGCCAGCTA ACGAGAGCTT CCTGAACGGC AACCTGGCCG
2051 GAGCTTCTCT GAGCCCACTG CACACAAAGA CCTACCAGGC CTCTAGCCAG
2101 CCCGGCTCCA CATCTAAGGA CCTGACCAAC AACAACATCC CACACCTGCT
2151 GTCTCCCAAG GAGGCTAAGA GCAAGACCGA GTTCGACTTT AACATCGATC
2201 CCAAGCCTAG CGAGGGCCCT GGAACAAAGT ACCTGAAGAG CAACTCCCGC
2251 TCTCAGCAGA ACAGGCACTC CTTCATGGAG TCCTCTCAGT CTAAGGCCGG
2301 CACCCTGCAG CCAAACGAGA AGCAGAGCAG ACACTCCTAC ATCGATACCA
2351 TCCCCCAGAG CTCCAGAAGC CCTTCCTACC GGACAAAGGC CAAGAGCCAC
2401 GGCGCTCTGT CTGACAGCAA GTCCGTGTCT AACCTGTCCG AGGCTAGGGC
2451 TCAGATCGCT GAGCCCAGCA CCTCCAGGTA CTTTCCTTCT AGCTGTCTGG
2501 ACCTGAACTC TCCTACAAGC CCAACACCCA CCCGCCACAG CGATACAAGG
2551 ACCCTGCTGT CTCCAAGCGG CAGGAACAAC AGGAACGAGG GAACCCTGGA
2601 TTCTCGCAGG ACCACAACCC GGCACAGCAA GACAATGGAG GAGCTGAAGC
2651 TGCCAGAGCA CATGGACTCC TCTCACTCCC ACTCTCTGAG CGCCCCCCAC
2701 GAGTCCTTCT CTTACGGCCT GGGATACACC TCCCCCTTCA GCTCCCAGCA
2751 GAGGCCCCAC AGGCACTCTA TGTACGTGAC ACGCGACAAG GTGAGGGCCA
2801 AGGGCCTGGA TGGAAGCCTG TCCATCGGCC AGGGAATGGC CGCTAGGGCT
2851 AACTCCCTGC AGCTGCTGTC TCCTCAGCCA GGAGAGCAGC TGCCACCAGA
2901 GATGACCGTG GCTCGCTCTA GCGTGAAGGA GACAAGCAGG GAGGGCACCT
2951 CCTCTTTCCA CACACGCCAG AAGTCCGAGG GCGGAGTGTA CCACGACCCC
3001 CACTCTGACG ATGGAACAGC TCCTAAGGAG AACAGGCACC TGTACAACGA
3051 TCCCGTGCCT AGACGGGTGG GCTCCTTCTA CAGAGTGCCA TCTCCCCGGC
3101 CTGACAACAG CTTTCACGAG AACAACGTGT CCACCCGCGT GAGCTCCCTG
3151 CCATCTGAGT CTAGCTCCGG AACAAACCAC TCTAAGAGGC AGCCCGCCTT
3201 CGATCCTTGG AAGAGCCCAG AGAACATCTC TCACAGCGAG CAGCTGAAGG
3251 AGAAGGAGAA GCAGGGCTTC TTTAGAAGCA TGAAGAAGAA GAAGAAGAAG
3301 AGCCAGACCG TGCCTAACTC CGACTCTCCA GATCTGCTGA CCCTGCAGAA
3351 GTCCATCCAC AGCGCCTCCA CACCATCTAG CCGCCCTAAG GAGTGGAGGC
3401 CTGAGAAGAT CAGCGATCTG CAGACACAGA GCCAGCCACT GAAGTCCCTG
3451 CGGAAGCTGC TGCACCTGTC CTCTGCCAGC AACCACCCCG CTAGCTCCGA
3501 CCCAAGATTC CAGCCCCTGA CAGCCCAGCA GACCAAGAAC TCTTTTAGCG
3551 AGATCCGGAT CCACCCTCTG TCCCAGGCTT CTGGCGGATC TAGCAACATC
3601 AGACAGGAGC CAGCTCCAAA GGGCCGGCCC GCTCTGCAGC TGCCTGGCCA
3651 GATGGACCCA GGATGGCACG TGTCCTCTGT GACAAGGTCC GCCACCGAGG
3701 GACCATCCTA CTCTGAGCAG CTGGGCGCTA AGTCTGGCCC TAACGGACAC
3751 CCATACAATA GGACTAATCG CAGCAGAATG CCCAACCTGA ACGACCTCAA
3801 GGAAACAGCA CTCTGATAAG CGGCCGCAAC TCGAGACTCT AGACGACTGT
3851 GCCTTCTAGT TGCCAGCCAT CTGTTGTTTG CCCCTCCCCC GTGCCTTCCT
3901 TGACCCTGGA AGGTGCCACT CCCACTGTCC TTTCCTAATA AAATGAGGAA
3951 ATTGCATCGC ATTGTCTGAG TAGGTGTCAT TCTATTCTGG GGGGTGGGGT
4001 GGGGCAGGAC AGCAAGGGGG AGGATTGGGA AGACAATAGC AGGCATGCTG
4051 GGGATGCGGT GGGCTCTATG GCCGCGGGCC GCAGGAACCC CTAGTGATGG
4101 AGTTGGCCAC TCCCTCTCTG CGCGCTCGCT CGCTCACTGA GGCCGGGCGA
4151 CCAAAGGTCG CCCGACGCCC GGGCTTTGCC CGGGCGGCCT CAGTGAGCGA
4201 GCGAGCGCGC AGCTGCCTGC AGGGGCGCCT GATGCGGTAT TTTCTCCTTA
4251 CGCATCTGTG CGGTATTTCA CACCGCATAC GTCAAAGCAA CCATAGTACG
4301 CGCCCTGTAG CGGCGCATTA AGCGCGGCGG GTGTGGTGGT TACGCGCAGC
4351 GTGACCGCTA CACTTGCCAG CGCCCTAGCG CCCGCTCCTT TCGCTTTCTT
4401 CCCTTCCTTT CTCGCCACGT TCGCCGGCTT TCCCCGTCAA GCTCTAAATC
4451 GGGGGCTCCC TTTAGGGTTC CGATTTAGTG CTTTACGGCA CCTCGACCCC
4501 AAAAAACTTG ATTTGGGTGA TGGTTCACGT AGTGGGCCAT CGCCCTGATA
4551 GACGGTTTTT CGCCCTTTGA CGTTGGAGTC CACGTTCTTT AATAGTGGAC
4601 TCTTGTTCCA AACTGGAACA ACACTCAACC CTATCTCGGG CTATTCTTTT
4651 GATTTATAAG GGATTTTGCC GATTTCGGCC TATTGGTTAA AAAATGAGCT
4701 GATTTAACAA AAATTTAACG CGAATTTTAA CAAAATATTA ACGTTTACAA
4751 TTTTATGGTG CACTCTCAGT ACAATCTGCT CTGATGCCGC ATAGTTAAGC
4801 CAGCCCCGAC ACCCGCCAAC ACCCGCTGAC GCGCCCTGAC GGGCTTGTCT
4851 GCTCCCGGCA TCCGCTTACA GACAAGCTGT GACCGTCTCC GGGAGCTGCA
4901 TGTGTCAGAG GTTTTCACCG TCATCACCGA AACGCGCGAG ACGAAAGGGC
4951 CTCGTGATAC GCCTATTTTT ATAGGTTAAT GTCATGATAA TAATGGTTTC
5001 TTAGACGTCA GGTGGCACTT TTCGGGGAAA TGTGCGCGGA ACCCCTATTT
5051 GTTTATTTTT CTAAATACAT TCAAATATGT ATCCGCTCAT GAGACAATAA
5101 CCCTGATAAA TGCTTCAATA ATATTGAAAA AGGAAGAGTA TGAGTATTCA
5151 ACATTTCCGT GTCGCCCTTA TTCCCTTTTT TGCGGCATTT TGCCTTCCTG
5201 TTTTTGCTCA CCCAGAAACG CTGGTGAAAG TAAAAGATGC TGAAGATCAG
5251 TTGGGTGCAC GAGTGGGTTA CATCGAACTG GATCTCAACA GCGGTAAGAT
5301 CCTTGAGAGT TTTCGCCCCG AAGAACGTTT TCCAATGATG AGCACTTTTA
5351 AAGTTCTGCT ATGTGGCGCG GTATTATCCC GTATTGACGC CGGGCAAGAG
5401 CAACTCGGTC GCCGCATACA CTATTCTCAG AATGACTTGG TTGAGTACTC
5451 ACCAGTCACA GAAAAGCATC TTACGGATGG CATGACAGTA AGAGAATTAT
5501 GCAGTGCTGC CATAACCATG AGTGATAACA CTGCGGCCAA CTTACTTCTG
5551 ACAACGATCG GAGGACCGAA GGAGCTAACC GCTTTTTTGC ACAACATGGG
5601 GGATCATGTA ACTCGCCTTG ATCGTTGGGA ACCGGAGCTG AATGAAGCCA
5651 TACCAAACGA CGAGCGTGAC ACCACGATGC CTGTAGCAAT GGCAACAACG
5701 TTGCGCAAAC TATTAACTGG CGAACTACTT ACTCTAGCTT CCCGGCAACA
5751 ATTAATAGAC TGGATGGAGG CGGATAAAGT TGCAGGACCA CTTCTGCGCT
5801 CGGCCCTTCC GGCTGGCTGG TTTATTGCTG ATAAATCTGG AGCCGGTGAG
5851 CGTGGGTCTC GCGGTATCAT TGCAGCACTG GGGCCAGATG GTAAGCCCTC
5901 CCGTATCGTA GTTATCTACA CGACGGGGAG TCAGGCAACT ATGGATGAAC
5951 GAAATAGACA GATCGCTGAG ATAGGTGCCT CACTGATTAA GCATTGGTAA
6001 CTGTCAGACC AAGTTTACTC ATATATACTT TAGATTGATT TAAAACTTCA
6051 TTTTTAATTT AAAAGGATCT AGGTGAAGAT CCTTTTTGAT AATCTCATGA
6101 CCAAAATCCC TTAACGTGAG TTTTCGTTCC ACTGAGCGTC AGACCCCGTA
6151 GAAAAGATCA AAGGATCTTC TTGAGATCCT TTTTTTCTGC GCGTAATCTG
6201 CTGCTTGCAA ACAAAAAAAC CACCGCTACC AGCGGTGGTT TGTTTGCCGG
6251 ATCAAGAGCT ACCAACTCTT TTTCCGAAGG TAACTGGCTT CAGCAGAGCG
6301 CAGATACCAA ATACTGTCCT TCTAGTGTAG CCGTAGTTAG GCCACCACTT
6351 CAAGAACTCT GTAGCACCGC CTACATACCT CGCTCTGCTA ATCCTGTTAC
6401 CAGTGGCTGC TGCCAGTGGC GATAAGTCGT GTCTTACCGG GTTGGACTCA
6451 AGACGATAGT TACCGGATAA GGCGCAGCGG TCGGGCTGAA CGGGGGGTTC
6501 GTGCACACAG CCCAGCTTGG AGCGAACGAC CTACACCGAA CTGAGATACC
6551 TACAGCGTGA GCTATGAGAA AGCGCCACGC TTCCCGAAGG GAGAAAGGCG
6601 GACAGGTATC CGGTAAGCGG CAGGGTCGGA ACAGGAGAGC GCACGAGGGA
6651 GCTTCCAGGG GGAAACGCCT GGTATCTTTA TAGTCCTGTC GGGTTTCGCC
6701 ACCTCTGACT TGAGCGTCGA TTTTTGTGAT GCTCGTCAGG GGGGCGGAGC
6751 CTATGGAAAA ACGCCAGCAA CGCGGCCTTT TTACGGTTCC TGGCCTTTTG
6801 CTGGCCTTTT GCTCACATGT
AAVC-Syn-mBPIP-TATκ28-eGFP
L-ITR: 1-141
Syn-1 promoter: 159-730
Bip-TATκ28-EGFP: 754-1659
bGHp(A): 1684-1911
R-ITR: 1923-2063
Amp(R): 2980-3837
pUC origin: 3988-4655
SEQ ID NO: 120
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGAC TACAAACCGA GTATCTGCAG AGGGCCCTGC GTATGAGTGC
  201 AAGTGGGTTT TAGGACCAGG ATGAGGCGGG GTGGGGGTGC CTACCTGACG
  251 ACCGACCCCG ACCCACTGGA CAAGCACCCA ACCCCCATTC CCCAAATTGC
  301 GCATCCCCTA TCAGAGAGGG GGAGGGGAAA CAGGATGCGG CGAGGCGCGT
  351 GCGCACTGCC AGCTTCAGCA CCGCGGACAG TGCCTTCGCC CCCGCCTGGC
  401 GGCGCGCGCC ACCGCCGCCT CAGCACTGAA GGCGCGCTGA CGTCACTCGC
  451 CGGTCCCCCG CAAACTCCCC TTCCCGGCCA CCTTGGTCGC GTCCGCGCCG
  501 CCGCCGGCCC AGCCGGACCG CACCACGCGA GGCGCGAGAT AGGGGGGCAC
  551 GGGCGCGACC ATCTGCGCTG CGGCGCCGGC GACTCAGCGC TGCCTCAGTC
  601 TGCGGTGGGC AGCGGAGGAG TCGTGTCGTG CCTGAGAGCG CAGCTGTGCT
  651 CCTGGGCACC GCGCAGTCCG CCCCCGCGGC TCCTGGCCAG ACCACCCCTA
  701 GGACCCCCTG CCCCAAGTCG CAGCCTTCGA GCTAGCCAAA GCTTGCCGCC
  751 ACCATGAAAC TGTCCCTGGT CGCCGCCATG CTGCTCCTCC TGTCACTGGT
  801 CGCCGCTATG CTGCTCCTCC TCTCCGCTGC TCGGGCTGGG GACGCTGCTC
  851 AGCCAGCTAG GAGAGCTAGG AGGACCAAGC TGGCTGCTTA CGCTAGGAAG
  901 GCTGCTAGGC AGGCTAGAGC TGGAGGAGGC GGATCTATGG TGAGCAAGGG
  951 AGAGGAGCTG TTCACAGGCG TGGTGCCCAT CCTGGTGGAG CTGGACGGAG
1001 ATGTGAACGG CCACAAGTTT AGCGTGTCCG GAGAGGGAGA GGGCGACGCT
1051 ACCTACGGAA AGCTGACACT GAAGTTCATC TGCACCACAG GCAAGCTGCC
1101 CGTGCCTTGG CCAACCCTGG TGACCACACT GACATACGGC GTGCAGTGTT
1151 TTTCCAGGTA CCCAGACCAC ATGAAGCAGC ACGATTTCTT TAAGTCTGCC
1201 ATGCCCGAGG GATACGTGCA GGAGCGGACC ATCTTCTTTA AGGACGATGG
1251 CAACTACAAG ACCCGCGCTG AGGTGAAGTT CGAGGGAGAC ACACTGGTGA
1301 ACAGGATCGA GCTGAAGGGC ATCGACTTTA AGGAGGATGG AAACATCCTG
1351 GGCCACAAGC TGGAGTACAA CTACAACAGC CACAACGTGT ACATCATGGC
1401 CGATAAGCAG AAGAACGGAA TCAAGGTGAA CTTCAAGATC AGACACAACA
1451 TCGAGGACGG CTCCGTGCAG CTGGCTGATC ACTACCAGCA GAACACCCCT
1501 ATCGGAGACG GACCCGTGCT GCTGCCTGAT AACCACTACC TGTCCACACA
1551 GTCTGCCCTG AGCAAGGACC CAAACGAGAA GCGGGATCAC ATGGTGCTGC
1601 TGGAATTTGT GACTGCTGCT GGTATTACAC TGGGTATGGA TGAACTCTAT
1651 AAATGATAAG CGGCCGCAAC TCGAGACTCT AGACGACTGT GCCTTCTAGT
1701 TGCCAGCCAT CTGTTGTTTG CCCCTCCCCC GTGCCTTCCT TGACCCTGGA
1751 AGGTGCCACT CCCACTGTCC TTTCCTAATA AAATGAGGAA ATTGCATCGC
1801 ATTGTCTGAG TAGGTGTCAT TCTATTCTGG GGGGTGGGGT GGGGCAGGAC
1851 AGCAAGGGGG AGGATTGGGA AGACAATAGC AGGCATGCTG GGGATGCGGT
1901 GGGCTCTATG GCCGCGGGCC GCAGGAACCC CTAGTGATGG AGTTGGCCAC
1951 TCCCTCTCTG CGCGCTCGCT CGCTCACTGA GGCCGGGCGA CCAAAGGTCG
2001 CCCGACGCCC GGGCTTTGCC CGGGCGGCCT CAGTGAGCGA GCGAGCGCGC
2051 AGCTGCCTGC AGGGGCGCCT GATGCGGTAT TTTCTCCTTA CGCATCTGTG
2101 CGGTATTTCA CACCGCATAC GTCAAAGCAA CCATAGTACG CGCCCTGTAG
2151 CGGCGCATTA AGCGCGGCGG GTGTGGTGGT TACGCGCAGC GTGACCGCTA
2201 CACTTGCCAG CGCCCTAGCG CCCGCTCCTT TCGCTTTCTT CCCTTCCTTT
2251 CTCGCCACGT TCGCCGGCTT TCCCCGTCAA GCTCTAAATC GGGGGCTCCC
2301 TTTAGGGTTC CGATTTAGTG CTTTACGGCA CCTCGACCCC AAAAAACTTG
2351 ATTTGGGTGA TGGTTCACGT AGTGGGCCAT CGCCCTGATA GACGGTTTTT
2401 CGCCCTTTGA CGTTGGAGTC CACGTTCTTT AATAGTGGAC TCTTGTTCCA
2451 AACTGGAACA ACACTCAACC CTATCTCGGG CTATTCTTTT GATTTATAAG
2501 GGATTTTGCC GATTTCGGCC TATTGGTTAA AAAATGAGCT GATTTAACAA
2551 AAATTTAACG CGAATTTTAA CAAAATATTA ACGTTTACAA TTTTATGGTG
2601 CACTCTCAGT ACAATCTGCT CTGATGCCGC ATAGTTAAGC CAGCCCCGAC
2651 ACCCGCCAAC ACCCGCTGAC GCGCCCTGAC GGGCTTGTCT GCTCCCGGCA
2701 TCCGCTTACA GACAAGCTGT GACCGTCTCC GGGAGCTGCA TGTGTCAGAG
2751 GTTTTCACCG TCATCACCGA AACGCGCGAG ACGAAAGGGC CTCGTGATAC
2801 GCCTATTTTT ATAGGTTAAT GTCATGATAA TAATGGTTTC TTAGACGTCA
2851 GGTGGCACTT TTCGGGGAAA TGTGCGCGGA ACCCCTATTT GTTTATTTTT
2901 CTAAATACAT TCAAATATGT ATCCGCTCAT GAGACAATAA CCCTGATAAA
2951 TGCTTCAATA ATATTGAAAA AGGAAGAGTA TGAGTATTCA ACATTTCCGT
3001 GTCGCCCTTA TTCCCTTTTT TGCGGCATTT TGCCTTCCTG TTTTTGCTCA
3051 CCCAGAAACG CTGGTGAAAG TAAAAGATGC TGAAGATCAG TTGGGTGCAC
3101 GAGTGGGTTA CATCGAACTG GATCTCAACA GCGGTAAGAT CCTTGAGAGT
3151 TTTCGCCCCG AAGAACGTTT TCCAATGATG AGCACTTTTA AAGTTCTGCT
3201 ATGTGGCGCG GTATTATCCC GTATTGACGC CGGGCAAGAG CAACTCGGTC
3251 GCCGCATACA CTATTCTCAG AATGACTTGG TTGAGTACTC ACCAGTCACA
3301 GAAAAGCATC TTACGGATGG CATGACAGTA AGAGAATTAT GCAGTGCTGC
3351 CATAACCATG AGTGATAACA CTGCGGCCAA CTTACTTCTG ACAACGATCG
3401 GAGGACCGAA GGAGCTAACC GCTTTTTTGC ACAACATGGG GGATCATGTA
3451 ACTCGCCTTG ATCGTTGGGA ACCGGAGCTG AATGAAGCCA TACCAAACGA
3501 CGAGCGTGAC ACCACGATGC CTGTAGCAAT GGCAACAACG TTGCGCAAAC
3551 TATTAACTGG CGAACTACTT ACTCTAGCTT CCCGGCAACA ATTAATAGAC
3601 TGGATGGAGG CGGATAAAGT TGCAGGACCA CTTCTGCGCT CGGCCCTTCC
3651 GGCTGGCTGG TTTATTGCTG ATAAATCTGG AGCCGGTGAG CGTGGGTCTC
3701 GCGGTATCAT TGCAGCACTG GGGCCAGATG GTAAGCCCTC CCGTATCGTA
3751 GTTATCTACA CGACGGGGAG TCAGGCAACT ATGGATGAAC GAAATAGACA
3801 GATCGCTGAG ATAGGTGCCT CACTGATTAA GCATTGGTAA CTGTCAGACC
3851 AAGTTTACTC ATATATACTT TAGATTGATT TAAAACTTCA TTTTTAATTT
3901 AAAAGGATCT AGGTGAAGAT CCTTTTTGAT AATCTCATGA CCAAAATCCC
3951 TTAACGTGAG TTTTCGTTCC ACTGAGCGTC AGACCCCGTA GAAAAGATCA
4001 AAGGATCTTC TTGAGATCCT TTTTTTCTGC GCGTAATCTG CTGCTTGCAA
4051 ACAAAAAAAC CACCGCTACC AGCGGTGGTT TGTTTGCCGG ATCAAGAGCT
4101 ACCAACTCTT TTTCCGAAGG TAACTGGCTT CAGCAGAGCG CAGATACCAA
4151 ATACTGTCCT TCTAGTGTAG CCGTAGTTAG GCCACCACTT CAAGAACTCT
4201 GTAGCACCGC CTACATACCT CGCTCTGCTA ATCCTGTTAC CAGTGGCTGC
4251 TGCCAGTGGC GATAAGTCGT GTCTTACCGG GTTGGACTCA AGACGATAGT
4301 TACCGGATAA GGCGCAGCGG TCGGGCTGAA CGGGGGGTTC GTGCACACAG
4351 CCCAGCTTGG AGCGAACGAC CTACACCGAA CTGAGATACC TACAGCGTGA
4401 GCTATGAGAA AGCGCCACGC TTCCCGAAGG GAGAAAGGCG GACAGGTATC
4451 CGGTAAGCGG CAGGGTCGGA ACAGGAGAGC GCACGAGGGA GCTTCCAGGG
4501 GGAAACGCCT GGTATCTTTA TAGTCCTGTC GGGTTTCGCC ACCTCTGACT
4551 TGAGCGTCGA TTTTTGTGAT GCTCGTCAGG GGGGCGGAGC CTATGGAAAA
4601 ACGCCAGCAA CGCGGCCTTT TTACGGTTCC TGGCCTTTTG CTGGCCTTTT
4651 GCTCACATGT
AAVC-Syn- mBPIP-TATκ28-NLS-eGFP
L-ITR: 1-141
Syn-1 promoter: 159-730
Bip-TATκ28-NLS-eGFP: 754-1719
bGHp(A): 1744-1971
R-ITR: 1983-2123
Amp(R): 3040-3897
pUC origin: 4048-4715
SEQ ID NO: 121
      1 CCTGCAGGCA GCTGCGCGCT CGCTCGCTCA CTGAGGCCGC CCGGGCAAAG
    51 CCCGGGCGTC GGGCGACCTT TGGTCGCCCG GCCTCAGTGA GCGAGCGAGC
  101 GCGCAGAGAG GGAGTGGCCA ACTCCATCAC TAGGGGTTCC TGCGGCCTAA
  151 GGCAATTGAC TACAAACCGA GTATCTGCAG AGGGCCCTGC GTATGAGTGC
  201 AAGTGGGTTT TAGGACCAGG ATGAGGCGGG GTGGGGGTGC CTACCTGACG
  251 ACCGACCCCG ACCCACTGGA CAAGCACCCA ACCCCCATTC CCCAAATTGC
  301 GCATCCCCTA TCAGAGAGGG GGAGGGGAAA CAGGATGCGG CGAGGCGCGT
  351 GCGCACTGCC AGCTTCAGCA CCGCGGACAG TGCCTTCGCC CCCGCCTGGC
  401 GGCGCGCGCC ACCGCCGCCT CAGCACTGAA GGCGCGCTGA CGTCACTCGC
  451 CGGTCCCCCG CAAACTCCCC TTCCCGGCCA CCTTGGTCGC GTCCGCGCCG
  501 CCGCCGGCCC AGCCGGACCG CACCACGCGA GGCGCGAGAT AGGGGGGCAC
  551 GGGCGCGACC ATCTGCGCTG CGGCGCCGGC GACTCAGCGC TGCCTCAGTC
  601 TGCGGTGGGC AGCGGAGGAG TCGTGTCGTG CCTGAGAGCG CAGCTGTGCT
  651 CCTGGGCACC GCGCAGTCCG CCCCCGCGGC TCCTGGCCAG ACCACCCCTA
  701 GGACCCCCTG CCCCAAGTCG CAGCCTTCGA GCTAGCCAAA GCTTGCCGCC
  751 ACCATGAAAC TCAGTCTGGT CGCCGCTATG CTCCTGCTCC TCTCCCTGGT
  801 CGCCGCTATG CTCCTGCTCC TGTCTGCTGC CCGCGCTGGG GACGCTGCTC
  851 AGCCAGCTAG GAGAGCTAGG AGGACCAAGC TGGCTGCTTA CGCTAGAAAG
  901 GCTGCTAGGC AGGCTAGAGC TGGAGGAGGA GGATCCATGG CTCCCAAGAA
  951 GAAGAGGAAG GTGCGCTACC CCGCCTTCCT GTACAAGGTG GCTACCATGG
1001 TGTCTAAGGG AGAGGAGCTG TTTACAGGCG TGGTGCCCAT CCTGGTGGAG
1051 CTGGACGGAG ATGTGAACGG CCACAAGTTC AGCGTGTCCG GAGAGGGAGA
1101 GGGCGACGCC ACCTACGGAA AGCTGACACT GAAGTTTATC TGCACCACAG
1151 GCAAGCTGCC CGTGCCTTGG CCAACCCTGG TGACCACACT GACATACGGC
1201 GTGCAGTGTT TCTCTCGGTA CCCTGACCAC ATGAAGCAGC ACGATTTCTT
1251 TAAGAGCGCC ATGCCAGAGG GATACGTGCA GGAGAGGACA ATCTTCTTTA
1301 AGGACGATGG CAACTACAAG ACCAGAGCTG AGGTGAAGTT CGAGGGAGAC
1351 ACACTGGTGA ACCGGATCGA GCTGAAGGGC ATCGACTTTA AGGAGGATGG
1401 AAACATCCTG GGCCACAAGC TGGAGTACAA CTACAACAGC CACAACGTGT
1451 ACATCATGGC CGATAAGCAG AAGAACGGAA TCAAGGTGAA CTTTAAGATC
1501 CGCCACAACA TCGAGGACGG CTCCGTGCAG CTGGCTGATC ACTACCAGCA
1551 GAACACCCCA ATCGGAGACG GACCCGTGCT GCTGCCTGAT AACCACTACC
1601 TGTCTACACA GAGCGCCCTG TCCAAGGACC CTAACGAGAA GAGGGATCAC
1651 ATGGTCCTCC TGGAATTTGT GACTGCTGCT GGGATTACTC TCGGTATGGA
1701 TGAACTGTAT AAATGATAAG CGGCCGCAAC TCGAGACTCT AGACGACTGT
1751 GCCTTCTAGT TGCCAGCCAT CTGTTGTTTG CCCCTCCCCC GTGCCTTCCT
1801 TGACCCTGGA AGGTGCCACT CCCACTGTCC TTTCCTAATA AAATGAGGAA
1851 ATTGCATCGC ATTGTCTGAG TAGGTGTCAT TCTATTCTGG GGGGTGGGGT
1901 GGGGCAGGAC AGCAAGGGGG AGGATTGGGA AGACAATAGC AGGCATGCTG
1951 GGGATGCGGT GGGCTCTATG GCCGCGGGCC GCAGGAACCC CTAGTGATGG
2001 AGTTGGCCAC TCCCTCTCTG CGCGCTCGCT CGCTCACTGA GGCCGGGCGA
2051 CCAAAGGTCG CCCGACGCCC GGGCTTTGCC CGGGCGGCCT CAGTGAGCGA
2101 GCGAGCGCGC AGCTGCCTGC AGGGGCGCCT GATGCGGTAT TTTCTCCTTA
2151 CGCATCTGTG CGGTATTTCA CACCGCATAC GTCAAAGCAA CCATAGTACG
2201 CGCCCTGTAG CGGCGCATTA AGCGCGGCGG GTGTGGTGGT TACGCGCAGC
2251 GTGACCGCTA CACTTGCCAG CGCCCTAGCG CCCGCTCCTT TCGCTTTCTT
2301 CCCTTCCTTT CTCGCCACGT TCGCCGGCTT TCCCCGTCAA GCTCTAAATC
2351 GGGGGCTCCC TTTAGGGTTC CGATTTAGTG CTTTACGGCA CCTCGACCCC
2401 AAAAAACTTG ATTTGGGTGA TGGTTCACGT AGTGGGCCAT CGCCCTGATA
2451 GACGGTTTTT CGCCCTTTGA CGTTGGAGTC CACGTTCTTT AATAGTGGAC
2501 TCTTGTTCCA AACTGGAACA ACACTCAACC CTATCTCGGG CTATTCTTTT
2551 GATTTATAAG GGATTTTGCC GATTTCGGCC TATTGGTTAA AAAATGAGCT
2601 GATTTAACAA AAATTTAACG CGAATTTTAA CAAAATATTA ACGTTTACAA
2651 TTTTATGGTG CACTCTCAGT ACAATCTGCT CTGATGCCGC ATAGTTAAGC
2701 CAGCCCCGAC ACCCGCCAAC ACCCGCTGAC GCGCCCTGAC GGGCTTGTCT
2751 GCTCCCGGCA TCCGCTTACA GACAAGCTGT GACCGTCTCC GGGAGCTGCA
2801 TGTGTCAGAG GTTTTCACCG TCATCACCGA AACGCGCGAG ACGAAAGGGC
2851 CTCGTGATAC GCCTATTTTT ATAGGTTAAT GTCATGATAA TAATGGTTTC
2901 TTAGACGTCA GGTGGCACTT TTCGGGGAAA TGTGCGCGGA ACCCCTATTT
2951 GTTTATTTTT CTAAATACAT TCAAATATGT ATCCGCTCAT GAGACAATAA
3001 CCCTGATAAA TGCTTCAATA ATATTGAAAA AGGAAGAGTA TGAGTATTCA
3051 ACATTTCCGT GTCGCCCTTA TTCCCTTTTT TGCGGCATTT TGCCTTCCTG
3101 TTTTTGCTCA CCCAGAAACG CTGGTGAAAG TAAAAGATGC TGAAGATCAG
3151 TTGGGTGCAC GAGTGGGTTA CATCGAACTG GATCTCAACA GCGGTAAGAT
3201 CCTTGAGAGT TTTCGCCCCG AAGAACGTTT TCCAATGATG AGCACTTTTA
3251 AAGTTCTGCT ATGTGGCGCG GTATTATCCC GTATTGACGC CGGGCAAGAG
3301 CAACTCGGTC GCCGCATACA CTATTCTCAG AATGACTTGG TTGAGTACTC
3351 ACCAGTCACA GAAAAGCATC TTACGGATGG CATGACAGTA AGAGAATTAT
3401 GCAGTGCTGC CATAACCATG AGTGATAACA CTGCGGCCAA CTTACTTCTG
3451 ACAACGATCG GAGGACCGAA GGAGCTAACC GCTTTTTTGC ACAACATGGG
3501 GGATCATGTA ACTCGCCTTG ATCGTTGGGA ACCGGAGCTG AATGAAGCCA
3551 TACCAAACGA CGAGCGTGAC ACCACGATGC CTGTAGCAAT GGCAACAACG
3601 TTGCGCAAAC TATTAACTGG CGAACTACTT ACTCTAGCTT CCCGGCAACA
3651 ATTAATAGAC TGGATGGAGG CGGATAAAGT TGCAGGACCA CTTCTGCGCT
3701 CGGCCCTTCC GGCTGGCTGG TTTATTGCTG ATAAATCTGG AGCCGGTGAG
3751 CGTGGGTCTC GCGGTATCAT TGCAGCACTG GGGCCAGATG GTAAGCCCTC
3801 CCGTATCGTA GTTATCTACA CGACGGGGAG TCAGGCAACT ATGGATGAAC
3851 GAAATAGACA GATCGCTGAG ATAGGTGCCT CACTGATTAA GCATTGGTAA
3901 CTGTCAGACC AAGTTTACTC ATATATACTT TAGATTGATT TAAAACTTCA
3951 TTTTTAATTT AAAAGGATCT AGGTGAAGAT CCTTTTTGAT AATCTCATGA
4001 CCAAAATCCC TTAACGTGAG TTTTCGTTCC ACTGAGCGTC AGACCCCGTA
4051 GAAAAGATCA AAGGATCTTC TTGAGATCCT TTTTTTCTGC GCGTAATCTG
4101 CTGCTTGCAA ACAAAAAAAC CACCGCTACC AGCGGTGGTT TGTTTGCCGG
4151 ATCAAGAGCT ACCAACTCTT TTTCCGAAGG TAACTGGCTT CAGCAGAGCG
4201 CAGATACCAA ATACTGTCCT TCTAGTGTAG CCGTAGTTAG GCCACCACTT
4251 CAAGAACTCT GTAGCACCGC CTACATACCT CGCTCTGCTA ATCCTGTTAC
4301 CAGTGGCTGC TGCCAGTGGC GATAAGTCGT GTCTTACCGG GTTGGACTCA
4351 AGACGATAGT TACCGGATAA GGCGCAGCGG TCGGGCTGAA CGGGGGGTTC
4401 GTGCACACAG CCCAGCTTGG AGCGAACGAC CTACACCGAA CTGAGATACC
4451 TACAGCGTGA GCTATGAGAA AGCGCCACGC TTCCCGAAGG GAGAAAGGCG
4501 GACAGGTATC CGGTAAGCGG CAGGGTCGGA ACAGGAGAGC GCACGAGGGA
4551 GCTTCCAGGG GGAAACGCCT GGTATCTTTA TAGTCCTGTC GGGTTTCGCC
4601 ACCTCTGACT TGAGCGTCGA TTTTTGTGAT GCTCGTCAGG GGGGCGGAGC
4651 CTATGGAAAA ACGCCAGCAA CGCGGCCTTT TTACGGTTCC TGGCCTTTTG
4701 CTGGCCTTTT GCTCACATGT
DNA sequence for mBPIP-TATκ28-CDKL5-107 (human optimized)
SEQ ID NO: 122
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGAACCTGAGCGAGGGCAACAATGCCAATTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGAATCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTAATCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGAATATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATAACAGGACCAATAGATCCAGG
ATGCCCAATCTGAACGATCTGAAGGAGACAGCCCTGTGA
DNA sequence for CDKL5-107 (human optimized)
SEQ ID NO: 123
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGAACCTGAGCGAGGGCAACAATGCCAATTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGAATCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTAATCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGAATATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATAACAGGACCAATAGA
TCCAGGATGCCCAATCTGAACGATCTGAAGGAGACAGCCCTG
DNA sequence for MBIP-TATκ28-CDKL5_107-FH [1-7NQ]
(human optimized)
SEQ ID NO: 124
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

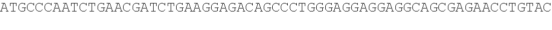

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107 [1-7NQ] (human optimized)
SEQ ID NO: 125
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5_107-FH [2-7NQ] (human optimized)
SEQ ID NO: 126
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGAACCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

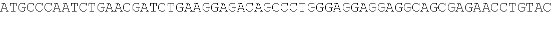

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107[2-7NQ] (human optimized)
SEQ ID NO: 127
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGAACCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5107-FH [1,3-7NQ]
(human optimized)
SEQ ID NO: 128
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCAATTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

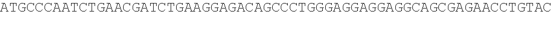

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107 [1,3-7NQ] (human optimized)
SEQ ID NO: 129
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCAATTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5_107-FH [1-2,4-7NQ]
(human optimized)
SEQ ID NO: 130
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGAATCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

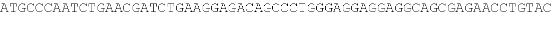

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107 [1-2,4-7NQ] (human optimized)
SEQ ID NO: 131
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGAATCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5_107-FH [1-3,5-7NQ]
(human optimized)
SEQ ID NO: 132
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTAATCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

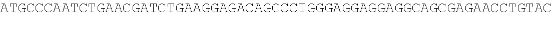

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107[1-3,5-7NQ] (human optimized)
SEQ ID NO: 133
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTAATCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5_107-FH [1-4,6-7NQ]
(human optimized)
SEQ ID NO: 134
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGAATATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

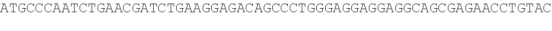

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107[1-4,6-7NQ] (human optimized)
SEQ ID NO: 135
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGAATATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5_107-FH [1-5,7NQ]
(human optimized)
SEQ ID NO: 136
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATAACAGGACCCAGAGATCCAGG

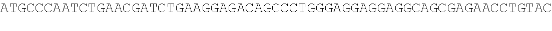

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107[1-5,7NQ] (human optimized)
SEQ ID NO: 137
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATAACAGGACCCAGAGA
TCCAGGATGCCCAATCTGAACGATCTGAAGGAGACAGCCCTG
DNA sequence for MBIP-TATκ28-CDKL5_107-FH [1-6NQ]
(human optimized)
SEQ ID NO: 138
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCAATAGATCCAGG

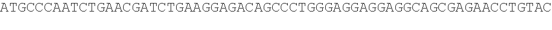

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107[1-6NQ] (human optimized)
SEQ ID NO: 139
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCAATAGA
TCCAGGATGCCCAATCTGAACGATCTGAAGGAGACAGCCCTG
DNA sequence for MBIP-TATκ28-CDKL5_107-FH [2NQ]
(human optimized)
SEQ ID NO: 140
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGAACCTGAGCGAGGGCAACAATGCCCATTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGAATCTGTCCGTGGGCCTGCCTAGGGCCGACGAGGGCCTG
CCAGCAAACGAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTAATCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAATAACGTGAGCACCCGGGTGAGCAGC
CTGCCATCTGAGTCTAGCTCCGGCACAAACCACTCTAAGAGGCAGCCCGCCTTTGACCCTTGG
AAGAGCCCAGAGAATATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATAACAGGACCAATAGATCCAGG

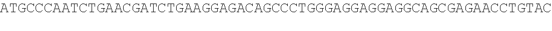

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107 [2NQ] (human optimized)
SEQ ID NO: 141
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGAACCTGAGCGAGGGCAACAATGCCCATTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGAATCTGTCCGTGGGCCTGCCTAGGGCCGACGAG
GGCCTGCCAGCAAACGAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTAATCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAATAACGTGAGCACCCGGGTG
AGCAGCCTGCCATCTGAGTCTAGCTCCGGCACAAACCACTCTAAGAGGCAGCCCGCCTTTGAC
CCTTGGAAGAGCCCAGAGAATATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATAACAGGACCAATAGA
TCCAGGATGCCCAATCTGAACGATCTGAAGGAGACAGCCCTG
DNA sequence for MBIP-TATκ28-CDKL5_107-FH [1-10NQ] (human optimized)
SEQ ID NO: 142
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCA [symbol] GAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAAT [symbol] GTGAGCACCCGGGTGAGCAGC
CTGCCATCTGAGTCTAGCTCCGGCACA [symbol] CACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

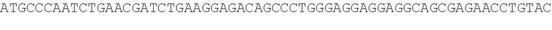

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107[1-10NQ] (human optimized)
SEQ ID NO: 143
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCA [symbol] GAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAAT [symbol] GTGAGCACCCGGGTG
AGCAGCCTGCCATCTGAGTCTAGCTCCGGCACA [symbol] CACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5_107-FH [1-7,9-10NQ]
(human optimized)
SEQ ID NO: 144
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCA [symbol] GAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAAT [symbol] GTGAGCACCCGGGTGAGCAGC
CTGCCATCTGAGTCTAGCTCCGGCACA [symbol] CACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

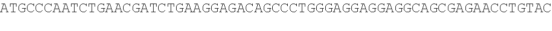

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107 [1-7,9-10NQ] (human optimized)
SEQ ID NO: 145
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCA [symbol] GAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAAT [symbol] GTGAGCACCCGGGTG
AGCAGCCTGCCATCTGAGTCTAGCTCCGGCACA [symbol] CACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5_107-FH [1-8, 10NQ]
(human optimized)
SEQ ID NO: 146
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCA [symbol] GAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAAT [symbol] GTGAGCACCCGGGTGAGCAGC
CTGCCATCTGAGTCTAGCTCCGGCACA [symbol] CACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

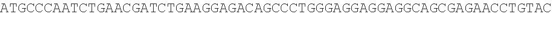

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107[1-8,10NQ] (human optimized)
SEQ ID NO: 147
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCA [symbol] GAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAAT [symbol] GTGAGCACCCGGGTG
AGCAGCCTGCCATCTGAGTCTAGCTCCGGCACA [symbol] CACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for MBIP-TATκ28-CDKL5_107-FH [1-9NQ]
(human optimized)
SEQ ID NO: 148
ATGAAGCTGTCCCTGGTGGCCGCTATGCTGCTGCTGCTGTCTCTGGTCGCTGCCATGTTATTA
CTGCTGTCTGCCGCTAGGGCCGGGGACGCAGCACAGCCCGCAAGAAGAGCAAGAAGAACTAAA
CTGGCCGCTTACGCAAGGAAGGCAGCAAGACAGGCAAGAGCAGGCGGCGGCGGCTCCAAGATC
CCCAATATCGGCAACGTGATGAATAAGTTCGAGATCCTGGGAGTGGTGGGAGAGGGAGCCTAC
GGCGTGGTGCTGAAGTGCAGACACAAGGAGACACACGAGATCGTGGCCATCAAGAAGTTTAAG
GACAGCGAGGAGAATGAGGAGGTGAAGGAGACAACCCTGCGCGAGCTGAAGATGCTGCGGACA
CTGAAGCAGGAGAACATCGTGGAGCTGAAGGAGGCCTTCCGGAGAAGGGGCAAGCTGTACCTG
GTGTTTGAGTATGTGGAGAAGAACATGCTGGAGCTGCTGGAGGAGATGCCTAATGGCGTGCCC
CCTGAGAAGGTGAAGTCCTACATCTATCAGCTGATCAAGGCCATCCACTGGTGCCACAAGAAC
GATATCGTGCACCGCGACATCAAGCCCGAGAACCTGCTGATCTCCCACAATGACGTGCTGAAG
CTGTGCGACTTCGGCTTTGCCCGGCAGCTGAGCGAGGGCAACAATGCCCAGTACACAGAGTAT
GTGGCCACCCGCTGGTACAGAAGCCCCGAGCTGCTGCTGGGCGCCCCCTATGGCAAGAGCGTG
GATATGTGGTCCGTGGGCTGCATCCTGGGCGAGCTGTCTGATGGCCAGCCTCTGTTCCCAGGC
GAGAGCGAGATCGACCAGCTGTTTACCATCCAGAAGGTGCTGGGCCCTCTGCCAAGCGAGCAG
ATGAAGCTGTTCTACTCCAACCCAAGGTTCCACGGCCTGAGGTTTCCAGCCGTGAATCACCCT
CAGAGCCTGGAGCGCCGGTATCTGGGCATCCTGAACTCCGTGCTGCTGGACCTGATGAAGAAC
CTGCTGAAGCTGGACCCCGCCGACAGATACCTGACCGAGCAGTGTCTGAATCACCCTACATTT
CAGACCCAGAGACTGCTGGATAGGAGCCCTTCCCGCTCTGCCAAGCGGAAGCCATATCACGTG
GAGAGCAGCACCCTGTCCAATCGCAACCAGGCCGGCAAGTCTACAGCCCTGCAGAGCCACCAC
CGGAGCAACTCCAAGGATATCCAGCAGCTGTCCGTGGGCCTGCCTAGGGCCGACGAGGGCCTG
CCAGCA [symbol] GAGAGCTTCCTGAATGGAAACCTGGCAGGAGCCTCTCTGAGCCCACTGCACACA
AAGACCTACCAGGCCTCTAGCCAGCCCGGCTCCACATCTAAGGACCTGACCAACAATAACATC
CCACACCTGCTGTCTCCCAAGGAGGCCAAGAGCAAGACCGAGTTCGACTTCAACATCGACCCA
AAGCCTAGCGAGGGACCTGGCACAAAGTATCTGAAGAGCAACAGCCGGAGCCAGCAGAATAGG
CACTCCTTCATGGAGTCCTCTCAGTCTAAGGCCGGCACCCTGCAGCCAAACGAGAAGCAGAGC
AGGCACTCCTACATCGACACCATCCCACAGAGCAGCCGGAGCCCCTCCTATCGGACAAAGGCC
AAGTCTCACGGCGCCCTGTCTGATAGCAAGTCCGTGTCTCAGCTGAGCGAGGCCAGAGCCCAG
ATCGCAGAGCCCAGCACCTCCAGGTACTTTCCTTCTAGCTGTCTGGATCTGAACTCTCCTACA
AGCCCAACACCCACCAGACACAGCGACACAAGGACCCTGCTGTCTCCAAGCGGCAGAAATAAC
AGGAACGAGGGCACCCTGGACAGCCGGCGGACCACAACCAGGCACAGCAAGACAATGGAGGAG
CTGAAGCTGCCAGAGCACATGGATTCCTCTCACTCCCACTCTCTGAGCGCCCCCCACGAGTCC
TTCTCTTACGGCCTGGGCTATACCTCCCCCTTCAGCAGCCAGCAGCGCCCCCACCGGCACTCT
ATGTACGTGACAAGAGATAAGGTGAGGGCAAAGGGCCTGGACGGCAGCCTGTCCATCGGACAG
GGAATGGCAGCCCGGGCCAACTCCCTGCAGCTGCTGTCTCCTCAGCCAGGAGAGCAGCTGCCA
CCAGAGATGACCGTGGCACGGAGCAGCGTGAAGGAGACAAGCAGGGAGGGCACCTCCTCTTTC
CACACAAGACAGAAGTCCGAGGGCGGCGTGTATCACGATCCCCACTCTGACGATGGCACAGCC
CCTAAGGAGAACAGGCACCTGTACAATGACCCCGTGCCTAGGAGGGTGGGCTCCTTCTATCGC
GTGCCATCTCCCCGGCCTGATAATAGCTTTCACGAGAAT [symbol] GTGAGCACCCGGGTGAGCAGC
CTGCCATCTGAGTCTAGCTCCGGCACA [symbol] CACTCTAAGAGGCAGCCCGCCTTTGACCCTTGG
AAGAGCCCAGAGCAGATCTCTCACAGCGAGCAGCTGAAGGAGAAGGAGAAGCAGGGCTTCTTT
CGCAGCATGAAGAAGAAGAAGAAGAAGAGCCAGACCGTGCCTAACTCCGATTCTCCAGACCTG
CTGACCCTGCAGAAGTCCATCCACAGCGCCTCCACACCCTCTAGCAGACCTAAGGAGTGGAGG
CCTGAGAAGATCAGCGACCTGCAGACCCAGAGCCAGCCACTGAAGTCCCTGCGGAAGCTGCTG
CACCTGTCCTCTGCCAGCAACCACCCAGCCAGCTCCGATCCAAGGTTCCAGCCCCTGACAGCC
CAGCAGACCAAGAACAGCTTCAGCGAGATCAGAATCCACCCTCTGTCCCAGGCCTCTGGAGGC
TCTAGCAACATCAGGCAGGAGCCAGCACCAAAGGGCCGGCCCGCCCTGCAGCTGCCTGGCCAG
ATGGACCCAGGCTGGCACGTGTCCTCTGTGACAAGATCCGCCACCGAGGGCCCATCCTACTCT
GAGCAGCTGGGAGCAAAGAGCGGACCTAATGGACACCCATATCAGAGGACCCAGAGATCCAGG

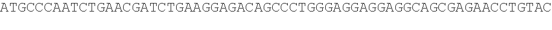

TTCCAGGGCGATTATAAGGACCACGATGGCGACTACAAGGACCACGACATTGACTACAAGGAC
GACGACGATAAAGACGGAGCACCCCATCACCACCACCATCATTGA
DNA sequence for CDKL5_107[1-9NQ] (human optimized)
SEQ ID NO: 149
AAGATCCCCAATATCGGCAACGTGATGAATAAGTTCGAGATCCTGGGAGTGGTGGGAGAGGGA
GCCTACGGCGTGGTGCTGAAGTGCAGACACAAGGAGACACACGAGATCGTGGCCATCAAGAAG
TTTAAGGACAGCGAGGAGAATGAGGAGGTGAAGGAGACAACCCTGCGCGAGCTGAAGATGCTG
CGGACACTGAAGCAGGAGAACATCGTGGAGCTGAAGGAGGCCTTCCGGAGAAGGGGCAAGCTG
TACCTGGTGTTTGAGTATGTGGAGAAGAACATGCTGGAGCTGCTGGAGGAGATGCCTAATGGC
GTGCCCCCTGAGAAGGTGAAGTCCTACATCTATCAGCTGATCAAGGCCATCCACTGGTGCCAC
AAGAACGATATCGTGCACCGCGACATCAAGCCCGAGAACCTGCTGATCTCCCACAATGACGTG
CTGAAGCTGTGCGACTTCGGCTTTGCCCGGCAGCTGAGCGAGGGCAACAATGCCCAGTACACA
GAGTATGTGGCCACCCGCTGGTACAGAAGCCCCGAGCTGCTGCTGGGCGCCCCCTATGGCAAG
AGCGTGGATATGTGGTCCGTGGGCTGCATCCTGGGCGAGCTGTCTGATGGCCAGCCTCTGTTC
CCAGGCGAGAGCGAGATCGACCAGCTGTTTACCATCCAGAAGGTGCTGGGCCCTCTGCCAAGC
GAGCAGATGAAGCTGTTCTACTCCAACCCAAGGTTCCACGGCCTGAGGTTTCCAGCCGTGAAT
CACCCTCAGAGCCTGGAGCGCCGGTATCTGGGCATCCTGAACTCCGTGCTGCTGGACCTGATG
AAGAACCTGCTGAAGCTGGACCCCGCCGACAGATACCTGACCGAGCAGTGTCTGAATCACCCT
ACATTTCAGACCCAGAGACTGCTGGATAGGAGCCCTTCCCGCTCTGCCAAGCGGAAGCCATAT
CACGTGGAGAGCAGCACCCTGTCCAATCGCAACCAGGCCGGCAAGTCTACAGCCCTGCAGAGC
CACCACCGGAGCAACTCCAAGGATATCCAGCAGCTGTCCGTGGGCCTGCCTAGGGCCGACGAG
GGCCTGCCAGCA [symbol] GAGAGCTTCCTGAATGGAAACCTGGCAGGAGCCTCTCTGAGCCCACTG
CACACAAAGACCTACCAGGCCTCTAGCCAGCCCGGCTCCACATCTAAGGACCTGACCAACAAT
AACATCCCACACCTGCTGTCTCCCAAGGAGGCCAAGAGCAAGACCGAGTTCGACTTCAACATC
GACCCAAAGCCTAGCGAGGGACCTGGCACAAAGTATCTGAAGAGCAACAGCCGGAGCCAGCAG
AATAGGCACTCCTTCATGGAGTCCTCTCAGTCTAAGGCCGGCACCCTGCAGCCAAACGAGAAG
CAGAGCAGGCACTCCTACATCGACACCATCCCACAGAGCAGCCGGAGCCCCTCCTATCGGACA
AAGGCCAAGTCTCACGGCGCCCTGTCTGATAGCAAGTCCGTGTCTCAGCTGAGCGAGGCCAGA
GCCCAGATCGCAGAGCCCAGCACCTCCAGGTACTTTCCTTCTAGCTGTCTGGATCTGAACTCT
CCTACAAGCCCAACACCCACCAGACACAGCGACACAAGGACCCTGCTGTCTCCAAGCGGCAGA
AATAACAGGAACGAGGGCACCCTGGACAGCCGGCGGACCACAACCAGGCACAGCAAGACAATG
GAGGAGCTGAAGCTGCCAGAGCACATGGATTCCTCTCACTCCCACTCTCTGAGCGCCCCCCAC
GAGTCCTTCTCTTACGGCCTGGGCTATACCTCCCCCTTCAGCAGCCAGCAGCGCCCCCACCGG
CACTCTATGTACGTGACAAGAGATAAGGTGAGGGCAAAGGGCCTGGACGGCAGCCTGTCCATC
GGACAGGGAATGGCAGCCCGGGCCAACTCCCTGCAGCTGCTGTCTCCTCAGCCAGGAGAGCAG
CTGCCACCAGAGATGACCGTGGCACGGAGCAGCGTGAAGGAGACAAGCAGGGAGGGCACCTCC
TCTTTCCACACAAGACAGAAGTCCGAGGGCGGCGTGTATCACGATCCCCACTCTGACGATGGC
ACAGCCCCTAAGGAGAACAGGCACCTGTACAATGACCCCGTGCCTAGGAGGGTGGGCTCCTTC
TATCGCGTGCCATCTCCCCGGCCTGATAATAGCTTTCACGAGAAT [symbol] GTGAGCACCCGGGTG
AGCAGCCTGCCATCTGAGTCTAGCTCCGGCACA [symbol] CACTCTAAGAGGCAGCCCGCCTTTGAC
CCTTGGAAGAGCCCAGAGCAGATCTCTCACAGCGAGCAGCTGAAGGAGAAGGAGAAGCAGGGC
TTCTTTCGCAGCATGAAGAAGAAGAAGAAGAAGAGCCAGACCGTGCCTAACTCCGATTCTCCA
GACCTGCTGACCCTGCAGAAGTCCATCCACAGCGCCTCCACACCCTCTAGCAGACCTAAGGAG
TGGAGGCCTGAGAAGATCAGCGACCTGCAGACCCAGAGCCAGCCACTGAAGTCCCTGCGGAAG
CTGCTGCACCTGTCCTCTGCCAGCAACCACCCAGCCAGCTCCGATCCAAGGTTCCAGCCCCTG
ACAGCCCAGCAGACCAAGAACAGCTTCAGCGAGATCAGAATCCACCCTCTGTCCCAGGCCTCT
GGAGGCTCTAGCAACATCAGGCAGGAGCCAGCACCAAAGGGCCGGCCCGCCCTGCAGCTGCCT
GGCCAGATGGACCCAGGCTGGCACGTGTCCTCTGTGACAAGATCCGCCACCGAGGGCCCATCC
TACTCTGAGCAGCTGGGAGCAAAGAGCGGACCTAATGGACACCCATATCAGAGGACCCAGAGA
TCCAGGATGCCCAATCTGAACGATCTGAAGGAGACAGCCCTG
DNA sequence for TATκ11 (human optimized)
SEQ ID NO: 150
TACGCCCGGAAGGCCGCCCGGCAGGCCAGAGCC
DNA sequence for TATk28 (human optimized)
SEQ ID NO: 151
GACGCAGCACAGCCCGCAAGAAGAGCAAGAAGAACTAAACTGGCCGCTTACGCAAGGAAGGCA
GCAAGACAGGCAAGAGCA
DNA sequence for Antennapedia CPP (human optimized)
SEQ ID NO: 152
CGGCAGATCAAGATTTGGTTCCAGAACCGGAGAATGAAGTGGAAGAAG
DNA sequence for Transportan CPP (human optimized)
SEQ ID NO: 153
GCCGGCTACCTGCTGGGCAAGATCAACCTGAAGGCCCTGGCCGCCCTGGCCAAGAAGATCCTG
DNA sequence for P97 CPP (human optimized)
SEQ ID NO: 154
GACAGCTCCCACGCCTTCACCCTGGATGAGCTGCGG
DNA sequence for mBIP (human optimized)
SEQ ID NO: 155
ATGAAGCTGTCCCTGGTGGCCGCTATGCTGCTGCTGCTGTCTCTGGTCGCTGCCATGTTATTA
CTGCTGTCTGCCGCTAGGGCC
IGF
SEQ ID NO: 156
AYRPSETLCGGELVDTLQFVCGDRGFYFSRPASRVSRRSRGIVEECCFRSCDLALLETYCATP
AKSE
IGF F26S
SEQ ID NO: 157
AYRPSETLCGGELVDTLQFVCGDRGSYFSRPASRVSRRSRGIVEECCFRSCDLALLETYCATP
AKSE
IGF Y27L
SEQ ID NO: 158
AYRPSETLCGGELVDTLQFVCGDRGFLFSRPASRVSRRSRGIVEECCFRSCDLALLETYCATP
AKSE
IGF V43L
SEQ ID NO: 159
AYRPSETLCGGELVDTLQFVCGDRGFYFSRPASRVSRRSRGILEECCFRSCDLALLETYCATP
AKSE
IGF F48T
SEQ ID NO: 160
AYRPSETLCGGELVDTLQFVCGDRGFYFSRPASRVSRRSRGIVEECCTRSCDLALLETYCATP
AKSE
IGF R49S
SEQ ID NO: 161
AYRPSETLCGGELVDTLQFVCGDRGFYFSRPASRVSRRSRGIVEECCFSSCDLALLETYCATP
AKSE
IGF S50I
SEQ ID NO: 162
AYRPSETLCGGELVDTLQFVCGDRGFYFSRPASRVSRRSRGIVEECCFRICDLALLETYCATP
AKSE
IGF A54R
SEQ ID NO: 163
AYRPSETLCGGELVDTLQFVCGDRGFYFSRPASRVSRRSRGIVEECCFRSCDLRLLETYCATP
AKSE
IGF L55R
SEQ ID NO: 164
AYRPSETLCGGELVDTLQFVCGDRGFYFSRPASRVSRRSRGIVEECCFRSCDLARLETYCATP
AKSE
IGF F26S, Y27L, V43L, F48T, R49S, S50I, A54R, L55R
SEQ ID NO: 165
AYRPSETLCGGELVDTLQFVCGDRGSLFSRPASRVSRRSRGILEECCTSICDLRRLETYCATP
AKSE
IGF ΔA1-7, Y27L, K65R
SEQ ID NO: 166
TLCGGELVDTLQFVCGDRGFLFSRPASRVSRRSRGIVEECCFRSCDLALLETYCATPARSE
TATκκ28 CPP
SEQ ID NO: 167
DAAQPARRAARTKLAAYARKAARQARA
mvBIP
SEQ ID NO: 168
MVKLSLVAAMLLLLSLVAAMLLLLSAARA
Exemplary DNA sequence for mvBIP
SEQ ID NO: 169
ATGAAGCTGTCCCTGGTGGCCGCTATGCTGCTGCTGCTGTCTCTGGTCGCTGCCATGTTATTA
CTGCTGTCTGCCGCTAGGGCC
Exemplary DNA sequence for TATκκ28
SEQ ID NO: 170
TCTGATGCTGCCCAGCCTGCTAGAAGGGCCGCCAGGACAAAACTGGCCGCCTATGCCAGAAAA
GCCGCCAGACAGGCCAGAGCC
Exemplary DNA sequence for TATκκ28
SEQ ID NO: 171
AGCGACGCCGCTCAACCAGCTCGACGCGCCGCCAGAACCAAGCTGGCCGCCTACGCCCGGAAG
GCCGCCAGACAGGCCAGAGCC
Exemplary DNA sequence for TATκκ28
SEQ ID NO: 172
AGCGACGCCGCCCAGCCCGCCAGAAGAGCCGCCAGAACCAAGCTGGCCGCCTACGCCAGAAAG
GCCGCCAGACAGGCCAGAGCC
Exemplary DNA sequence for TATκκ28
SEQ ID NO: 173
TCTGATGCCGCCCAGCCTGCCAGACGGGCTGCACGGACGAAGCTGGCCGCCTACGCCAGAAAG
GCGGCCAGACAGGCCAGAGCC
TwinStrep-3cV2-TATκ28-hCDKL5-Flag-His-HPC4
(Amino Acid Sequence)
SEQ ID NO: 174
MSAWSHPQFEKGGGSGGGSGGSAWSHPQFEKGSLEVLFQGPDAAQPARRARRTKLAAYARKAA
RQARAGGGGSKIPNIGNVMNKFEILGVVGEGAYGVVLKCRHKETHEIVAIKKFKDSEENEEVK
ETTLRELKMLRTLKQENIVELKEAFRRRGKLYLVFEYVEKNMLELLEEMPNGVPPEKVKSYIY
QLIKAIHWCHKNDIVHRDIKPENLLISHNDVLKLCDFGFARNLSEGNNANYTEYVATRWYRSP
ELLLGAPYGKSVDMWSVGCILGELSDGQPLFPGESEIDQLFTIQKVLGPLPSEQMKLFYSNPR
FHGLRFPAVNHPQSLERRYLGILNSVLLDLMKNLLKLDPADRYLTEQCLNHPTFQTQRLLDRS
PSRSAKRKPYHVESSTLSNRNQAGKSTALQSHHRSNSKDIQNLSVGLPRADEGLPANESFLNG
NLAGASLSPLHTKTYQASSQPGSTSKDLTNNNIPHLLSPKEAKSKTEFDFNIDPKPSEGPGTK
YLKSNSRSQQNRHSFMESSQSKAGTLQPNEKQSRHSYIDTIPQSSRSPSYRTKAKSHGALSDS
KSVSNLSEARAQIAEPSTSRYFPSSCLDLNSPTSPTPTRHSDTRTLLSPSGRNNRNEGTLDSR
RTTTRHSKTMEELKLPEHMDSSHSHSLSAPHESFSYGLGYTSPFSSQQRPHRHSMYVTRDKVR
AKGLDGSLSIGQGMAARANSLQLLSPQPGEQLPPEMTVARSSVKETSREGTSSFHTRQKSEGG
VYHDPHSDDGTAPKENRHLYNDPVPRRVGSFYRVPSPRPDNSFHENNVSTRVSSLPSESSSGT
NHSKRQPAFDPWKSPENISHSEQLKEKEKQGFFRSMKKKKKKSQTVPNSDSPDLLTLQKSIHS
ASTPSSRPKEWRPEKISDLQTQSQPLKSLRKLLHLSSASNHPASSDPRFQPLTAQQTKNSFSE
IRIHPLSQASGGSSNIRQEPAPKGRPALQLPGQMDPGWHVSSVTRSATEGPSYSEQLGAKSGP
NGHPYNRTNRSRMPNLNDLKETALGAGGGGSLEVLFQGPDYKDHDGDYKDHDIDYKDDDDKDG
APHHHHHHEDQVDPRLIDGK
TwinStrep-3cV2-TATκ28-hCDKL5-Flag-His-HPC4 (DNA Sequence)
SEQ ID NO: 175
ATGTCAGCGTGGTCGCATCCTCAATTCGAAAAGGGCGGCGGTTCGGGTGGAGGAAGTGGCGGA
TCGGCCTGGTCTCACCCGCAATTCGAAAAGGGTTCCCTcGAgGTcCTTTTtCAAGGTCCcGAC
GCTGCTCAGCCTGCTCGCCGTGCCAGGAGAACCAAGCTGGCTGCCTACGCTCGTAAGGCTGCT
AGACAAGCTAGGGCCGGTGGCGGAGGTAGCAAGATCCCAAACATCGGTAACGTGATGAACAAG
TTCGAGATCCTGGGCGTGGTCGGTGAAGGCGCTTACGGAGTGGTCCTGAAGTGCAGGCACAAG
GAGACCCACGAAATCGTGGCCATCAAGAAGTTCAAGGACTCTGAGGAAAACGAGGAAGTCAAA
GAGACCACTCTGCGTGAACTGAAGATGCTGAGGACTCTGAAGCAGGAGAACATCGTCGAGCTG
AAGGAAGCTTTCCGCCGTAGGGGAAAGCTGTACCTGGTGTTCGAGTACGTCGAAAAGAACATG
CTGGAGCTGCTGGAGGAAATGCCAAACGGTGTGCCTCCCGAAAAGGTCAAGAGCTACATCTAC
CAGCTGATCAAGGCCATCCACTGGTGCCACAAGAACGACATCGTGCACCGTGACATCAAGCCT
GAGAACCTGCTGATCAGCCACAACGACGTCCTGAAGCTGTGCGACTTCGGTTTCGCTAGGAAC
CTGTCTGAGGGCAACAACGCTAACTACACTGAATACGTGGCCACCCGTTGGTACAGGTCTCCA
GAGCTGCTGCTGGGTGCCCCTTACGGCAAGTCTGTGGACATGTGGTCTGTCGGATGCATCCTG
GGTGAACTGAGCGACGGACAGCCCCTGTTCCCAGGAGAGTCTGAAATCGACCAGCTGTTCACC
ATCCAGAAGGTCCTGGGCCCCCTGCCAAGCGAGCAGATGAAGCTGTTCTACTCTAACCCCCGT
TTCCACGGACTGAGGTTCCCTGCTGTGAACCACCCCCAGAGCCTGGAAAGACGCTACCTGGGT
ATCCTGAACTCTGTCCTGCTGGACCTGATGAAGAACCTGCTGAAGCTGGACCCTGCTGACCGC
TACCTGACCGAGCAGTGCCTGAACCACCCCACTTTCCAGACCCAGAGACTGCTGGACCGCAGC
CCCTCTCGTTCAGCCAAGAGGAAGCCATACCACGTGGAATCCAGCACCCTGAGCAACCGTAAC
CAGGCTGGCAAGTCCACTGCCCTGCAGAGCCACCACAGGTCCAACAGCAAGGACATCCAAAAC
CTGTCAGTGGGACTGCCAAGGGCTGACGAGGGACTGCCAGCCAACGAATCCTTCCTGAACGGC
AACCTGGCTGGAGCCTCTCTGTCACCACTGCACACTAAGACCTACCAGGCTTCTTCACAGCCT
GGTTCCACTAGCAAGGACCTGACCAACAACAACATCCCACACCTGCTGTCTCCTAAGGAAGCT
AAATCAAAGACCGAGTTCGACTTCAACATCGACCCTAAGCCCTCCGAGGGACCTGGTACTAAG
TACCTGAAGTCTAACTCAAGATCCCAGCAGAACCGCCACTCATTCATGGAGTCCAGCCAGTCC
AAGGCTGGTACCCTGCAGCCCAACGAAAAGCAGTCCCGCCACAGCTACATCGACACCATCCCT
CAGTCTTCACGTAGCCCCTCTTACAGGACTAAGGCTAAGAGCCACGGCGCCCTGTCAGACTCC
AAGAGCGTGTCTAACCTGTCTGAGGCTAGAGCCCAGATCGCCGAACCTTCAACCTCCCGCTAC
TTCCCCTCCAGCTGCCTGGACCTGAACTCTCCCACTTCACCAACTCCTACCAGACACTCCGAC
ACTCGCACCCTGCTGAGCCCATCTGGTAGAAACAACCGCAACGAGGGCACCCTGGACTCACGT
AGGACCACTACCCGTCACTCCAAGACTATGGAGGAACTGAAGCTGCCAGAGCACATGGACTCT
TCACACTCACACTCCCTGAGCGCTCCTCACGAATCTTTCTCATACGGCCTGGGATACACCAGC
CCATTCTCCAGCCAGCAGCGTCCTCACAGGCACTCTATGTACGTGACTAGAGACAAGGTCCGC
GCTAAGGGACTGGACGGTTCCCTGTCTATCGGTCAGGGAATGGCTGCTAGGGCCAACTCTCTG
CAGCTGCTGTCACCCCAGCCAGGAGAGCAGCTGCCACCTGAAATGACCGTGGCTAGATCTTCA
GTCAAGGAGACTTCCCGCGAAGGCACCTCCAGCTTCCACACTAGACAGAAGTCAGAGGGCGGA
GTGTACCACGACCCTCACTCTGACGACGGAACTGCTCCCAAGGAAAACCGCCACCTGTACAAC
GACCCTGTGCCCAGACGCGTCGGATCCTTCTACCGTGTCCCAAGCCCTAGGCCCGACAACTCT
TTCCACGAGAACAACGTGAGCACCAGAGTCTCTTCACTGCCCTCTGAATCCAGCTCTGGCACT
AACCACTCAAAGCGCCAGCCTGCTTTCGACCCCTGGAAGTCCCCAGAGAACATCTCTCACTCA
GAACAGCTGAAGGAGAAGGAAAAGCAGGGATTCTTCCGCTCAATGAAGAAGAAGAAGAAGAAG
TCCCAGACCGTGCCCAACTCCGACAGCCCAGACCTGCTGACCCTGCAGAAGTCAATCCACTCT
GCCTCAACTCCTTCATCCAGACCCAAGGAGTGGCGCCCCGAAAAGATCTCCGACCTGCAGACT
CAGTCCCAGCCACTGAAGAGCCTGCGTAAGCTGCTGCACCTGAGCTCTGCTTCCAACCACCCT
GCCTCATCCGACCCACGTTTCCAGCCTCTGACTGCTCAGCAGACCAAGAACTCCTTCAGCGAG
ATCAGGATCCACCCACTGTCCCAGGCTAGCGGTGGCAGCTCTAACATCCGTCAGGAACCAGCT
CCTAAGGGACGTCCAGCTCTGCAGCTGCCTGGTCAGATGGACCCAGGCTGGCACGTGTCATCC
GTCACTAGATCAGCTACCGAGGGACCATCTTACTCAGAACAGCTGGGTGCCAAGTCAGGCCCC
AACGGACACCCATACAACCGCACCAACCGTTCCAGGATGCCTAACCTGAACGACCTGAAGGAG
ACTGCTCTGGGgGCCGGAGGTGGCGGATCCCTgGAaGTgCTgTTcCAgGGcCCTGACTACAAG
GACCACGACGGTGACTACAAAGATCACGACATCGACTACAAGGACGACGACGACAAGGACGGT
GCCCCACACCACCACCACCACCACGAAGATCAGGTGGATCCTCGCCTGATCGATGGCAAGTAA
TwinStrep-3cV2-TATκ28-hCDKL5-Flag-His-TwinStrep
(Amino Acid Sequence)
SEQ ID NO: 176
MSAWSHPQFEKGGGSGGGSGGSAWSHPQFEKGSLEVLFQGPDAAQPARRARRTKLAAYARKAA
RQARAGGGGSKIPNIGNVMNKFEILGVVGEGAYGVVLKCRHKETHEIVAIKKFKDSEENEEVK
ETTLRELKMLRTLKQENIVELKEAFRRRGKLYLVFEYVEKNMLELLEEMPNGVPPEKVKSYIY
QLIKAIHWCHKNDIVHRDIKPENLLISHNDVLKLCDFGFARNLSEGNNANYTEYVATRWYRSP
ELLLGAPYGKSVDMWSVGCILGELSDGQPLFPGESEIDQLFTIQKVLGPLPSEQMKLFYSNPR
FHGLRFPAVNHPQSLERRYLGILNSVLLDLMKNLLKLDPADRYLTEQCLNHPTFQTQRLLDRS
PSRSAKRKPYHVESSTLSNRNQAGKSTALQSHHRSNSKDIQNLSVGLPRADEGLPANESFLNG
NLAGASLSPLHTKTYQASSQPGSTSKDLTNNNIPHLLSPKEAKSKTEFDFNIDPKPSEGPGTK
YLKSNSRSQQNRHSFMESSQSKAGTLQPNEKQSRHSYIDTIPQSSRSPSYRTKAKSHGALSDS
KSVSNLSEARAQIAEPSTSRYFPSSCLDLNSPTSPTPTRHSDTRTLLSPSGRNNRNEGTLDSR
RTTTRHSKTMEELKLPEHMDSSHSHSLSAPHESFSYGLGYTSPFSSQQRPHRHSMYVTRDKVR
AKGLDGSLSIGQGMAARANSLQLLSPQPGEQLPPEMTVARSSVKETSREGTSSFHTRQKSEGG
VYHDPHSDDGTAPKENRHLYNDPVPRRVGSFYRVPSPRPDNSFHENNVSTRVSSLPSESSSGT
NHSKRQPAFDPWKSPENISHSEQLKEKEKQGFFRSMKKKKKKSQTVPNSDSPDLLTLQKSIHS
ASTPSSRPKEWRPEKISDLQTQSQPLKSLRKLLHLSSASNHPASSDPRFQPLTAQQTKNSFSE
IRIHPLSQASGGSSNIRQEPAPKGRPALQLPGQMDPGWHVSSVTRSATEGPSYSEQLGAKSGP
NGHPYNRTNRSRMPNLNDLKETALGAGGGGSLEVLFQGPDYKDHDGDYKDHDIDYKDDDDKDG
APHHHHHHSAWSHPQFEKGGGSGGGSGGSAWSHPQFEK*
TwinStrep-3cV2-TATκ28-hCDKL5-Flag-His-TwinStrep (DNA Sequence)
SEQ ID NO: 177
ATGTCAGCGTGGTCGCATCCTCAATTCGAAAAGGGCGGCGGTTCGGGTGGAGGAAGTGGCGGA
TCGGCCTGGTCTCACCCGCAATTCGAAAAGGGTTCCCTcGAgGTcCTgTTtCAgGGcCCcGAC
GCTGCTCAGCCTGCTCGCCGTGCCAGGAGAACCAAGCTGGCTGCCTACGCTCGTAAGGCTGCT
AGACAAGCTAGGGCCGGTGGCGGAGGTAGCAAGATCCCAAACATCGGTAACGTGATGAACAAG
TTCGAGATCCTGGGCGTGGTCGGTGAAGGCGCTTACGGAGTGGTCCTGAAGTGCAGGCACAAG
GAGACCCACGAAATCGTGGCCATCAAGAAGTTCAAGGACTCTGAGGAAAACGAGGAAGTCAAA
GAGACCACTCTGCGTGAACTGAAGATGCTGAGGACTCTGAAGCAGGAGAACATCGTCGAGCTG
AAGGAAGCTTTCCGCCGTAGGGGAAAGCTGTACCTGGTGTTCGAGTACGTCGAAAAGAACATG
CTGGAGCTGCTGGAGGAAATGCCAAACGGTGTGCCTCCCGAAAAGGTCAAGAGCTACATCTAC
CAGCTGATCAAGGCCATCCACTGGTGCCACAAGAACGACATCGTGCACCGTGACATCAAGCCT
GAGAACCTGCTGATCAGCCACAACGACGTCCTGAAGCTGTGCGACTTCGGTTTCGCTAGGAAC
CTGTCTGAGGGCAACAACGCTAACTACACTGAATACGTGGCCACCCGTTGGTACAGGTCTCCA
GAGCTGCTGCTGGGTGCCCCTTACGGCAAGTCTGTGGACATGTGGTCTGTCGGATGCATCCTG
GGTGAACTGAGCGACGGACAGCCCCTGTTCCCAGGAGAGTCTGAAATCGACCAGCTGTTCACC
ATCCAGAAGGTCCTGGGCCCCCTGCCAAGCGAGCAGATGAAGCTGTTCTACTCTAACCCCCGT
TTCCACGGACTGAGGTTCCCTGCTGTGAACCACCCCCAGAGCCTGGAAAGACGCTACCTGGGT
ATCCTGAACTCTGTCCTGCTGGACCTGATGAAGAACCTGCTGAAGCTGGACCCTGCTGACCGC
TACCTGACCGAGCAGTGCCTGAACCACCCCACTTTCCAGACCCAGAGACTGCTGGACCGCAGC
CCCTCTCGTTCAGCCAAGAGGAAGCCATACCACGTGGAATCCAGCACCCTGAGCAACCGTAAC
CAGGCTGGCAAGTCCACTGCCCTGCAGAGCCACCACAGGTCCAACAGCAAGGACATCCAAAAC
CTGTCAGTGGGACTGCCAAGGGCTGACGAGGGACTGCCAGCCAACGAATCCTTCCTGAACGGC
AACCTGGCTGGAGCCTCTCTGTCACCACTGCACACTAAGACCTACCAGGCTTCTTCACAGCCT
GGTTCCACTAGCAAGGACCTGACCAACAACAACATCCCACACCTGCTGTCTCCTAAGGAAGCT
AAATCAAAGACCGAGTTCGACTTCAACATCGACCCTAAGCCCTCCGAGGGACCTGGTACTAAG
TACCTGAAGTCTAACTCAAGATCCCAGCAGAACCGCCACTCATTCATGGAGTCCAGCCAGTCC
AAGGCTGGTACCCTGCAGCCCAACGAAAAGCAGTCCCGCCACAGCTACATCGACACCATCCCT
CAGTCTTCACGTAGCCCCTCTTACAGGACTAAGGCTAAGAGCCACGGCGCCCTGTCAGACTCC
AAGAGCGTGTCTAACCTGTCTGAGGCTAGAGCCCAGATCGCCGAACCTTCAACCTCCCGCTAC
TTCCCCTCCAGCTGCCTGGACCTGAACTCTCCCACTTCACCAACTCCTACCAGACACTCCGAC
ACTCGCACCCTGCTGAGCCCATCTGGTAGAAACAACCGCAACGAGGGCACCCTGGACTCACGT
AGGACCACTACCCGTCACTCCAAGACTATGGAGGAACTGAAGCTGCCAGAGCACATGGACTCT
TCACACTCACACTCCCTGAGCGCTCCTCACGAATCTTTCTCATACGGCCTGGGATACACCAGC
CCATTCTCCAGCCAGCAGCGTCCTCACAGGCACTCTATGTACGTGACTAGAGACAAGGTCCGC
GCTAAGGGACTGGACGGTTCCCTGTCTATCGGTCAGGGAATGGCTGCTAGGGCCAACTCTCTG
CAGCTGCTGTCACCCCAGCCAGGAGAGCAGCTGCCACCTGAAATGACCGTGGCTAGATCTTCA
GTCAAGGAGACTTCCCGCGAAGGCACCTCCAGCTTCCACACTAGACAGAAGTCAGAGGGCGGA
GTGTACCACGACCCTCACTCTGACGACGGAACTGCTCCCAAGGAAAACCGCCACCTGTACAAC
GACCCTGTGCCCAGACGCGTCGGATCCTTCTACCGTGTCCCAAGCCCTAGGCCCGACAACTCT
TTCCACGAGAACAACGTGAGCACCAGAGTCTCTTCACTGCCCTCTGAATCCAGCTCTGGCACT
AACCACTCAAAGCGCCAGCCTGCTTTCGACCCCTGGAAGTCCCCAGAGAACATCTCTCACTCA
GAACAGCTGAAGGAGAAGGAAAAGCAGGGATTCTTCCGCTCAATGAAGAAGAAGAAGAAGAAG
TCCCAGACCGTGCCCAACTCCGACAGCCCAGACCTGCTGACCCTGCAGAAGTCAATCCACTCT
GCCTCAACTCCTTCATCCAGACCCAAGGAGTGGCGCCCCGAAAAGATCTCCGACCTGCAGACT
CAGTCCCAGCCACTGAAGAGCCTGCGTAAGCTGCTGCACCTGAGCTCTGCTTCCAACCACCCT
GCCTCATCCGACCCACGTTTCCAGCCTCTGACTGCTCAGCAGACCAAGAACTCCTTCAGCGAG
ATCAGGATCCACCCACTGTCCCAGGCTAGCGGTGGCAGCTCTAACATCCGTCAGGAACCAGCT
CCTAAGGGACGTCCAGCTCTGCAGCTGCCTGGTCAGATGGACCCAGGCTGGCACGTGTCATCC
GTCACTAGATCAGCTACCGAGGGACCATCTTACTCAGAACAGCTGGGTGCCAAGTCAGGCCCC
AACGGACACCCATACAACCGCACCAACCGTTCCAGGATGCCTAACCTGAACGACCTGAAGGAG
ACTGCTCTGGGgGCCGGAGGTGGCGGATCCCTGGAAGTGCTTTTCCAAGGTCCcGACTACAAG
GACCACGACGGTGACTACAAAGATCACGACATCGACTACAAGGACGACGACGACAAGGACGGT
GCCCCACACCACCACCACCACCACTCTGCATGGTCGCATCCTCAATTCGAGAAGGGGGGTGGC
AGCGGAGGGGGTTCCGGCGGATCAGCCTGGAGTCACCCACAGTTTGAAAAATAA
```

1. A composition comprising:
a gene therapy delivery system; and
a CDKL5 polynucleotide encoding a CDKL5 polypeptide, wherein the CDKL5 polypeptide has at least 98% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 26.

2. The composition of claim 1, wherein the CDKL5 polypeptide has at least 98% sequence identity to SEQ ID NO: 1 or SEQ ID NO: 26.

3. The composition of claim 1, wherein the CDKL5 polynucleotide has at least 90% sequence identity to SEQ ID NO: 123.

4. The composition of claim 1, wherein the CDKL5 polypeptide has at least 98% sequence identity to SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

5. The composition of claim 1, wherein the CDKL5 polypeptide has at least 98% sequence identity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 or SEQ ID NO: 25.

6. The composition of claim 1, wherein the CDKL5 polynucleotide has at least 90% sequence identity to SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147 or 1 SEQ ID NO: 149.

7. The composition of claim 1, wherein the gene therapy delivery system comprises one or more of a viral vector, a liposome, a lipid-nucleic acid nanoparticle, an exosome and a gene editing system.

8. The composition of claim 7, wherein the gene editing system comprises one or more of Clustered Regularly Interspaced Short Palindromic Repeat (CRISPR) associated protein 9 (CRISPR-Cas-9), Transcription activator-like effector nuclease (TALEN) or ZNF (Zinc finger protein).

9. (canceled)

10. The composition of claim 1, wherein the gene therapy delivery system comprises a viral vector comprising one or more of an adenoviral vector, an adeno-associated viral vector, a lentiviral vector, a retroviral vector, a poxviral vector or a herpes simplex viral vector.

11. The composition of claim 9, wherein the viral vector comprises a viral polynucleotide operably linked to the CDKL5 polynucleotide.

12. The composition of claim 11, wherein the viral vector comprises at least one inverted terminal repeat (ITR).

13. The composition of claim 11, further comprising one or more of an SV40 intron, a polyadenylation signal or a stabilizing element.

14. The composition of claim 11, further comprising a promoter.

15. The composition of claim 14, wherein the promoter has at least 90% sequence identity to SEQ ID NO: 29 or SEQ ID NO: 30.

16. The composition of claim 1, further comprising a polynucleotide encoding a cell-penetrating polypeptide.

17. The composition of claim 16, wherein the cell-penetrating polypeptide has at least 90% sequence identity to SEQ ID NO: 32, SEQ ID NO: 34, SEQ ID NO: 35, SEQ ID NO: 36, SEQ ID NO: 37 or SEQ ID NO: 167.

18. (canceled)

19. The composition of claim 1, further comprising a polynucleotide encoding a leader signal polypeptide.

20. The composition of claim 19, wherein the leader signal polypeptide has at least 90% sequence identity to SEQ ID NO: 38, SEQ ID NO: 39, SEQ ID NO: 40, SEQ ID NO: 41, SEQ ID NO: 42, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166 or SEQ ID NO: 168.

21. (canceled)

22. A pharmaceutical formulation comprising the composition of claim 1; and a pharmaceutically acceptable carrier.

23. A method of treating a CDKL5-mediated neurological disorder, the method comprising administering the formulation of claim 22 to a patient in need thereof.

24-77. (canceled)
